(19) United States
(12) Patent Application Publication
(10) Pub. No.: US 2025/0249052 A1
(43) Pub. Date: Aug. 7, 2025

(54) COMPOSITIONS AND METHODS FOR MUSCLE DISORDERS

(71) Applicant: KATE THERAPEUTICS, INC., San Diego, CA (US)

(72) Inventors: Mark Fielden, San Diego, CA (US); Mohammadsharif Tabebordbar, San Diego, CA (US); Jana Jenquin, San Diego, CA (US); Brian Spencer, San Diego, CA (US)

(21) Appl. No.: 18/854,776

(22) PCT Filed: Apr. 7, 2023
(86) PCT No.: PCT/US2023/065520

§ 371 Date: Oct. 7, 2024

Related U.S. Application Data
(60) Provisional application No. 63/328,962, filed on Apr. 8, 2022.
(60) Provisional application No. 63/328,606, filed on Apr. 7, 2022.

(51) Int. Cl. A61K 35/76; C12N 15/113; C12N 15/86

(52) U.S. Cl. CPC A61K 35/76; C12N 15/113; C12N 15/86; C12N 2310/141; C12N 2330/51; C12N 2750/14145

(57) ABSTRACT

The invention discloses approaches to reduce expression of a gene in a cell.

### SEQUENCE LISTING

This application contains a sequence listing in electronic form as an extensible Markup Language (XML) form filed via the Patent Center and is hereby incorporated by reference in its entirety. The XML-formatted sequence listing, created on Apr. 6, 2023, is named KATE-013-02WO-Scq.xml, and is 1.13 MB in size.

### FIELD OF THE INVENTION

The invention relates to the medical treatment of muscle weakening disorders, including facioscapulohumeral muscular dystrophy.

### BACKGROUND

Genetic disorders are a major source of disease burden, and many of them have few or no medical or curative treatments. Along with other genetically linked muscle wasting disorders, Facioscapulohumeral Muscular Dystrophy (“FSHD”) is a genetic disorder that causes progressively increasing weakness and atrophy of the muscles. FSHD is so named because the muscles in the face, shoulder, and upper arm are the most affected, but it also affects other muscles in the body, including the legs, eyes, heart, hip, or abdominal muscles. FSHD causes progressively worsening symptoms that may result in asymmetric weakness

FSHD is a genetic disorder caused by a genetic mutation that leads to inappropriate expression of the DUX4 gene on chromosome 4. DUX4 is the double homeobox protein 4 gene. FSHD can be inherited by just one parent because it is an autosomal dominant genetic disorder. FSHD often affects patients before the age of 20 and has an estimated prevalence in the United States of 4 cases per 100,000 individuals. There is currently no medical treatment to arrest or reverse the muscular effects of FSHD.

### SUMMARY

Aspects of the invention provide a method of inhibiting expression of a gene in a cell, the method comprising administering to a subject the adeno-associated virus (AAV) vector comprising a promoter sequence, a miRNA strand that binds to DUX4-FL, and a capsid protein comprising at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide. Methods of inhibiting that gene expression may include providing the AAV vector with any of the indicated permutations of the embodiment of the invention.

For example, the promoter sequence is a promoter sequence transcribed by RNA polymerase II or III, U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or TH promoter sequence.

The miRNA strand or miRNA scaffold may be selected from the group of miDUX4, miRN92, miRNA-17, miRNA-18a, miRNA-19a, miRNA-20a, miRNA-19b-1, mi-RNA-26a, miRNA-126, miRNA-335, let-7a and let-7b, miRNA-34, miR-34a, miRNA-10b, miRNA-208, miRNA-499, miRNA-195, miRNA-29a, miRNA-29b, or miRNA-29c.

The miRNA strand may be selected from the group of SEQ ID NOs. 1250-1305.

The miRNA strand may further comprise 5-6 thymidines at the 5′ end.

Advantageously, the capsid protein comprises at least one modification that results in reduced liver-tropism of the AAV vector. The capsid protein may comprise at least one modification that results in preferential targeting of the AAV vector to muscle tissue.

The capsid protein may be selected from the sequences in Tables 1-4.

The vector may further comprises a nuclear export sequence enabling nuclear spreading.

Aspects of the invention also provide a methods of inhibiting expression of a gene in a cell by administering AAV vectors of the invention. Methods may comprise administering to a subject an adeno-associated virus (AAV) vector comprising a promoter sequence, a miRNA strand that binds to DUX4-FL, and a capsid protein comprising at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

TABLE 1
MyoAAV (eMyoAAV) Capsid Variants

| | | SEQ | | SEQ |
|---|---|---|---|---|
| | | ID | | ID |
| RANK | N-MER MOTIF | NO.: | ENCODING SEQUENCE | NO.: |
| 1 | AEGRGDQYTR | 177 | GCTGAGGGTAGAGGAGACCAGTATACTCGT | 181 |
| 2 | GPGRGDQTTL | 178 | GGGCCGGGTAGAGGAGACCAGACTACGTTG | 182 |
| 3 | AEGRGDQYTR | 177 | GCGGAAGGCAGAGGAGACCAATACACAAGG | 183 |
| 4 | ATGRGDLGQA | 179 | GCGACTGGTAGAGGAGACCTGGGTCAGGCT | 184 |
| 5 | AVARGDQGLI | 180 | GCGGTGGCGAGAGGAGACCAGGGTCTTATT | 185 |
| 6 | NISRGDQGYQ | 6 | AACATCTCCAGAGGAGACCAAGGTTACCAA | 186 |
| 7 | APARGDQGSQ | 7 | GCGCCGGCTAGAGGAGACCAGGGGAGTCAG | 187 |
| 8 | AVSRGDRMEF | 8 | GCCGTTAGCAGAGGAGACCGGATGGAATTC | 188 |
| 9 | QMGRGDMGIK | 10 | CAGATGGGTAGAGGAGACATGGGGATTAAG | 189 |
| 10 | EYRRGDKADI | 11 | GAGTATCGGAGAGGAGACAAGGCGGATATT | 190 |
| 11 | ESRRGDKEPL | 12 | GAGAGTCGGAGAGGAGACAAGGAGCCGCTG | 191 |
| 12 | AHMRGDLGGT | 13 | GCACACATGAGAGGAGACCTAGGCGGCACG | 192 |
| 13 | VWQRGDKMDM | 14 | GTGTGGCAAAGAGGAGACAAAATGGACATG | 193 |
| 14 | SIGRGDTGHM | 15 | TCTATTGGGAGAGGAGACACGGGTCATATG | 194 |
| 15 | NIARGDAGQY | 16 | AATATTGCTAGAGGAGACGCTGGTCAGTAT | 195 |
| 16 | AVARGDQGLI | 180 | GCAGTAGCAAGAGGAGACCAAGGCTTAATC | 196 |
| 17 | SVSRGDQGLH | 17 | TCGGTCTCGAGAGGAGACCAAGGATTGCAC | 197 |
| 18 | EYRRGDKADI | 11 | GAATACAGGAGAGGAGACAAAGCAGACATC | 198 |
| 19 | AHMRGDLGGT | 13 | GCGCATATGAGAGGAGACTTGGGGGGGACT | 199 |
| 20 | QIGRGDITHG | 18 | CAGATTGGTAGAGGAGACATTACTCATGGG | 200 |
| 21 | EVRRGDLIIGT | 19 | GAAGTCAGAAGAGGAGACTTGCACGGGACA | 201 |
| 22 | SVSRGDVHTM | 20 | AGTGTCTCAAGAGGAGACGTGCACACGATG | 202 |
| 23 | MVTRGDLGTR | 21 | ATGGTGACTAGAGGAGACCTTGGTACGCGG | 203 |
| 24 | NGGRGDTTHF | 22 | AACGGCGGGAGAGGAGACACGACGCACTTC | 204 |
| 25 | TMGRGDMNSL | 23 | ACGATGGGGAGAGGAGACATGAATTCGCTT | 205 |
| 26 | AISRGDQGLS | 24 | GCGATTAGTAGAGGAGACCAGGGTCTTTCT | 206 |
| 27 | NDARGDKSTY | 25 | AACGACGCAAGAGGAGACAAATCCACATAC | 207 |
| 28 | VGLRGDLTGS | 26 | GTGGGGCTGAGAGGAGACTTGACGGGTTCG | 208 |
| 29 | EMRRGDLGTS | 27 | GAGATGAGGAGAGGAGACCTGGGGACGAGT | 209 |
| 30 | AMSRGDMGMA | 28 | GCGATGAGTAGAGGAGACATGGGGATGGCG | 210 |
| 31 | ATGRGDLGQA | 179 | GCAACGGGCAGAGGAGACTTAGGCCAAGCA | 211 |
| 32 | NVARGDQVNY | 29 | AACGTAGCAAGAGGAGACCAAGTTAACTAC | 212 |
| 33 | SIGRGDTGHM | 15 | AGCATCGGAAGAGGAGACACCGGCCACATG | 213 |
| 34 | ASVRGDLSGS | 30 | GCATCCGTAAGAGGAGACCTATCAGGCTCA | 214 |
| 35 | SAARGDTERL | 31 | AGCGCCGCGAGAGGAGACACTGAACGGCTA | 215 |
| 36 | AMSRGDMGMA | 28 | GCCATGTCAAGAGGAGACATGGGTATGGCT | 216 |
| 37 | SPSRGDQGRT | 9 | AGTCCATCGAGAGGAGACCAAGGACGCACT | 217 |
| 38 | VPGRGDLNTM | 32 | GTGCCTGGGAGAGGAGACCTTAATACTATG | 218 |
| 39 | GPGRGDQTTL | 178 | GGTCCTGGGAGAGGAGACCAAACGACCCTT | 219 |
| 40 | TSVRGDHGTL | 33 | ACATCGGTCAGAGGAGACCACGGAACATTG | 220 |
| 41 | TPSRGDLGQT | 34 | ACGCCTTCGAGAGGAGACCTTGGGCAGACT | 221 |
| 42 | AGHRGDTGVI | 35 | GCTGGACACAGAGGAGACACAGGAGTCATC | 222 |
| 43 | APARGDQGSM | 36 | GCGCCGGCGAGAGGAGACCAGGGTAGTATG | 223 |
| 44 | MSLRGDLNGS | 37 | ATGTCGTTGAGAGGAGACTTGAATGGGTCG | 224 |
| 45 | EAKRGDVHSI | 38 | GAGGCTAAGAGAGGAGACGTGCATTCTATT | 225 |
| 46 | SAQRGDVQAV | 39 | AGTGCTCAGAGAGGAGACGTGCAGGCGGTG | 226 |
| 47 | STARGDQGDR | 40 | AGTACGGCGAGAGGAGACCAGGGGGATAGG | 227 |
| 48 | QISRGDLGIN | 41 | CAGATTAGTAGAGGAGACCTGGGGATTAAT | 228 |
| 49 | TFTRGDMTMN | 42 | ACGTTCACAAGAGGAGACATGACGATGAAC | 229 |
| 50 | QEGRGDLNMR | 43 | CAAGAAGGAAGAGGAGACCTGAACATGAGG | 230 |
| 51 | AHARGDTSSL | 44 | GCACACGCAAGAGGAGACACCAGCTCCCTG | 231 |
| 52 | TNGRGDAGTL | 45 | ACGAATGGGAGAGGAGACGCTGGGACTCTG | 232 |
| 53 | AFGRGDQGQL | 46 | GCTTTCGGCAGAGGAGACCAAGGGCAACTA | 233 |
| 54 | ADGRGDRSSL | 47 | GCTGATGGGAGAGGAGACCGTTCGTCTCTG | 234 |
| 55 | DGRRGDGHSL | 48 | GACGGACGCAGAGGAGACGGACACAGCCTT | 235 |
| 56 | EKLRGDLHST | 49 | GAGAAGTTGAGAGGAGACCTTCATTCGACT | 236 |
| 57 | EARRGDASAM | 50 | GAGGCGCGTAGAGGAGACGCTTCGGCGATG | 237 |
| 58 | MGTRGDKMDF | 51 | ATGGGAACGAGAGGAGACAAAATGGACTTC | 238 |
| 59 | STSRGDRESY | 52 | TCGACTTCGAGAGGAGACCGGGAGTCGTAT | 239 |
| 60 | CQPRGDTTRC | 53 | TGTCAGCCGAGAGGAGACACGACTCGGTGT | 240 |
| 61 | MHTRGDKMDF | 54 | ATGCACACGAGAGGAGACAAAATGGACTTC | 241 |
| 62 | YSARGDTSGL | 55 | TACAGTGCCAGAGGAGACACAAGTGGCCTG | 242 |
| 63 | STMRGDHEKL | 56 | TCGACGATGAGAGGAGACCATGAGAAGTTG | 243 |
| 64 | TQMRGDGVTL | 57 | ACCCAAATGAGAGGAGACGGGGTCACACTA | 244 |
| 65 | TTRRGDMGDN | 58 | ACTACTCGTAGAGGAGACATGGGGGATAAT | 245 |
| 66 | ANGRGDRLEL | 59 | GCGAATGGTAGAGGAGACAGGCTGGAGTTG | 246 |
| 67 | AVSRGDRMEF | 8 | GCTGTGTCTAGAGGAGACAGGATGGAGTTT | 247 |
| 68 | ENQRGDLSGR | 60 | GAGAATCAGAGAGGAGACTTGTCGGGTCGG | 248 |
| 69 | STGRGDLGQA | 61 | TCGACTGGTAGAGGAGACCTGGGGCAGGCT | 249 |
| 70 | MHTRGDKMDF | 54 | ATGCATACTAGAGGAGACAAGATGGATTTT | 250 |
| 71 | SSARGDYSEV | 62 | AGTAGTGCGAGAGGAGACTATAGTGAGGTG | 251 |
| 72 | AFGRGDQGQL | 46 | GCGTTTGGGAGAGGAGACCAGGGTCAGCTT | 252 |
| 73 | STARGDAATY | 63 | AGTACGGCGAGAGGAGACGCGGCGACTTAT | 253 |
| 74 | VNTRGDTQKL | 64 | GTCAACACAAGAGGAGACACTCAAAAACTT | 254 |
| 75 | AMSRGDHASL | 65 | GCGATGAGTAGAGGAGACCATGCGTCGCTT | 255 |
| 76 | QAARGDVNKL | 66 | CAGGCTGCTAGAGGAGACGTTAATAAGCTG | 256 |
| 77 | RGTRGDTVEL | 67 | AGGGGTACGAGAGGAGACACGGTTGAGTTG | 257 |
| 78 | TTRRGDMGDN | 58 | ACCACCCGGAGAGGAGACATGGGAGACAAC | 258 |
| 79 | ASTRGDYAGV | 68 | GCTAGTACGAGAGGAGACTATGCGGGTGTT | 259 |
| 80 | DDRRGDKLPL | 69 | GACGACAGGAGAGGAGACAAACTGCCCCTT | 260 |
| 81 | SGSRGDLNAV | 70 | TCGGGGTCGAGAGGAGACCTGAATGCGGTG | 261 |
| 82 | EYRRGDQQIQ | 71 | GAGTATAGGAGAGGAGACCAGCAGATTCAG | 262 |
| 83 | TYVRGDRAEV | 72 | ACGTACGTCAGAGGAGACAGAGCAGAAGTG | 263 |
| 84 | QSLRGDLNGS | 73 | CAATCACTAAGAGGAGACTTGAACGGATCC | 264 |
| 85 | EGRRGDTTSL | 74 | GAGGGTCGGAGAGGAGACACGACGTCGTTG | 265 |
| 86 | AENRGDRSSL | 75 | GCGGAGAATAGAGGAGACCGGTCGAGTTTG | 266 |
| 87 | VAGRGDRSEL | 76 | GTGGCTGGGAGAGGAGACCGTAGTGAGCTG | 267 |
| 88 | DPARGDIGAR | 77 | GATCCGGCGAGAGGAGACATTGGTGCGCGT | 268 |
| 89 | EMRRGDLGTS | 27 | GAAATGCGCAGAGGAGACTTGGGAACTTCA | 269 |
| 90 | VWQRGDKMDM | 14 | GTTTGGCAGAGAGGAGACAAGATGGATATG | 270 |
| 91 | VIGRGDKALQ | 78 | GTGATTGGGAGAGGAGACAAGGCGCTTCAG | 271 |
| 92 | SFERGDKNSL | 79 | AGCTTCGAAAGAGGAGACAAAAACTCTCTT | 272 |
| 93 | QIARGDIASV | 80 | CAGATTGCTAGAGGAGACATTGCTAGTGTG | 273 |
| 94 | TISRGDLGGA | 81 | ACGATTAGTAGAGGAGACCTGGGTGGGGCT | 274 |
| 95 | AMIRGDMTHS | 82 | GCGATGATTAGAGGAGACATGACGCATAGT | 275 |
| 96 | TSVRGDHGTL | 33 | ACGAGTGTGAGAGGAGACCATGGGACGCTG | 276 |
| 97 | TMRRGDLNDS | 83 | ACGATGCGGAGAGGAGACCTGAATGATAGT | 277 |
| 98 | AYSRGDLGNH | 84 | GCCTACTCCAGAGGAGACCTAGGCAACCAC | 278 |
| 99 | EERRGDTHRL | 85 | GAGGAGCGGAGAGGAGACACTCATCGGCTG | 279 |
| 100 | SQLRGDAGTI | 86 | TCACAACTAAGAGGAGACGCGGGGACAATC | 280 |
| 101 | AMVRGDAHQL | 87 | GCGATGGTGAGAGGAGACGCGCATCAGCTG | 281 |
| 102 | TTSRGDLNSV | 88 | ACGACTAGTAGAGGAGACTTGAATTCGGTT | 282 |
| 103 | WEQRGDMIGK | 89 | TGGGAGCAGAGAGGAGACATGATTGGGAAG | 283 |
| 104 | YTTRGDLQSN | 90 | TACACCACCAGAGGAGACCTCCAATCAAAC | 284 |
| 105 | MSQRGDLSHQ | 91 | ATGTCGCAGAGAGGAGACCTTTCGCATCAG | 285 |
| 106 | ASTRGDTTHM | 92 | GCGAGTACTAGAGGAGACACGACGCATATG | 286 |
| 107 | SDTRGDTHRL | 93 | TCAGACACCAGAGGAGACACCCACAGACTG | 287 |
| 108 | TLGRGDQYTS | 94 | ACGCTGGGTAGAGGAGACCAGTATACGAGT | 288 |
| 109 | SYARGDVREV | 95 | TCGTATGCGAGAGGAGACGTTAGGGAGGTT | 289 |
| 110 | ETRRGDLSQN | 96 | GAGACTCGTAGAGGAGACCTGAGTCAGAAT | 290 |
| 111 | TIGRGDLMDK | 97 | ACAATCGGGAGAGGAGACCTGATGGACAAA | 291 |
| 112 | SYTRGDLSQN | 98 | AGTTACACCAGAGGAGACCTTAGCCAAAAC | 292 |
| 113 | SKLRGDASEI | 99 | TCGAAGCTGAGAGGAGACGCTAGTGAGATT | 293 |
| 114 | SMARGDKAEI | 100 | TCGATGGCGAGAGGAGACAAGGCGGAGATT | 294 |
| 115 | GAVRGDHSSL | 101 | GGAGCCGTCAGAGGAGACCACTCATCGTTA | 295 |
| 116 | METRGDMSNR | 102 | ATGGAGACGAGAGGAGACATGAGTAATCGT | 296 |
| 117 | AYTRGDKDAL | 103 | GCGTACACAAGAGGAGACAAAGACGCACTA | 297 |
| 118 | ENRRGDITQS | 104 | GAGAATCGGAGAGGAGACATTACGCAGTCG | 298 |
| 119 | STGRGDLGQA | 61 | TCCACGGGCAGAGGAGACCTTGGACAAGCA | 299 |
| 120 | GAVRGDHSSL | 101 | GGTGCTGTTAGAGGAGACCATAGTAGTCTT | 300 |
| 121 | SQLRGDAGTI | 86 | TCTCAGCTTAGAGGAGACGCTGGTACGATT | 301 |
| 122 | MLLRGDKNDF | 105 | ATGCTCCTAAGAGGAGACAAAAACGACTTC | 302 |
| 123 | KVQRGDASDW | 106 | AAGGTGCAGAGAGGAGACGCGAGTGATTGG | 303 |
| 124 | AFTRGDMQTA | 107 | GCGTTTACGAGAGGAGACATGCAGACTGCT | 304 |
| 125 | QPKRGDVQAQ | 108 | CAGCCTAAGAGAGGAGACGTTCAGGCTCAG | 305 |
| 126 | ASLRGDMGVS | 109 | GCTAGTCTGAGAGGAGACATGGGGGTGAGT | 306 |
| 127 | AYSRGDKESF | 110 | GCCTACTCCAGAGGAGACAAAGAATCGTTC | 307 |
| 128 | RFQRGDLTDA | 111 | CGGTTCCAAAGAGGAGACTTGACCGACGCT | 308 |
| 129 | SDARGDTMKL | 112 | TCTGATGCTAGAGGAGACACGATGAAGCTG | 309 |
| 130 | SGARGDTVSL | 113 | AGCGGGGCCAGAGGAGACACAGTAAGTCTC | 310 |
| 131 | DEKRGDQKHL | 114 | GATGAGAAGAGAGGAGACCAGAAGCATCTT | 311 |
| 132 | NGVRGDVQNF | 115 | AACGGAGTAAGAGGAGACGTCCAAAACTTC | 312 |
| 133 | MTLRGDIGGS | 116 | ATGACGTTGAGAGGAGACCTGGGTGGGTCG | 313 |
| 134 | EARRGDASAM | 50 | GAGGCTAGGAGAGGAGACGCTAGTGCGATG | 314 |
| 135 | GQERGDLGTR | 117 | GGGCAGGAGAGAGGAGACCTGGGGACICGG | 315 |
| 136 | RGARGDLVDA | 118 | AGAGGCGCCAGAGGAGACCTAGTCGACGCT | 316 |
| 137 | QYSRGDHTDL | 119 | CAATACTCCAGAGGAGACCACACCGACCTT | 317 |
| 138 | MNTRGDVHAM | 120 | ATGAACACCAGAGGAGACGTGCACGCTATG | 318 |
| 139 | TVRRGDLATE | 121 | ACGGTACGGAGAGGAGACCTAGCAACCGAA | 319 |
| 140 | NIARGDAGQY | 16 | AATATTGCGAGAGGAGACGCTGGTCAGTAT | 320 |
| 141 | SVGRGDKADI | 122 | TCTGTTGGGAGAGGAGACAAGGCTGATATT | 321 |
| 142 | SHNRGDTGTM | 123 | TCACACAACAGAGGAGACACCGGAACCATG | 322 |
| 143 | TNTRGDKESV | 124 | ACGAACACCAGAGGAGACAAAGAATCAGTA | 323 |
| 144 | SIGRGDQYTI | 125 | TCGATTGGTAGAGGAGACCAGTATACGATT | 324 |
| 145 | QTSRGDAGSW | 126 | CAGACGAGTAGAGGAGACGCGGGGTCTTGG | 325 |
| 146 | AENRGDRSSL | 75 | GCTGAAAACAGAGGAGACCGAAGCAGCCTA | 326 |
| 147 | AISRGDVQSL | 127 | GCGATTTCGAGAGGAGACGTTCAGTCGTTG | 327 |
| 148 | NAQRGDHGQL | 128 | AACGCACAAAGAGGAGACCACGGGCAACTG | 328 |
| 149 | LNSRGDQASV | 129 | CTCAACTCGAGAGGAGACCAAGCCTCCGTC | 329 |
| 150 | SGNRGDIGTF | 130 | TCGGGAAACAGAGGAGACATAGGTACATTC | 330 |
| 151 | NVGRGDQATM | 131 | AACGTGGGAAGAGGAGACCAAGCCACAATG | 331 |
| 152 | SYSRGDTGRL | 132 | TCGTATTCTAGAGGAGACACTGGTCGGCTT | 332 |
| 153 | SHLRGDQALA | 133 | AGCCACTTAAGAGGAGACCAAGCGTTGGCC | 333 |
| 154 | YFARGDMTMN | 134 | TACTTCGCCAGAGGAGACATGACTATGAAC | 334 |
| 155 | QSQRGDLNPM | 135 | CAGTCGCAGAGAGGAGACCTTAATCCTATG | 335 |
| 156 | EQRRGDKTEL | 136 | GAGCAGAGGAGAGGAGACAAGACGGAGCTG | 336 |
| 157 | VSARGDAGQL | 137 | GTGTCTGCGAGAGGAGACGCGGGTCAGTTG | 337 |
| 158 | SPSRGDQGRT | 9 | TCTCCGTCTAGAGGAGACCAGGGGCGGACG | 338 |
| 159 | EASRGDKGTH | 138 | GAGGCTAGTAGAGGAGACAAGGGTACGCAT | 339 |
| 160 | LSTRGDMGMQ | 139 | CTGTCTACTAGAGGAGACATGGGTATGCAG | 340 |
| 161 | ANMRGDQLHT | 140 | GCGAATATGAGAGGAGACCAGCTGCATACT | 341 |
| 162 | ENTRGDLTHA | 141 | GAGAATACGAGAGGAGACCTTACTCATGCG | 342 |
| 163 | IERRGDSAGL | 142 | ATTGAGCGTAGAGGAGACTCGGCGGGGCTG | 343 |
| 164 | SAMRGDGVSL | 143 | AGTGCGATGAGAGGAGACGGAGTGAGCTTG | 344 |
| 165 | TVSRGDQGLS | 144 | ACGGTTTCGAGAGGAGACCAGGGGTTGTCT | 345 |
| 166 | SIVRGDLNST | 145 | TCGATCGTCAGAGGAGACCTGAACTCCACG | 346 |
| 167 | DVARGDKTNF | 146 | GACGTAGCGAGAGGAGACAAAACAAACTTC | 347 |
| 168 | WSQRGDLSGT | 147 | TGGAGCCAAAGAGGAGACCTGAGTGGAACC | 348 |
| 169 | LNQRGDVSNM | 148 | CTGAACCAAAGAGGAGACGTTTCCAACATG | 349 |
| 170 | AALRGDLSGS | 149 | GCCGCCCTAAGAGGAGACCTATCCGGCTCA | 350 |
| 171 | RVARGDITDI | 150 | AGGGTTGCGAGAGGAGACATTACGGATATT | 351 |
| 172 | STNRGDLNQV | 151 | TCGACGAACAGAGGAGACCTCAACCAAGTT | 352 |
| 173 | DSRRGDSVSL | 152 | GATTCGCGGAGAGGAGACTCIGTGAGTCIT | 353 |
| 174 | SVGRGDQSQM | 153 | AGTGTTGGGAGAGGAGACCAGTCTCAGATG | 354 |
| 175 | SNTRGDTNSL | 154 | TCCAACACGAGAGGAGACACAAACTCCCTA | 355 |
| 176 | DNRRGDGTTM | 155 | GATAATCGGAGAGGAGACGGTACGACTATG | 356 |
| 177 | TPSRGDQGRL | 156 | ACGCCTTCGAGAGGAGACCAAGGAAGACTA | 357 |
| 178 | NYSRGDSMTL | 157 | AACTACAGCAGAGGAGACTCAATGACGCTT | 358 |
| 179 | MQARGDAGTL | 158 | ATGCAGGCTAGAGGAGACGCTGGGACTCTG | 359 |
| 180 | TQSRGDLSGA | 159 | ACGCAGTCGAGAGGAGACTTGTCTGGTGCG | 360 |
| 181 | FAQRGDLTGV | 160 | TTTGCGCAGAGAGGAGACTTGACTGGGGTT | 361 |
| 182 | APVRGDLIGT | 161 | GCGCCGGTGAGAGGAGACCIGATTGGTACG | 362 |
| 183 | SNTRGDTNSL | 154 | TCGAATACTAGAGGAGACACGAATAGTTTG | 363 |
| 184 | EPKRGDLSNT | 162 | GAGCCGAAGAGAGGAGACTTGAGTAATACG | 364 |
| 185 | SVGRGDTYPL | 163 | TCGGTTGGGAGAGGAGACACTTATCCTCTG | 365 |
| 186 | QTARGDMTGH | 164 | CAAACAGCCAGAGGAGACATGACTGGCCAC | 366 |
| 187 | AGARGDLENR | 165 | GCTGGTGCGAGAGGAGACTTGGAGAATCGG | 367 |
| 188 | MSTRGDLNNV | 166 | ATGAGCACTAGAGGAGACCTAAACAACGTC | 368 |
| 189 | EKQRGDLNSM | 167 | GAAAAACAAAGAGGAGACCTCAACAGCATG | 369 |
| 190 | IKTRGDLGHE | 168 | ATTAAGACGAGAGGAGACCTTGGTCATGAG | 370 |
| 191 | IGRRGDMELS | 169 | ATTGGGAGGAGAGGAGACATGGAGTTGTCT | 371 |
| 192 | TTGRGDLKEV | 170 | ACTACGGGTAGAGGAGACTTGAAGGAGGTT | 372 |
| 193 | TDKRGDQNTV | 171 | ACGGATAAGAGAGGAGACCAGAATACTGTG | 373 |
| 194 | SNVRGDREAV | 172 | TCGAATGTTAGAGGAGACCGGGAGGCGGTT | 374 |
| 195 | SVGRGDQSQM | 153 | TCAGTCGGCAGAGGAGACCAAAGTCAAATG | 375 |
| 196 | YANRGDLSHQ | 173 | TACGCCAACAGAGGAGACCTGAGTCACCAA | 376 |
| 197 | QISRGDLGIN | 41 | CAAATATCGAGAGGAGACCTAGGTATCAAC | 377 |
| 198 | SMMRGDAGQL | 174 | TCGATGATGAGAGGAGACGCTGGGCAGTTG | 378 |
| 199 | EARRGDSQGL | 175 | GAGGCGCGGAGAGGAGACAGTCAGGGGCTG | 379 |
| 200 | MPTRGDQTKF | 176 | ATGCCCACAAGAGGAGACCAAACCAAATTC | 380 |

TABLE 2
Enhanced MyoAAV (eMyoAAV) Capsid Variants

| | | SEQ | | SEQ |
|---|---|---|---|---|
| | N-MER | ID | | ID |
| RANK | INSERT | NO.: | ENCODING SEQUENCE | NO.: |
| 1 | SNSRGDYNSL | 381 | TCAAACTCCAGAGGAGACTACAACTCCTTG | 557 |
| 2 | SSARGDRDYL | 397 | TCGTCGGCTAGAGGAGACCGTGATTATCTG | 558 |
| 3 | STVRGDYTSV | 398 | AGCACGGTCAGAGGAGACTACACGTCCGTG | 559 |
| 4 | QERRGDYTSM | 382 | CAGGAGCGGAGAGGAGACTATACGAGTATG | 560 |
| 5 | NGGRGDTTHF | 386 | AACGGCGGGAGAGGAGACACGACGCACTTC | 561 |
| 6 | ASTRGDHGVL | 383 | GCGAGTACTAGAGGAGACCATGGTGTGTTG | 562 |
| 7 | SYQRGDQHNM | 399 | TCGTACCAAAGAGGAGACCAACACAACATG | 563 |
| 8 | NDTRGDRSYM | 400 | AACGACACAAGAGGAGACCGATCTTACATG | 564 |
| 9 | ENRRGDFNNT | 384 | GAAAACAGGAGAGGAGACTTCAACAACACT | 565 |
| 10 | SGSRGDLSGH | 401 | TCCGGCTCGAGAGGAGACTTGTCAGGACAC | 566 |
| 11 | ENRRGDITQS | 393 | GAGAATCGGAGAGGAGACATTACGCAGTCG | 567 |
| 12 | DKPRGDRQLL | 402 | GATAAGCCGAGAGGAGACCGGCAGTTGTTG | 568 |
| 13 | EERRGDTHRL | 392 | GAGGAGCGGAGAGGAGACACTCATCGGCTG | 569 |
| 14 | AMIRGDMTHS | 391 | GCGATGATTAGAGGAGACATGACGCATAGT | 570 |
| 15 | SNNRGDYTTM | 403 | TCAAACAACAGAGGAGACTACACGACAATG | 571 |
| 16 | QGGRGDLSTL | 404 | CAGGGTGGGAGAGGAGACTTGTCGACGTTG | 572 |
| 17 | SKDRGDYQTL | 405 | TCGAAGGATAGAGGAGACTATCAGACTCTT | 573 |
| 18 | ATARGDQMKL | 406 | GCTACGGCGAGAGGAGACCAGATGAAGCTG | 574 |
| 19 | QHMRGDLTGA | 407 | CAGCATATGAGAGGAGACTTGACTGGTGCG | 575 |
| 20 | DGTRGDLGRY | 408 | GACGGCACGAGAGGAGACCTTGGAAGGTAC | 576 |
| 21 | DSRRGDYANL | 409 | GACTCTCGGAGAGGAGACTACGCAAACCTC | 577 |
| 22 | SAMRGDYTST | 410 | TCGGCGATGAGAGGAGACTATACTAGTACT | 578 |
| 23 | ENRRGDAQLL | 411 | GAGAATCGGAGAGGAGACGCGCAGCTGCTG | 579 |
| 24 | TTLRGDHSQL | 412 | ACGACGCTGAGAGGAGACCATAGTCAGCTT | 580 |
| 25 | DAGRGDMHRM | 413 | GACGCAGGGAGAGGAGACATGCACCGTATG | 581 |
| 26 | QHTRGDLSST | 414 | CAGCATACGAGAGGAGACTTGAGTTCTACT | 582 |
| 27 | QVYRGDRESV | 415 | CAAGTTTACAGAGGAGACAGGGAATCCGTG | 583 |
| 28 | SSQRGDLAGT | 416 | TCGAGTCAGAGAGGAGACTTGGCTGGTACT | 584 |
| 29 | TDVRGDRGTF | 417 | ACGGATGTTAGAGGAGACAGGGGGACGTTT | 585 |
| 30 | SSVRGDREVT | 418 | TCGTCTGTGAGAGGAGACCGGGAGGTGACG | 586 |
| 31 | ENRRGDLTNA | 419 | GAGAATAGGAGAGGAGACCTGACTAATGCG | 587 |
| 32 | SYARGDVHSI | 420 | TCGTATGCGAGAGGAGACGTTCATTCTATT | 588 |
| 33 | DSSRGDLNLR | 421 | GACAGCTCAAGAGGAGACCTCAACCTCCGG | 589 |
| 34 | SNARGDYSNM | 422 | TCGAACGCGAGAGGAGACTACTCAAACATG | 590 |
| 35 | SSVRGDHSVL | 423 | TCGTCTGTGAGAGGAGACCATTCGGTGCTT | 591 |
| 36 | SAFRGDLHAT | 424 | TCGGCTTTTAGAGGAGACCTGCATGCTACT | 592 |
| 37 | SAMRGDYTST | 410 | TCCGCCATGAGAGGAGACTACACAAGCACG | 593 |
| 38 | NAQRGDHGQL | 394 | AACGCACAAAGAGGAGACCACGGGCAACTG | 594 |
| 39 | SDRRGDQLLY | 425 | TCGGATCGGAGAGGAGACCAGCTGTTGTAT | 595 |
| 40 | SGVRGDRLAV | 426 | AGCGGCGTAAGAGGAGACCGCCTAGCCGTA | 596 |
| 41 | ASLRGDLSST | 427 | GCCAGCCTAAGAGGAGACCTCAGCTCAACG | 597 |
| 42 | TNGRGDLHGM | 428 | ACGAACGGAAGAGGAGACCTTCACGGAATG | 598 |
| 43 | TNTRGDHGML | 429 | ACGAATACGAGAGGAGACCATGGGATGCTG | 599 |
| 44 | SSARGDRDYL | 397 | AGTTCCGCCAGAGGAGACCGGGACTACCTC | 600 |
| 45 | EDRRGDLLRT | 430 | GAGGATCGGAGAGGAGACCTTTTGAGGACT | 601 |
| 46 | SSLRGDLLHS | 431 | TCGTCGCTGAGAGGAGACCTGTTGCATTCT | 602 |
| 47 | ENRRGDLTNA | 419 | GAAAACCGTAGAGGAGACCTCACGAACGCT | 603 |
| 48 | DGRRGDRESI | 432 | GACGGCAGGAGAGGAGACCGGGAATCGATC | 604 |
| 49 | TGTRGDTMTW | 433 | ACTGGAACCAGAGGAGACACAATGACATGG | 605 |
| 50 | STVRGDYTSV | 398 | TCGACTGTTAGAGGAGACTATACTTCTGTT | 606 |
| 51 | SATRGDHNVL | 434 | TCGGCAACGAGAGGAGACCACAACGTACTG | 607 |
| 52 | TDRRGDSGTL | 435 | ACGGATCGGAGAGGAGACAGTGGTACTCTG | 608 |
| 53 | TSARGDLIHT | 436 | ACGAGTGCTAGAGGAGACCTGATTCATACG | 609 |
| 54 | SGVRGDRLAV | 426 | AGTGGGGTGAGAGGAGACCGGCTGGCGGTG | 610 |
| 55 | AVERGDRLML | 437 | GCGGTTGAGAGAGGAGACCGTCTTATGCTG | 611 |
| 56 | DRSRGDTHVL | 438 | GACAGGTCGAGAGGAGACACGCACGTACTC | 612 |
| 57 | ESTRGDLRVV | 439 | GAGTCGACGAGAGGAGACCTTCGTGTTGTG | 613 |
| 58 | TTIRGDYREL | 440 | ACGACGATTAGAGGAGACTATCGTGAGTTG | 614 |
| 59 | TFIRGDLAGA | 441 | ACGTTCATCAGAGGAGACCTGGCAGGTGCG | 615 |
| 60 | MTSRGDLTTY | 442 | ATGACGAGCAGAGGAGACTTGACAACATAC | 616 |
| 61 | ADRRGDQLYS | 443 | GCGGATCGTAGAGGAGACCAGCTTTATTCG | 617 |
| 62 | AHMRGDLGGT | 385 | GCACACATGAGAGGAGACCTAGGCGGCACG | 618 |
| 63 | GYVRGDLGQH | 444 | GGGTACGTCAGAGGAGACCTGGGGCAACAC | 619 |
| 64 | DASRGDRTSL | 445 | GATGCGAGTAGAGGAGACAGGACTAGTTTG | 620 |
| 65 | TEVRGDRSQV | 446 | ACGGAGGTGAGAGGAGACAGGTCGCAGGTT | 621 |
| 66 | SHLRGDQALA | 395 | TCGCATCTGAGAGGAGACCAGGCTCTTGCG | 622 |
| 67 | SEVRGDRLSI | 447 | TCGGAGGTGAGAGGAGACCGGTTGAGTATT | 623 |
| 68 | SAARGDTERL | 387 | AGCGCCGCGAGAGGAGACACTGAACGGCTA | 624 |
| 69 | SDRRGDLSGA | 448 | TCAGACCGTAGAGGAGACCTGTCGGGCGCT | 625 |
| 70 | SVTRGDRVVI | 449 | AGCGTCACAAGAGGAGACAGAGTAGTCATC | 626 |
| 71 | SEGRGDRMAL | 450 | TCGGAGGGGAGAGGAGACAGGATGGCTCTT | 627 |
| 72 | SSTRGDHLSL | 451 | TCGTCTACTAGAGGAGACCATCTTTCGCTG | 628 |
| 73 | AMIRGDMTHS | 391 | GCTATGATCAGAGGAGACATGACCCACTCC | 629 |
| 74 | SSTRGDQIYV | 452 | TCGTCGACGAGAGGAGACCAGATTTATGTG | 630 |
| 75 | EERRGDTHRL | 392 | GAGGAGCGGAGAGGAGACACTCATCGGTTG | 631 |
| 76 | SNSRGDYNSL | 381 | TCGAATTCGAGAGGAGACTATAATAGTCTT | 632 |
| 77 | SAQRDRMGSP | 453 | TCCGCACAAAGGGATCGTATGGGTTCTCCG | 633 |
| 78 | TGTRGDIATF | 454 | ACGGGTACGAGAGGAGACATTGCGACTTTT | 634 |
| 79 | SMQRGDLLST | 455 | TCGATGCAGAGAGGAGACCTGCTTTCTACT | 635 |
| 80 | EDVRGDRSKL | 456 | GAGGATGTGAGAGGAGACAGGAGTAAGCTT | 636 |
| 81 | AHMRGDLGGT | 385 | GCGCATATGAGAGGAGACTTGGGGGGGACT | 637 |
| 82 | TSVRGDQSQY | 457 | ACGAGTGTGAGAGGAGACCAGAGTCAGTAT | 638 |
| 83 | ADRRGDTGLF | 458 | GCGGATCGGAGAGGAGACACGGGGTTGTTT | 639 |
| 84 | NTYRGDRDSL | 459 | AACACTTACAGAGGAGACCGAGACAGCCTG | 640 |
| 85 | TDVRGDRGTF | 417 | ACGGATGTTAGAGGAGACCGGGGGACGTTT | 641 |
| 86 | SRGRGDLNDL | 460 | TCTCGTGGGAGAGGAGACCTGAATGATTTG | 642 |
| 87 | SNVRGDREAV | 396 | AGCAACGTAAGAGGAGACAGGGAAGCCGTC | 643 |
| 88 | MDRRGDASIL | 461 | ATGGACAGGAGAGGAGACGCCTCCATACTG | 644 |
| 89 | SIWRGDRTEV | 462 | TCGATATGGAGAGGAGACCGTACAGAAGTC | 645 |
| 90 | NSQRGDYSGM | 463 | AACTCACAAAGAGGAGACTACAGTGGAATG | 646 |
| 91 | SSVRGDIGGI | 464 | TCTAGCGTAAGAGGAGACATCGGGGGGATC | 647 |
| 92 | ENQRGDLSGR | 390 | GAGAATCAGAGAGGAGACTTGTCGGGTCGG | 648 |
| 93 | EIRRGDLLAQ | 465 | GAGATTCGGAGAGGAGACCTTCTGGCTCAG | 649 |
| 94 | SNIRGDHAVM | 466 | TCGAACATCAGAGGAGACCACGCAGTCATG | 650 |
| 95 | LDTRGDRSQV | 467 | CTGGATACGAGAGGAGACCGGAGTCAGGTT | 651 |
| 96 | ANTRGDHQNF | 468 | GCGAATACGAGAGGAGACCATCAGAATTTT | 652 |
| 97 | QVYRGDRESV | 415 | CAGGTGTATAGAGGAGACCGTGAGTCTGTT | 653 |
| 98 | TSVRGDLSMN | 469 | ACGTCGGTGAGAGGAGACCTTTCGATGAAT | 654 |
| 99 | ASTRGDHGVL | 383 | GCCTCAACGAGAGGAGACCACGGAGTTCTC | 655 |
| 100 | TTTRGDYLNT | 470 | ACGACGACGAGAGGAGACTATCTTAATACG | 656 |
| 101 | AERRGDIKEY | 471 | GCGGAGAGGAGAGGAGACATTAAGGAGTAT | 657 |
| 102 | SHARGDLGST | 472 | TCGCATGCTAGAGGAGACTTGGGTTCGACT | 658 |
| 103 | TSQRGDYVSL | 473 | ACGTCTCAGAGAGGAGACTATGTTTCTCTT | 659 |
| 104 | GPGRGDQTTL | 556 | GGGCCGGGTAGAGGAGACCAGACTACGTTG | 660 |
| 105 | VNVRGDRGEV | 474 | GTGAATGTGAGAGGAGACCGTGGTGAGGTG | 661 |
| 106 | SYRRGDHDQL | 475 | TCGTATCGTAGAGGAGACCATGATCAGCTT | 662 |
| 107 | SGTRGDRVDL | 476 | AGCGGCACAAGAGGAGACAGGGTAGACCTC | 663 |
| 108 | SHFRGDLHTS | 477 | TCCCACTTCAGAGGAGACTTGCACACGTCC | 664 |
| 109 | SDRRGDLSVP | 478 | TCGGATCGTAGAGGAGACCTTTCGGTGCCT | 665 |
| 110 | SGTRGDTLIL | 479 | AGCGGCACCAGAGGAGACACCCTTATACTC | 666 |
| 111 | ENTRGDLHGK | 480 | GAAAACACCAGAGGAGACCTCCACGGCAAA | 667 |
| 112 | DRARGDTHVL | 481 | GATCGGGCGAGAGGAGACACGCATGTGCTG | 668 |
| 113 | ASGRGDIAGL | 482 | GCGTCTGGTAGAGGAGACATTGCGGGGCTT | 669 |
| 114 | EGRRGDLFQA | 483 | GAGGGTCGTAGAGGAGACCTGTTTCAGGCG | 670 |
| 115 | RSERGDRLEI | 484 | AGATCCGAAAGAGGAGACAGGCTAGAAATC | 671 |
| 116 | AAYRGDAHVL | 485 | GCGGCCTACAGAGGAGACGCACACGTGCTC | 672 |
| 117 | NDSRGDQHRL | 486 | AACGACTCGAGAGGAGACCAACACCGATTG | 673 |
| 118 | SSARGDHSQL | 487 | TCATCGGCAAGAGGAGACCACTCTCAACTG | 674 |
| 119 | TNERGDRLSI | 488 | ACGAATGAGAGAGGAGACCGTCTTTCGATT | 675 |
| 120 | SNSRGDYTSV | 489 | AGCAACTCCAGAGGAGACTACACCAGTGTC | 676 |
| 121 | NVTRGDHTVM | 490 | AACGTCACAAGAGGAGACCACACCGTTATG | 677 |
| 122 | TMVRGDVKGL | 491 | ACGATGGTGAGAGGAGACGTTAAGGGGCTT | 678 |
| 123 | TGTRGDIATF | 454 | ACCGGGACAAGAGGAGACATAGCCACGTTC | 679 |
| 124 | VQLRGDLAST | 492 | GTGCAGCTGAGAGGAGACTTGGCTTCTACT | 680 |
| 125 | MNRRGDYSEQ | 493 | ATGAACCGGAGAGGAGACTACTCGGAACAA | 681 |
| 126 | ANTRGDLSPV | 494 | GCGAATACTAGAGGAGACCTGAGTCCGGTT | 682 |
| 127 | TGMRGDAGSL | 495 | ACGGGTATGAGAGGAGACGCTGGGAGTCTT | 683 |
| 128 | LDRRGDFSSA | 496 | CTAGACCGCAGAGGAGACTTCAGCTCCGCG | 684 |
| 129 | MYHRGDMTSV | 497 | ATGTACCACAGAGGAGACATGACTTCGGTG | 685 |
| 130 | GSMRGDLHST | 498 | GGGTCGATGAGAGGAGACTTGCATTCGACG | 686 |
| 131 | SSSRGDFSSV | 499 | TCGTCGTCCAGAGGAGACTTCAGCTCTGTT | 687 |
| 132 | TGTRGDLHTY | 500 | ACGGGTACGAGAGGAGACTTGCATACTTAT | 688 |
| 133 | YTQRGDLATV | 501 | TACACCCAAAGAGGAGACCTTGCCACCGTC | 689 |
| 134 | TDVRGDRMYV | 502 | ACGGATGTTAGAGGAGACCGGATGTATGTG | 690 |
| 135 | TSVRGDHGTL | 388 | ACATCGGTCAGAGGAGACCACGGAACATTG | 691 |
| 136 | SNSRGDLSVN | 503 | TCGAACTCCAGAGGAGACCTATCGGTAAAC | 692 |
| 137 | QSGRGDQMTL | 504 | CAGTCTGGGAGAGGAGACCAGATGACGTTG | 693 |
| 138 | MNTRGDLQQS | 505 | ATGAACACCAGAGGAGACTTGCAACAATCG | 694 |
| 139 | AGSRGDLQTV | 506 | GCGGGATCAAGAGGAGACCTACAAACGGTG | 695 |
| 140 | SNMRGDQTYV | 507 | TCGAACATGAGAGGAGACCAAACTTACGTC | 696 |
| 141 | TPSRGDMSNV | 508 | ACGCCAAGTAGAGGAGACATGTCCAACGTC | 697 |
| 142 | NSTRGDLHMA | 509 | AACAGCACAAGAGGAGACCTACACATGGCA | 698 |
| 143 | ANTRGDLSVS | 510 | GCTAACACCAGAGGAGACCTCTCAGTCAGT | 699 |
| 144 | TDRRGDTGLL | 511 | ACCGACCGAAGAGGAGACACCGGCTTACTG | 700 |
| 145 | DGVRGDRATW | 512 | GACGGCGTAAGAGGAGACCGTGCAACATGG | 701 |
| 146 | ENRRGDFNNT | 384 | GAGAATCGTAGAGGAGACTTTAATAATACG | 702 |
| 147 | EPSRGDRMVL | 513 | GAGCCTAGTAGAGGAGACAGGATGGTGTTG | 703 |
| 148 | SGVRGDRIII | 514 | AGCGGCGTTAGAGGAGACCGGATCATAATC | 704 |
| 149 | TSMRGDYTHV | 515 | ACGTCGATGAGAGGAGACTATACGCATGTT | 705 |
| 150 | ASSRGDLTVI | 516 | GCGTCGAGTAGAGGAGACTTGACTGTTATT | 706 |
| 151 | ETRRGDLSLQ | 517 | GAGACGCGTAGAGGAGACCTGTCGCTTCAG | 707 |
| 152 | NNARGDQSMH | 518 | AACAACGCAAGAGGAGACCAATCAATGCAC | 708 |
| 153 | TDRRGDSSAL | 519 | ACGGATAGGAGAGGAGACTCTAGTGCGCTT | 709 |
| 154 | SSQRGDLTGV | 520 | TCAAGCCAAAGAGGAGACTTGACAGGTGTG | 710 |
| 155 | QTARGDVMVH | 521 | CAGACGGCTAGAGGAGACGTTATGGTTCAT | 711 |
| 156 | VSQRGDLNAV | 522 | GTGTCGCAGAGAGGAGACCTGAATGCTGTT | 712 |
| 157 | TMTRGDLAAN | 523 | ACGATGACCAGAGGAGACCTCGCTGCTAAC | 713 |
| 158 | NASRGDHSSL | 524 | AATGCTTCGAGAGGAGACCATTCGTCGTTG | 714 |
| 159 | SNTRGDMGLT | 525 | TCAAACACCAGAGGAGACATGGGCCTCACG | 715 |
| 160 | MYSRGDTHSL | 526 | ATGTATTCGAGAGGAGACACTCATAGTCTG | 716 |
| 161 | ADQRGDRAPL | 527 | GCGGATCAGAGAGGAGACAGGGCTCCGCTT | 717 |
| 162 | QERRGDYTSM | 382 | CAAGAAAGGAGAGGAGACTACACCTCTATG | 718 |
| 163 | SQNRGDLANT | 528 | TCCCAAAACAGAGGAGACCTAGCCAACACG | 719 |
| 164 | EKLRGDLHST | 389 | GAGAAGTTGAGAGGAGACCTTCATTCGACT | 720 |
| 165 | QGGRGDLSTL | 404 | CAAGGCGGAAGAGGAGACCTGAGTACACTG | 721 |
| 166 | SNRRGDTEMQ | 529 | TCGAATAGGAGAGGAGACACTGAGATGCAG | 722 |
| 167 | SGSRGDVSAL | 530 | TCGGGTAGTAGAGGAGACGTGAGTGCTTTG | 723 |
| 168 | ESTRGDRGTL | 531 | GAGTCGACGAGAGGAGACCGTGGTACGCTG | 724 |
| 169 | SAVRGDAALH | 532 | TCGGCGGTGAGAGGAGACGCGGCGCTTCAT | 725 |
| 170 | LSSRGDVNRL | 533 | CTGAGCAGCAGAGGAGACGTTAACCGCCTT | 726 |
| 171 | EQRRGDIQTI | 534 | GAGCAGAGGAGAGGAGACATTCAGACTATT | 727 |
| 172 | AAYRGDAHVL | 485 | GCTGCGTATAGAGGAGACGCGCATGTTCTT | 728 |
| 173 | SPVRGDHGAL | 535 | TCGCCGGTGAGAGGAGACCATGGGGCTTTG | 729 |
| 174 | DSRRGDYANL | 409 | GATTCGAGGAGAGGAGACTATGCGAATCTG | 730 |
| 175 | LSRRGDYQEL | 536 | TTGAGTCGGAGAGGAGACTATCAGGAGTTG | 731 |
| 176 | SYARGDVHSI | 420 | TCCTACGCAAGAGGAGACGTCCACTCCATC | 732 |
| 177 | DSRRGDASYH | 537 | GACTCACGCAGAGGAGACGCGTCGTACCAC | 733 |
| 178 | NHQRGDLSSS | 538 | AACCACCAAAGAGGAGACCTGAGCTCGAGT | 734 |
| 179 | SSTRGDHLSL | 451 | TCCTCAACGAGAGGAGACCACCTGTCTTTG | 735 |
| 180 | SWNRGDISGL | 539 | TCGTGGAACAGAGGAGACATATCTGGCCTT | 736 |
| 181 | TGTRGDLGTM | 540 | ACGGGTACTAGAGGAGACCTGGGGACGATG | 737 |
| 182 | QQLRGDTHTL | 541 | CAGCAGCTTAGAGGAGACACGCATACTCTT | 738 |
| 183 | AGLRGDRDSL | 542 | GCGGGGTTGAGAGGAGACCGTGATTCGCTT | 739 |
| 184 | QVYRGDRDQL | 543 | CAGGTTTATAGAGGAGACCGGGATCAGTTG | 740 |
| 185 | AGVRGDRVTI | 544 | GCAGGAGTAAGAGGAGACAGAGTGACCATC | 741 |
| 186 | NSARGDLLHS | 545 | AACTCAGCCAGAGGAGACCTTCTGCACTCC | 742 |
| 187 | NHSRGDLTGV | 546 | AACCACAGTAGAGGAGACCTGACAGGCGTT | 743 |
| 188 | NAYRGDTSAF | 547 | AACGCTTACAGAGGAGACACATCCGCGTTC | 744 |
| 189 | SANRGDIMHT | 548 | TCGGCTAACAGAGGAGACATAATGCACACG | 745 |
| 190 | QSARGDLVSY | 549 | CAATCCGCCAGAGGAGACCTCGTCAGTTAC | 746 |
| 191 | DSRRGDASYH | 537 | GATAGTAGGAGAGGAGACGCTAGTTATCAT | 747 |
| 192 | SFVRGDVRTL | 550 | TCGTTTGTGAGAGGAGACGTTCGTACGCTG | 748 |
| 193 | SDMRGDRSVY | 551 | AGCGACATGAGAGGAGACCGATCTGTGTAC | 749 |
| 194 | SGIRGDRYPI | 552 | TCCGGAATCAGAGGAGACCGCTACCCAATA | 750 |
| 195 | SNVRGDMAHS | 553 | TCCAACGTCAGAGGAGACATGGCTCACAGC | 751 |
| 196 | TEVRGDRSQV | 446 | ACTGAGGTTAGAGGAGACCGTTCGCAGGTT | 752 |
| 197 | ASSRGDLQQT | 554 | GCGAGCAGCAGAGGAGACCTGCAACAAACG | 753 |
| 198 | QSARGDLVSY | 549 | CAGTCTGCTAGAGGAGACCTTGTGTCTTAT | 754 |
| 199 | DASRGDRTSL | 445 | GACGCATCTAGAGGAGACCGAACATCCCTC | 755 |
| 200 | NSVRGDLNQT | 555 | AACAGTGTCAGAGGAGACCTCAACCAAACT | 756 |

TABLE 3
Top Ranking Skeletal Muscle Specific
n-mer inserts and/or RGD Motifs

| Variant | SEQ ID NO.: |
|---|---|
| TTGRGDQARL | 757 |
| TTLRGDHSQL | 758 |
| TNLRGDYQLT | 759 |
| STSRGDYVTH | 760 |
| MPARGDLVQT | 761 |
| SGVRGDRLAV | 762 |
| SNERGDQHRL | 763 |
| STLRGDYAVS | 764 |
| SNARGDIMTT | 765 |
| EASRGDLQMR | 766 |
| TYARGDLSGH | 767 |
| DSSRGDRSSV | 768 |
| SQSRGDLVPL | 769 |
| SATRGDYSIL | 770 |
| QATRGDLQHT | 771 |
| HDDRGDRRML | 772 |
| SSHRGDLLAT | 773 |
| SYQRGDQHNM | 774 |
| SHQRGDYNTL | 775 |
| MDQRGDMSVV | 776 |
| QIARGDLHVP | 777 |
| TYTRGDQHMM | 778 |
| DASRGDRTSL | 779 |
| SDRRGDLHPQ | 780 |
| SATRGDHSTL | 781 |
| AYQRGDFTSV | 782 |
| TGNRGDLQSV | 783 |
| AWRRGDLENA | 784 |
| NAARGDHGLL | 785 |
| SYSRGDHQAL | 786 |
| DGVRGDRTVV | 787 |
| SSERGDKMST | 788 |
| TSMRGDYTHV | 789 |
| SGVRGDRIII | 790 |
| ENRRGDAQLL | 791 |
| TVYRGDREQH | 792 |
| LDRRGDLLNS | 793 |
| ESVRGDLRST | 794 |
| DKYRGDQRGL | 795 |
| TDRRGDAHIL | 796 |
| SQTRGDTSLM | 797 |
| DATRGDTRML | 798 |
| SMQRGDLTHA | 799 |
| SGTRGDTLIL | 800 |
| ANTRGDHQNF | 801 |
| TSQRGDHTQL | 802 |
| SHWRGDTSSL | 803 |
| ISGRGDRQEL | 804 |
| SVLRGDIGTI | 805 |
| QDSRGDLSGK | 806 |
| KPIRGDGAEL | 807 |
| TYMRGDLQHT | 808 |
| TELRGDLSST | 809 |
| SAMRGDHAVL | 810 |
| SWVRGDASTL | 811 |
| QYARGDPHAL | 812 |
| STNRGDIVVH | 813 |
| VNIRGDLHQT | 814 |
| REYRGDSSIL | 815 |
| STLRGDGSMM | 816 |
| SSQRGDITAH | 817 |
| TEFRGDRSTV | 818 |
| TSTRGDYVTV | 819 |
| NHLRGDVSAI | 820 |
| LERRGDLVST | 821 |
| NEYRGDHSHP | 822 |
| NWQRGDTSTL | 823 |
| TDSRGDRTVL | 824 |
| YAVRGDQHTI | 825 |
| FGVRGDTRDL | 826 |
| SSARGDTGML | 827 |
| SAMRGDQMGQ | 828 |
| SHTRGDLVTT | 829 |
| VTTRGDTAIL | 830 |
| EPTRGDRHSI | 831 |
| GSQRGDRLVL | 832 |
| QHYRGDLSTT | 833 |
| METRGDRQQL | 834 |
| EALRGDKVVL | 835 |
| NAVRGDQSYV | 836 |
| SPLRGDNRAL | 837 |
| ENRRGDTHAL | 838 |
| NSQRGDLTGT | 839 |
| QYAPSQGTV | 840 |
| DNSRGDHSRL | 841 |
| NTHRGDYASI | 842 |
| HTTRGDQQPL | 843 |
| EWRRGDQTAL | 844 |
| TAVRGDQTTV | 845 |
| EYLRGDRISQ | 846 |
| SRYRGDQETI | 847 |
| ASLRGDTVTL | 848 |
| VGVRGDSHVL | 849 |
| TNSRGDHTSL | 850 |
| VDQRGDQRAL | 851 |
| ASVRGDRLDV | 852 |
| TSYRGDLSSN | 853 |
| TSDRGDSSNR | 854 |
| SYMRGDLDMR | 855 |
| TDVRGDRSMI | 856 |
| SYVRGDSSTV | 857 |
| QVYRGDRESV | 858 |
| TDRRGDAVSF | 859 |
| TNSRGDLHTT | 860 |
| EAIRGDFKTL | 861 |
| TGIRGDTAGL | 862 |
| TSSRGDVQSL | 863 |
| SSRRGDTNEL | 864 |
| SNARGDHTPL | 865 |
| SELRGDRSPY | 866 |
| VHARGDTSIM | 867 |
| SALRGDHGTL | 868 |
| QTGRGDIAGM | 869 |
| TVARGDQIRL | 870 |
| TNSRGDSVAL | 871 |
| QNARGDSAVI | 872 |
| HYLRGDTTTL | 873 |
| ANARGDLQNT | 874 |
| QNIRGDIRTV | 875 |
| AALRGDHGQM | 876 |
| TTDRGDLSAR | 877 |
| LNVRGDLVHT | 878 |
| YSTRGDHTVL | 879 |
| TNARGDLTVS | 880 |
| TNARGDYTAL | 881 |
| AEGRGDTRGS | 882 |
| TNIRGDQSSV | 883 |
| VDRRGDTMTM | 884 |
| QIGRGDVTPL | 885 |
| MQARGDLLGV | 886 |
| NTQRGDYASV | 887 |
| SDTRGDRITQ | 888 |
| SAIRGDYRDV | 889 |
| VGVRGDVHPL | 890 |
| TVLRGDAERL | 891 |
| ASTRGDLSGQ | 892 |
| DSRRGDGQNL | 893 |
| IDKRGDIQGL | 894 |
| TVTRGDLLVN | 895 |
| DSTRGDRLGL | 896 |
| AANRGDYASL | 897 |
| RDYRGDTSSL | 898 |
| SNIRGDASLH | 899 |
| NLQRGDRTDF | 900 |
| NASRGDHSSM | 901 |
| TSMRGDYTHV | 902 |
| TSRRGDLNDV | 903 |
| QSHRGDVQSL | 904 |
| STTRGDYQSV | 905 |
| YSTRGDHAML | 906 |
| TSARGDLIHT | 907 |
| ATARGDRESF | 908 |
| ASTRGDYSTM | 909 |
| SNNRGDVQRL | 910 |
| ERLRGDQHII | 911 |
| SSIRGDHATL | 912 |
| NPTRGDIATV | 913 |
| MSTRGDLSVN | 914 |
| MDRRGDITYA | 915 |
| NATRGDLMSA | 916 |
| SDHRGDRTYQ | 917 |
| NWVRGDLSTA | 918 |
| SSHRGDLSAT | 919 |
| NATRGDYREI | 920 |
| DTLRGDLQTA | 921 |
| IAVRGDLTPV | 922 |
| TLLRGDLAHS | 923 |
| MNTRGDHTVL | 924 |
| TGHRGDIAVY | 925 |
| TSNRGDQMSN | 926 |
| SATRGDHTAI | 927 |
| SSVRGDREHA | 928 |
| TNSRGDLVVH | 929 |
| ETRRGDLSSQ | 930 |
| TYARGDITLT | 931 |
| SASRGDTLHL | 932 |
| DNQRGDLSSR | 933 |
| TQSRGDLRDA | 934 |
| ENRRGDLTNA | 935 |
| TSIRGDISSV | 936 |
| ISARGDTMTL | 937 |
| SRVRGDQDHW | 938 |
| SGNRGDIASY | 939 |
| NEQRGDRYNH | 940 |
| EALRGDVRAL | 941 |
| VDRRGDYTQM | 942 |
| NNVRGDYTSV | 943 |
| QTYRGDASVL | 944 |
| SNYRGDHGSL | 945 |
| TSSRGDLREV | 946 |
| QYTRGDVSNL | 947 |
| SDLRGDVRER | 948 |
| QNARGDTTAL | 949 |
| NSLRGDRDTM | 950 |
| SNSRGDQAQV | 951 |
| EATRGDRSTL | 952 |
| VPQRGDITNL | 953 |
| DKIRGDLRSI | 954 |
| SAARGDYSIV | 955 |
| VGLRGDLIGQ | 956 |
| QAGRGDLTVI | 957 |
| SKTRGDFQEA | 958 |
| GTARGDYSML | 959 |
| ISTRGDLVTI | 960 |
| TGTRGDREYM | 961 |
| MTTRGDLSIT | 962 |
| STSRGDMTSV | 963 |
| GPARGDFTSS | 964 |
| VDRRGDLGHV | 965 |
| NSGRGDMSQL | 966 |
| SQGRGDLERY | 967 |
| VFGRGDQYTL | 968 |
| IVGRGDLHLQ | 969 |
| SYSRGDTSRL | 970 |
| MSSRGDRATI | 971 |
| MSSRGDLTRV | 972 |
| QHTRGDFGNL | 973 |
| ETRRGDLSSQ | 974 |
| TGTRGDLRAI | 975 |
| QMARGDLSTA | 976 |
| LVERGDRIMN | 977 |
| GLVRGDLNAR | 978 |
| NNPRGDLSSV | 979 |
| HEKRGDLSNS | 980 |
| SQARGDLHQT | 981 |
| FTGRGDLRTA | 982 |
| QSIRGDTGHL | 983 |
| SAGRGDMSVL | 984 |
| SERRGDEHKW | 985 |
| TTGRGDLVSL | 986 |
| LGLRGDQIGQ | 987 |
| VALRGDLTHQ | 988 |
| MIGRGDQAPL | 989 |
| FQARGDHSTL | 990 |
| NSVRGDHSSL | 991 |
| WPNRGDLQST | 992 |
| SASRGDYQTL | 993 |
| TMQRGDLHTI | 994 |
| TALRGDLAGT | 995 |
| VNARGDFSQT | 996 |
| GWIRGDRTSM | 997 |
| TFSRGDHTVL | 998 |
| SGARGDLVGV | 999 |
| NNVRGDYTSV | 1000 |
| QTARGDHVVL | 1001 |
| TLIRGDLTRS | 1002 |
| VTTRGDRLEL | 1003 |

TABLE 4
Top Ranking Skeletal Muscle Specific
n-mer inserts and/or RGD Motifs

| Variant | SEQ ID NO.: |
|---|---|
| EASRGDLQMR | 1004 |
| TYARGDLSGH | 1005 |
| DSSRGDRSSV | 1006 |
| SQSRGDLVPL | 1007 |
| MPARGDLVQT | 1008 |
| SGVRGDRLAV | 1009 |
| SNERGDQHRL | 1010 |
| STLRGDYAVS | 1011 |
| SNARGDIMTT | 1012 |
| TTGRGDQARL | 1013 |
| TTLRGDHSQL | 1014 |
| TNLRGDYQLT | 1015 |
| STSRGDYVTH | 1016 |
| SATRGDYSIL | 1017 |
| QATRGDLQHT | 1018 |
| HDDRGDRRML | 1019 |
| SSHRGDLLAT | 1020 |
| SYQRGDQHNM | 1021 |
| SHQRGDYNTL | 1022 |
| MDQRGDMSVV | 1023 |
| QIARGDLHVP | 1024 |
| TYTRGDQHMM | 1025 |
| DASRGDRTSL | 1026 |
| SDRRGDLHPQ | 1027 |
| SATRGDHSTL | 1028 |
| AYQRGDFTSV | 1029 |
| TGNRGDLQSV | 1030 |
| AWRRGDLENA | 1031 |
| NAARGDHGLL | 1032 |
| SYSRGDHQAL | 1033 |
| DGVRGDRTVV | 1034 |
| SSERGDKMST | 1035 |
| TSMRGDYTHV | 1036 |
| SGVRGDRIII | 1037 |
| ENRRGDAQLL | 1038 |
| TVYRGDREQH | 1039 |
| LDRRGDLLNS | 1040 |
| ESVRGDLRST | 1041 |
| DKYRGDQRGL | 1042 |
| TDRRGDAHIL | 1043 |
| SQTRGDTSLM | 1044 |
| DATRGDTRML | 1045 |
| SMQRGDLTHA | 1046 |
| SGTRGDTLIL | 1047 |
| ANTRGDHQNF | 1048 |
| TSQRGDHTQL | 1049 |
| SHWRGDTSSL | 1050 |
| ISGRGDRQEL | 1051 |
| SVLRGDIGTI | 1052 |
| QDSRGDLSGK | 1053 |
| KPIRGDGAEL | 1054 |
| TYMRGDLQHT | 1055 |
| TELRGDLSST | 1056 |
| SAMRGDHAVL | 1057 |
| SWVRGDASTL | 1058 |
| QYARGDPHAL | 1059 |
| STNRGDIVVH | 1060 |
| VNIRGDLHQT | 1061 |
| REYRGDSSIL | 1062 |
| STLRGDGSMM | 1063 |
| SSQRGDITAH | 1064 |
| TEFRGDRSTV | 1065 |
| TSTRGDYVTV | 1066 |
| NHLRGDVSAI | 1067 |
| LERRGDLVST | 1068 |
| NEYRGDHSHP | 1069 |
| NWQRGDTSTL | 1070 |
| TDSRGDRTVL | 1071 |
| YAVRGDQHTI | 1072 |
| FGVRGDTRDL | 1073 |
| SSARGDTGML | 1074 |
| SAMRGDQMGQ | 1075 |
| SHTRGDLVTT | 1076 |
| VTTRGDTAIL | 1077 |
| EPTRGDRHSI | 1078 |
| GSQRGDRLVL | 1079 |
| QHYRGDLSTT | 1080 |
| METRGDRQQL | 1081 |
| EALRGDKVVL | 1082 |
| NAVRGDQSYV | 1083 |
| SPLRGDNRAL | 1084 |
| ENRRGDTHAL | 1085 |
| NSQRGDLTGT | 1086 |
| DNSRGDHSRL | 1087 |
| NTHRGDYASI | 1088 |
| HTTRGDQQPL | 1089 |
| EWRRGDQTAL | 1090 |
| TAVRGDQTTV | 1091 |
| EYLRGDRISQ | 1092 |
| SRYRGDQETI | 1093 |
| ASLRGDTVTL | 1094 |
| VGVRGDSHVL | 1095 |
| TNSRGDHTSL | 1096 |
| VDQRGDQRAL | 1097 |
| ASVRGDRLDV | 1098 |
| TSYRGDLSSN | 1099 |
| TSDRGDSSNR | 1100 |
| SYMRGDLDMR | 1101 |
| TDVRGDRSMI | 1102 |
| SYVRGDSSTV | 1103 |
| QVYRGDRESV | 1104 |
| TDRRGDAVSF | 1105 |
| TNSRGDLHTT | 1106 |
| EAIRGDFKTL | 1107 |
| TGIRGDTAGL | 1108 |
| TSSRGDVQSL | 1109 |
| SSRRGDTNEL | 1110 |
| SNARGDHTPL | 1111 |
| SELRGDRSPY | 1112 |
| VHARGDTSIM | 1113 |
| SALRGDHGTL | 1114 |
| QTGRGDIAGM | 1115 |
| TVARGDQIRL | 1116 |
| TNSRGDSVAL | 1117 |
| QNARGDSAVI | 1118 |
| HYLRGDTTTL | 1119 |
| ANARGDLQNT | 1120 |
| QNIRGDIRTV | 1121 |
| AALRGDHGQM | 1122 |
| TTDRGDLSAR | 1123 |
| LNVRGDLVHT | 1124 |
| YSTRGDHTVL | 1125 |
| TNARGDLTVS | 1126 |
| TNARGDYTAL | 1127 |
| AEGRGDTRGS | 1128 |
| TNIRGDQSSV | 1129 |
| VDRRGDTMTM | 1130 |
| QIGRGDVTPL | 1131 |
| MQARGDLLGV | 1132 |
| NTQRGDYASV | 1133 |
| SDTRGDRITQ | 1134 |
| SAIRGDYRDV | 1135 |
| VGVRGDVHPL | 1136 |
| TVLRGDAERL | 1137 |
| ASTRGDLSGQ | 1138 |
| DSRRGDGQNL | 1139 |
| IDKRGDIQGL | 1140 |
| TVTRGDLLVN | 1141 |
| DSTRGDRLGL | 1142 |
| AANRGDYASL | 1143 |
| RDYRGDTSSL | 1144 |
| SNIRGDASLH | 1145 |
| NLQRGDRTDF | 1146 |
| NASRGDHSSM | 1147 |
| TSMRGDYTHV | 1148 |
| TSRRGDLNDV | 1149 |
| QSHRGDVQSL | 1150 |
| STTRGDYQSV | 1151 |
| YSTRGDHAML | 1152 |
| TSARGDLIHT | 1153 |
| ATARGDRESF | 1154 |
| ASTRGDYSTM | 1155 |
| SNNRGDVQRL | 1156 |
| ERLRGDQHII | 1157 |
| SSIRGDHATL | 1158 |
| NPTRGDIATV | 1159 |
| MSTRGDLSVN | 1160 |
| MDRRGDITYA | 1161 |
| NATRGDLMSA | 1162 |
| SDHRGDRTYQ | 1163 |
| NWVRGDLSTA | 1164 |
| SSHRGDLSAT | 1165 |
| NATRGDYREI | 1166 |
| DTLRGDLQTA | 1167 |
| IAVRGDLTPV | 1168 |
| TLLRGDLAHS | 1169 |
| MNTRGDHTVL | 1170 |
| TGHRGDIAVY | 1171 |
| TSNRGDQMSN | 1172 |
| SATRGDHTAI | 1173 |
| SSVRGDREHA | 1174 |
| TNSRGDLVVH | 1175 |
| ETRRGDLSSQ | 1176 |
| TYARGDITLT | 1177 |
| SASRGDTLHL | 1178 |
| DNQRGDLSSR | 1179 |
| TQSRGDLRDA | 1180 |
| ENRRGDLTNA | 1181 |
| TSIRGDISSV | 1182 |
| ISARGDTMTL | 1183 |
| SRVRGDQDHW | 1184 |
| SGNRGDIASY | 1185 |
| NEQRGDRYNH | 1186 |
| EALRGDVRAL | 1187 |
| VDRRGDYTQM | 1188 |
| NNVRGDYTSV | 1189 |
| QTYRGDASVL | 1190 |
| SNYRGDHGSL | 1191 |
| TSSRGDLREV | 1192 |
| QYTRGDVSNL | 1193 |
| SDLRGDVRER | 1194 |
| QNARGDTTAL | 1195 |
| NSLRGDRDTM | 1196 |
| SNSRGDQAQV | 1197 |
| EATRGDRSTL | 1198 |
| VPQRGDITNL | 1199 |
| DKIRGDLRSI | 1200 |
| SAARGDYSIV | 1201 |
| VGLRGDLIGQ | 1202 |
| QAGRGDLTVI | 1203 |
| SKTRGDFQEA | 1204 |
| GTARGDYSML | 1205 |
| ISTRGDLVTI | 1206 |
| TGTRGDREYM | 1207 |
| MTTRGDLSIT | 1208 |
| STSRGDMTSV | 1209 |
| GPARGDFTSS | 1210 |
| VDRRGDLGHV | 1211 |
| NSGRGDMSQL | 1212 |
| SQGRGDLERY | 1213 |
| VFGRGDQYTL | 1214 |
| IVGRGDLHLQ | 1215 |
| SYSRGDTSRL | 1216 |
| MSSRGDRATI | 1217 |
| MSSRGDLTRV | 1218 |
| QHTRGDFGNL | 1219 |
| ETRRGDLSSQ | 1220 |
| TGTRGDLRAI | 1221 |
| QMARGDLSTA | 1222 |
| LVERGDRIMN | 1223 |
| GLVRGDLNAR | 1224 |
| NNPRGDLSSV | 1225 |
| HEKRGDLSNS | 1226 |
| SQARGDLHQT | 1227 |
| FTGRGDLRTA | 1228 |
| QSIRGDTGHL | 1229 |
| SAGRGDMSVL | 1230 |
| SERRGDEHKW | 1231 |
| TTGRGDLVSL | 1232 |
| LGLRGDQIGQ | 1233 |
| VALRGDLTHQ | 1234 |
| MIGRGDQAPL | 1235 |
| FQARGDHSTL | 1236 |
| NSVRGDHSSL | 1237 |
| WPNRGDLQST | 1238 |
| SASRGDYQTL | 1239 |
| TMQRGDLHTI | 1240 |
| TALRGDLAGT | 1241 |
| VNARGDFSQT | 1242 |
| GWIRGDRTSM | 1243 |
| TFSRGDHTVL | 1244 |
| SGARGDLVGV | 1245 |
| NNVRGDYTSV | 1246 |
| QTARGDHVVL | 1247 |
| TLIRGDLTRS | 1248 |
| VTTRGDRLEL | 1249 |

TABLE 5
Exemplary nucleic acids comprising the mature guide
strand of miDUX4, mi405 or mi1155 and the
miR-122 binding site or the miR-208 binding site
are provided below.

| | | | Location | Nucleotide Sequence |
|---|---|---|---|---|
| SEQ | | miR | of | (lowercase letter spacers to |
| ID | | binding | binding | facilitate proper folding of |
| NO: | miDUX4 | site | site | the per-miRNA stem) |
| 1250 | mi405 | miR-122 | Loop | CTCGAGTGAGCGATCCAGGATTCAGATC |
| | | | | TGGTTTCTATTTAGTGTGATAATGGTGTT |
| | | | | TAAACCAGATCTGAATCCTGGACTGCCTA |
| | | | | CTAGT |
| 1251 | mi1155 | miR-122 | Loop | CTCGAGTGAGCGACAGGCGCAACCTCTC |
| | | | | CTAGAATATTTAGTGTGATAATGGTGTTT |
| | | | | CTAGGAGAGGTTGCGCCTGCTGCCTACT |
| | | | | AGT |
| 1252 | mi405 | miR-122 | 5′end | CTCGAGTATTTAGTGTGATAATGGTGTTT |
| | | | | ctcggaTGAGCGATCCAGGATTCAGATCTGG |
| | | | | TTTCTGAAAGCCACAGATGGGAAACCAG |
| | | | | ATCTGAATCCTGGACTGCCTACTAGT |
| 1253 | mi1155 | miR-122 | 5′end | CTCGAGTATTTAGTGTGATAATGGTGTTT |
| | | | | ctcgggTGAGCGACAGGCGCAACCTCTCCTA |
| | | | | GAACTGTAAAGCCACAGATGGGTTCTAG |
| | | | | GAGAGGTTGCGCCTGCTGCCTACTAGT |
| 1254 | mi405 | miR-122 | 3′ end | CTCGAGTGAGCGATCCAGGATTCAGATC |
| | | | | TGGTTTCTGAAAGCCACAGATGGGAAAC |
| | | | | CAGATCTGAATCCTGGACTGCcTactagaTAT |
| | | | | TTAGTGTGATAATGGTGTTTACTAGT |
| 1255 | mi1155 | miR-122 | 3′ end | CTCGAGTGAGCGACAGGCGCAACCTCTC |
| | | | | CTAGAACTGTAAAGCCACAGATGGGTTC |
| | | | | TAGGAGAGGTTGCGCCTGCTGCCTactagaT |
| | | | | ATTTAGTGTGATAATGGTGTTTACTAGT |
| 1256 | mi405 | miR-208 | Loop | CTCGAGTGAGCGATCCAGGATTCAGATC |
| | | | | TGGTTTCTACGAGCCTTTTGCTCGTCTTA |
| | | | | TGGAAACCAGATCTGAATCCTGGACTGCC |
| | | | | TACTAGT |
| 1257 | mi1155 | miR-208 | Loop | CTCGAGTGAGCGACAGGCGCAACCTCTC |
| | | | | CTAGAACTACGAGCCTTTTGCTCGTCTTA |
| | | | | TGGTTCTAGGAGAGGTTGCGCCTGCTGC |
| | | | | CTACTAGT |
| 1258 | mi405 | miR-208 | 5′end | CTCGAGACGAGCCTTTTGCTCGTCTTATctc |
| | | | | gggTGAGCGATCCAGGATTCAGATCTGGT |
| | | | | TTCTGTAAAGCCACAGATGGGAAACCAG |
| | | | | ATCTGAATCCTGGACTGCCTACTAGT |
| 1259 | mi1155 | miR-208 | 5′end | CTCGAGTGAGCGATCCAGGATTCAGATC |
| | | | | TGGTTTCTGTAAAGCCACAGATGGGAAA |
| | | | | CCAGATCTGAATCCTGGACTGCCTactagaA |
| | | | | CGAGCCTTTTGCTCGTCTTATACTAGT |
| 1260 | mi405 | miR-208 | 3′ end | CTCGAGTGAGCGATCCAGGATTCAGATC |
| | | | | TGGTTTCTGTAAAGCCACAGATGGGAAA |
| | | | | CCAGATCTGAATCCTGGACTGCCTACTAG |
| | | | | T |
| 1261 | mi1155 | miR-208 | 3′ end | CTCGAGTGAGCGACAGGCGCAACCTCTC |
| | | | | CTAGAACTGTAAAGCCACAGATGGGTTC |
| | | | | TAGGAGAGGTTGCGCCTGCTGCCTactagaA |
| | | | | CGAGCCTTTTGCTCGTCTTATACTAGT |
| 1262 | mi405 | 5′ miR- | 3′ miR- | CTCGAGTATTTAGTGTGATAATGGTGTTT |
| | | 122 | 208 | ctcggqTGAGCGATCCAGGATTCAGATCTG |
| | | | | GTTTCTGTAAAGCCACAGATGGGAAACC |
| | | | | AGATCTGAATCCTGGACTGCCTactagaACG |
| | | | | AGCCTTTTGCTCGTCTTATACTAGT |
| 1263 | mi1155 | 5′ miR- | 3′ miR- | CTCGAGTATTTAGTGTGATAATGGTGTTT |
| | | 122, | 208 | ctcggqAGCGACAGGCGCAACCTCTCCTAGA |
| | | | | ACTGTAAAGCCACAGATGGGTTCTAGGA |
| | | | | GAGGTTGCGCCTGCTGCCTactagaACGAGC |
| | | | | CTTTTGCTCGTCTTATACTAGT |
| 1264 | mi405 | miR-122, | 5′ (both) | CTCGAGTATTTAGTGTGATAATGGTGTTT |
| | | miR-208 | | ACGAGCCTTTTGCTCGTCTTATctcgggTGA |
| | | | | GCGATCCAGGATTCAGATCTGGTTTCTGT |
| | | | | AAAGCCACAGATGGGAAACCAGATCTGA |
| | | | | ATCCTGGACTGCCTACTAGT |
| 1265 | mi1155 | miR-122, | 5′ (both) | CTCGAGTATTTAGTGTGATAATGGTGTTT |
| | | miR-208 | | ACGAGCCTTTTGCTCGTCTTATctcGOTGA |
| | | | | GCGACAGGCGCAACCTCTCCTAGAACTGT |
| | | | | AAAGCCACAGATGGGTTCTAGGAGAGGT |
| | | | | TGCGCCTGCTGCCTACTAGT |
| 1266 | mi405 | miR-122, | 3′ (both) | CTCGAGTGAGCGATCCAGGATTCAGATC |
| | | miR208 | | TGGTTTCTGTAAAGCCACAGATGGGAAA |
| | | | | CCAGATCTGAATCCTGGACTGCCTactagaT |
| | | | | ATTTAGTGTGATAATGGTGTTTACGAGCC |
| | | | | TTTTGCTCGTCTTAT ACTAGT |
| 1267 | mi1155 | miR-122, | 3′ (both) | CTCGAGTGAGCGACAGGCGCAACCTCTC |
| | | miR208 | | CTAGAACTGTAAAGCCACAGATGGGTTC |
| | | | | TAGGAGAGGTTGCGCCTGCTGCcTactagaT |
| | | | | ATTTAGTGTGATAATGGTGTTTACGAGCC |
| | | | | TTTTGCTCGTCTTAT ACTAGT |
| 1268 | mi405 | miR-122 | | CTCGAGACGAGCCTTTTGCTCGTCTTATct |
| | | loop, 5′ | | cgggTGAGCGATCCAGGATTCAGATCTGGT |
| | | miR-208 | | TTCTTATTTAGTGTGATAATGGTGTTTGG |
| | | | | AAACCAGATCTGAATCCTGGACTGCCTAC |
| | | | | TAGT |
| 1269 | mi1155 | miR-122 | | CTCGAGACGAGCCTTTTCCTCGTCTTATctc |
| | | loop, | | gggTGAGCGACAGGCGCAACCTCTCCTAG |
| | | | | AACTTATTTAGTGTGATAATGGTGTTTGG |
| | | | | TTCTAGGAGAGGTTGCGCCTGCTGCCTA |
| | | | | CTAGT |
| 1270 | mi405 | miR-122 | | CTCGAGTGAGCGATCCAGGATTCAGATC |
| | | Loop, 3′ | | TGGTTTCTTATTTAGTGTGATAATGGTGT |
| | | miR-208 | | TTGGAAACCAGATCTGAATCCTGGACTGC |
| | | | | CTactagaACGAGCCTTTTGCTCGTCTTATAC |
| | | | | TAGT |
| 1271 | mi1155 | miR-122 | | CTCGAGTGAGCGACAGGCGCAACCTCTC |
| | | Loop, 3′ | | CTAGAACTATTTAGTGTGATAATGGTGTT |
| | | miR-208 | | TGGTTCTAGGAGAGGTTGCGCCTGCTGC |
| | | | | CTactagaACGAGCCTTTTGCTCGTCTTATAC |
| | | | | TAGT |
| 1272 | mi405 | miR-208 | | CTCGAGTATTTAGTGTGATAATGGTGTT? |
| | | loop, 5′ | | ctcgggTGAGCGATCCAGGATTCAGATCTGG |
| | | miR-122 | | TTTCTACGAGCCTTTTGCTCGTCTTAT |
| | | | | GGAAACCAGATCTGAATCCTGGACTGCCT |
| | | | | ACT AGT |
| 1273 | mi1155 | miR-208 | | CTCGAGTATTTAGTGTGATAATGGTGTTT |
| | | loop, 5′ | | ctcgggTGAGCGACAGGCGCAACCTCTCCTA |
| | | miR-122 | | GAACTACGAGCCTTTTGCTCGTCTTATGG |
| | | | | TTCTAGGAGAGGTTGCGCCTGCTGCCTA |
| | | | | CTAGT |
| 1274 | mi405 | miR-208 | | CTCGAGTGAGCGATCCAGGATTCAGATC |
| | | loop, 3′ | | TGGTTTCTACGAGCCTTTTGCTCGTCTTA |
| | | miR-122 | | TGGAAACCAGATCTGAATCCTGGACTGCC |
| | | | | TactagaTATTTAGTGTGATAATGGTGTTTA |
| | | | | CTAGT |
| 1275 | mi1155 | miR-208 | | CTCGAGTGAGCGATCCAGGATTCAGATC |
| | | loop, 3′ | | TGGTTTCTTTTAGTGTGATAATGGTGTTT |
| | | miR-122 | | GACGAGCTTTTTGCTCGTCTTATGGAAAC |
| | | | | CAGATCTGAATCCTGGACTGCCTACTAGT |
| 1276 | mi405 | miR-122 | Loop | CTCGAGTGAGCGACAGGCGCAACCTCTC |
| | | miR-122 | (both) | CTAGAACTTTTAGTGTGATAATGGTGTTT |
| | | | | GACGAGCTTTTTGCTCGTCTTATGGTTCT |
| | | | | AGGAGAGGTTGCGCCTGCTGCCTACTAG |
| | | | | T |
| 1277 | mi1155 | miR-122 | Loop | CTCGAGTGAGCGACAGGCGCAACCTCTC |
| | | miR-122 | (both) | CTAGAACTTTTAGTGTGATAATGGTGTTT |
| | | | | GACGAGCTTTTTGCTCGTCTTATGGTTCT |
| | | | | AGGAGAGGTTGCGCCTGCTGCCTACTAG |
| | | | | T |
| 1278 | mi405 | miR-122 | Loop | CUCGAGUGAGCGAUCCAGGAUUCAGAU |
| | | | | CUGGUUUQUAUUUAGUGUGAJAAUGGU |
| | | | | GUUUAAACCAGAUCUGAAUCCUGGACUG |
| | | | | CCUACUAGU |
| 1279 | mi1155 | miR-122 | Loop | CUCGAGUGAGCGACAGGCGCAACCUCUC |
| | | | | CUAGAAUAUUUAGUGUGAUAAUGGUGU |
| | | | | UUCUAGGAGAGGUUGCGCCUGCUGCCU |
| | | | | ACUAGU |
| 1280 | mi405 | miR-122 | 5′end | CUCGACUAUUUAGUGUGAUAAJGGUGUU |
| | | | | UCUcggguGAGCGAUCCAGGAUUCAGAUC |
| | | | | UGGUUUCUGAAAGCCACAGAUGGGAAA |
| | | | | CCAGAUCUGAAUCCUGGACUGCCUACUA |
| | | | | GU |
| 1281 | mi1155 | miR-122 | 5′end | CUCGALAUUUAGUGUGAUAAUGGUGUU |
| | | | | UCUcggguGAGCGACAGGCGCAACCUCUCC |
| | | | | UAGAACUGUAAAGCCACAGAUGGGUUC |
| | | | | UAGGAGAGGUUGCGCCUGCUGCCUACU |
| | | | | AGU |
| 1282 | mi405 | miR-122 | 3′ end | CUCGAGUGAGCGAUCCAGGAUUCAGAU |
| | | | | CUGGUUUCUGAAAGCCACAGAUGGGAA |
| | | | | ACCAGAUCUGAAUCCUGGACUGcCuacuaga |
| | | | | UAUUUAGUGUGAUAAUGGUGUUUACUA |
| | | | | GU |
| 1283 | mi1155 | miR-122 | 3′ end | CUCGAGUGAGCGACAGGCGCAACCUCUC |
| | | | | CUAGAACUGUAAAGCCACAGAUGGGUUC |
| | | | | UAGGAGAGGUUGCGCCUGCUGcCuacUaga |
| | | | | UAUUUAGUG UGAUAAUGGUGUUU |
| | | | | ACUAGU |
| 1284 | mi405 | miR-208 | Loop | CUCGAGUGAGCGAUCCAGGAUUCAGAU |
| | | | | CUGGUUUCUACGAGCCUUUUGCUCGUCU |
| | | | | UAUGGAAACCAGAUCUGAAUCCUGGACU |
| | | | | GCCUACUAGU |
| 1285 | mi1155 | miR-208 | Loop | CUCGAGUGAGCGACAGGCGCAACCUCUC |
| | | | | CUAGAACUCGAGCcUUUUGCUCUUAUGG |
| | | | | UUCUAGGAGAGGUUGCGCCUGCUGCCU |
| | | | | ACUAGU |
| 1286 | mi405 | miR-208 | 5′end | CUCGAGACGAGCCUUUUGCUCGUCUUAU |
| | | | | CUcggguGAGCGAUCCAGGAUUCAGAUCU |
| | | | | GGUUUCUGUAAAGCCACAGAUGGGAAA |
| | | | | CCAGAUCUGAAUCCUGGACUGCCUAC |
| | | | | UAGU |
| 1287 | mi1155 | miR-208 | 5′end | CUCGACACGAGCUUUUUGCUCGUCUUAU |
| | | | | CUcggqUGAGCGACAGGCGCAACCUCUCC |
| | | | | UAGAACUGUAAAGCCACAGAUGGGUUC |
| | | | | UAGGAGAGGUUGCGCCUGCUGCCUAC |
| | | | | UAGU |
| 1288 | mi405 | miR-208 | 3′ end | CUCGAGUGAGCGAUCCAGGAUUCAGAU |
| | | | | CUGGUUUCUGUAAAGCCACAGAUGGGA |
| | | | | AACCAGAUCUGAAUCCUGGACUGCCUacU |
| | | | | agaACGAGCCUUUUGCUCGUCUUAUA |
| | | | | CUAGU |
| 1289 | mi1155 | miR-208 | 3′ end | CUCGAGUGAGCGACAGGCGCAACCUCUC |
| | | | | CUAGAACUGUAAAGCCACAGAUGGGUUC |
| | | | | UAGGAGAGGUUGCGCCUGCUGcCuacUaga |
| | | | | ACGAGCCUUUUGCUCGUCUUAUAC |
| | | | | UAGU |
| 1290 | mi405 | 5′ miR- | | CUCGAGUAUUUAGUGUGAUAAUGGUGU |
| | | 122, 3′ | | UUcUcggguGAGCGAUCCAGGAUUCAGAUC |
| | | miR-208 | | UGGUUUCUGUAAAGCCACAGAUGGGAA |
| | | | | ACCAGAUCUGAAUCCUGGACUGCCUacUa |
| | | | | gaACGAGCCUUUUUGCUCGUCUUAUACUA |
| | | | | GU |
| 1291 | mi1155 | 5′ miR- | | CUCGAGUAUUUAGUGUGAUAAUGGUGU |
| | | 122, miR- | | UUCUcggguGAGCGACAGGCGCAACCUCUC |
| | | 208 | | CUAGAACUGUAAAGCCACAGAUGGGUUC |
| | | | | UAGGAGAGGUUGCGCCUGCUGCCUacuag |
| | | | | aACGAGCCUUUUGCUCGUCUUAUACUAG |
| | | | | U |
| 1292 | mi405 | miR-122, | 5′ (both) | CUCGAGUAUUUAGUGUGAUAAUGGUGU |
| | | | | UUACGAGCCUUUUGCUCGUCUUAUCUcg |
| | | | | UGAGCGAUCCAGGAUUCAGAUCUGGUU |
| | | | | UCUGUAAAGCCACAGAUGGGAAACCAGA |
| | | | | UCUGAAUCCUGGACUGCCUACUAGU |
| 1293 | mi1155 | miR-122, | 5′ (both) | CUCGAGUAUUUAGUGUGAUAAUGGUGU |
| | | miR-208 | | UUACGAGCCUUUUGCUCGUCUUAUCUcO |
| | | | | goUGAGCGACAGGCGCAACCUCUCCUAG |
| | | | | AACUGUAAAGCCACAGAUGGGUUCUAG |
| | | | | GAGAGGUUGCGCCUGCUGCCUACUAGU |
| 1294 | mi405 | miR-122, | 3′ (both) | CUCGAGUGAGCGAUCCAGGAUUCAGAU |
| | | miR-208 | | CUGGUUUCUGUAAAGCCACAGAUGGGA |
| | | | | AACCAGAUCUGAAUCCUGGACUGCCUacu |
| | | | | agalAUUUAGUGUGAUAAUGGUGUUUACG |
| | | | | AGCCUUUUGCUCGUCUUAUACUAGU |
| 1295 | mi1155 | miR-122, | 3′ (both) | CUCGAGUGAGCGACAGGCGCAACCUCUC |
| | | miR-208 | | CUAGAACUGUAAAGCCACAGAUGGGUUC |
| | | | | UAGGAGAGGUUGCGCCUGCUGCCU |
| | | | | acUagaUAUUUAGUGUGAUAAUGGUGUUU |
| | | | | ACGAGCCUUUUGCUCGUCUUAUACUAGU |
| 1296 | mi405 | miR-122, | Loop | CUCGAGACGAGCCUUUUGCUCGUCUUAU |
| | | 5′ miR- | | CUcOggUGAGCGAUCCAGGAUUCAGAUCU |
| | | 208 | | GGUUUCUUAUUUAGUGUGAUAAUGGUG |
| | | | | UUUGGAAACCAGAUCUGAAUCCUGGACU |
| | | | | GCCUACUAGU |
| 1297 | mi1155 | miR-122, | Loop | CUCGAGACGAGCCUUUUGCUCGUCUUAU |
| | | 5′ miR- | | CUcggqUGAGCGACAGGCGCAACCUCUCC |
| | | 208 | | UAGAACUAUUUAGUGUGAUAAUGGUGU |
| | | | | UUGGUUCUAGGAGAGGUUGCGCCUGCU |
| | | | | GCCUACUAGU |
| 1298 | mi405 | miR-122, | Loop | CUCGAGUGAGCGAUCCAGGAUUCAGAU |
| | | 3′ miR- | | CUGGUUUCUUAUUUAGUGUGAUAAUGG |
| | | 208 | | UGUUUGGAAACCAGAUCUGAAUCCUGG |
| | | | | ACUGCCUacUagaACGAGCCUUUUGCUCGU |
| | | | | CUVAUACUAGU |
| 1299 | mi1155 | miR-122, | Loop | CUCGAGUGAGCGACAGGCGCAACCUCUC |
| | | 3′ miR- | | CUAGAACUU |
| | | 208 | | AUUUAGUGUGAUAAUGGUGUVUGGUUC |
| | | | | UAGGAGA |
| | | | | GGUUGCGCCUGCUGCCUacuagaACGAGCC |
| | | | | UUUUGCU CGUCUUAUACUAGU |
| 1300 | mi405 | miR-208, | Loop | CUCGAGUAUUUAGUGUGAUAAUGGUGU |
| | | 5′ miR- | | UUCUcgguGAGCGAUCCAGGAUUCAGAUC |
| | | 122 | | UGGUUUCUACGAGCCUUUUGCUCGUCU |
| | | | | UAUGGAAACCAGAUCUGAAUCCUGGACU |
| | | | | GCCUACUAGU |
| 1301 | mi1155 | miR-208, | Loop | CUCGAGUAUUUAGUGUGAUAAUGGUGU |
| | | 5′ miR- | | GUUUcUcgggUGAGCGACAGGCGCAACCU |
| | | 122 | | CUCCUAGAACUACGAGCCUUUUGCUCGU |
| | | | | CUUAUGGUUCUAGGAGAGGUUGCGCC |
| | | | | UGCUGCCUACUAGU |
| 1302 | mi405 | miR-208, | Loop | CUCGAGUGAGCGAUCCAGGAUUCAGAU |
| | | 3′ miR- | | CUGGUUUCUACGAGCCUUUUGCUCGUCU |
| | | 122 | | UAUGGAAACCAGAUCUGAAUCCUGGACU |
| | | | | GCCUacUagaUAUUUAGUGUGAUAAUGGU |
| | | | | GUUUACUAGU |
| 1303 | mi1155 | miR-208 | Loop | CUCGAGUGAGCGACAGGCGCAACCUCUC |
| | | loop, 3′ | | CUAGAACUACGAGCCUUUUGCUCGUCUU |
| | | miR-122 | | AUGGUUCUAGGAGAGGUUGCGCCUGCU |
| | | | | GCCUacUagaUAUUUAGUGUGAUAAUGGU |
| | | | | GUUUACUAGU |
| 1304 | mi405 | miR-122 | Loop | CUCGAGUGAGCGAUCCAGGAUUCAGAU |
| | | miR-122 | (both) | CUGGUUUCUUUUAGUGUGAUAAUGGUG |
| | | | | UUUGACGAGCUUUUUGCUCGUCUUAUG |
| | | | | GAAACCAGAUCUGAAUCCUGGACUGCCU |
| | | | | ACUAGU |
| 1305 | mi1155 | miR-122 | Loop | CUCGAGUGAGCGACAGGCGCAACCUCUC |
| | | miR-122 | (both) | CUAGAACUUUUAGUGUGAUAAUGGUGU |
| | | | | UUGACGAGCUUUUUGCUCGUCUUAUGG |
| | | | | UUCUAGGAGAGGUUGCGCCUGCUGCCU |
| | | | | ACUAGU |

TABLE 6
Additional exemplary miRNA.

| miRNA ID | Accession | RPM | Chromosome | Start | End | Strand | Confidence |
|---|---|---|---|---|---|---|---|
| hsa-mir-99b | MI0000746 | 1742 | chr19 | 51692612 | 51692681 | + | ✓ |
| hsa-mir-99a | MI0000101 | 26393 | chr21 | 16539089 | 16539169 | + | ✓ |
| hsa-mir-9986 | MI0032316 | — | chr2 | 132433711 | 132433820 | + | — |
| hsa-mir-9985 | MI0032313 | 1873 | chrY | 4606120 | 4606228 | + | ✓ |
| hsa-mir-9983 | MI0033670 | 3 | chr6 | 39074314 | 39074381 | + | — |
| hsa-mir-9903 | MI0031831 | 1 | chr8 | 98393645 | 98393738 | − | ✓ |
| hsa-mir-9902-2 | MI0041071 | 406 | chr12 | 122709332 | 122709424 | − | — |
| hsa-mir-9902-1 | MI0031830 | 406 | chr12 | 122695907 | 122695999 | − | — |
| hsa-mir-9901 | MI0031829 | 13 | chr16 | 34163578 | 34163673 | + | — |
| hsa-mir-9900 | MI0031828 | — | chr3 | 112737089 | 112737181 | + | — |
| hsa-mir-9899 | MI0031827 | — | chr2 | 149330278 | 149330373 | − | — |
| hsa-mir-9898 | MI0031826 | 3 | chr12 | 52898889 | 52898984 | − | — |
| hsa-mir-9851 | MI0039502 | — | chr10 | 116827488 | 116827568 | − | — |
| hsa-mir-98 | MI0000100 | 45139 | chrX | 53556223 | 53556341 | − | ✓ |
| hsa-mir-9718 | MI0030983 | — | chr14 | 60648648 | 60648711 | + | — |
| hsa-mir-96 | MI0000098 | 483 | chr7 | 129774692 | 129774769 | − | ✓ |
| hsa-mir-9500 | MI0029185 | — | chr2 | 218823090 | 218823154 | + | — |
| hsa-mir-95 | MI0000097 | 164 | chr4 | 8005301 | 8005381 | − | — |
| hsa-mir-944 | MI0005769 | 80 | chr3 | 189829922 | 189830009 | + | — |
| hsa-mir-943 | MI0005768 | — | chr4 | 1986384 | 1986477 | − | — |
| sa-mir-942 | MI0005767 | 2 | chr1 | 117094643 | 117094728 | + | ✓ |
| hsa-mir-941-5 | MI0031520 | 57 | chr20 | 63919868 | 63919939 | + | ✓ |
| hsa-mir-941-4 | MI0005766 | 57 | chr20 | 63919756 | 63919827 | + | ✓ |
| hsa-mir-941-3 | MI0005765 | 57 | chr20 | 63919561 | 63919632 | + | ✓ |
| hsa-mir-941-2 | MI0005764 | 57 | chr20 | 63919505 | 63919576 | + | ✓ |
| hsa-mir-941-1 | MI0005763 | 57 | chr20 | 63919449 | 63919520 | + | ✓ |
| hsa-mir-940 | MI0005762 | — | chr16 | 2271747 | 2271840 | + | — |
| hsa-mir-939 | MI0005761 | — | chr8 | 144394149 | 144394230 | − | — |
| hsa-mir-938 | MI0005760 | 53 | chr10 | 29602264 | 29602346 | − | — |
| hsa-mir-937 | MI0005759 | 18 | chr8 | 143812957 | 143813042 | − | — |
| hsa-mir-936 | MI0005758 | — | chr10 | 104048089 | 104048186 | − | — |
| hsa-mir-935 | MI0005757 | 11 | chr19 | 53982307 | 53982397 | + | — |
| hsa-mir-934 | MI0005756 | 5 | chrX | 136550878 | 136550960 | + | — |
| hsa-mir-933 | MI0005755 | — | chr2 | 175167633 | 175167709 | − | — |
| hsa-mir-93 | MI0000095 | 2001 | chr7 | 100093768 | 100093847 | − | ✓ |
| hsa-mir-92b | MI0003560 | 4600 | chr1 | 155195177 | 155195272 | + | ✓ |
| hsa-mir-92a-2 | MI0000094 | 7185 | chrX | 134169538 | 134169612 | − | ✓ |
| hsa-mir-92a-1 | MI0000093 | 8600 | chr13 | 91351314 | 91351391 | + | ✓ |
| hsa-mir-924 | MI0005716 | 100 | chr18 | 39622123 | 39622175 | − | — |
| hsa-mir-922 | MI0005714 | 17 | chr3 | 197674496 | 197674576 | − | — |
| hsa-mir-921 | MI0005713 | — | chr1 | 166154743 | 166154798 | − | — |
| hsa-mir-920 | MI0005712 | — | chr12 | 24212421 | 24212495 | + | — |
| hsa-mir-9-3 | MI0000468 | 1073 | chr15 | 89368017 | 89368106 | + | ✓ |
| hsa-mir-9-2 | MI0000467 | 1073 | chr5 | 88666853 | 88666939 | − | ✓ |
| hsa-mir-9-1 | MI0000466 | 1073 | chr1 | 156420341 | 156420429 | − | ✓ |
| hsa-mir-892c | MI0022560 | 2 | chrX | 145992750 | 145992826 | − | — |
| hsa-mir-892b | MI0005538 | — | chrX | 145997198 | 145997274 | − | — |
| hsa-mir-892a | MI0005528 | — | chrX | 145996669 | 145996743 | − | — |
| hsa-mir-891b | MI0005534 | — | chrX | 146001053 | 146001131 | − | — |
| hsa-mir-891a | MI0005524 | — | chrX | 146027794 | 146027872 | − | — |
| hsa-mir-890 | MI0005533 | 2 | chrX | 145994275 | 145994351 | − | — |
| hsa-mir-889 | MI0005540 | 26 | chr14 | 101047901 | 101047979 | + | ✓ |
| hsa-mir-888 | MI0005537 | 784 | chrX | 145994784 | 145994860 | − | ✓ |
| hsa-mir-887 | MI0005562 | 6 | chr5 | 15935182 | 15935260 | + | ✓ |
| hsa-mir-885 | MI0005560 | 4 | chr3 | 10394489 | 10394562 | − | ✓ |
| hsa-mir-877 | MI0005561 | 70 | chr6 | 30584332 | 30584417 | + | — |
| hsa-mir-876 | MI0005542 | 28 | chr9 | 28863626 | 28863706 | − | ✓ |
| hsa-mir-875 | MI0005541 | — | chr8 | 99536786 | 99536861 | − | — |
| hsa-mir-874 | MI0005532 | 23 | chr5 | 137647572 | 137647649 | − | ✓ |
| hsa-mir-873 | MI0005564 | 8 | chr9 | 28888879 | 28888955 | − | ✓ |
| hsa-mir-8485 | MI0027288 | 1000 | chr2 | 50696172 | 50696262 | − | — |
| hsa-mir-8089 | MI0025925 | 28 | chr5 | 181043403 | 181043484 | − | — |
| hsa-mir-8088 | MI0025924 | — | chrX | 52336557 | 52336642 | − | — |
| hsa-mir-8087 | MI0025923 | 65 | chr11 | 27514970 | 27515047 | − | — |
| hsa-mir-8086 | MI0025922 | 209 | chr10 | 28289258 | 28289350 | − | — |
| hsa-mir-8085 | MI0025921 | — | chr19 | 44758657 | 44758721 | + | — |
| hsa-mir-8084 | MI0025920 | — | chr8 | 93029751 | 93029839 | + | — |
| hsa-mir-8083 | MI0025919 | — | chr1 | 153689705 | 153689793 | − | — |
| hsa-mir-8082 | MI0025918 | — | chr4 | 113152282 | 113152362 | + | — |
| hsa-mir-8081 | MI0025917 | 472 | chr9 | 106600928 | 106601022 | + | — |
| hsa-mir-8080 | MI0025916 | 15 | chr2 | 79866495 | 79866583 | − | — |
| hsa-mir-8079 | MI0025915 | — | chr13 | 44196129 | 44196200 | − | — |
| hsa-mir-8078 | MI0025914 | — | chr18 | 112256 | 112339 | − | — |
| hsa-mir-8077 | MI0025913 | — | chr19 | 42351131 | 42351205 | + | — |
| hsa-mir-8076 | MI0025912 | — | chr3 | 113432118 | 113432200 | − | — |
| hsa-mir-8075 | MI0025911 | — | chr13 | 113262920 | 113262999 | + | — |
| hsa-mir-8074 | MI0025910 | — | chr19 | 51206929 | 51207009 | − | — |
| hsa-mir-8073 | MI0025909 | — | chr13 | 110340958 | 110341029 | + | — |
| hsa-mir-8072 | MI0025908 | 5 | chr12 | 123364764 | 123364843 | − | — |
| hsa-mir-8071-2 | MI0026417 | — | chr14 | 105640168 | 105640232 | + | — |
| hsa-mir-8071-1 | MI0025907 | — | chr14 | 105621116 | 105621180 | + | — |
| hsa-mir-8070 | MI0025906 | — | chr11 | 11783135 | 11783222 | − | — |
| hsa-mir-8069-2 | MI0031519 | 12 | chr21 | 13724189 | 13724274 | + | — |
| hsa-mir-8069-1 | MI0025905 | 12 | chr21 | 6859171 | 6859256 | + | — |
| hsa-mir-8068 | MI0025904 | — | chr11 | 28477481 | 28477548 | − | — |
| hsa-mir-8067 | MI0025903 | — | chr15 | 62304658 | 62304734 | − | — |
| hsa-mir-8066 | MI0025902 | — | chr4 | 101240795 | 101240872 | − | — |
| hsa-mir-8065 | MI0025901 | 180 | chr16 | 5632467 | 5632566 | + | — |
| hsa-mir-8064 | MI0025900 | 3 | chr3 | 52846463 | 52846552 | − | — |
| hsa-mir-8063 | MI0025899 | — | chr15 | 36972821 | 36972901 | − | — |
| hsa-mir-8062 | MI0025898 | — | chr20 | 7371608 | 7371692 | + | — |
| hsa-mir-8061 | MI0025897 | — | chr19 | 54645309 | 54645383 | + | — |
| hsa-mir-8060 | MI0025896 | — | chr3 | 96359964 | 96360039 | + | — |
| hsa-mir-8059 | MI0025895 | — | chr17 | 50768650 | 50768730 | + | — |
| hsa-mir-8058 | MI0025894 | 8 | chr16 | 82688931 | 82689019 | + | — |
| hsa-mir-8057 | MI0025893 | 5 | chr18 | 26591467 | 26591535 | − | — |
| hsa-mir-8056 | MI0025892 | — | chr5 | 173347455 | 173347536 | + | — |
| hsa-mir-8055 | MI0025891 | — | chr8 | 6622124 | 6622220 | − | — |
| hsa-mir-8054 | MI0025890 | — | chr11 | 23419105 | 23419190 | − | — |
| hsa-mir-8053 | MI0025889 | — | chr4 | 47652669 | 47652743 | + | — |
| hsa-mir-8052 | MI0025888 | — | chr11 | 130666735 | 130666803 | + | — |
| hsa-mir-802 | MI0003906 | 266 | chr21 | 35720715 | 35720808 | + | — |
| hsa-mir-7978 | MI0025754 | — | chr4 | 21464700 | 21464758 | − | — |
| hsa-mir-7977 | MI0025753 | 58 | chr3 | 176515103 | 176515151 | + | — |
| hsa-mir-7976 | MI0025752 | — | chr3 | 127587111 | 127587176 | − | ✓ |
| hsa-mir-7975 | MI0025751 | 121 | chr19 | 55123225 | 55123292 | − | — |
| hsa-mir-7974 | MI0025750 | 21 | chr19 | 11495544 | 11495622 | − | — |
| hsa-mir-7973-2 | MI0025749 | — | chr15 | 51314032 | 51314107 | − | — |
| hsa-mir-7973-1 | MI0025748 | — | chr15 | 51314034 | 51314109 | + | — |
| hsa-mir-7856 | MI0025526 | — | chr1 | 86357632 | 86357687 | − | — |
| hsa-mir-7855 | MI0025525 | 35 | chr14 | 64785626 | 64785686 | − | — |
| hsa-mir-7854 | MI0025524 | — | chr16 | 81533902 | 81533966 | + | — |
| hsa-mir-7853 | MI0025523 | 11 | chr6 | 6169304 | 6169435 | − | — |
| hsa-mir-7852 | MI0025522 | — | chr1 | 107897223 | 107897304 | + | — |
| hsa-mir-7851 | MI0025521 | 837 | chr12 | 42323700 | 42323859 | − | — |
| hsa-mir-7850 | MI0025520 | 64 | chr19 | 2630715 | 2630793 | + | — |
| hsa-mir-7849 | MI0025519 | — | chr4 | 146408583 | 146408688 | + | — |
| hsa-mir-7848 | MI0025518 | — | chr8 | 133046481 | 133046581 | − | — |
| hsa-mir-7847 | MI0025517 | 24 | chr11 | 1880045 | 1880147 | + | — |
| hsa-mir-7846 | MI0025516 | 4 | chr1 | 12166943 | 12167038 | + | — |
| hsa-mir-7845 | MI0025515 | 4 | chr2 | 207166400 | 207166498 | + | — |
| hsa-mir-7844 | MI0025514 | — | chr12 | 94571231 | 94571352 | − | — |
| hsa-mir-7843 | MI0025510 | — | chr14 | 72516820 | 72516898 | + | — |
| hsa-mir-7706 | MI0025242 | 1 | chr15 | 85380596 | 85380662 | + | — |
| hsa-mir-7705 | MI0025241 | — | chr8 | 100702968 | 100703024 | − | — |
| hsa-mir-7704 | MI0025240 | 29 | chr2 | 176188843 | 176188901 | + | — |
| hsa-mir-7703 | MI0025239 | 4 | chr14 | 24143489 | 24143565 | − | — |
| hsa-mir-7702 | MI0025238 | — | chr9 | 111271156 | 111271214 | − | — |
| hsa-mir-770 | MI0005118 | — | chr14 | 100852390 | 100852487 | + | ✓ |
| hsa-mir-769 | MI0003834 | 120 | chr19 | 46018932 | 46019049 | + | ✓ |
| hsa-mir-767 | MI0003763 | 30 | chrX | 152393421 | 152393529 | − | ✓ |
| hsa-mir-766 | MI0003836 | 10 | chrX | 119646738 | 119646848 | − | ✓ |
| hsa-mir-765 | MI0005116 | 8 | chr1 | 156936131 | 156936244 | − | — |
| hsa-mir-764 | MI0003944 | — | chrX | 114639435 | 114639519 | + | — |
| hsa-mir-762 | MI0003892 | — | chr16 | 30893903 | 30893985 | + | — |
| hsa-mir-761 | MI0003941 | 40 | chr1 | 51836344 | 51836402 | − | — |
| hsa-mir-760 | MI0005567 | 9 | chr1 | 93846832 | 93846911 | + | — |
| hsa-mir-759 | MI0004065 | — | chr13 | 52810050 | 52810140 | + | — |
| hsa-mir-758 | MI0003757 | 2 | chr14 | 101026020 | 101026107 | + | ✓ |
| hsa-mir-7515 | MI0024354 | 20 | chr2 | 6650373 | 6650439 | + | — |
| hsa-mir-744 | MI0005559 | 324 | chr17 | 12081899 | 12081996 | + | ✓ |
| hsa-mir-718 | MI0012489 | 2 | chrX | 154019920 | 154019989 | − | — |
| hsa-mir-7162 | MI0023623 | — | chr10 | 30368597 | 30368665 | − | — |
| hsa-mir-7161 | MI0023619 | 107 | chr6 | 158609707 | 158609790 | + | — |
| hsa-mir-7160 | MI0023621 | — | chr8 | 2076589 | 2076640 | + | — |
| hsa-mir-7159 | MI0023620 | 50 | chr6 | 33899135 | 33899200 | + | — |
| hsa-mir-7158 | MI0023618 | — | chr2 | 5974662 | 5974732 | + | — |
| hsa-mir-7157 | MI0023617 | — | chr2 | 140586626 | 140586685 | − | — |
| hsa-mir-7156 | MI0023616 | — | chr1 | 77060143 | 77060202 | + | — |
| hsa-mir-7155 | MI0023615 | — | chr11 | 64341849 | 64341904 | − | — |
| hsa-mir-7154 | MI0023614 | — | chr11 | 45691704 | 45691776 | − | ✓ |
| hsa-mir-7153 | MI0023613 | — | chr18 | 11654885 | 11654941 | − | — |
| hsa-mir-7152 | MI0023612 | — | chr10 | 71790747 | 71790800 | + | — |
| hsa-mir-7151 | MI0023611 | — | chr10 | 67403351 | 67403410 | − | — |
| hsa-mir-7150 | MI0023610 | — | chr9 | 123485529 | 123485622 | − | — |
| hsa-mir-7114 | MI0022965 | — | chr9 | 137450026 | 137450086 | − | — |
| hsa-mir-7113 | MI0022964 | — | chr11 | 68032864 | 68032922 | + | — |
| hsa-mir-7112 | MI0022963 | — | chr8 | 144262673 | 144262737 | − | — |
| hsa-mir-7111 | MI0022962 | — | chr6 | 35470508 | 35470579 | + | — |
| hsa-mir-7110 | MI0022961 | 578 | chr3 | 123161794 | 123161879 | + | — |
| hsa-mir-711 | MI0012488 | — | chr3 | 48578902 | 48578977 | − | — |
| hsa-mir-7109 | MI0022960 | 2 | chr22 | 31621467 | 31621531 | − | — |
| hsa-mir-7108 | MI0022959 | 100 | chr19 | 2434914 | 2435000 | − | — |
| hsa-mir-7107 | MI0022958 | 13 | chr12 | 121444273 | 121444352 | − | — |
| hsa-mir-7106 | MI0022957 | — | chr12 | 113159113 | 113159177 | − | — |
| hsa-mir-708 | MI0005543 | 184 | chr11 | 79402022 | 79402109 | − | ✓ |
| hsa-mir-7-3 | MI0000265 | 3279 | chr19 | 4770670 | 4770779 | + | — |
| hsa-mir-7-2 | MI0000264 | 3265 | chr15 | 88611825 | 88611934 | + | ✓ |
| hsa-mir-7-1 | MI0000263 | 3664 | chr9 | 83969748 | 83969857 | − | ✓ |
| hsa-mir-6895 | MI0022742 | 4 | chrX | 53195411 | 53195488 | − | — |
| hsa-mir-6894 | MI0022741 | 1 | chrX | 53198889 | 53198945 | − | — |
| hsa-mir-6893 | MI0022740 | — | chr8 | 144435551 | 144435619 | − | — |
| hsa-mir-6892 | MI0022739 | 35 | chr7 | 143382686 | 143382800 | + | — |
| hsa-mir-6891 | MI0022738 | 12 | chr6 | 31355224 | 31355316 | − | — |
| hsa-mir-6890 | MI0022737 | — | chr3 | 49099854 | 49099914 | − | — |
| hsa-mir-6889 | MI0022736 | — | chr22 | 41252992 | 41253050 | − | — |
| hsa-mir-6888 | MI0022735 | 2 | chr2 | 159186835 | 159186901 | + | — |
| hsa-mir-6887 | MI0022734 | — | chr19 | 35122700 | 35122764 | + | — |
| hsa-mir-6886 | MI0022733 | — | chr19 | 11113474 | 11113534 | + | — |
| hsa-mir-6885 | MI0022732 | — | chr19 | 6389638 | 6389703 | − | — |
| hsa-mir-6884 | MI0022731 | — | chr17 | 40026332 | 40026409 | − | — |
| hsa-mir-6883 | MI0022730 | — | chr17 | 8144994 | 8145071 | − | — |
| hsa-mir-6882 | MI0022729 | 29 | chr15 | 74840642 | 74840707 | − | — |
| hsa-mir-6881 | MI0022728 | — | chr15 | 74411357 | 74411432 | − | — |
| hsa-mir-6880 | MI0022727 | 2 | chr12 | 124337181 | 124337242 | − | — |
| hsa-mir-6879 | MI0022726 | 162 | chr11 | 65018505 | 65018570 | + | — |
| hsa-mir-6878 | MI0022725 | 69 | chr1 | 150492345 | 150492410 | + | — |
| hsa-mir-6877 | MI0022724 | — | chr9 | 133051996 | 133052059 | + | — |
| hsa-mir-6876 | MI0022723 | 12 | chr8 | 25345402 | 25345474 | + | — |
| hsa-mir-6875 | MI0022722 | 49 | chr7 | 100868036 | 100868107 | + | — |
| hsa-mir-6874 | MI0022721 | 18 | chr7 | 5711840 | 5711910 | − | — |
| hsa-mir-6873 | MI0022720 | 72 | chr6 | 33287227 | 33287289 | − | — |
| hsa-mir-6872 | MI0022719 | — | chr3 | 50273236 | 50273297 | + | — |
| hsa-mir-6871 | MI0022718 | — | chr20 | 41169023 | 41169078 | + | — |
| hsa-mir-6870 | MI0022717 | 2 | chr20 | 10649636 | 10649695 | − | — |
| hsa-mir-6869 | MI0022716 | — | chr20 | 1392900 | 1392961 | − | — |
| hsa-mir-6868 | MI0022715 | — | chr17 | 76098019 | 76098076 | − | — |
| hsa-mir-6867 | MI0022714 | 4 | chr17 | 40193597 | 40193663 | + | — |
| hsa-mir-6866 | MI0022713 | — | chr17 | 40161933 | 40162001 | + | — |
| hsa-mir-6865 | MI0022712 | — | chr17 | 4970086 | 4970150 | − | — |
| hsa-mir-6864 | MI0022711 | — | chr17 | 4969702 | 4969771 | − | — |
| hsa-mir-6863 | MI0022710 | — | chr16 | 56904264 | 56904353 | + | — |
| hsa-mir-6862-2 | MI0026415 | — | chr16 | 28724252 | 28724321 | + | — |
| hsa-mir-6862-1 | MI0022709 | — | chr16 | 28390982 | 28391051 | − | — |
| hsa-mir-6861 | MI0022708 | 6 | chr12 | 112163258 | 112163321 | − | — |
| hsa-mir-6860 | MI0022707 | — | chr11 | 67045643 | 67045708 | + | — |
| hsa-mir-6859-4 | MI0031521 | — | chr16 | 17052 | 17119 | − | — |
| hsa-mir-6859-3 | MI0026421 | — | chr15 | 101973524 | 101973591 | + | — |
| hsa-mir-6859-2 | MI0026420 | — | chr1 | 187891 | 187958 | − | — |
| hsa-mir-6859-1 | MI0022705 | — | chr1 | 17369 | 17436 | − | — |
| hsa-mir-6858 | MI0022704 | 19 | chrX | 154450320 | 154450386 | + | — |
| hsa-mir-6857 | MI0022703 | — | chrX | 53405673 | 53405765 | − | — |
| hsa-mir-6856 | MI0022702 | 47 | chr9 | 130626297 | 130626363 | + | — |
| hsa-mir-6855 | MI0022701 | — | chr9 | 129869605 | 129869671 | + | ✓ |
| hsa-mir-6854 | MI0022700 | — | chr9 | 98229149 | 98229217 | − | — |
| hsa-mir-6853 | MI0022699 | — | chr9 | 35732922 | 35732995 | + | — |
| hsa-mir-6852 | MI0022698 | — | chr9 | 35710676 | 35710741 | − | — |
| hsa-mir-6851 | MI0022697 | 2 | chr9 | 33467869 | 33467935 | − | — |
| hsa-mir-6850 | MI0022696 | — | chr8 | 144791931 | 144791991 | − | — |
| hsa-mir-6849 | MI0022695 | — | chr8 | 144400277 | 144400345 | − | — |
| hsa-mir-6848 | MI0022694 | — | chr8 | 144317246 | 144317315 | − | — |
| hsa-mir-6847 | MI0022693 | — | chr8 | 144079874 | 144079942 | + | — |
| hsa-mir-6846 | MI0022692 | 20 | chr8 | 144057321 | 144057380 | − | — |
| hsa-mir-6845 | MI0022691 | — | chr8 | 143837756 | 143837816 | − | — |
| hsa-mir-6844 | MI0022690 | — | chr8 | 124508515 | 124508576 | − | — |
| hsa-mir-6843 | MI0022689 | — | chr8 | 27610601 | 27610751 | − | — |
| hsa-mir-6842 | MI0022688 | 34 | chr8 | 27433370 | 27433434 | + | — |
| hsa-mir-6841 | MI0022687 | — | chr8 | 24953796 | 24953867 | − | — |
| hsa-mir-6840 | MI0022686 | — | chr7 | 100356651 | 100356721 | + | — |
| hsa-mir-6839 | MI0022685 | — | chr7 | 64679064 | 64679176 | + | — |
| hsa-mir-6838 | MI0022684 | — | chr7 | 44073378 | 44073433 | − | — |
| hsa-mir-6837 | MI0022683 | — | chr7 | 44051766 | 44051829 | + | — |
| hsa-mir-6836 | MI0022682 | 10 | chr7 | 2257515 | 2257577 | − | — |
| hsa-mir-6835 | MI0022680 | — | chr6 | 34240673 | 34240736 | + | — |
| hsa-mir-6834 | MI0022679 | — | chr6 | 33290245 | 33290325 | + | — |
| hsa-mir-6833 | MI0022678 | 1 | chr6 | 32179816 | 32179876 | + | — |
| hsa-mir-6832 | MI0022677 | 12 | chr6 | 31633787 | 31633858 | + | — |
| hsa-mir-6831 | MI0022676 | — | chr5 | 140563671 | 140563751 | − | — |
| hsa-mir-6830 | MI0022675 | — | chr5 | 132217849 | 132217918 | − | — |
| hsa-mir-6829 | MI0022674 | — | chr3 | 195882329 | 195882395 | − | — |
| hsa-mir-6828 | MI0022673 | — | chr3 | 170423103 | 170423162 | + | — |
| hsa-mir-6827 | MI0022672 | — | chr3 | 134367804 | 134367862 | − | — |
| hsa-mir-6826 | MI0022671 | 3 | chr3 | 129272146 | 129272243 | + | — |
| hsa-mir-6825 | MI0022670 | — | chr3 | 127575266 | 127575331 | − | — |
| hsa-mir-6824 | MI0022669 | — | chr3 | 48633636 | 48633698 | − | — |
| hsa-mir-6823 | MI0022668 | — | chr3 | 48549961 | 48550021 | − | — |
| hsa-mir-6822 | MI0022667 | 3 | chr3 | 39138206 | 39138266 | + | — |
| hsa-mir-6821 | MI0022666 | 5 | chr22 | 49962866 | 49962939 | + | — |
| hsa-mir-6820 | MI0022665 | — | chr22 | 37967563 | 37967624 | + | ✓ |
| hsa-mir-6819 | MI0022664 | — | chr22 | 36286847 | 36286907 | − | — |
| hsa-mir-6818 | MI0022663 | 5 | chr22 | 30007049 | 30007113 | + | — |
| hsa-mir-6817 | MI0022662 | — | chr22 | 25455646 | 25455711 | + | — |
| hsa-mir-6816 | MI0022661 | — | chr22 | 20114686 | 20114751 | − | — |
| hsa-mir-6815 | MI0022660 | — | chr21 | 45478266 | 45478326 | + | — |
| hsa-mir-6814 | MI0022659 | — | chr21 | 41746772 | 41746841 | − | — |
| hsa-mir-6813 | MI0022658 | — | chr20 | 64076955 | 64077010 | − | — |
| hsa-mir-6812 | MI0022657 | 90 | chr20 | 45425510 | 45425573 | + | — |
| hsa-mir-6811 | MI0022656 | — | chr2 | 237510931 | 237510988 | + | — |
| hsa-mir-6810 | MI0022655 | — | chr2 | 218341911 | 218341980 | + | — |
| hsa-mir-6809 | MI0022654 | 2 | chr2 | 217900513 | 217900628 | − | — |
| hsa-mir-6808 | MI0022653 | — | chr1 | 1339650 | 1339708 | − | — |
| hsa-mir-6807 | MI0022652 | 8 | chr19 | 58550285 | 58550376 | + | — |
| hsa-mir-6806 | MI0022651 | — | chr19 | 58334688 | 58334751 | + | — |
| hsa-mir-6805 | MI0022650 | 28 | chr19 | 55388181 | 55388242 | + | — |
| hsa-mir-6804 | MI0022649 | — | chr19 | 55230885 | 55230952 | − | — |
| hsa-mir-6803 | MI0022648 | 9 | chr19 | 55245186 | 55245250 | − | — |
| hsa-mir-6802 | MI0022647 | — | chr19 | 55239912 | 55239976 | − | — |
| hsa-mir-6801 | MI0022646 | — | chr19 | 52222020 | 52222098 | + | — |
| hsa-mir-6800 | MI0022645 | 1 | chr19 | 49832018 | 49832099 | + | — |
| hsa-mir-6799 | MI0022644 | — | chr19 | 49791866 | 49791934 | + | — |
| hsa-mir-6798 | MI0022643 | — | chr19 | 49009906 | 49009972 | + | — |
| hsa-mir-6797 | MI0022642 | 6 | chr19 | 41869627 | 41869698 | + | — |
| hsa-mir-6796 | MI0022641 | 2 | chr19 | 40369846 | 40369907 | + | — |
| hsa-mir-6795 | MI0022640 | 3 | chr19 | 15179283 | 15179350 | − | — |
| hsa-mir-6794 | MI0022639 | 26 | chr19 | 12852260 | 12852327 | + | — |
| hsa-mir-6793 | MI0022638 | — | chr19 | 10828973 | 10829035 | + | — |
| hsa-mir-6792 | MI0022637 | 7 | chr19 | 7617439 | 7617505 | + | — |
| hsa-mir-6791 | MI0022636 | 5 | chr19 | 6736712 | 6736778 | − | — |
| hsa-mir-6790 | MI0022635 | — | chr19 | 6392921 | 6392983 | − | — |
| hsa-mir-6789 | MI0022634 | 51 | chr19 | 2235829 | 2235926 | − | — |
| hsa-mir-6788 | MI0022633 | 65 | chr18 | 10759584 | 10759649 | − | — |
| hsa-mir-6787 | MI0022632 | — | chr17 | 82236668 | 82236728 | + | — |
| hsa-mir-6786 | MI0022631 | 13 | chr17 | 81693757 | 81693869 | + | — |
| hsa-mir-6785 | MI0022630 | 2 | chr17 | 75498548 | 75498628 | + | — |
| hsa-mir-6784 | MI0022629 | — | chr17 | 45114367 | 45114433 | − | — |
| hsa-mir-6783 | MI0022628 | — | chr17 | 44934618 | 44934681 | − | — |
| hsa-mir-6782 | MI0022627 | — | chr17 | 44207771 | 44207839 | − | — |
| hsa-mir-6781 | MI0022626 | — | chr17 | 42823880 | 42823943 | − | — |
| hsa-mir-6780b | MI0022681 | — | chr6 | 43434542 | 43434620 | + | — |
| hsa-mir-6780a | MI0022625 | — | chr17 | 42708084 | 42708151 | − | — |
| hsa-mir-6779 | MI0022624 | 6 | chr17 | 38914979 | 38915042 | + | — |
| hsa-mir-6778 | MI0022623 | 5 | chr17 | 18340814 | 18340886 | − | — |
| hsa-mir-6777 | MI0022622 | — | chr17 | 17813480 | 17813545 | − | — |
| hsa-mir-6776 | MI0022621 | — | chr17 | 2692861 | 2692919 | − | — |
| hsa-mir-6775 | MI0022620 | — | chr16 | 87834592 | 87834660 | − | — |
| hsa-mir-6774 | MI0022619 | — | chr16 | 85918347 | 85918416 | + | — |
| hsa-mir-6773 | MI0022618 | — | chr16 | 68233426 | 68233499 | − | — |
| hsa-mir-6772 | MI0022617 | — | chr16 | 57772289 | 57772352 | − | — |
| hsa-mir-6771 | MI0022616 | 5 | chr16 | 50292616 | 50292675 | + | — |
| hsa-mir-6770-3 | MI0026419 | — | chr16 | 18379351 | 18379410 | − | ✓ |
| hsa-mir-6770-2 | MI0026418 | — | chr16 | 16329305 | 16329364 | + | ✓ |
| hsa-mir-6770-1 | MI0022615 | — | chr16 | 14930820 | 14930879 | + | ✓ |
| hsa-mir-6769b | MI0022706 | — | chr1 | 206474803 | 206474864 | + | — |
| hsa-mir-6769a | MI0022614 | — | chr16 | 4671318 | 4671390 | + | — |
| hsa-mir-6768 | MI0022613 | — | chr16 | 2463967 | 2464038 | + | — |
| hsa-mir-6767 | MI0022612 | — | chr16 | 2445392 | 2445457 | + | — |
| hsa-mir-6766 | MI0022611 | — | chr15 | 89326739 | 89326810 | − | — |
| hsa-mir-6765 | MI0022610 | 9 | chr14 | 105150778 | 105150864 | − | — |
| hsa-mir-6764 | MI0022609 | 2 | chr14 | 100277357 | 100277417 | + | — |
| hsa-mir-6763 | MI0022608 | 5 | chr12 | 132581997 | 132582061 | + | — |
| hsa-mir-6762 | MI0022607 | — | chr12 | 113291523 | 113291608 | + | — |
| hsa-mir-6761 | MI0022606 | 1 | chr12 | 111799834 | 111799905 | + | — |
| hsa-mir-6760 | MI0022605 | — | chr12 | 111304142 | 111304209 | + | — |
| hsa-mir-676 | MI0016436 | — | chrX | 70022857 | 70022923 | + | ✓ |
| hsa-mir-6759 | MI0022604 | — | chr12 | 57748618 | 57748682 | − | — |
| hsa-mir-6758 | MI0022603 | — | chr12 | 57512688 | 57512750 | + | ✓ |
| hsa-mir-6757 | MI0022602 | 3 | chr12 | 53056944 | 53057012 | + | — |
| hsa-mir-6756 | MI0022601 | 21 | chr11 | 119312950 | 119313012 | − | — |
| hsa-mir-6755 | MI0022600 | — | chr11 | 86278333 | 86278398 | + | — |
| hsa-mir-6754 | MI0022599 | — | chr11 | 71473503 | 71473568 | + | — |
| hsa-mir-6753 | MI0022598 | 13 | chr11 | 68044794 | 68044957 | + | — |
| hsa-mir-6752 | MI0022597 | 17 | chr11 | 67490245 | 67490315 | + | — |
| hsa-mir-6751 | MI0022596 | 41 | chr11 | 65129916 | 65129978 | − | — |
| hsa-mir-6750 | MI0022595 | 2 | chr11 | 64898363 | 64898437 | − | — |
| hsa-mir-675 | MI0005416 | 20 | chr11 | 1996759 | 1996831 | − | ✓ |
| hsa-mir-6749 | MI0022594 | 7 | chr11 | 64902387 | 64902455 | − | — |
| hsa-mir-6748 | MI0022593 | — | chr11 | 62789815 | 62789885 | + | — |
| hsa-mir-6747 | MI0022592 | 5 | chr11 | 62567011 | 62567071 | − | — |
| hsa-mir-6746 | MI0022591 | 2 | chr11 | 61878216 | 61878278 | − | — |
| hsa-mir-6745 | MI0022590 | — | chr11 | 47179611 | 47179737 | − | — |
| hsa-mir-6744 | MI0022589 | — | chr11 | 1256605 | 1256670 | + | — |
| hsa-mir-6743 | MI0022588 | — | chr11 | 209336 | 209406 | + | — |
| hsa-mir-6742 | MI0022587 | — | chr1 | 228397048 | 228397109 | − | — |
| hsa-mir-6741 | MI0022586 | — | chr1 | 225922080 | 225922142 | − | — |
| hsa-mir-6740 | MI0022585 | 4 | chr1 | 202003124 | 202003236 | + | — |
| hsa-mir-6739 | MI0022584 | — | chr1 | 201863373 | 201863447 | + | — |
| hsa-mir-6738 | MI0022583 | — | chr1 | 155951273 | 155951336 | − | — |
| hsa-mir-6737 | MI0022582 | — | chr1 | 153962351 | 153962420 | − | — |
| hsa-mir-6736 | MI0022581 | 6 | chr1 | 145850587 | 145850645 | − | — |
| hsa-mir-6735 | MI0022580 | 1 | chr1 | 43448539 | 43448611 | + | — |
| hsa-mir-6734 | MI0022579 | 1 | chr1 | 43364648 | 43364715 | − | — |
| hsa-mir-6733 | MI0022578 | — | chr1 | 43171652 | 43171712 | − | — |
| hsa-mir-6732 | MI0022577 | 2 | chr1 | 37480230 | 37480289 | + | — |
| hsa-mir-6731 | MI0022576 | 2 | chr1 | 24919345 | 24919416 | − | — |
| hsa-mir-6730 | MI0022575 | 51 | chr1 | 12578957 | 12579023 | − | — |
| hsa-mir-6729 | MI0022574 | — | chr1 | 12029158 | 12029222 | + | — |
| hsa-mir-6728 | MI0022573 | 84 | chr1 | 8866502 | 8866590 | − | — |
| hsa-mir-6727 | MI0022572 | 5 | chr1 | 1312502 | 1312566 | − | — |
| hsa-mir-6726 | MI0022571 | — | chr1 | 1296110 | 1296170 | − | — |
| hsa-mir-6724-4 | MI0031518 | 1 | chr21 | 8432530 | 8432621 | + | — |
| hsa-mir-6724-3 | MI0031517 | 1 | chr21 | 8388362 | 8388453 | + | — |
| hsa-mir-6724-2 | MI0031516 | 1 | chr21 | 8249505 | 8249596 | + | — |
| hsa-mir-6724-1 | MI0022559 | 1 | chr21 | 8205315 | 8205406 | + | — |
| hsa-mir-6722 | MI0022557 | — | chr9 | 136746893 | 136746970 | − | — |
| hsa-mir-6721 | MI0022556 | 2 | chr6 | 32170030 | 32170116 | − | — |
| hsa-mir-6720 | MI0022555 | 1 | chr6 | 1390314 | 1390411 | − | ✓ |
| hsa-mir-6719 | MI0022554 | 33 | chr19 | 39829716 | 39829802 | − | — |
| hsa-mir-6718 | MI0022553 | — | chr18 | 3885353 | 3885432 | + | — |
| hsa-mir-6717 | MI0022551 | — | chr14 | 21023314 | 21023386 | − | — |
| hsa-mir-6716 | MI0022550 | 13 | chr11 | 118644000 | 118644079 | + | — |
| hsa-mir-6715b | MI0022549 | — | chr10 | 112299612 | 112299688 | − | — |
| hsa-mir-6715a | MI0022548 | 24 | chr10 | 112299612 | 112299690 | + | — |
| hsa-mir-671 | MI0003760 | 77 | chr7 | 151238421 | 151238538 | + | ✓ |
| hsa-mir-670 | MI0003933 | — | chr11 | 43559656 | 43559753 | + | — |
| hsa-mir-668 | MI0003761 | 7 | chr14 | 101055258 | 101055323 | + | — |
| hsa-mir-665 | MI0005563 | 14 | chr14 | 100875033 | 100875104 | + | — |
| hsa-mir-664b | MI0019134 | 2 | chrX | 154768596 | 154768656 | + | ✓ |
| hsa-mir-664a | MI0006442 | 64 | chr1 | 220200538 | 220200619 | − | ✓ |
| hsa-mir-663b | MI0006336 | 9 | chr2 | 132256966 | 132257080 | − | — |
| hsa-mir-663a | MI0003672 | 10 | chr20 | 26208186 | 26208278 | − | — |
| hsa-mir-662 | MI0003670 | — | chr16 | 770183 | 770277 | + | — |
| hsa-mir-661 | MI0003669 | 130 | chr8 | 143945191 | 143945279 | − | — |
| hsa-mir-660 | MI0003684 | 510 | chrX | 50013241 | 50013337 | + | ✓ |
| hsa-mir-659 | MI0003683 | 1 | chr22 | 37847678 | 37847774 | − | — |
| hsa-mir-658 | MI0003682 | 1 | chr22 | 37844272 | 37844371 | − | — |
| hsa-mir-657 | MI0003681 | 7 | chr17 | 81125276 | 81125373 | − | — |
| hsa-mir-656 | MI0003678 | — | chr14 | 101066724 | 101066801 | + | — |
| hsa-mir-655 | MI0003677 | 6 | chr14 | 101049550 | 101049646 | + | — |
| hsa-mir-654 | MI0003676 | 5 | chr14 | 101040219 | 101040299 | + | ✓ |
| hsa-mir-653 | MI0003674 | — | chr7 | 93482760 | 93482855 | − | ✓ |
| hsa-mir-6529 | MI0039501 | — | chr3 | 120084564 | 120084648 | − | — |
| hsa-mir-652 | MI0003667 | 189 | chrX | 110055329 | 110055426 | + | ✓ |
| hsa-mir-6516 | MI0025513 | 2 | chr17 | 77089417 | 77089497 | + | — |
| hsa-mir-6515 | MI0022227 | 4 | chr19 | 12940484 | 12940540 | + | — |
| hsa-mir-6514 | MI0022226 | — | chr11 | 62792702 | 62792771 | − | — |
| hsa-mir-6513 | MI0022225 | 2 | chr2 | 218280125 | 218280188 | − | — |
| hsa-mir-6512 | MI0022224 | 2 | chr2 | 177313806 | 177313882 | − | — |
| hsa-mir-6511b-2 | MI0023431 | 6 | chr16 | 15134075 | 15134145 | − | — |
| hsa-mir-6511b-1 | MI0022552 | 5 | chr16 | 2106669 | 2106753 | − | — |
| hsa-mir-6511a-4 | MI0023566 | 3 | chr16 | 18344013 | 18344079 | − | ✓ |
| hsa-mir-6511a-3 | MI0023565 | 3 | chr16 | 16368876 | 16368942 | + | ✓ |
| hsa-mir-6511a-2 | MI0023564 | 3 | chr16 | 16324588 | 16324654 | + | ✓ |
| hsa-mir-6511a-1 | MI0022223 | 3 | chr16 | 14925937 | 14926003 | + | ✓ |
| hsa-mir-6510 | MI0022222 | 5 | chr17 | 41517164 | 41517217 | − | — |
| hsa-mir-651 | MI0003666 | 7 | chrX | 8126965 | 8127061 | + | ✓ |
| hsa-mir-6509 | MI0022221 | — | chr7 | 135206994 | 135207078 | − | — |
| hsa-mir-6508 | MI0022220 | — | chr21 | 39447010 | 39447069 | + | — |
| hsa-mir-6507 | MI0022219 | 2 | chr10 | 98924499 | 98924568 | − | — |
| hsa-mir-6506 | MI0022218 | — | chr16 | 15611030 | 15611095 | − | — |
| hsa-mir-6505 | MI0022217 | 1 | chr12 | 48132797 | 48132867 | + | — |
| hsa-mir-6504 | MI0022216 | — | chr16 | 81611348 | 81611408 | + | — |
| hsa-mir-6503 | MI0022215 | — | chr11 | 60209071 | 60209156 | − | — |
| hsa-mir-6502 | MI0022214 | — | chr12 | 66251082 | 66251157 | + | — |
| hsa-mir-6501 | MI0022213 | — | chr21 | 33550662 | 33550728 | + | — |
| hsa-mir-6500 | MI0022211 | 58 | chr1 | 51060018 | 51060103 | + | — |
| hsa-mir-650 | MI0003665 | — | chr22 | 22822776 | 22822871 | + | — |
| hsa-mir-6499 | MI0022209 | 7 | chr5 | 151522087 | 151522148 | − | — |
| hsa-mir-649 | MI0003664 | — | chr22 | 21034176 | 21034272 | − | — |
| hsa-mir-648 | MI0003663 | 31 | chr22 | 17980868 | 17980961 | − | — |
| hsa-mir-647 | MI0003662 | 42 | chr20 | 63942631 | 63942726 | − | — |
| hsa-mir-646 | MI0003661 | — | chr20 | 60308474 | 60308567 | + | — |
| hsa-mir-645 | MI0003660 | 21 | chr20 | 50585786 | 50585879 | + | — |
| hsa-mir-644a | MI0003659 | — | chr20 | 34466325 | 34466418 | + | — |
| hsa-mir-643 | MI0003658 | 216 | chr19 | 52281797 | 52281893 | + | ✓ |
| hsa-mir-642b | MI0016685 | — | chr19 | 45674932 | 45675008 | − | ✓ |
| hsa-mir-642a | MI0003657 | — | chr19 | 45674928 | 45675024 | + | ✓ |
| hsa-mir-641 | MI0003656 | 4 | chr19 | 40282543 | 40282641 | − | — |
| hsa-mir-640 | MI0003655 | 36 | chr19 | 19435063 | 19435158 | + | — |
| hsa-mir-639 | MI0003654 | 78 | chr19 | 14529543 | 14529640 | + | — |
| hsa-mir-638 | MI0003653 | 9 | chr19 | 10718404 | 10718503 | + | — |
| hsa-mir-637 | MI0003652 | 37 | chr19 | 3961414 | 3961512 | − | — |
| hsa-mir-636 | MI0003651 | 37 | chr17 | 76736450 | 76736548 | − | — |
| hsa-mir-635 | MI0003650 | — | chr17 | 68424451 | 68424548 | − | — |
| hsa-mir-634 | MI0003649 | — | chr17 | 66787072 | 66787168 | + | — |
| hsa-mir-633 | MI0003648 | — | chr17 | 62944215 | 62944312 | + | — |
| hsa-mir-632 | MI0003647 | 10 | chr17 | 32350109 | 32350202 | + | — |
| hsa-mir-631 | MI0003645 | — | chr15 | 75353611 | 75353685 | − | — |
| hsa-mir-630 | MI0003644 | 3442 | chr15 | 72587217 | 72587313 | + | — |
| hsa-mir-629 | MI0003643 | 99 | chr15 | 70079372 | 70079468 | − | ✓ |
| hsa-mir-628 | MI0003642 | 4 | chr15 | 55372940 | 55373034 | − | — |
| hsa-mir-627 | MI0003641 | 3 | chr15 | 42199570 | 42199666 | − | ✓ |
| hsa-mir-626 | MI0003640 | — | chr15 | 41691585 | 41691678 | + | — |
| hsa-mir-625 | MI0003639 | 24 | chr14 | 65471102 | 65471186 | + | ✓ |
| hsa-mir-624 | MI0003638 | — | chr14 | 31014646 | 31014742 | − | — |
| hsa-mir-623 | MI0003637 | — | chr13 | 99356131 | 99356228 | + | — |
| hsa-mir-622 | MI0003636 | 5 | chr13 | 90231182 | 90231277 | + | — |
| hsa-mir-621 | MI0003635 | 11 | chr13 | 40810766 | 40810861 | + | — |
| hsa-mir-620 | MI0003634 | 20 | chr12 | 116148560 | 116148654 | − | — |
| hsa-mir-619 | MI0003633 | 201 | chr12 | 108836908 | 108837006 | − | — |
| hsa-mir-618 | MI0003632 | 13 | chr12 | 80935736 | 80935833 | − | — |
| hsa-mir-617 | MI0003631 | 38 | chr12 | 80832533 | 80832629 | − | — |
| hsa-mir-6165 | MI0021472 | 39 | chr17 | 49510817 | 49510900 | + | — |
| hsa-mir-616 | MI0003629 | 1 | chr12 | 57519163 | 57519259 | − | ✓ |
| hsa-mir-615 | MI0003628 | 117 | chr12 | 54033950 | 54034045 | + | ✓ |
| hsa-mir-614 | MI0003627 | — | chr12 | 12915829 | 12915918 | + | — |
| hsa-mir-6134 | MI0021279 | 38 | chrX | 28495555 | 28495663 | − | — |
| hsa-mir-6133 | MI0021278 | 1 | chr7 | 133290881 | 133290988 | + | — |
| hsa-mir-6132 | MI0021277 | — | chr7 | 117020211 | 117020319 | + | — |
| hsa-mir-6131 | MI0021276 | — | chr5 | 10478037 | 10478145 | + | — |
| hsa-mir-6130 | MI0021275 | 335 | chr21 | 23079284 | 23079392 | + | — |
| hsa-mir-613 | MI0003626 | 24 | chr12 | 12764649 | 12764743 | + | — |
| hsa-mir-6129 | MI0021274 | 8 | chr17 | 49288346 | 49288454 | − | — |
| hsa-mir-6128 | MI0021272 | 20 | chr11 | 56743873 | 56743981 | + | — |
| hsa-mir-6127 | MI0021271 | 87 | chr1 | 22633258 | 22633366 | − | — |
| hsa-mir-6126 | MI0021260 | 68 | chr16 | 3485381 | 3485469 | − | — |
| hsa-mir-6125 | MI0021259 | 4 | chr12 | 62260359 | 62260454 | + | — |
| hsa-mir-6124 | MI0021258 | 214 | chr11 | 12163683 | 12163767 | + | — |
| hsa-mir-612 | MI0003625 | 45 | chr11 | 65444458 | 65444557 | + | — |
| hsa-mir-611 | MI0003624 | 4 | chr11 | 61792495 | 61792561 | − | — |
| hsa-mir-610 | MI0003623 | 13 | chr11 | 28056815 | 28056910 | + | — |
| hsa-mir-6090 | MI0020367 | — | chr11 | 128522390 | 128522449 | + | — |
| hsa-mir-609 | MI0003622 | — | chr10 | 104218789 | 104218883 | − | — |
| hsa-mir-6089-2 | MI0023563 | 2 | chrY | 2609191 | 2609254 | + | — |
| hsa-mir-6089-1 | MI0020366 | 2 | chrX | 2609191 | 2609254 | + | — |
| hsa-mir-6088 | MI0020365 | 24 | chr19 | 45436654 | 45436704 | + | — |
| hsa-mir-6086 | MI0020363 | 83 | chrX | 13590292 | 13590346 | + | — |
| hsa-mir-6085 | MI0020362 | — | chr15 | 62343029 | 62343138 | + | — |
| hsa-mir-6084 | MI0020361 | — | chr1 | 20633679 | 20633788 | + | — |
| hsa-mir-6083 | MI0020360 | 6 | chr3 | 124374332 | 124374437 | + | — |
| hsa-mir-6082 | MI0020359 | 22 | chr4 | 171186184 | 171186292 | + | — |
| hsa-mir-6081 | MI0020358 | — | chr9 | 95065350 | 95065446 | + | — |
| hsa-mir-6080 | MI0020357 | — | chr17 | 64780759 | 64780824 | + | — |
| hsa-mir-608 | MI0003621 | — | chr10 | 100974985 | 100975084 | + | — |
| hsa-mir-6079 | MI0020356 | — | chr1 | 43838622 | 43838683 | + | — |
| hsa-mir-6078 | MI0020355 | — | chr10 | 3991160 | 3991259 | + | — |
| hsa-mir-6077 | MI0020354 | — | chr1 | 148388282 | 148388363 | + | — |
| hsa-mir-6076 | MI0020353 | 21 | chr14 | 49966399 | 49966511 | + | — |
| hsa-mir-6075 | MI0020352 | 24 | chr5 | 1510762 | 1510856 | − | — |
| hsa-mir-6074 | MI0020351 | 49 | chr12 | 66023620 | 66023726 | − | — |
| hsa-mir-6073 | MI0020350 | 4 | chr11 | 15969533 | 15969621 | − | — |
| hsa-mir-6072 | MI0020349 | — | chr10 | 2076019 | 2076089 | − | — |
| hsa-mir-6071 | MI0020348 | — | chr2 | 85783600 | 85783677 | − | — |
| hsa-mir-6070 | MI0020347 | — | chr21 | 43609887 | 43609989 | − | — |
| hsa-mir-607 | MI0003620 | — | chr10 | 96828669 | 96828764 | − | — |
| hsa-mir-6069 | MI0020346 | 244 | chr22 | 35336721 | 35336799 | − | — |
| hsa-mir-6068 | MI0020345 | — | chr1 | 63326925 | 63326984 | − | — |
| hsa-mir-606 | MI0003619 | — | chr10 | 75552458 | 75552553 | + | — |
| hsa-mir-605 | MI0003618 | 20 | chr10 | 51299573 | 51299655 | + | ✓ |
| hsa-mir-604 | MI0003617 | — | chr10 | 29545004 | 29545097 | − | — |
| hsa-mir-603 | MI0003616 | 1 | chr10 | 24275685 | 24275781 | + | — |
| hsa-mir-602 | MI0003615 | 4 | chr9 | 137838419 | 137838516 | + | — |
| hsa-mir-601 | MI0003614 | — | chr9) | 123402525 | 123402603 | − | — |
| hsa-mir-600 | MI0003613 | — | chr9 | 123111546 | 123111643 | − | — |
| hsa-mir-599 | MI0003611 | 3 | chr8 | 99536636 | 99536730 | − | — |
| hsa-mir-598 | MI0003610 | 138 | chr8 | 11035206 | 11035302 | − | ✓ |
| hsa-mir-597 | MI0003609 | — | chr8 | 9741672 | 9741768 | + | ✓ |
| hsa-mir-596 | MI0003608 | — | chr8 | 1817231 | 1817307 | + | — |
| hsa-mir-595 | MI0003607 | — | chr7 | 158532718 | 158532813 | − | — |
| hsa-mir-593 | MI0003605 | — | chr7 | 128081861 | 128081960 | + | — |
| hsa-mir-592 | MI0003604 | — | chr7 | 127058088 | 127058184 | − | — |
| hsa-mir-591 | MI0003603 | — | chr7 | 96219662 | 96219756 | − | — |
| hsa-mir-590 | MI0003602 | 270 | chr7 | 74191198 | 74191294 | + | ✓ |
| hsa-mir-589 | MI0003599 | 8 | chr7 | 5495819 | 5495917 | − | ✓ |
| hsa-mir-588 | MI0003597 | 4 | chr6 | 126484631 | 126484713 | + | — |
| hsa-mir-587 | MI0003595 | — | chr6 | 106784125 | 106784220 | + | — |
| hsa-mir-586 | MI0003594 | — | chr6 | 45197674 | 45197770 | − | — |
| hsa-mir-585 | MI0003592 | 5 | chr5 | 169263601 | 169263694 | − | ✓ |
| hsa-mir-584 | MI0003591 | 97 | chr5 | 149062313 | 149062409 | − | ✓ |
| hsa-mir-583 | MI0003590 | — | chr5 | 96079138 | 96079212 | + | — |
| hsa-mir-582 | MI0003589 | 151 | chr5 | 59703606 | 59703703 | − | ✓ |
| hsa-mir-581 | MI0003588 | 3 | chr5 | 53951504 | 53951599 | − | — |
| hsa-mir-580 | MI0003587 | — | chr5 | 36147892 | 36147988 | − | — |
| hsa-mir-579 | MI0003586 | 4 | chr5 | 32394378 | 32394475 | − | ✓ |
| hsa-mir-5787 | MI0019797 | — | chr3 | 50227436 | 50227490 | + | — |
| hsa-mir-578 | MI0003585 | — | chr4 | 165386242 | 165386337 | + | — |
| hsa-mir-577 | MI0003584 | 13 | chr4 | 114656759 | 114656854 | + | ✓ |
| hsa-mir-576 | MI0003583 | 15 | chr4 | 109488698 | 109488795 | + | ✓ |
| hsa-mir-575 | MI0003582 | 81 | chr4 | 82753337 | 82753430 | − | — |
| hsa-mir-574 | MI0003581 | 780 | chr4 | 38868032 | 38868127 | + | ✓ |
| hsa-mir-5739 | MI0019412 | 50 | chr22 | 28459869 | 28459948 | + | — |
| hsa-mir-573 | MI0003580 | — | chr4 | 24520192 | 24520290 | − | — |
| hsa-mir-572 | MI0003579 | 6 | chr4 | 11368827 | 11368921 | + | — |
| hsa-mir-571 | MI0003578 | — | chr4 | 350157 | 350252 | + | — |
| hsa-mir-5708 | MI0019316 | 55 | chr8 | 80241389 | 80241473 | + | — |
| hsa-mir-5707 | MI0019315 | — | chr7 | 158591616 | 158591696 | + | — |
| hsa-mir-5706 | MI0019314 | — | chr5 | 119154637 | 119154716 | + | — |
| hsa-mir-5705 | MI0019313 | — | chr4 | 87300495 | 87300583 | − | — |
| hsa-mir-5704 | MI0019312 | — | chr3 | 131985855 | 131985931 | − | — |
| hsa-mir-5703 | MI0019310 | 6 | chr2 | 227472132 | 227472187 | + | — |
| hsa-mir-5702 | MI0019309 | — | chr2 | 226658710 | 226658793 | − | — |
| hsa-mir-5701-3 | MI0031522 | — | chr15 | 21951242 | 21951323 | + | — |
| hsa-mir-5701-2 | MI0019593 | — | chr15 | 21513959 | 21514040 | + | — |
| hsa-mir-5701-1 | MI0019308 | — | chr15 | 20940252 | 20940333 | + | — |
| hsa-mir-5700 | MI0019307 | — | chr12 | 94561789 | 94561859 | + | — |
| hsa-mir-570 | MI0003577 | 4 | chr3 | 195699401 | 195699497 | + | — |
| hsa-mir-5699 | MI0019306 | 6 | chr10 | 641689 | 641778 | − | — |
| hsa-mir-5698 | MI0019305 | — | chr1 | 154104521 | 154104592 | − | — |
| hsa-mir-5697 | MI0019304 | — | chr1 | 9967381 | 9967458 | + | — |
| hsa-mir-5696 | MI0019303 | — | chr2 | 101309450 | 101309534 | + | — |
| hsa-mir-5695 | MI0019302 | 1 | chr19 | 12920320 | 12920404 | + | — |
| hsa-mir-5694 | MI0019301 | 2 | chr14 | 67441855 | 67441930 | − | — |
| hsa-mir-5693 | MI0019300 | 4 | chr13 | 51348567 | 51348639 | − | — |
| hsa-mir-5692c-2 | MI0019289 | 3 | chr7 | 97964405 | 97964481 | − | — |
| hsa-mir-5692c-1 | MI0019288 | — | chr5 | 135802985 | 135803075 | − | — |
| hsa-mir-5692b | MI0019311 | — | chr21 | 42950928 | 42951014 | − | — |
| hsa-mir-5692a-2 | MI0019298 | — | chr8 | 12719132 | 12719190 | + | — |
| hsa-mir-5692a-1 | MI0019297 | — | chr7 | 97963658 | 97963726 | + | — |
| hsa-mir-5691 | MI0019296 | — | chr11 | 9090312 | 9090379 | − | — |
| hsa-mir-5690 | MI0019295 | — | chr6 | 35664717 | 35664789 | − | — |
| hsa-mir-569 | MI0003576 | — | chr3 | 171106664 | 171106759 | − | — |
| hsa-mir-5689 | MI0019294 | 9 | chr6 | 10439717 | 10439794 | + | — |
| hsa-mir-5688 | MI0019292 | — | chr3 | 85385710 | 85385792 | + | — |
| hsa-mir-5687 | MI0019291 | — | chr5 | 55508850 | 55508926 | − | — |
| hsa-mir-5685 | MI0019287 | 21 | chr6 | 53276993 | 53277071 | + | — |
| hsa-mir-5684 | MI0019285 | 46 | chr19 | 12787128 | 12787192 | + | — |
| hsa-mir-5683 | MI0019284 | — | chr6 | 6169334 | 6169409 | + | — |
| hsa-mir-5682 | MI0019282 | — | chr3 | 121049640 | 121049715 | + | ✓ |
| hsa-mir-5681b | MI0019293 | — | chr8 | 74548550 | 74548609 | − | — |
| hsa-mir-5681a | MI0019281 | — | chr8 | 74548543 | 74548617 | + | — |
| hsa-mir-5680 | MI0019280 | — | chr8 | 102125432 | 102125515 | + | — |
| hsa-mir-568 | MI0003574 | — | chr3 | 114316475 | 114316569 | − | — |
| hsa-mir-567 | MI0003573 | 70 | chr3 | 112112801 | 112112898 | + | — |
| hsa-mir-564 | MI0003570 | — | chr3 | 44861888 | 44861981 | + | — |
| hsa-mir-563 | MI0003569 | — | chr3 | 15873771 | 15873849 | + | — |
| hsa-mir-562 | MI0003568 | — | chr2 | 232172653 | 232172747 | + | — |
| hsa-mir-561 | MI0003567 | 32 | chr2 | 188297492 | 188297588 | + | ✓ |
| hsa-mir-5591 | MI0019151 | — | chr4 | 39411910 | 39411974 | + | — |
| hsa-mir-5590 | MI0019150 | — | chr2 | 134857820 | 134857873 | + | — |
| hsa-mir-559 | MI0003565 | 9 | chr2 | 47377675 | 47377770 | + | — |
| hsa-mir-5589 | MI0019148 | — | chr19 | 10038354 | 10038413 | + | — |
| hsa-mir-5588 | MI0019147 | 97 | chr3 | 185253210 | 185253272 | − | — |
| hsa-mir-5587 | MI0019144 | 3 | chr16 | 535316 | 535368 | + | — |
| hsa-mir-5586 | MI0019143 | — | chr14 | 59646962 | 59647020 | − | — |
| hsa-mir-5585 | MI0019142 | 320 | chr1 | 32086949 | 32087007 | + | — |
| hsa-mir-5584 | MI0019141 | — | chr1 | 44545493 | 44545552 | + | — |
| hsa-mir-5583-2 | MI0019140 | — | chr18 | 39676719 | 39676777 | − | ✓ |
| hsa-mir-5583-1 | MI0019139 | — | chr18 | 39676721 | 39676779 | + | ✓ |
| hsa-mir-5582 | MI0019138 | — | chr11 | 46753125 | 46753192 | − | — |
| hsa-mir-5581 | MI0019136 | — | chr1 | 37500935 | 37500994 | − | — |
| hsa-mir-5580 | MI0019135 | — | chr14 | 53948427 | 53948484 | − | — |
| hsa-mir-558 | MI0003564 | 134 | chr2 | 32532153 | 32532246 | + | — |
| hsa-mir-5579 | MI0019133 | — | chr11 | 79422169 | 79422226 | − | — |
| hsa-mir-5572 | MI0019117 | 94 | chr15 | 80581103 | 80581239 | + | — |
| hsa-mir-5571 | MI0019115 | 22 | chr22 | 22886267 | 22886379 | + | — |
| hsa-mir-557 | MI0003563 | — | chr1 | 168375524 | 168375621 | + | — |
| hsa-mir-556 | MI0003562 | 3 | chr1 | 162342546 | 162342640 | + | ✓ |
| hsa-mir-555 | MI0003561 | — | chr1 | 155346350 | 155346445 | − | — |
| hsa-mir-554 | MI0003559 | — | chr1 | 151545796 | 151545891 | + | — |
| hsa-mir-553 | MI0003558 | — | chr1 | 100281241 | 100281308 | + | — |
| hsa-mir-552 | MI0003557 | — | chr1 | 34669599 | 34669694 | − | ✓ |
| hsa-mir-551b | MI0003575 | 70 | chr3 | 168551854 | 168551949 | + | ✓ |
| hsa-mir-551a | MI0003556 | 7 | chr1 | 3560695 | 3560790 | − | — |
| hsa-mir-550b-2 | MI0016687 | 2 | chr7 | 32732981 | 32733077 | − | — |
| hsa-mir-550b-1 | MI0016686 | 2 | chr7 | 30289794 | 30289890 | − | — |
| hsa-mir-550a-3 | MI0003762 | 16 | chr7 | 29680734 | 29680828 | − | ✓ |
| hsa-mir-550a-2 | MI0003601 | 16 | chr7 | 32732981 | 32733077 | + | ✓ |
| hsa-mir-550a-1 | MI0003600 | 16 | chr7 | 30289794 | 30289890 | + | ✓ |
| hsa-mir-549a | MI0003679 | — | chr15 | 80841978 | 80842073 | − | — |
| hsa-mir-548z | MI0016688 | 3 | chr12 | 64622509 | 64622605 | − | — |
| hsa-mir-548y | MI0016595 | 1 | chr14 | 47760995 | 47761104 | − | ✓ |
| hsa-mir-548x-2 | MI0016833 | 8 | chr13 | 65966330 | 65966429 | − | — |
| hsa-mir-548x | MI0014244 | 3 | chr21 | 18686090 | 18686164 | − | ✓ |
| hsa-mir-548w | MI0014222 | 3 | chr16 | 26025237 | 26025310 | + | — |
| hsa-mir-548v | MI0014174 | 9 | chr8 | 17681578 | 17681657 | − | ✓ |
| hsa-mir-548u | MI0014168 | 17 | chr6 | 57390132 | 57390212 | + | — |
| hsa-mir-548t | MI0014164 | 2 | chr4 | 173268160 | 173268233 | + | — |
| hsa-mir-548s | MI0014141 | 5 | chr2 | 11767444 | 11767525 | + | — |
| hsa-mir-548q | MI0010637 | 5 | chr10 | 12725254 | 12725353 | − | — |
| hsa-mir-548p | MI0006420 | 7 | chr5 | 100816482 | 100816565 | − | — |
| hsa-mir-5480-2 | MI0016746 | 15 | chr20 | 38516563 | 38516632 | + | ✓ |
| hsa-mir-5480 | MI0006402 | 5 | chr7 | 102405742 | 102405855 | − | — |
| hsa-mir-548n | MI0006399 | 7 | chr7 | 34940760 | 34940834 | − | — |
| hsa-mir-548m | MI0006400 | — | chrX | 95063141 | 95063226 | − | — |
| hsa-mir-5481 | MI0006361 | 8 | chr11 | 94466495 | 94466580 | − | — |
| hsa-mir-548k | MI0006354 | 15 | chr11 | 70283955 | 70284070 | + | ✓ |
| hsa-mir-548j | MI0006345 | 1 | chr22 | 26555212 | 26555323 | − | ✓ |
| hsa-mir-5481-4 | MI0006424 | 16 | chrX | 84225752 | 84225828 | − | — |
| hsa-mir-5481-3 | MI0006423 | 17 | chr8 | 8088941 | 8089089 | − | — |
| hsa-mir-5481-2 | MI0006422 | 17 | chr4 | 9556168 | 9556316 | − | — |
| hsa-mir-5481-1 | MI0006421 | 17 | chr3 | 125790404 | 125790552 | − | — |
| hsa-mir-548h-5 | MI0016751 | 2 | chr6 | 131792172 | 131792231 | + | — |
| hsa-mir-548h-4 | MI0006414 | 4 | chr8 | 27048853 | 27048963 | − | ✓ |
| hsa-mir-548h-3 | MI0006413 | 6 | chr17 | 13543529 | 13543646 | − | ✓ |
| hsa-mir-548h-2 | MI0006412 | 3 | chr16 | 11306440 | 11306527 | − | ✓ |
| hsa-mir-548h-1 | MI0006411 | 2 | chr14 | 64095024 | 64095125 | − | — |
| hsa-mir-548g | MI0006395 | — | chr4 | 147344629 | 147344717 | − | — |
| hsa-mir-548f-5 | MI0006378 | — | chrX | 32641474 | 32641559 | − | — |
| hsa-mir-548f-4 | MI0006377 | 8 | chr7 | 147378017 | 147378121 | − | — |
| hsa-mir-548f-3 | MI0006376 | 3 | chr5 | 110513829 | 110513915 | − | — |
| hsa-mir-548f-2 | MI0006375 | 2 | chr2 | 212426263 | 212426360 | − | — |
| hsa-mir-548f-1 | MI0006374 | 3 | chr10 | 54607874 | 54607957 | − | — |
| hsa-mir-548c | MI0006344 | 14 | chr10 | 110988926 | 110989013 | + | ✓ |
| hsa-mir-548d-2 | MI0003671 | 15 | chr17 | 67471489 | 67471585 | − | ✓ |
| hsa-mir-548d-1 | MI0003668 | 12 | chr8 | 123348034 | 123348130 | − | ✓ |
| hsa-mir-548c | MI0003630 | 13 | chr12 | 64622509 | 64622605 | + | — |
| hsa-mir-548bc | MI0032315 | 1 | chr17 | 39297400 | 39297509 | + | — |
| hsa-mir-548bb | MI0029321 | — | chr3 | 60617805 | 60617870 | − | — |
| hsa-mir-548ba | MI0025747 | 8 | chr2 | 49059603 | 49059658 | + | — |
| hsa-mir-548b | MI0003596 | 35 | chr6 | 119069047 | 119069143 | − | ✓ |
| hsa-mir-548az | MI0022212 | 8 | chr8 | 119325171 | 119325265 | + | — |
| hsa-mir-548ay | MI0022210 | 8 | chr3 | 32506283 | 32506389 | − | ✓ |
| hsa-mir-548ax | MI0019286 | 1 | chrX | 11318614 | 11318686 | − | — |
| hsa-mir-548aw | MI0019283 | — | chr9 | 132945707 | 132945771 | + | — |
| hsa-mir-548av | MI0019152 | 25 | chr18 | 72853321 | 72853382 | − | — |
| hsa-mir-548au | MI0019145 | 8 | chr9 | 93594841 | 93594894 | + | — |
| hsa-mir-548at | MI0019137 | — | chr17 | 42494773 | 42494830 | + | ✓ |
| hsa-mir-548as | MI0019132 | 1 | chr13 | 92490163 | 92490220 | + | — |
| hsa-mir-548ar | MI0019131 | — | chr13 | 114244505 | 114244561 | + | ✓ |
| hsa-mir-548aq | MI0019130 | 3 | chr3 | 185767847 | 185767904 | − | ✓ |
| hsa-mir-548ap | MI0017875 | 2 | chr15 | 85825635 | 85825730 | + | ✓ |
| hsa-mir-548ao | MI0017871 | — | chr8 | 41271048 | 41271143 | − | — |
| hsa-mir-548an | MI0016907 | — | chrX | 106639814 | 106639896 | + | ✓ |
| hsa-mir-548am | MI0016904 | 16 | chrX | 16627012 | 16627085 | − | ✓ |
| hsa-mir-548a1 | MI0016851 | 1 | chr11 | 74399237 | 74399333 | + | — |
| hsa-mir-548ak | MI0016840 | 3 | chr10 | 12130760 | 12130816 | − | — |
| hsa-mir-548aj-2 | MI0016815 | 2 | chrX | 38023895 | 38023986 | − | — |
| hsa-mir-548aj-1 | MI0016814 | 2 | chr6 | 132115192 | 132115263 | − | — |
| hsa-mir-548a1 | MI0016813 | — | chr6 | 99124609 | 99124696 | + | — |
| hsa-mir-548ah | MI0016796 | 7 | chr4 | 76575551 | 76575626 | + | — |
| hsa-mir-548ag-2 | MI0016794 | 4 | chr20 | 60564562 | 60564625 | + | ✓ |
| hsa-mir-548ag-1 | MI0016793 | 3 | chr4 | 60922619 | 60922684 | + | — |
| hsa-mir-548ae-2 | MI0016780 | 10 | chr5 | 58530043 | 58530109 | − | ✓ |
| hsa-mir-548ae-1 | MI0016779 | 15 | chr2 | 184378975 | 184379044 | + | — |
| hsa-mir-548ad | MI0016770 | 8 | chr2 | 35471405 | 35471486 | + | — |
| hsa-mir-548ac | MI0016762 | 6 | chr1 | 116560024 | 116560111 | − | — |
| hsa-mir-548ab | MI0016752 | 7 | chr3 | 103524033 | 103524116 | − | — |
| hsa-mir-548aa-2 | MI0016690 | 1 | chr17 | 67471489 | 67471585 | + | — |
| hsa-mir-548aa-1 | MI0016689 | 1 | chr8 | 123348034 | 123348130 | + | — |
| hsa-mir-548a-3 | MI0003612 | 18 | chr8 | 104484369 | 104484465 | − | ✓ |
| hsa-mir-548a-2 | MI0003598 | 25 | chr6 | 135239160 | 135239256 | + | — |
| hsa-mir-548a-1 | MI0003593 | 21 | chr6 | 18571784 | 18571880 | + | — |
| hsa-mir-545 | MI0003516 | 11 | chrX | 74287104 | 74287209 | − | ✓ |
| hsa-mir-544b | MI0014159 | 1 | chr3 | 124732439 | 124732516 | + | — |
| hsa-mir-544a | MI0003515 | 2 | chr14 | 101048658 | 101048748 | + | — |
| hsa-mir-543 | MI0005565 | 6 | chr14 | 101031987 | 101032064 | + | — |
| hsa-mir-542 | MI0003686 | 174 | chrX | 134541341 | 134541437 | − | ✓ |
| hsa-mir-541 | MI0005539 | 28 | chr14 | 101064495 | 101064578 | + | ✓ |
| hsa-mir-539 | MI0003514 | 4 | chr14 | 101047321 | 101047398 | + | ✓ |
| hsa-mir-532 | MI0003205 | 322 | chrX | 50003148 | 50003238 | + | ✓ |
| hsa-mir-527 | MI0003179 | 27 | chr19 | 53754018 | 53754102 | + | — |
| hsa-mir-526b | MI0003150 | 1 | chr19 | 53694393 | 53694475 | + | — |
| hsa-mir-526a-2 | MI0003168 | — | chr19 | 53726922 | 53726986 | + | — |
| hsa-mir-526a-1 | MI0003157 | 23 | chr19 | 53706252 | 53706336 | + | — |
| hsa-mir-525 | MI0003152 | 8 | chr19 | 53697533 | 53697617 | + | — |
| hsa-mir-524 | MI0003160 | 2 | chr19 | 53711002 | 53711088 | + | — |
| hsa-mir-523 | MI0003153 | 3 | chr19 | 53698385 | 53698471 | + | — |
| hsa-mir-522 | MI0003177 | 8 | chr19 | 53751211 | 53751297 | + | ✓ |
| hsa-mir-521-2 | MI0003163 | 13 | chr19 | 53716594 | 53716680 | + | — |
| hsa-mir-521-1 | MI0003176 | — | chr19 | 53748636 | 53748722 | + | — |
| hsa-mir-520h | MI0003175 | 18 | chr19 | 53742512 | 53742599 | + | — |
| hsa-mir-520g | MI0003166 | 17 | chr19 | 53722166 | 53722255 | + | — |
| hsa-mir-520f | MI0003146 | 4 | chr19 | 53682159 | 53682245 | + | — |
| hsa-mir-520e | MI0003143 | 33 | chr19 | 53675711 | 53675797 | + | — |
| hsa-mir-520d | MI0003164 | — | chr19 | 53720096 | 53720182 | + | — |
| hsa-mir-520c | MI0003158 | 6 | chr19 | 53707453 | 53707539 | + | ✓ |
| hsa-mir-520b | MI0003155 | 9 | chr19 | 53701227 | 53701287 | + | — |
| hsa-mir-520a | MI0003149 | 4 | chr19 | 53690881 | 53690965 | + | ✓ |
| hsa-mir-519e | MI0003145 | 10 | chr19 | 53679940 | 53680023 | + | — |
| hsa-mir-519d | MI0003162 | 3 | chr19 | 53713347 | 53713434 | + | — |
| hsa-mir-519c | MI0003148 | 14 | chr19 | 53686469 | 53686555 | + | — |
| hsa-mir-519b | MI0003151 | 8 | chr19 | 53695213 | 53695293 | + | ✓ |
| hsa-mir-519a-2 | MI0003182 | 8 | chr19 | 53762344 | 53762430 | + | ✓ |
| hsa-mir-519a-1 | MI0003178 | 11 | chr19 | 53752397 | 53752481 | + | ✓ |
| hsa-mir-5197 | MI0018176 | — | chr5 | 143679860 | 143679971 | + | — |
| hsa-mir-5196 | MI0018175 | 20 | chr19 | 35345513 | 35345627 | + | — |
| hsa-mir-5195 | MI0018174 | — | chr14 | 106850885 | 106850999 | − | — |
| hsa-mir-5194 | MI0018173 | — | chr8 | 130008334 | 130008453 | − | — |
| hsa-mir-5193 | MI0018172 | 2 | chr3 | 49806137 | 49806245 | − | — |
| hsa-mir-5192 | MI0018171 | 4 | chr2 | 62205826 | 62205917 | + | — |
| hsa-mir-5191 | MI0018170 | 64 | chr1 | 201719508 | 201719627 | + | — |
| hsa-mir-5190 | MI0018169 | — | chr18 | 13459947 | 13460026 | + | — |
| hsa-mir-518f | MI0003154 | 6 | chr19 | 53700015 | 53700101 | + | — |
| hsa-mir-518e | MI0003169 | 3 | chr19 | 53729838 | 53729925 | + | — |
| hsa-mir-518d | MI0003171 | — | chr19 | 53734877 | 53734963 | + | — |
| hsa-mir-518c | MI0003159 | 5 | chr19 | 53708735 | 53708835 | + | — |
| hsa-mir-518b | MI0003156 | 6 | chr19 | 53702737 | 53702819 | + | — |
| hsa-mir-518a-2 | MI0003173 | — | chr19 | 53739333 | 53739419 | + | — |
| hsa-mir-518a-1 | MI0003170 | — | chr19 | 53731006 | 53731090 | + | — |
| hsa-mir-5189 | MI0018168 | — | chr16 | 88468918 | 88469031 | + | — |
| hsa-mir-5188 | MI0018167 | — | chr12 | 124915547 | 124915659 | + | — |
| hsa-mir-5187 | MI0018166 | 1 | chr1 | 161227186 | 161227261 | + | — |
| hsa-mir-5186 | MI0018165 | 13 | chr3 | 151565876 | 151565995 | − | — |
| hsa-mir-517c | MI0003174 | 1 | chr19 | 53741313 | 53741407 | + | — |
| hsa-mir-517b | MI0003165 | 1 | chr19 | 53721076 | 53721142 | + | — |
| hsa-mir-517a | MI0003161 | 1 | chr19 | 53712268 | 53712354 | + | — |
| hsa-mir-516b-2 | MI0003167 | — | chr19 | 53725442 | 53725526 | + | — |
| hsa-mir-516b-1 | MI0003172 | — | chr19 | 53736845 | 53736934 | + | — |
| hsa-mir-516a-2 | MI0003181 | 32 | chr19 | 53761133 | 53761222 | + | — |
| hsa-mir-516a-1 | MI0003180 | 31 | chr19 | 53756741 | 53756830 | + | — |
| hsa-mir-515-2 | MI0003147 | 7 | chr19 | 53685009 | 53685091 | + | — |
| hsa-mir-515-1 | MI0003144 | 7 | chr19 | 53679003 | 53679085 | + | — |
| hsa-mir-514b | MI0014251 | 573 | chrX | 147250151 | 147250230 | − | ✓ |
| hsa-mir-514a-3 | MI0003200 | 434 | chrX | 147284641 | 147284728 | − | ✓ |
| hsa-mir-514a-2 | MI0003199 | 434 | chrX | 147281943 | 147282030 | − | ✓ |
| hsa-mir-514a-1 | MI0003198 | 431 | chrX | 147279247 | 147279344 | − | ✓ |
| hsa-mir-513c | MI0006649 | 23 | chrX | 147189704 | 147189787 | − | ✓ |
| hsa-mir-513b | MI0006648 | 24 | chrX | 147199044 | 147199127 | − | — |
| hsa-mir-513a-2 | MI0003192 | 20 | chrX | 147225826 | 147225952 | − | ✓ |
| hsa-mir-513a-1 | MI0003191 | 23 | chrX | 147213463 | 147213591 | − | ✓ |
| hsa-mir-512-2 | MI0003141 | — | chr19 | 53669157 | 53669254 | + | ✓ |
| hsa-mir-512-1 | MI0003140 | — | chr19 | 53666679 | 53666762 | + | ✓ |
| hsa-mir-511 | MI0003127 | — | chr10 | 17845107 | 17845193 | + | ✓ |
| hsa-mir-5100 | MI0019116 | 107 | chr10 | 42997563 | 42997681 | + | — |
| hsa-mir-510 | MI0003197 | 20 | chrX | 147272335 | 147272408 | − | ✓ |
| hsa-mir-5094 | MI0017983 | — | chr15 | 89850637 | 89850721 | − | — |
| hsa-mir-5093 | MI0017982 | 38 | chr16 | 85306226 | 85306325 | − | — |
| hsa-mir-5092 | MI0017981 | — | chr3 | 125151465 | 125151552 | − | — |
| hsa-mir-5091 | MI0017980 | 6 | chr4 | 13627865 | 13627957 | + | — |
| hsa-mir-5090 | MI0017979 | 2 | chr7 | 102465742 | 102465826 | + | — |
| hsa-mir-509-3 | MI0005717 | 352 | chrX | 147259652 | 147259726 | − | ✓ |
| hsa-mir-509-2 | MI0005530 | 259 | chrX | 147258760 | 147258850 | − | ✓ |
| hsa-mir-509-1 | MI0003196 | 259 | chrX | 147260532 | 147260625 | − | ✓ |
| hsa-mir-5089 | MI0017978 | 34 | chr17 | 46973017 | 46973100 | + | — |
| hsa-mir-5088 | MI0017977 | — | chr19 | 49682117 | 49682195 | + | — |
| hsa-mir-5087 | MI0017976 | — | chr1 | 148334479 | 148334554 | − | — |
| hsa-mir-508 | MI0003195 | 347 | chrX | 147236913 | 147237027 | − | ✓ |
| hsa-mir-507 | MI0003194 | 20 | chrX | 147230984 | 147231077 | − | — |
| hsa-mir-506 | MI0003193 | 55 | chrX | 147230720 | 147230843 | − | ✓ |
| hsa-mir-505 | MI0003190 | 86 | chrX | 139924148 | 139924231 | − | — |
| hsa-mir-5047 | MI0017932 | — | chr17 | 64501214 | 64501313 | − | — |
| hsa-mir-504 | MI0003189 | 4 | chrX | 138667711 | 138667793 | − | ✓ |
| hsa-mir-503 | MI0003188 | 132 | chrX | 134546328 | 134546398 | − | ✓ |
| hsa-mir-502 | MI0003186 | 58 | chrX | 50014598 | 50014683 | + | ✓ |
| hsa-mir-5011 | MI0017879 | — | chr18 | 67081584 | 67081686 | + | — |
| hsa-mir-5010 | MI0017878 | 1 | chr17 | 42514188 | 42514307 | + | ✓ |
| hsa-mir-501 | MI0003185 | 53 | chrX | 50009722 | 50009805 | + | ✓ |
| hsa-mir-500b | MI0015903 | 13 | chrX | 50010672 | 50010750 | + | — |
| hsa-mir-500a | MI0003184 | 63 | chrX | 50008431 | 50008514 | + | ✓ |
| hsa-mir-5009 | MI0017877 | — | chr15 | 89883931 | 89884030 | − | — |
| hsa-mir-5008 | MI0017876 | 1 | chr1 | 227941590 | 227941683 | − | — |
| hsa-mir-5007 | MI0017874 | — | chr13 | 55174454 | 55174548 | + | — |
| hsa-mir-5006 | MI0017873 | 8 | chr13 | 41568286 | 41568395 | − | — |
| hsa-mir-5004 | MI0017870 | 7 | chr6 | 33438331 | 33438437 | + | — |
| hsa-mir-5003 | MI0017869 | 12 | chr5 | 172662165 | 172662263 | + | — |
| hsa-mir-5002 | MI0017868 | 9 | chr3 | 124132929 | 124133025 | + | — |
| hsa-mir-5001 | MI0017867 | — | chr2 | 232550474 | 232550573 | − | ✓ |
| hsa-mir-5000 | MI0017866 | 11 | chr2 | 75090812 | 75090914 | + | ✓ |
| hsa-mir-499b | MI0017396 | 6 | chr20 | 34990400 | 34990472 | − | — |
| hsa-mir-499a | MI0003183 | 382 | chr20 | 34990376 | 34990497 | + | ✓ |
| hsa-mir-4999 | MI0017865 | 13 | chr19 | 8389290 | 8389380 | − | — |
| hsa-mir-498 | MI0003142 | 54 | chr19 | 53674197 | 53674320 | + | — |
| hsa-mir-497 | MI0003138 | 267 | chr17 | 7017911 | 7018022 | − | — |
| hsa-mir-496 | MI0003136 | — | chr14 | 101060573 | 101060674 | + | — |
| hsa-mir-495 | MI0003135 | 91 | chr14 | 101033755 | 101033836 | + | ✓ |
| hsa-mir-494 | MI0003134 | 10 | chr14 | 101029634 | 101029714 | + | — |
| hsa-mir-493 | MI0003132 | 10 | chr14 | 100869060 | 100869148 | + | ✓ |
| hsa-mir-492 | MI0003131 | — | chr12 | 94834398 | 94834513 | + | — |
| hsa-mir-491 | MI0003126 | 4 | chr9 | 20716105 | 20716188 | + | ✓ |
| hsa-mir-490 | MI0003125 | 13 | chr7 | 136903167 | 136903294 | + | ✓ |
| hsa-mir-489 | MI0003124 | — | chr7 | 93483936 | 93484019 | − | — |
| hsa-mir-488 | MI0003123 | 16 | chr1 | 177029363 | 177029445 | − | ✓ |
| hsa-mir-487b | MI0003530 | 23 | chr14 | 101046455 | 101046538 | + | — |
| hsa-mir-487a | MI0002471 | — | chr14 | 101052446 | 101052525 | + | ✓ |
| hsa-mir-486-2 | MI0023622 | 645 | chr8 | 41660444 | 41660507 | + | ✓ |
| hsa-mir-486-1 | MI0002470 | 650 | chr8 | 41660441 | 41660508 | − | ✓ |
| hsa-mir-485 | MI0002469 | 9 | chr14 | 101055419 | 101055491 | + | ✓ |
| hsa-mir-484 | MI0002468 | 435 | chr16 | 15643294 | 15643372 | + | — |
| hsa-mir-483 | MI0002467 | 83 | chr11 | 2134134 | 2134209 | − | ✓ |
| hsa-mir-4804 | MI0017452 | 3 | chr5 | 72878591 | 72878663 | + | — |
| hsa-mir-4803 | MI0017451 | — | chr5 | 72169467 | 72169540 | + | — |
| hsa-mir-4802 | MI0017450 | — | chr4 | 40502040 | 40502119 | − | ✓ |
| hsa-mir-4801 | MI0017449 | — | chr4 | 37241910 | 37241991 | − | — |
| hsa-mir-4800 | MI0017448 | 4 | chr4 | 2250077 | 2250156 | − | — |
| hsa-mir-4799 | MI0017446 | — | chr4 | 147782595 | 147782668 | + | — |
| hsa-mir-4798 | MI0017445 | — | chr4 | 7310450 | 7310524 | + | — |
| hsa-mir-4797 | MI0017444 | — | chr3 | 197293878 | 197293948 | − | — |
| hsa-mir-4796 | MI0017443 | — | chr3 | 114743445 | 114743525 | − | — |
| hsa-mir-4795 | MI0017442 | — | chr3 | 87226189 | 87226277 | − | — |
| hsa-mir-4794 | MI0017441 | — | chr1 | 64579847 | 64579923 | + | — |
| hsa-mir-4793 | MI0017440 | 8 | chr3 | 48644194 | 48644280 | − | — |
| hsa-mir-4791 | MI0017438 | 3 | chr3 | 19314848 | 19314931 | − | — |
| hsa-mir-4790 | MI0017437 | 52 | chr3 | 5250177 | 5250255 | − | — |
| hsa-mir-4789 | MI0017436 | — | chr3 | 175369540 | 175369621 | + | — |
| hsa-mir-4788 | MI0017435 | 4 | chr3 | 134437827 | 134437906 | + | — |
| hsa-mir-4787 | MI0017434 | 4 | chr3 | 50675080 | 50675163 | + | — |
| hsa-mir-4786 | MI0017433 | — | chr2 | 239943015 | 239943094 | − | ✓ |
| hsa-mir-4785 | MI0017430 | — | chr2 | 160407810 | 160407882 | − | — |
| hsa-mir-4784 | MI0017429 | — | chr2 | 131491160 | 131491236 | − | — |
| hsa-mir-4783 | MI0017428 | — | chr2 | 127423537 | 127423618 | − | — |
| hsa-mir-4782 | MI0017427 | — | chr2 | 113721290 | 113721368 | − | — |
| hsa-mir-4781 | MI0017426 | — | chr1 | 54054079 | 54054154 | + | — |
| hsa-mir-4780 | MI0017424 | — | chr2 | 88082519 | 88082599 | − | — |
| hsa-mir-4779 | MI0017423 | 11 | chr2 | 86193026 | 86193108 | − | — |
| hsa-mir-4778 | MI0017422 | 2 | chr2 | 66358249 | 66358328 | − | ✓ |
| hsa-mir-4777 | MI0017421 | — | chr2 | 231362708 | 231362793 | + | — |
| hsa-mir-4776-2 | MI0017420 | 114 | chr2 | 212926257 | 212926336 | − | — |
| hsa-mir-4776-1 | MI0017419 | 8 | chr2 | 212926257 | 212926336 | + | — |
| hsa-mir-4775 | MI0017418 | — | chr2 | 207754807 | 207754881 | + | — |
| hsa-mir-4774 | MI0017417 | — | chr2 | 168582943 | 168583018 | + | — |
| hsa-mir-4773-2 | MI0017416 | 18 | chr2 | 151368334 | 151368411 | − | — |
| hsa-mir-4773-1 | MI0017415 | 17 | chr2 | 151368334 | 151368411 | + | — |
| hsa-mir-4772 | MI0017414 | 1 | chr2 | 102432289 | 102432366 | + | — |
| hsa-mir-4771-2 | MI0017413 | — | chr2 | 111771061 | 111771134 | − | — |
| hsa-mir-4771-1 | MI0017412 | — | chr2 | 87194786 | 87194859 | + | — |
| hsa-mir-4770 | MI0017411 | — | chrX | 6383906 | 6383963 | − | — |
| hsa-mir-4769 | MI0017410 | — | chrX | 47587429 | 47587505 | + | — |
| hsa-mir-4768 | MI0017409 | — | chrX | 17425881 | 17425954 | + | — |
| hsa-mir-4767 | MI0017408 | 2 | chrX | 7147860 | 7147937 | + | — |
| hsa-mir-4766 | MI0017407 | 1 | chr22 | 40813883 | 40813958 | − | — |
| hsa-mir-4765 | MI0017406 | — | chr2 | 32635255 | 32635331 | + | — |
| hsa-mir-4764 | MI0017405 | — | chr22 | 33436582 | 33436669 | − | — |
| hsa-mir-4763 | MI0017404 | 5 | chr22 | 46113566 | 46113657 | + | — |
| hsa-mir-4762 | MI0017403 | — | chr22 | 45760524 | 45760598 | + | — |
| hsa-mir-4761 | MI0017402 | 8 | chr22 | 19963753 | 19963834 | + | ✓ |
| hsa-mir-4760 | MI0017401 | — | chr21 | 40212352 | 40212431 | − | — |
| hsa-mir-4759 | MI0017400 | — | chr21 | 26953961 | 26954043 | + | — |
| hsa-mir-4758 | MI0017399 | — | chr20 | 62332487 | 62332557 | − | — |
| hsa-mir-4757 | MI0017398 | — | chr2 | 19348429 | 19348505 | + | — |
| hsa-mir-4756 | MI0017397 | 11 | chr20 | 54068408 | 54068485 | − | — |
| hsa-mir-4755 | MI0017395 | — | chr20 | 34049119 | 34049190 | + | ✓ |
| hsa-mir-4754 | MI0017394 | — | chr19 | 58386770 | 58386858 | − | — |
| hsa-mir-4753 | MI0017392 | 1 | chr1 | 235190034 | 235190116 | − | — |
| hsa-mir-4752 | MI0017391 | — | chr19 | 54282109 | 54282180 | + | — |
| hsa-mir-4751 | MI0017390 | — | chr19 | 49933064 | 49933137 | + | — |
| hsa-mir-4750 | MI0017389 | — | chr19 | 49888175 | 49888230 | + | — |
| hsa-mir-4749 | MI0017388 | 9 | chr19 | 49854591 | 49854651 | + | — |
| hsa-mir-4748 | MI0017387 | — | chr19 | 10780254 | 10780335 | + | — |
| hsa-mir-4747 | MI0017386 | — | chr19 | 4932687 | 4932740 | + | — |
| hsa-mir-4746 | MI0017385 | — | chr19 | 4445978 | 4446048 | + | — |
| hsa-mir-4745 | MI0017384 | — | chr19 | 804940 | 805001 | + | — |
| hsa-mir-4744 | MI0017382 | — | chr18 | 49049687 | 49049768 | − | — |
| hsa-mir-4743 | MI0017381 | — | chr18 | 48670600 | 48670668 | + | — |
| hsa-mir-4742 | MI0017380 | — | chr1 | 224398227 | 224398311 | − | — |
| hsa-mir-4741 | MI0017379 | — | chr18 | 22933349 | 22933438 | + | — |
| hsa-mir-4740 | MI0017378 | 26 | chr17 | 81400716 | 81400778 | − | — |
| hsa-mir-4739 | MI0017377 | 166 | chr17 | 79707176 | 79707249 | − | — |
| hsa-mir-4738 | MI0017376 | 3 | chr17 | 75784521 | 75784607 | − | — |
| hsa-mir-4737 | MI0017374 | — | chr17 | 60043025 | 60043105 | − | — |
| hsa-mir-4736 | MI0017373 | 282 | chr17 | 58335976 | 58336022 | − | — |
| hsa-mir-4735 | MI0017372 | 75 | chr1 | 196582413 | 196582481 | − | — |
| hsa-mir-4734 | MI0017371 | 2 | chr17 | 38702262 | 38702331 | − | — |
| hsa-mir-4733 | MI0017370 | — | chr17 | 31094350 | 31094425 | − | — |
| hsa-mir-4732 | MI0017369 | 1 | chr17 | 28861655 | 28861730 | − | — |
| hsa-mir-4731 | MI0017368 | — | chr17 | 15251627 | 15251696 | − | — |
| hsa-mir-4730 | MI0017367 | 8 | chr17 | 80419418 | 80419493 | + | — |
| hsa-mir-4729 | MI0017366 | — | chr17 | 59366083 | 59366154 | + | — |
| hsa-mir-4728 | MI0017365 | — | chr17 | 39726495 | 39726561 | + | — |
| hsa-mir-4727 | MI0017364 | — | chr17 | 38825838 | 38825892 | + | — |
| hsa-mir-4726 | MI0017363 | — | chr17 | 38719691 | 38719748 | + | — |
| hsa-mir-4725 | MI0017362 | 24 | chr17 | 31575269 | 31575358 | + | — |
| hsa-mir-4724 | MI0017361 | 2 | chr17 | 31534883 | 31534971 | + | — |
| hsa-mir-4723 | MI0017359 | 1 | chr17 | 28360654 | 28360734 | + | — |
| hsa-mir-4722 | MI0017357 | 11 | chr16 | 88716278 | 88716337 | − | — |
| hsa-mir-4721 | MI0017356 | — | chr16 | 28843919 | 28844007 | − | — |
| hsa-mir-4720 | MI0017355 | — | chr16 | 81385018 | 81385093 | + | — |
| hsa-mir-4719 | MI0017354 | 41 | chr16 | 76868936 | 76869019 | + | — |
| hsa-mir-4718 | MI0017353 | — | chr16 | 12720321 | 12720371 | + | — |
| hsa-mir-4717 | MI0017352 | — | chr16 | 2274620 | 2274691 | + | — |
| hsa-mir-4716 | MI0017350 | 5 | chr15 | 49169070 | 49169153 | − | — |
| hsa-mir-4715 | MI0017349 | — | chr15 | 25848747 | 25848825 | − | — |
| hsa-mir-4714 | MI0017348 | — | chr15 | 98784426 | 98784502 | + | — |
| hsa-mir-4713 | MI0017347 | 4 | chr15 | 51242190 | 51242264 | + | — |
| hsa-mir-4712 | MI0017346 | — | chr15 | 50360329 | 50360410 | + | — |
| hsa-mir-4711 | MI0017345 | — | chr1 | 59733227 | 59733296 | − | — |
| hsa-mir-4710 | MI0017344 | 2 | chr14 | 104677694 | 104677749 | − | — |
| hsa-mir-4709 | MI0017342 | 4 | chr14 | 74480133 | 74480204 | − | — |
| hsa-mir-4708 | MI0017341 | 8 | chr14 | 65335117 | 65335183 | − | — |
| hsa-mir-4707 | MI0017340 | — | chr14 | 22956950 | 22957029 | − | — |
| hsa-mir-4706 | MI0017339 | 8 | chr14 | 65044688 | 65044769 | + | — |
| hsa-mir-4705 | MI0017338 | — | chr13 | 102045934 | 102046004 | − | — |
| hsa-mir-4704 | MI0017337 | — | chr13 | 66218250 | 66218324 | + | — |
| hsa-mir-4703 | MI0017336 | — | chr13 | 51552589 | 51552667 | + | — |
| hsa-mir-4701 | MI0017334 | — | chr12 | 48771975 | 48772037 | − | — |
| hsa-mir-4700 | MI0017333 | — | chr12 | 120723193 | 120723266 | + | — |
| hsa-mir-4699 | MI0017332 | 29 | chr12 | 81158388 | 81158461 | + | — |
| hsa-mir-4698 | MI0017331 | — | chr12 | 47187812 | 47187891 | + | — |
| hsa-mir-4697 | MI0017330 | — | chr11 | 133898504 | 133898581 | − | — |
| hsa-mir-4696 | MI0017329 | — | chr11 | 74720268 | 74720337 | − | — |
| hsa-mir-4695 | MI0017328 | 2 | chr1 | 18883202 | 18883275 | − | — |
| hsa-mir-4694 | MI0017327 | 75 | chr11 | 19760004 | 19760083 | − | — |
| hsa-mir-4693 | MI0017326 | 5 | chr11 | 103849906 | 103849980 | + | — |
| hsa-mir-4692 | MI0017325 | — | chr11 | 72783530 | 72783592 | + | — |
| hsa-mir-4691 | MI0017324 | 9 | chr11 | 68033897 | 68033981 | + | — |
| hsa-mir-4690 | MI0017323 | — | chr11 | 65636310 | 65636369 | + | — |
| hsa-mir-4689 | MI0017322 | 30 | chr1 | 5862672 | 5862741 | − | — |
| hsa-mir-4688 | MI0017321 | 50 | chr11 | 46376402 | 46376484 | + | — |
| hsa-mir-4687 | MI0017319 | 5 | chr11 | 3856062 | 3856141 | + | — |
| hsa-mir-4686 | MI0017318 | 18 | chr11 | 2173063 | 2173138 | + | — |
| hsa-mir-4685 | MI0017317 | 26 | chr10 | 98431292 | 98431360 | − | — |
| hsa-mir-4684 | MI0017316 | 2 | chr1 | 22719517 | 22719598 | + | — |
| hsa-mir-4683 | MI0017315 | 1 | chr10 | 35641172 | 35641252 | − | — |
| hsa-mir-4682 | MI0017314 | — | chr10 | 119958513 | 119958592 | + | — |
| hsa-mir-4681 | MI0017313 | 19 | chr10 | 119377972 | 119378043 | + | — |
| hsa-mir-4680 | MI0017312 | — | chr10 | 110898090 | 110898155 | + | — |
| hsa-mir-4679-2 | MI0017311 | 4 | chr10 | 89063335 | 89063411 | − | — |
| hsa-mir-4679-1 | MI0017310 | 4 | chr10 | 89063336 | 89063410 | + | — |
| hsa-mir-4678 | MI0017309 | — | chr10 | 87503881 | 87503954 | + | — |
| hsa-mir-4677 | MI0017308 | 1 | chr1 | 243346176 | 243346255 | + | ✓ |
| hsa-mir-4676 | MI0017307 | — | chr10 | 72721029 | 72721100 | + | — |
| hsa-mir-4675 | MI0017306 | — | chr10 | 20551970 | 20552046 | + | — |
| hsa-mir-4674 | MI0017305 | 8 | chr9 | 136546173 | 136546259 | − | — |
| hsa-mir-4673 | MI0017304 | — | chr9 | 136519568 | 136519626 | − | — |
| hsa-mir-4672 | MI0017303 | 46 | chr9 | 127869415 | 127869495 | − | — |
| hsa-mir-4671 | MI0017302 | — | chr1 | 234306467 | 234306539 | + | — |
| hsa-mir-4670 | MI0017301 | — | chr9 | 92527984 | 92528058 | − | — |
| hsa-mir-4669 | MI0017300 | 2 | chr9 | 134379411 | 134379472 | + | — |
| hsa-mir-4668 | MI0017298 | — | chr9 | 111932100 | 111932169 | + | — |
| hsa-mir-4667 | MI0017297 | 3 | chr9 | 35608094 | 35608159 | + | — |
| hsa-mir-4666b | MI0019299 | — | chrX | 29574278 | 29574358 | + | — |
| hsa-mir-4666a | MI0017296 | — | chr1 | 228462074 | 228462152 | + | — |
| hsa-mir-4665 | MI0017295 | — | chr9 | 6007826 | 6007904 | + | — |
| hsa-mir-4664 | MI0017294 | — | chr8 | 143733083 | 143733153 | − | — |
| hsa-mir-4663 | MI0017292 | 110 | chr8 | 123215788 | 123215863 | − | — |
| hsa-mir-4662b | MI0017293 | 1 | chr8 | 124821978 | 124822058 | − | — |
| hsa-mir-4662a | MI0017290 | 2 | chr8 | 124821985 | 124822051 | + | — |
| hsa-mir-4661 | MI0017289 | — | chr8 | 91205485 | 91205559 | + | — |
| hsa-mir-4660 | MI0017288 | 22 | chr8 | 9048445 | 9048518 | + | — |
| hsa-mir-466 | MI0014157 | 13 | chr3 | 31161704 | 31161787 | − | — |
| hsa-mir-4659b | MI0017291 | — | chr8 | 6745168 | 6745240 | − | — |
| hsa-mir-4659a | MI0017287 | 2 | chr8 | 6745164 | 6745244 | + | — |
| hsa-mir-4658 | MI0017286 | — | chr7 | 100156605 | 100156669 | − | — |
| hsa-mir-4657 | MI0017285 | — | chr7 | 44881748 | 44881800 | − | — |
| hsa-mir-4656 | MI0017284 | 11 | chr7 | 4788565 | 4788639 | − | — |
| hsa-mir-4655 | MI0017283 | 2 | chr7 | 1844180 | 1844253 | − | — |
| hsa-mir-4654 | MI0017282 | — | chr1 | 162157107 | 162157182 | + | — |
| hsa-mir-4653 | MI0017281 | — | chr7 | 101159473 | 101159555 | + | — |
| hsa-mir-4652 | MI0017280 | — | chr7 | 93716928 | 93717005 | + | — |
| hsa-mir-4651 | MI0017279 | 5 | chr7 | 75915197 | 75915269 | + | — |
| hsa-mir-4650-2 | MI0017278 | — | chr7 | 72697903 | 72697978 | + | — |
| hsa-mir-4650-1 | MI0017277 | — | chr7 | 67114322 | 67114397 | − | — |
| hsa-mir-4649 | MI0017276 | — | chr7 | 44110849 | 44110912 | + | — |
| hsa-mir-4648 | MI0017275 | — | chr7 | 2527074 | 2527145 | + | — |
| hsa-mir-4647 | MI0017274 | 5 | chr6 | 44254206 | 44254285 | − | — |
| hsa-mir-4646 | MI0017273 | — | chr6 | 31701029 | 31701091 | − | — |
| hsa-mir-4645 | MI0017272 | — | chr6 | 2854031 | 2854107 | − | — |
| hsa-mir-4644 | MI0017271 | 5 | chr6 | 170330761 | 170330844 | + | — |
| hsa-mir-4643 | MI0017270 | — | chr6 | 91521660 | 91521737 | + | — |
| hsa-mir-4642 | MI0017269 | — | chr6 | 44435641 | 44435722 | + | — |
| hsa-mir-4641 | MI0017268 | 33 | chr6 | 41598723 | 41598788 | + | — |
| hsa-mir-4640 | MI0017267 | 3 | chr6 | 30890883 | 30890972 | + | — |
| hsa-mir-4639 | MI0017266 | — | chr6 | 16141556 | 16141624 | + | — |
| hsa-mir-4638 | MI0017265 | 1 | chr5 | 181222566 | 181222633 | − | — |
| hsa-mir-4637 | MI0017264 | — | chr5 | 14825929 | 14826012 | − | — |
| hsa-mir-4636 | MI0017263 | — | chr5 | 9053816 | 9053895 | − | — |
| hsa-mir-4635 | MI0017262 | 2 | chr5 | 1062896 | 1062974 | − | — |
| hsa-mir-4634 | MI0017261 | — | chr5 | 174751734 | 174751787 | + | — |
| hsa-mir-4633 | MI0017260 | — | chr5 | 129097688 | 129097766 | + | — |
| hsa-mir-4632 | MI0017259 | — | chr1 | 12191713 | 12191773 | + | — |
| hsa-mir-455 | MI0003513 | 353 | chr9 | 114209434 | 114209529 | + | — |
| hsa-mir-4540 | MI0016911 | — | chr9 | 36864254 | 36864308 | − | — |
| hsa-mir-454 | MI0003820 | 207 | chr17 | 59137758 | 59137872 | − | ✓ |
| hsa-mir-4539 | MI0016910 | 8 | chr14 | 105857513 | 105857572 | − | — |
| hsa-mir-4538 | MI0016909 | 2 | chr14 | 105858165 | 105858242 | − | — |
| hsa-mir-4537 | MI0016908 | 2 | chr14 | 105859484 | 105859553 | − | — |
| hsa-mir-4536-2 | MI0019149 | 70 | chrX | 55451495 | 55451582 | + | — |
| hsa-mir-4536-1 | MI0016906 | — | chrX | 55451495 | 55451582 | − | — |
| hsa-mir-4535 | MI0016903 | — | chr22 | 48780295 | 48780353 | + | — |
| hsa-mir-4534 | MI0016901 | — | chr22 | 37988794 | 37988853 | + | — |
| hsa-mir-4533 | MI0016900 | 22 | chr20 | 60478111 | 60478181 | + | — |
| hsa-mir-4531 | MI0016898 | 42 | chr19 | 44653686 | 44653732 | − | — |
| hsa-mir-4530 | MI0016897 | — | chr19 | 39409623 | 39409678 | − | — |
| hsa-mir-4529 | MI0016896 | — | chr18 | 55479221 | 55479298 | + | — |
| hsa-mir-4528 | MI0016895 | — | chr18 | 53237101 | 53237190 | + | — |
| hsa-mir-4527 | MI0016894 | — | chr18 | 47380496 | 47380565 | + | — |
| hsa-mir-4526 | MI0016893 | — | chr18 | 13611114 | 13611200 | + | — |
| hsa-mir-4525 | MI0016892 | 4 | chr17 | 82668233 | 82668307 | − | — |
| hsa-mir-4524b | MI0019114 | 45 | chr17 | 69099542 | 69099656 | + | — |
| hsa-mir-4524a | MI0016891 | — | chr17 | 69099564 | 69099632 | − | ✓ |
| hsa-mir-4523 | MI0016890 | — | chr17 | 29390662 | 29390730 | + | — |
| hsa-mir-4522 | MI0016889 | — | chr17 | 27293910 | 27293996 | − | — |
| hsa-mir-4521 | MI0016887 | 654 | chr17 | 8186945 | 8187004 | + | — |
| hsa-mir-4520-2 | MI0017358 | — | chr17 | 6655449 | 6655502 | + | — |
| hsa-mir-4520-1 | MI0016886 | — | chr17 | 6655440 | 6655509 | − | — |
| hsa-mir-452 | MI0001733 | 123 | chrX | 151959628 | 151959712 | − | ✓ |
| hsa-mir-451b | MI0017360 | — | chr17 | 28861371 | 28861438 | + | — |
| hsa-mir-451a | MI0001729 | 3855 | chr17 | 28861369 | 28861440 | − | — |
| hsa-mir-4519 | MI0016885 | — | chr16 | 30875266 | 30875323 | − | — |
| hsa-mir-4518 | MI0016884 | 2 | chr16 | 30503919 | 30504001 | + | — |
| hsa-mir-4517 | MI0016883 | 10 | chr16 | 28958583 | 28958661 | + | — |
| hsa-mir-4516 | MI0016882 | 229 | chr16 | 2133119 | 2133204 | + | — |
| hsa-mir-4515 | MI0016881 | 4 | chr15 | 83067335 | 83067415 | + | — |
| hsa-mir-4514 | MI0016880 | — | chr15 | 80997417 | 80997473 | − | — |
| hsa-mir-4513 | MI0016879 | 16 | chr15 | 74788672 | 74788757 | − | — |
| hsa-mir-4512 | MI0016878 | 11 | chr15 | 66496958 | 66497034 | − | — |
| hsa-mir-4511 | MI0016877 | 6 | chr15 | 65719246 | 65719332 | − | — |
| hsa-mir-4510 | MI0016876 | 237 | chr15 | 35926856 | 35926923 | + | — |
| hsa-mir-450b | MI0005531 | 27 | chrX | 134540185 | 134540262 | − | ✓ |
| hsa-mir-450a-2 | MI0003187 | 275 | chrX | 134540508 | 134540607 | − | ✓ |
| hsa-mir-450a-1 | MI0001652 | 275 | chrX | 134540341 | 134540431 | − | ✓ |
| hsa-mir-4509-3 | MI0016875 | — | chr15 | 28490752 | 28490845 | − | — |
| hsa-mir-4509-2 | MI0016874 | — | chr15 | 28426491 | 28426584 | + | — |
| hsa-mir-4509-1 | MI0016873 | — | chr15 | 23197827 | 23197920 | + | — |
| hsa-mir-4508 | MI0016872 | 31 | chr15 | 23562062 | 23562131 | − | — |
| hsa-mir-4507 | MI0016871 | — | chr14 | 105858124 | 105858175 | − | — |
| hsa-mir-4506 | MI0016869 | — | chr14 | 93948226 | 93948302 | − | — |
| hsa-mir-4505 | MI0016868 | — | chr14 | 73758747 | 73758819 | + | — |
| hsa-mir-4504 | MI0016867 | — | chr14 | 50299855 | 50299946 | − | — |
| hsa-mir-4503 | MI0016866 | 119 | chr14 | 36952309 | 36952391 | − | — |
| hsa-mir-4502 | MI0016865 | — | chr13 | 114273828 | 114273908 | + | — |
| hsa-mir-4501 | MI0016864 | — | chr13 | 96427229 | 96427292 | + | — |
| hsa-mir-4500 | MI0016863 | 106 | chr13 | 87618665 | 87618740 | − | — |
| hsa-mir-449c | MI0003823 | 9 | chr5 | 55172262 | 55172353 | − | — |
| hsa-mir-449b | MI0003673 | 120 | chr5 | 55170646 | 55170742 | − | — |
| hsa-mir-449a | MI0001648 | 102 | chr5 | 55170532 | 55170622 | − | — |
| hsa-mir-4499 | MI0016862 | — | chr13 | 20433778 | 20433846 | − | — |
| hsa-mir-4498 | MI0016860 | 46 | chr12 | 120155434 | 120155499 | − | — |
| hsa-mir-4497 | MI0016859 | 96 | chr12 | 109833348 | 109833436 | + | — |
| hsa-mir-4496 | MI0016858 | — | chr12 | 108635810 | 108635870 | + | — |
| hsa-mir-4495 | MI0016857 | — | chr12 | 97939056 | 97939121 | − | — |
| hsa-mir-4494 | MI0016856 | — | chr12 | 47364186 | 47364269 | − | — |
| hsa-mir-4493 | MI0016855 | 23 | chr11 | 123381440 | 123381512 | − | — |
| hsa-mir-4492 | MI0016854 | 12 | chr11 | 118910708 | 118910787 | + | — |
| hsa-mir-4491 | MI0016853 | — | chr11 | 111347757 | 111347824 | + | — |
| hsa-mir-4490 | MI0016852 | — | chr11 | 90555774 | 90555857 | − | — |
| hsa-mir-4489 | MI0016850 | — | chr11 | 65649192 | 65649253 | + | — |
| hsa-mir-4488 | MI0016849 | 67 | chr11 | 61508596 | 61508657 | + | — |
| hsa-mir-4487 | MI0016848 | — | chr11 | 47400970 | 47401042 | + | ✓ |
| hsa-mir-4486 | MI0016847 | — | chr11 | 19575310 | 19575372 | + | — |
| hsa-mir-4485 | MI0016846 | 20 | chr11 | 10508270 | 10508326 | − | — |
| hsa-mir-4484 | MI0016845 | 24 | chr10 | 125819740 | 125819822 | + | — |
| hsa-mir-4483 | MI0016844 | — | chr10 | 113777993 | 113778054 | − | — |
| hsa-mir-4482 | MI0016843 | — | chr10 | 104268336 | 104268405 | − | — |
| hsa-mir-4481 | MI0016842 | — | chr10 | 12653138 | 12653197 | − | — |
| hsa-mir-4480 | MI0016841 | 100 | chr10 | 12578753 | 12578823 | + | — |
| hsa-mir-448 | MI0001637 | — | chrX | 114823454 | 114823564 | + | — |
| hsa-mir-4479 | MI0016838 | — | chr9 | 136886733 | 136886803 | + | — |
| hsa-mir-4478 | MI0016831 | — | chr9 | 122120082 | 122120167 | − | — |
| hsa-mir-4477b | MI0016830 | 11 | chr9 | 41233755 | 41233835 | − | — |
| hsa-mir-4477a | MI0016829 | 3 | chr9 | 41233755 | 41233835 | + | — |
| hsa-mir-4476 | MI0016828 | — | chr9 | 36893462 | 36893531 | − | — |
| hsa-mir-4475 | MI0016827 | — | chr9 | 36823539 | 36823599 | − | — |
| hsa-mir-4474 | MI0016826 | — | chr9 | 20502265 | 20502342 | − | — |
| hsa-mir-4473 | MI0016825 | — | chr9 | 20411148 | 20411238 | − | — |
| hsa-mir-4472-2 | MI0016824 | 65 | chr12 | 116428252 | 116428318 | − | — |
| hsa-mir-4472-1 | MI0016823 | 24 | chr8 | 142176339 | 142176418 | + | — |
| hsa-mir-4471 | MI0016822 | — | chr8 | 100382763 | 100382845 | + | — |
| hsa-mir-4470 | MI0016821 | — | chr8 | 61714788 | 61714859 | + | — |
| hsa-mir-4469 | MI0016820 | — | chr8 | 42896197 | 42896275 | − | — |
| hsa-mir-4468 | MI0016819 | — | chr7 | 138123758 | 138123821 | + | — |
| hsa-mir-4467 | MI0016818 | — | chr7 | 102471469 | 102471531 | + | — |
| hsa-mir-4466 | MI0016817 | 1 | chr6 | 156779678 | 156779731 | − | — |
| hsa-mir-4465 | MI0016816 | — | chr6 | 140683814 | 140683883 | + | — |
| hsa-mir-4464 | MI0016812 | — | chr6 | 90312742 | 90312833 | + | — |
| hsa-mir-4463 | MI0016811 | — | chr6 | 75428407 | 75428473 | + | — |
| hsa-mir-4462 | MI0016810 | — | chr6 | 37555365 | 37555422 | − | — |
| hsa-mir-4460 | MI0016806 | — | chr5 | 129397062 | 129397147 | − | — |
| hsa-mir-4458 | MI0016804 | — | chr5 | 8460925 | 8460999 | + | — |
| hsa-mir-4457 | MI0016803 | — | chr5 | 1309310 | 1309377 | − | — |
| hsa-mir-4456 | MI0016802 | — | — | | | | |
| hsa-mir-4455 | MI0016801 | 30 | chr4 | 184938383 | 184938440 | − | — |
| hsa-mir-4454 | MI0016800 | 457 | chr4 | 163093574 | 163093628 | − | — |
| hsa-mir-4453 | MI0016799 | — | chr4 | 152536428 | 152536516 | + | — |
| hsa-mir-4452 | MI0016798 | 7 | chr4 | 86542482 | 86542552 | − | — |
| hsa-mir-4451 | MI0016797 | 13 | chr4 | 85722468 | 85722533 | + | — |
| hsa-mir-4450 | MI0016795 | — | chr4 | 76573568 | 76573632 | + | — |
| hsa-mir-4449 | MI0016792 | 943 | chr4 | 52712682 | 52712747 | + | — |
| hsa-mir-4448 | MI0016791 | 59 | chr3 | 183886800 | 183886885 | + | — |
| hsa-mir-4447 | MI0016790 | — | chr3 | 116850277 | 116850367 | − | — |
| hsa-mir-4446 | MI0016789 | 1 | chr3 | 113594876 | 113594942 | + | — |
| hsa-mir-4445 | MI0016788 | 100 | chr3 | 109602828 | 109602897 | + | — |
| hsa-mir-4444-2 | MI0019111 | 9 | chr3 | 75214476 | 75214549 | + | — |
| hsa-mir-4444-1 | MI0016787 | 9 | chr2 | 177212726 | 177212799 | + | — |
| hsa-mir-4443 | MI0016786 | 872 | chr3 | 48196564 | 48196616 | + | — |
| hsa-mir-4442 | MI0016785 | 6 | chr3 | 25664873 | 25664939 | − | — |
| hsa-mir-4441 | MI0016784 | — | chr2 | 239085827 | 239085926 | − | — |
| hsa-mir-4440 | MI0016783 | — | chr2 | 239068817 | 239068914 | − | — |
| hsa-mir-4439 | MI0016782 | — | chr2 | 225010461 | 225010540 | − | — |
| hsa-mir-4438 | MI0016781 | 138 | chr2 | 213758067 | 213758159 | + | — |
| hsa-mir-4437 | MI0016778 | — | chr2 | 181305593 | 181305652 | − | — |
| hsa-mir-4436b-2 | MI0019110 | 7 | chr2 | 110284853 | 110284943 | + | — |
| hsa-mir-4436b-1 | MI0017425 | 7 | chr2 | 110086433 | 110086523 | − | — |
| hsa-mir-4436a | MI0016776 | — | chr2 | 88812370 | 88812454 | + | — |
| hsa-mir-4435-2 | MI0016777 | — | chr2 | 111321013 | 111321086 | − | — |
| hsa-mir-4435-1 | MI0016775 | — | chr2 | 87629755 | 87629834 | + | — |
| hsa-mir-4434 | MI0016774 | — | chr2 | 64525513 | 64525565 | + | — |
| hsa-mir-4433b | MI0025511 | — | chr2 | 64340747 | 64340848 | − | — |
| hsa-mir-4433a | MI0016773 | 5 | chr2 | 64340759 | 64340839 | + | — |
| hsa-mir-4432 | MI0016772 | 16 | chr2 | 60387362 | 60387445 | − | — |
| hsa-mir-4431 | MI0016771 | 5 | chr2 | 52702522 | 52702615 | − | — |
| hsa-mir-4430 | MI0016769 | 12 | chr2 | 33418516 | 33418564 | + | — |
| hsa-mir-4429 | MI0016768 | 1989 | chr2 | 11540605 | 11540677 | − | — |
| hsa-mir-4428 | MI0016767 | — | chr1 | 237471119 | 237471191 | + | — |
| hsa-mir-4427 | MI0016766 | — | chr1 | 233624152 | 233624219 | + | — |
| hsa-mir-4426 | MI0016765 | — | chr1 | 192716328 | 192716390 | + | — |
| hsa-mir-4425 | MI0016764 | 14 | chr1 | 25023503 | 25023586 | + | — |
| hsa-mir-4424 | MI0016763 | — | chr1 | 178677749 | 178677834 | + | — |
| hsa-mir-4423 | MI0016760 | 10 | chr1 | 85133794 | 85133873 | + | ✓ |
| hsa-mir-4422 | MI0016759 | — | chr1 | 55225641 | 55225723 | + | — |
| hsa-mir-4421 | MI0016758 | — | chr1 | 51059837 | 51059905 | + | — |
| hsa-mir-4420 | MI0016757 | — | chr1 | 30739156 | 30739232 | − | — |
| hsa-mir-4418 | MI0016754 | 44 | chr1 | 22266239 | 22266300 | + | — |
| hsa-mir-4330 | MI0015902 | 11 | chrX | 151168222 | 151168326 | + | — |
| hsa-mir-433 | MI0001723 | 2 | chr14 | 100881886 | 100881978 | + | ✓ |
| hsa-mir-4329 | MI0015901 | 4 | chrX | 112780718 | 112780788 | − | — |
| hsa-mir-4328 | MI0015904 | 80 | chrX | 78901194 | 78901249 | − | — |
| hsa-mir-4327 | MI0015867 | 57 | chr21 | 30375294 | 30375378 | − | — |
| hsa-mir-4326 | MI0015866 | — | chr20 | 63286808 | 63286866 | + | — |
| hsa-mir-4325 | MI0015865 | — | chr20 | 57321502 | 57321591 | − | — |
| hsa-mir-4324 | MI0015854 | 56 | chr19 | 49308797 | 49308868 | − | — |
| hsa-mir-4323 | MI0015853 | — | chr19 | 42133445 | 42133513 | − | — |
| hsa-mir-4322 | MI0015851 | 9 | chr19 | 10230413 | 10230485 | + | — |
| hsa-mir-4321 | MI0015852 | — | chr19 | 2250639 | 2250718 | + | — |
| hsa-mir-4320 | MI0015849 | — | chr18 | 50126499 | 50126563 | − | — |
| hsa-mir-432 | MI0003133 | 55 | chr14 | 100884483 | 100884576 | + | — |
| hsa-mir-4319 | MI0015848 | — | chr18 | 44970082 | 44970166 | − | — |
| hsa-mir-4318 | MI0015847 | 62 | chr18 | 37657135 | 37657215 | + | — |
| hsa-mir-4317 | MI0015850 | 1 | chr18 | 6374361 | 6374425 | − | — |
| hsa-mir-4316 | MI0015845 | 209 | chr17 | 77396984 | 77397054 | − | — |
| hsa-mir-4315-2 | MI0015983 | — | chr17 | 64822030 | 64822102 | − | — |
| hsa-mir-4315-1 | MI0015844 | — | chr17 | 45475363 | 45475435 | − | — |
| hsa-mir-4314 | MI0015846 | 150 | chr17 | 8088056 | 8088147 | + | — |
| hsa-mir-4313 | MI0015843 | 28 | chr15 | 75762215 | 75762315 | − | — |
| hsa-mir-4312 | MI0015842 | 57 | chr15 | 68801850 | 68801925 | − | — |
| hsa-mir-4311 | MI0015841 | 56 | chr15 | 66040233 | 66040332 | + | — |
| hsa-mir-4310 | MI0015840 | 77 | chr15 | 41866495 | 41866551 | − | — |
| hsa-mir-431 | MI0001721 | — | chr14 | 100881007 | 100881120 | + | — |
| hsa-mir-4309 | MI0015837 | — | chr14 | 102539644 | 102539726 | + | — |
| hsa-mir-4308 | MI0015839 | 6 | chr14 | 54878113 | 54878193 | − | — |
| hsa-mir-4307 | MI0015838 | — | chr14 | 26908642 | 26908725 | + | — |
| hsa-mir-4306 | MI0015836 | 135 | chr13 | 99643059 | 99643149 | + | — |
| hsa-mir-4305 | MI0015835 | 214 | chr13 | 39664034 | 39664135 | − | — |
| hsa-mir-4304 | MI0015832 | 13 | chr12 | 123010667 | 123010728 | − | — |
| hsa-mir-4303 | MI0015834 | — | chr12 | 97995383 | 97995448 | − | — |
| hsa-mir-4302 | MI0015833 | — | chr12 | 25874020 | 25874079 | − | — |
| hsa-mir-4301 | MI0015828 | 8 | chr11 | 113450023 | 113450088 | − | — |
| hsa-mir-4300 | MI0015831 | — | chr11 | 81890741 | 81890836 | − | — |
| hsa-mir-4299 | MI0015829 | — | chr11 | 11656651 | 11656722 | − | — |
| hsa-mir-4298 | MI0015830 | 9 | chr11 | 1859464 | 1859536 | − | — |
| hsa-mir-4297 | MI0015824 | — | chr10 | 129843299 | 129843374 | − | — |
| hsa-mir-4296 | MI0015823 | 6 | chr10 | 125032783 | 125032870 | − | — |
| hsa-mir-4295 | MI0015822 | — | chr10 | 112634170 | 112634254 | + | — |
| hsa-mir-4294 | MI0015827 | 171 | chr10 | 48985512 | 48985587 | − | — |
| hsa-mir-4293 | MI0015826 | — | chr10 | 14383200 | 14383277 | − | — |
| hsa-mir-4292 | MI0015897 | — | chr9 | 136830957 | 136831023 | + | — |
| hsa-mir-4291 | MI0015900 | — | chr9 | 93819357 | 93819421 | + | — |
| hsa-mir-4290 | MI0015899 | 145 | chr9 | 90023441 | 90023535 | − | — |
| hsa-mir-429 | MI0001641 | 381 | chr1 | 1169005 | 1169087 | + | — |
| hsa-mir-4289 | MI0015898 | 852 | chr9 | 88745836 | 88745905 | − | — |
| hsa-mir-4288 | MI0015896 | 8 | chr8 | 28505116 | 28505182 | − | — |
| hsa-mir-4287 | MI0015895 | — | chr8 | 27886039 | 27886116 | − | — |
| hsa-mir-4286 | MI0015894 | 310 | chr8 | 10666978 | 10667070 | + | — |
| hsa-mir-4285 | MI0015891 | 7 | chr7 | 102293103 | 102293187 | + | — |
| hsa-mir-4284 | MI0015893 | 22 | chr7 | 73711317 | 73711397 | + | — |
| hsa-mir-4283-2 | MI0015982 | — | chr7 | 63621090 | 63621169 | + | — |
| hsa-mir-4283-1 | MI0015892 | — | chr7 | 56955785 | 56955864 | − | — |
| hsa-mir-4282 | MI0015890 | — | chr6 | 72967687 | 72967753 | − | — |
| hsa-mir-4281 | MI0015885 | 11 | chr5 | 176629439 | 176629500 | − | — |
| hsa-mir-4280 | MI0015889 | — | chr5 | 87114879 | 87114954 | − | — |
| hsa-mir-4279 | MI0015887 | 13 | chr5 | 31936102 | 31936159 | − | — |
| hsa-mir-4278 | MI0015888 | — | chr5 | 6827853 | 6827921 | − | — |
| hsa-mir-4277 | MI0015886 | — | chr5 | 1708785 | 1708868 | − | — |
| hsa-mir-4276 | MI0015882 | — | chr4 | 174423795 | 174423864 | + | — |
| hsa-mir-4275 | MI0015883 | 266 | chr4 | 28819582 | 28819668 | + | — |
| hsa-mir-4274 | MI0015884 | 33 | chr4 | 7460028 | 7460118 | + | — |
| hsa-mir-4273 | MI0015881 | — | chr3 | 75738280 | 75738363 | + | — |
| hsa-mir-4272 | MI0015880 | — | chr3 | 67225464 | 67225527 | + | — |
| hsa-mir-4271 | MI0015879 | 24 | chr3 | 49274120 | 49274186 | + | — |
| hsa-mir-4270 | MI0015878 | 14 | chr3 | 15496239 | 15496308 | − | — |
| hsa-mir-4269 | MI0015875 | — | chr2 | 239305462 | 239305545 | + | — |
| hsa-mir-4268 | MI0015874 | — | chr2 | 219906502 | 219906565 | − | — |
| hsa-mir-4267 | MI0015871 | — | chr2 | 110069961 | 110070042 | − | — |
| hsa-mir-4266 | MI0015870 | — | chr2 | 109313571 | 109313625 | − | — |
| hsa-mir-4265 | MI0015869 | 5 | chr2 | 109141490 | 109141588 | − | — |
| hsa-mir-4264 | MI0015877 | 877 | chr2 | 79649294 | 79649359 | − | — |
| hsa-mir-4263 | MI0015876 | — | chr2 | 27996367 | 27996449 | + | — |
| hsa-mir-4262 | MI0015872 | — | chr2 | 11836933 | 11836986 | − | — |
| hsa-mir-4261 | MI0015868 | — | chr2 | 10192614 | 10192671 | − | — |
| hsa-mir-4260 | MI0015859 | 350 | chr1 | 209623444 | 209623510 | − | — |
| hsa-mir-4259 | MI0015858 | 10 | chr1 | 159899979 | 159900079 | − | — |
| hsa-mir-4258 | MI0015857 | 9 | chr1 | 154975693 | 154975783 | + | — |
| hsa-mir-4257 | MI0015856 | 12 | chr1 | 150551929 | 150552014 | + | — |
| hsa-mir-4256 | MI0015855 | — | chr1 | 112461770 | 112461833 | − | — |
| hsa-mir-4255 | MI0015863 | — | chr1 | 37161563 | 37161634 | + | — |
| hsa-mir-4254 | MI0015862 | — | chr1 | 31758660 | 31758735 | − | — |
| hsa-mir-4253 | MI0015860 | — | chr1 | 22863159 | 22863226 | − | — |
| hsa-mir-4252 | MI0015864 | — | chr1 | 6429834 | 6429896 | − | — |
| hsa-mir-4251 | MI0015861 | — | chr1 | 3127975 | 3128035 | + | — |
| hsa-mir-425 | MI0001448 | 1131 | chr3 | 49020148 | 49020234 | − | ✓ |
| hsa-mir-424 | MI0001446 | 3666 | chrX | 134546614 | 134546711 | − | ✓ |
| hsa-mir-423 | MI0001445 | 3706 | chr17 | 30117079 | 30117172 | + | ✓ |
| hsa-mir-422a | MI0001444 | 7 | chr15 | 63870930 | 63871019 | − | — |
| hsa-mir-421 | MI0003685 | 120 | chrX | 74218377 | 74218461 | − | ✓ |
| hsa-mir-412 | MI0002464 | — | chr14 | 101065447 | 101065537 | + | — |
| hsa-mir-411 | MI0003675 | 86 | chr14 | 101023325 | 101023420 | + | ✓ |
| hsa-mir-410 | MI0002465 | 12 | chr14 | 101065912 | 101065991 | + | ✓ |
| hsa-mir-409 | MI0001735 | 3 | chr14 | 101065300 | 101065378 | + | ✓ |
| hsa-mir-3978 | MI0016996 | — | chrX | 110082118 | 110082218 | + | — |
| hsa-mir-3977 | MI0016995 | — | chr5 | 82840155 | 82840224 | + | — |
| hsa-mir-3976 | MI0016994 | — | chr18 | 5840695 | 5840833 | + | — |
| hsa-mir-3975 | MI0016993 | 100 | chr18 | 35591737 | 35591806 | + | — |
| hsa-mir-3974 | MI0016992 | — | chr12 | 17673299 | 17673394 | + | — |
| hsa-mir-3973 | MI0016991 | 200 | chr11 | 36010098 | 36010204 | + | — |
| hsa-mir-3972 | MI0016990 | 122 | chr1 | 17277889 | 17277975 | + | — |
| hsa-mir-3960 | MI0016964 | 95 | chr9 | 127785833 | 127785923 | + | — |
| hsa-mir-3945 | MI0016602 | — | chr4 | 184851013 | 184851110 | − | — |
| hsa-mir-3944 | MI0016601 | 10 | chr10 | 133371556 | 133371663 | − | ✓ |
| hsa-mir-3943 | MI0016600 | — | chr7 | 43150895 | 43150994 | + | — |
| hsa-mir-3942 | MI0016599 | — | chr15 | 35372256 | 35372364 | − | ✓ |
| hsa-mir-3941 | MI0016598 | — | chr10 | 122416965 | 122417067 | + | — |
| hsa-mir-3940 | MI0016597 | — | chr19 | 6416410 | 6416511 | − | — |
| hsa-mir-3939 | MI0016596 | — | chr6 | 166997807 | 166997912 | − | — |
| hsa-mir-3938 | MI0016594 | — | chr3 | 55852492 | 55852594 | − | — |
| hsa-mir-3937 | MI0016593 | — | chrX | 39661216 | 39661321 | + | — |
| hsa-mir-3936 | MI0016592 | — | chr5 | 132365490 | 132365599 | − | — |
| hsa-mir-3935 | MI0016591 | 3 | chr16 | 56245520 | 56245623 | + | — |
| hsa-mir-3934 | MI0016590 | 1 | chr6 | 33698128 | 33698234 | + | — |
| hsa-mir-3929 | MI0016439 | 238 | chr18 | 35934088 | 35934142 | − | — |
| hsa-mir-3928 | MI0016438 | 1 | chr22 | 31160062 | 31160119 | − | — |
| hsa-mir-3927 | MI0016435 | — | chr9 | 109511475 | 109511545 | − | — |
| hsa-mir-3926-2 | MI0016437 | 6 | chr8 | 12727237 | 12727299 | + | — |
| hsa-mir-3926-1 | MI0016434 | 5 | chr8 | 12727232 | 12727304 | − | — |
| hsa-mir-3925 | MI0016433 | — | chr6 | 36622436 | 36622512 | − | — |
| hsa-mir-3924 | MI0016432 | 100 | chr10 | 57304479 | 57304559 | − | — |
| hsa-mir-3923 | MI0016430 | — | chr3 | 79507887 | 79507969 | + | — |
| hsa-mir-3922 | MI0016429 | — | chr12 | 104591633 | 104591716 | + | — |
| hsa-mir-3921 | MI0016428 | 4 | chr3 | 99964314 | 99964398 | − | — |
| hsa-mir-3920 | MI0016427 | — | chr11 | 101519820 | 101519905 | − | — |
| hsa-mir-3919 | MI0016425 | — | chr3 | 159282646 | 159282734 | + | — |
| hsa-mir-3918 | MI0016424 | — | chr6 | 158764661 | 158764753 | − | — |
| hsa-mir-3917 | MI0016423 | 3 | chr1 | 25906362 | 25906454 | − | — |
| hsa-mir-3916 | MI0016422 | 14 | chr1 | 247201967 | 247202060 | − | — |
| hsa-mir-3915 | MI0016420 | — | chrX | 32583656 | 32583752 | − | — |
| hsa-mir-3914-2 | MI0016421 | — | chr7 | 71307674 | 71307768 | + | — |
| hsa-mir-3914-1 | MI0016419 | — | chr7 | 71307672 | 71307770 | − | — |
| hsa-mir-3913-2 | MI0016418 | 4 | chr12 | 69584723 | 69584822 | + | — |
| hsa-mir-3913-1 | MI0016417 | 2 | chr12 | 69584722 | 69584823 | − | — |
| hsa-mir-3912 | MI0016416 | — | chr5 | 171386656 | 171386760 | − | — |
| hsa-mir-3911 | MI0016415 | 1 | chr9 | 127690687 | 127690795 | − | — |
| hsa-mir-3910-2 | MI0016431 | 2 | chr9 | 91636264 | 91636345 | − | — |
| hsa-mir-3910-1 | MI0016414 | 2 | chr9 | 91636251 | 91636361 | + | — |
| hsa-mir-3909 | MI0016413 | — | chr22 | 35335640 | 35335758 | + | ✓ |
| hsa-mir-3908 | MI0016412 | 72 | chr12 | 123536409 | 123536534 | + | — |
| hsa-mir-3907 | MI0016410 | 36 | chr7 | 151433489 | 151433639 | − | — |
| hsa-mir-384 | MI0001145 | — | chrX | 76919273 | 76919360 | − | — |
| hsa-mir-383 | MI0000791 | 8 | chr8 | 14853438 | 14853510 | − | — |
| hsa-mir-382 | MI0000790 | 23 | chr14 | 101054306 | 101054381 | + | ✓ |
| hsa-mir-381 | MI0000789 | 22 | chr14 | 101045920 | 101045994 | + | ✓ |
| hsa-mir-380 | MI0000788 | 1 | chr14 | 101025017 | 101025077 | + | ✓ |
| hsa-mir-379 | MI0000787 | 263 | chr14 | 101022066 | 101022132 | + | ✓ |
| hsa-mir-378j | MI0021273 | 7 | chr17 | 37614931 | 37615039 | − | — |
| hsa-mir-3781 | MI0016902 | 5084 | chr22 | 41923222 | 41923297 | − | — |
| hsa-mir-378h | MI0016808 | 3791 | chr5 | 154829458 | 154829540 | + | — |
| hsa-mir-378g | MI0016761 | 4642 | | | | | |
| hsa-mir-378f | MI0016756 | 4762 | chr1 | 23929070 | 23929147 | + | — |
| hsa-mir-378e | MI0016750 | 3130 | chr5 | 170028488 | 170028566 | + | — |
| hsa-mir-378d-2 | MI0003840 | 4789 | chr8 | 93916022 | 93916119 | − | — |
| hsa-mir-378d-1 | MI0016749 | 3415 | chr4 | 5923275 | 5923328 | − | — |
| hsa-mir-378c | MI0015825 | 4978 | chr10 | 130962588 | 130962668 | − | — |
| hsa-mir-378b | MI0014154 | 3955 | chr3 | 10330229 | 10330285 | + | — |
| hsa-mir-378a | MI0000786 | 6009 | chr5 | 149732825 | 149732890 | + | ✓ |
| hsa-mir-377 | MI0000785 | 15 | chr14 | 101062050 | 101062118 | + | ✓ |
| hsa-mir-376c | MI0000776 | 214 | chr14 | 101039690 | 101039755 | + | ✓ |
| hsa-mir-376b | MI0002466 | 115 | chr14 | 101040436 | 101040535 | + | ✓ |
| hsa-mir-376a-2 | MI0003529 | 119 | chr14 | 101040069 | 101040148 | + | ✓ |
| hsa-mir-376a-1 | MI0000784 | 120 | chr14 | 101040782 | 101040849 | + | ✓ |
| hsa-mir-375 | MI0000783 | 3418 | chr2 | 219001645 | 219001708 | − | ✓ |
| hsa-mir-374c | MI0016684 | 8 | chrX | 74218549 | 74218618 | + | — |
| hsa-mir-374b | MI0005566 | 1108 | chrX | 74218547 | 74218618 | − | ✓ |
| hsa-mir-374a | MI0000782 | 1271 | chrX | 74287286 | 74287357 | − | ✓ |
| hsa-mir-373 | MI0000781 | 2 | chr19 | 53788705 | 53788773 | + | — |
| hsa-mir-372 | MI0000780 | 91 | chr19 | 53787890 | 53787956 | + | — |
| hsa-mir-371b | MI0017393 | 23 | chr19 | 53787677 | 53787742 | − | — |
| hsa-mir-371a | MI0000779 | — | chr19 | 53787675 | 53787741 | + | — |
| hsa-mir-3714 | MI0016135 | 5 | chr3 | 16933196 | 16933260 | + | — |
| hsa-mir-3713 | MI0016134 | — | | | | | |
| hsa-mir-370 | MI0000778 | 9 | chr14 | 100911139 | 100911213 | + | ✓ |
| hsa-mir-3692 | MI0016093 | — | chr6 | 157529132 | 157529200 | + | — |
| hsa-mir-3691 | MI0016092 | — | chr6 | 5148233 | 5148322 | − | — |
| hsa-mir-3690-2 | MI0023561 | — | chrY | 1293918 | 1293992 | + | — |
| hsa-mir-3690-1 | MI0016091 | — | chrX | 1293918 | 1293992 | + | — |
| hsa-mir-369 | MI0000777 | 53 | chr14 | 101065598 | 101065667 | + | ✓ |
| hsa-mir-3689f | MI0016837 | 25 | chr9 | 134850742 | 134850807 | − | — |
| hsa-mir-3689e | MI0016836 | — | chr9 | 134850570 | 134850641 | − | — |
| hsa-mir-3689d-2 | MI0016835 | — | chr9 | 134850277 | 134850356 | − | — |
| hsa-mir-3689d-1 | MI0016834 | — | chr9 | 134849609 | 134849682 | − | — |
| hsa-mir-3689c | MI0016832 | — | chr9 | 134849298 | 134849369 | − | — |
| hsa-mir-3689b | MI0016411 | — | chr9 | 134850125 | 134850272 | − | — |
| hsa-mir-3689a | MI0016090 | — | chr9 | 134849487 | 134849564 | − | — |
| hsa-mir-3688-2 | MI0017447 | 3 | chr4 | 159128805 | 159128891 | + | — |
| hsa-mir-3688-1 | MI0016089 | 73 | chr4 | 159128802 | 159128894 | − | — |
| hsa-mir-3686 | MI0016087 | — | chr8 | 129484057 | 129484142 | − | — |
| hsa-mir-3685 | MI0016086 | — | chr12 | 95309923 | 95309984 | + | — |
| hsa-mir-3684 | MI0016085 | — | chr4 | 98997387 | 98997460 | + | — |
| hsa-mir-3683 | MI0016084 | — | chr7 | 7066964 | 7067045 | − | — |
| hsa-mir-3682 | MI0016083 | 4 | chr2 | 53849122 | 53849205 | − | — |
| hsa-mir-3681 | MI0016082 | — | chr2 | 12199130 | 12199201 | + | — |
| hsa-mir-3680-2 | MI0019113 | 3 | chr16 | 29599179 | 29599265 | − | — |
| hsa-mir-3680-1 | MI0016081 | 3 | chr16 | 21506049 | 21506135 | − | — |
| hsa-mir-3679 | MI0016080 | — | chr2 | 134127125 | 134127192 | + | — |
| hsa-mir-3678 | MI0016079 | 10 | chr17 | 75406069 | 75406162 | + | — |
| hsa-mir-3677 | MI0016078 | 8 | chr16 | 2270713 | 2270772 | + | ✓ |
| hsa-mir-3675 | MI0016076 | — | chr1 | 16858949 | 16859021 | − | — |
| hsa-mir-3674 | MI0016075 | — | chr8 | 1801125 | 1801192 | + | — |
| hsa-mir-3672 | MI0016073 | — | chrX | 121370972 | 121371053 | + | — |
| hsa-mir-3671 | MI0016072 | — | chr1 | 65057755 | 65057842 | − | — |
| hsa-mir-3670-4 | MI0031514 | — | chr16 | 18488301 | 18488365 | − | — |
| hsa-mir-3670-3 | MI0031513 | — | chr16 | 18405698 | 18405762 | − | — |
| hsa-mir-3670-2 | MI0019112 | — | chr16 | 16306370 | 16306434 | + | — |
| hsa-mir-3670-1 | MI0016071 | — | chr16 | 14907717 | 14907781 | + | — |
| hsa-mir-367 | MI0000775 | 9 | chr4 | 112647874 | 112647941 | − | — |
| hsa-mir-3668 | MI0016069 | — | chr6 | 140205252 | 140205326 | + | — |
| hsa-mir-3667 | MI0016068 | — | chr22 | 49543393 | 49543466 | − | — |
| hsa-mir-3666 | MI0016067 | — | chr7 | 114653345 | 114653455 | + | — |
| hsa-mir-3665 | MI0016066 | 63 | chr13 | 77698012 | 77698116 | − | — |
| hsa-mir-3664 | MI0016065 | — | chr11 | 70872270 | 70872368 | − | — |
| hsa-mir-3663 | MI0016064 | 38 | chr10 | 117167678 | 117167774 | − | — |
| hsa-mir-3662 | MI0016063 | — | chr6 | 134979338 | 134979432 | − | — |
| hsa-mir-3661 | MI0016062 | 5 | chr5 | 134225757 | 134225852 | + | — |
| hsa-mir-3660 | MI0016061 | 29 | chr5 | 90016621 | 90016720 | − | — |
| hsa-mir-365b | MI0000769 | 1025 | chr17 | 31575411 | 31575521 | + | ✓ |
| hsa-mir-365a | MI0000767 | 1023 | chr16 | 14309285 | 14309371 | + | ✓ |
| hsa-mir-3659 | MI0016060 | — | chr1 | 38089231 | 38089329 | + | — |
| hsa-mir-3658 | MI0016058 | 3 | chr1 | 165907921 | 165907976 | + | — |
| hsa-mir-3657 | MI0016057 | — | chr12 | 112037599 | 112037715 | − | ✓ |
| hsa-mir-3655 | MI0016055 | — | chr5 | 140647844 | 140647926 | + | — |
| hsa-mir-3654 | MI0016054 | — | chr7 | 133034860 | 133034915 | − | — |
| hsa-mir-3652 | MI0016052 | 5 | chr12 | 103930425 | 103930555 | + | — |
| hsa-mir-3651 | MI0016051 | 3 | chr9 | 92292458 | 92292547 | − | — |
| hsa-mir-3650 | MI0016050 | — | chr5 | 38557502 | 38557561 | − | — |
| hsa-mir-3649 | MI0016049 | 28 | chr12 | 1660315 | 1660380 | − | — |
| hsa-mir-3648-2 | MI0031512 | 8 | chr21 | 8986999 | 8987178 | + | — |
| hsa-mir-3648-1 | MI0016048 | 8 | chr21 | 8208473 | 8208652 | + | — |
| hsa-mir-3646 | MI0016046 | 18 | chr20 | 44408120 | 44408203 | + | — |
| hsa-mir-363 | MI0000764 | 757 | chrX | 134169378 | 134169452 | − | ✓ |
| hsa-mir-3622b | MI0016014 | 6 | chr8 | 27701673 | 27701767 | − | — |
| hsa-mir-3622a | MI0016013 | 5 | chr8 | 27701677 | 27701759 | + | — |
| hsa-mir-3621 | MI0016012 | — | chr9 | 137169186 | 137169270 | − | — |
| hsa-mir-3620 | MI0016011 | — | chr1 | 228097263 | 228097341 | + | — |
| hsa-mir-362 | MI0000762 | 61 | chrX | 50008964 | 50009028 | + | ✓ |
| hsa-mir-3619 | MI0016009 | 2 | chr22 | 46091044 | 46091126 | + | — |
| hsa-mir-3618 | MI0016008 | 8 | chr22 | 20085746 | 20085833 | + | — |
| hsa-mir-3617 | MI0016007 | — | chr20 | 45705102 | 45705180 | − | — |
| hsa-mir-3616 | MI0016006 | 2 | chr20 | 47166967 | 47167058 | + | ✓ |
| hsa-mir-3615 | MI0016005 | 11 | chr17 | 74748613 | 74748699 | + | — |
| hsa-mir-3614 | MI0016004 | 19 | chr17 | 56891270 | 56891355 | − | ✓ |
| hsa-mir-3613 | MI0016003 | 321 | chr13 | 49996415 | 49996501 | − | ✓ |
| hsa-mir-3612 | MI0016002 | — | chr12 | 128294092 | 128294178 | + | — |
| hsa-mir-3611 | MI0016001 | 4 | chr10 | 35079598 | 35079680 | − | ✓ |
| hsa-mir-3610 | MI0016000 | 13 | chr8 | 116874728 | 116874800 | − | — |
| hsa-mir-361 | MI0000760 | 866 | chrX | 85903636 | 85903707 | − | ✓ |
| hsa-mir-3609 | MI0015999 | 73 | chr7 | 98881650 | 98881729 | + | — |
| hsa-mir-3606 | MI0015996 | 9 | chr2 | 188995630 | 188995692 | + | — |
| hsa-mir-3605 | MI0015995 | 1 | chr1 | 33332393 | 33332492 | − | — |
| hsa-mir-3529 | MI0017351 | — | chr15 | 88611847 | 88611924 | − | — |
| hsa-mir-34c | MI0000743 | 379 | chr11 | 111513439 | 111513515 | + | ✓ |
| hsa-mir-34b | MI0000742 | 43 | chr11 | 111512938 | 111513021 | + | ✓ |
| hsa-mir-34a | MI0000268 | 269 | chr1 | 9151668 | 9151777 | − | ✓ |
| hsa-mir-346 | MI0000826 | 3 | chr10 | 86264694 | 86264788 | − | — |
| hsa-mir-345 | MI0000825 | 123 | chr14 | 100307859 | 100307956 | + | ✓ |
| hsa-mir-342 | MI0000805 | 776 | chr14 | 100109655 | 100109753 | + | ✓ |
| hsa-mir-340 | MI0000802 | 913 | chr5 | 180015303 | 180015397 | − | ✓ |
| hsa-mir-33b | MI0003646 | 27 | chr17 | 17813836 | 17813931 | − | ✓ |
| hsa-mir-33a | MI0000091 | 256 | chr22 | 41900944 | 41901012 | + | ✓ |
| hsa-mir-339 | MI0000815 | 238 | chr7 | 1022933 | 1023026 | − | ✓ |
| hsa-mir-338 | MI0000814 | 97 | chr17 | 81125883 | 81125949 | − | ✓ |
| hsa-mir-337 | MI0000806 | 21 | chr14 | 100874493 | 100874585 | + | ✓ |
| hsa-mir-335 | MI0000816 | 3777 | chr7 | 130496111 | 130496204 | + | ✓ |
| hsa-mir-331 | MI0000812 | 226 | chr12 | 95308420 | 95308513 | + | — |
| hsa-mir-330 | MI0000803 | 70 | chr19 | 45638994 | 45639087 | − | ✓ |
| hsa-mir-329-2 | MI0001726 | — | chr14 | 101027100 | 101027183 | + | ✓ |
| hsa-mir-329-1 | MI0001725 | — | chr14 | 101026785 | 101026864 | + | ✓ |
| hsa-mir-328 | MI0000804 | 31 | chr16 | 67202321 | 67202395 | − | — |
| hsa-mir-326 | MI0000808 | 14 | chr11 | 75335092 | 75335186 | − | — |
| hsa-mir-325 | MI0000824 | — | chrX | 77005404 | 77005501 | − | — |
| hsa-mir-324 | MI0000813 | 210 | chr17 | 7223297 | 7223379 | − | ✓ |
| hsa-mir-323b | MI0014206 | — | chr14 | 101056219 | 101056300 | + | — |
| hsa-mir-323a | MI0000807 | 1 | chr14 | 101025732 | 101025817 | + | ✓ |
| hsa-mir-320e | MI0014234 | 4717 | chr19 | 46709282 | 46709354 | − | — |
| hsa-mir-320d-2 | MI0008192 | 2985 | chrX | 140926160 | 140926231 | − | — |
| hsa-mir-320d-1 | MI0008190 | 4526 | chr13 | 40727816 | 40727887 | − | — |
| hsa-mir-320c-2 | MI0008191 | 4285 | chr18 | 24321675 | 24321746 | + | — |
| hsa-mir-320c-1 | MI0003778 | 3570 | chr18 | 21683518 | 21683589 | + | — |
| hsa-mir-320b-2 | MI0003839 | 5249 | chr1 | 224257040 | 224257110 | − | — |
| hsa-mir-320b-1 | MI0003776 | 5177 | chr1 | 116671746 | 116671817 | + | — |
| hsa-mir-320a | MI0000542 | 5331 | chr8 | 22244966 | 22245037 | − | ✓ |
| hsa-mir-3202-2 | MI0014253 | 3 | chrX | 153981098 | 153981176 | − | — |
| hsa-mir-3202-1 | MI0014252 | 1 | chrX | 153981097 | 153981177 | + | — |
| hsa-mir-3201 | MI0014250 | — | chr22 | 48274364 | 48274415 | + | — |
| hsa-mir-3200 | MI0014249 | 3 | chr22 | 30731557 | 30731641 | + | ✓ |
| hsa-mir-32 | MI0000090 | 222 | chr9 | 109046229 | 109046298 | − | ✓ |
| hsa-mir-3199-2 | MI0014248 | — | chr22 | 27920526 | 27920611 | + | — |
| hsa-mir-3199-1 | MI0014247 | — | chr22 | 27920525 | 27920612 | − | — |
| hsa-mir-3198-2 | MI0017335 | — | chr12 | 54231397 | 54231476 | − | — |
| hsa-mir-3198-1 | MI0014246 | — | chr22 | 17764180 | 17764259 | − | — |
| hsa-mir-3197 | MI0014245 | — | chr21 | 41167557 | 41167629 | + | — |
| hsa-mir-3196 | MI0014241 | 12 | chr20 | 63238779 | 63238842 | + | — |
| hsa-mir-3195 | MI0014240 | 27 | chr20 | 62064802 | 62064885 | + | — |
| hsa-mir-3194 | MI0014239 | — | chr20 | 51452905 | 51452977 | − | — |
| hsa-mir-3193 | MI0014238 | — | chr20 | 31607186 | 31607240 | + | — |
| hsa-mir-3192 | MI0014237 | 457 | chr20 | 18470615 | 18470691 | + | — |
| hsa-mir-3191 | MI0014236 | — | chr19 | 47226944 | 47227019 | − | — |
| hsa-mir-3190 | MI0014235 | 12 | chr19 | 47226942 | 47227021 | + | — |
| hsa-mir-3189 | MI0014233 | 2 | chr19 | 18386562 | 18386634 | + | — |
| hsa-mir-3188 | MI0014232 | 1 | chr19 | 18282077 | 18282161 | + | — |
| hsa-mir-3187 | MI0014231 | 2 | chr19 | 813584 | 813653 | + | — |
| hsa-mir-3186 | MI0014229 | — | chr17 | 81451104 | 81451188 | − | — |
| hsa-mir-3185 | MI0014227 | 6 | chr17 | 48724408 | 48724475 | − | — |
| hsa-mir-3184 | MI0014226 | — | chr17 | 30117086 | 30117160 | − | — |
| hsa-mir-3183 | MI0014225 | — | chr17 | 1022476 | 1022559 | − | — |
| hsa-mir-3182 | MI0014224 | 37 | chr16 | 83508346 | 83508408 | + | — |
| hsa-mir-3181 | MI0014223 | 10 | chr16 | 50742305 | 50742377 | + | — |
| hsa-mir-3180-5 | MI0016409 | 15 | chr16 | 2135977 | 2136129 | − | — |
| hsa-mir-3180-4 | MI0016408 | 14 | chr16 | 15154850 | 15155002 | − | — |
| hsa-mir-3180-3 | MI0014217 | 13 | chr16 | 18402178 | 18402271 | − | ✓ |
| hsa-mir-3180-2 | MI0014215 | 13 | chr16 | 16309879 | 16309966 | + | ✓ |
| hsa-mir-3180-1 | MI0014214 | 13 | chr16 | 14911220 | 14911313 | + | ✓ |
| hsa-mir-3179-4 | MI0031510 | — | chr16 | 18494493 | 18494576 | − | — |
| hsa-mir-3179-3 | MI0014221 | — | chr16 | 18411894 | 18411977 | − | — |
| hsa-mir-3179-2 | MI0014216 | — | chr16 | 16300159 | 16300242 | + | — |
| hsa-mir-3179-1 | MI0014213 | — | chr16 | 14901508 | 14901591 | + | — |
| hsa-mir-3178 | MI0014212 | 37 | chr16 | 2531922 | 2532005 | − | — |
| hsa-mir-3177 | MI0014211 | — | chr16 | 1734985 | 1735066 | + | — |
| hsa-mir-3176 | MI0014210 | 51 | chr16 | 543277 | 543366 | + | — |
| hsa-mir-3175 | MI0014209 | 1 | chr15 | 92904399 | 92904475 | + | — |
| hsa-mir-3174 | MI0014208 | — | chr15 | 90006755 | 90006841 | + | — |
| hsa-mir-3173 | MI0014204 | 7 | chr14 | 95137919 | 95137986 | − | ✓ |
| hsa-mir-3171 | MI0014202 | — | chr14 | 27633205 | 27633278 | − | ✓ |
| hsa-mir-3170 | MI0014201 | — | chr13 | 98208524 | 98208600 | + | — |
| hsa-mir-3169 | MI0014200 | — | chr13 | 61199798 | 61199880 | − | — |
| hsa-mir-3168 | MI0014199 | 98 | chr13 | 41101019 | 41101100 | − | — |
| hsa-mir-3167 | MI0014198 | — | chr11 | 126988458 | 126988542 | − | — |
| hsa-mir-3166 | MI0014196 | — | chr11 | 88176502 | 88176593 | + | — |
| hsa-mir-3165 | MI0014195 | 1 | chr11 | 72072228 | 72072302 | − | — |
| hsa-mir-3164 | MI0014194 | — | chr11 | 69083176 | 69083258 | + | — |
| hsa-mir-3163 | MI0014193 | 4 | chr11 | 66934434 | 66934506 | − | — |
| hsa-mir-3162 | MI0014192 | 1 | chr11 | 59595077 | 59595158 | − | — |
| hsa-mir-3161 | MI0014191 | — | chr11 | 48096782 | 48096858 | + | — |
| hsa-mir-3160-2 | MI0014190 | 1 | chr11 | 46451807 | 46451887 | + | — |
| hsa-mir-3160-1 | MI0014189 | — | chr11 | 46451805 | 46451889 | − | — |
| hsa-mir-3159 | MI0014188 | 638 | chr11 | 18387787 | 18387860 | + | — |
| hsa-mir-3158-2 | MI0014187 | 2 | chr10 | 101601417 | 101601497 | − | ✓ |
| hsa-mir-3158-1 | MI0014186 | 2 | chr10 | 101601417 | 101601497 | + | ✓ |
| hsa-mir-3157 | MI0014185 | — | chr10 | 96064315 | 96064399 | − | ✓ |
| hsa-mir-3156-3 | MI0014242 | — | chr21 | 13406384 | 13406460 | − | — |
| hsa-mir-3156-2 | MI0014230 | — | chr18 | 14830166 | 14830242 | + | — |
| hsa-mir-3156-1 | MI0014184 | — | chr10 | 45164014 | 45164088 | + | — |
| hsa-mir-3155b | MI0016839 | 22 | chr10 | 6152207 | 6152262 | − | — |
| hsa-mir-3155a | MI0014183 | 2 | chr10 | 6152196 | 6152277 | + | ✓ |
| hsa-mir-3154 | MI0014182 | 1 | chr9 | 128244947 | 128245030 | − | — |
| hsa-mir-3153 | MI0014180 | — | chr9 | 89312225 | 89312306 | + | — |
| hsa-mir-3152 | MI0014179 | 19 | chr9 | 18573306 | 18573379 | + | — |
| hsa-mir-3151 | MI0014178 | — | chr8 | 103154614 | 103154689 | + | — |
| hsa-mir-3150b | MI0016426 | 5 | chr8 | 95072911 | 95072996 | − | — |
| hsa-mir-3150a | MI0014177 | 24 | chr8 | 95072914 | 95072993 | + | — |
| hsa-mir-3149 | MI0014176 | 130 | chr8 | 76966768 | 76966850 | − | — |
| hsa-mir-3148 | MI0014175 | — | chr8 | 29957272 | 29957348 | − | — |
| hsa-mir-3147 | MI0014173 | — | chr7 | 57405025 | 57405090 | + | — |
| hsa-mir-3146 | MI0014172 | — | chr7 | 19705358 | 19705436 | − | — |
| hsa-mir-3145 | MI0014170 | — | chr6 | 138435213 | 138435294 | − | — |
| hsa-mir-3144 | MI0014169 | 3 | chr6 | 120015179 | 120015257 | + | ✓ |
| hsa-mir-3143 | MI0014167 | 172 | chr6 | 27147626 | 27147688 | + | — |
| hsa-mir-3142 | MI0014166 | — | chr5 | 160474402 | 160474483 | + | — |
| hsa-mir-3141 | MI0014165 | 1 | chr5 | 154596012 | 154596072 | − | — |
| hsa-mir-3140 | MI0014163 | — | chr4 | 152489327 | 152489416 | − | ✓ |
| hsa-mir-3139 | MI0014162 | 48 | chr4 | 143343460 | 143343535 | + | — |
| hsa-mir-3138 | MI0014161 | 1 | chr4 | 10078611 | 10078692 | − | — |
| hsa-mir-3137 | MI0014160 | 28 | chr3 | 195134506 | 195134580 | − | — |
| hsa-mir-3136 | MI0014158 | — | chr3 | 69048958 | 69049035 | − | — |
| hsa-mir-3135b | MI0016809 | 71 | chr6 | 32749912 | 32749979 | − | — |
| hsa-mir-3135a | MI0014156 | 367 | chr3 | 20137565 | 20137641 | + | — |
| hsa-mir-3134 | MI0014155 | 14 | chr3 | 15697298 | 15697371 | − | — |
| hsa-mir-3133 | MI0014153 | — | chr2 | 241477905 | 241477982 | + | — |
| hsa-mir-3132 | MI0014152 | — | chr2 | 219549073 | 219549147 | − | — |
| hsa-mir-3131 | MI0014151 | — | chr2 | 219058688 | 219058750 | − | — |
| hsa-mir-3130-2 | MI0014148 | 3 | chr2 | 206783234 | 206783308 | + | ✓ |
| hsa-mir-3130-1 | MI0014147 | 3 | chr2 | 206783234 | 206783308 | − | ✓ |
| hsa-mir-3129 | MI0014146 | — | chr2 | 189133036 | 189133111 | − | — |
| hsa-mir-3128 | MI0014145 | — | chr2 | 177255945 | 177256010 | − | — |
| hsa-mir-3127 | MI0014144 | — | chr2 | 96798278 | 96798353 | + | — |
| hsa-mir-3126 | MI0014143 | — | chr2 | 69103682 | 69103755 | + | ✓ |
| hsa-mir-3125 | MI0014142 | 9 | chr2 | 12737367 | 12737444 | + | — |
| hsa-mir-3124 | MI0014140 | — | chr1 | 248826377 | 248826443 | + | — |
| hsa-mir-3123 | MI0014139 | 46 | chr1 | 241132272 | 241132346 | + | — |
| hsa-mir-3122 | MI0014138 | — | chr1 | 212077613 | 212077685 | + | — |
| hsa-mir-3121 | MI0014137 | — | chr1 | 180438314 | 180438390 | − | — |
| hsa-mir-3120 | MI0014136 | — | chr1 | 172138808 | 172138888 | + | — |
| hsa-mir-3119-2 | MI0014135 | — | chr1 | 170151378 | 170151462 | + | — |
| hsa-mir-3119-1 | MI0014134 | — | chr1 | 170151378 | 170151462 | − | — |
| hsa-mir-3118-4 | MI0014207 | — | chr15 | 21843750 | 21843824 | + | — |
| hsa-mir-3118-3 | MI0014133 | — | chr15 | 21406385 | 21406459 | + | — |
| hsa-mir-3118-2 | MI0014132 | — | chr15 | 20832795 | 20832869 | + | — |
| hsa-mir-3118-1 | MI0014131 | — | chr21 | 13644775 | 13644850 | − | — |
| hsa-mir-3117 | MI0014130 | — | chr1 | 66628440 | 66628517 | + | — |
| hsa-mir-3116-2 | MI0014129 | — | chr1 | 62078789 | 62078856 | − | — |
| hsa-mir-3116-1 | MI0014128 | — | chr1 | 62078786 | 62078859 | + | — |
| hsa-mir-3115 | MI0014127 | 29 | chr1 | 23044305 | 23044372 | + | — |
| hsa-mir-31 | MI0000089 | 1263 | chr9 | 21512115 | 21512185 | − | ✓ |
| hsa-mir-30e | MI0000749 | 10738 | chr1 | 40754355 | 40754446 | + | ✓ |
| hsa-mir-30d | MI0000255 | 11691 | chr8 | 134804876 | 134804945 | − | ✓ |
| hsa-mir-30c-2 | MI0000254 | 3223 | chr6 | 71376960 | 71377031 | − | ✓ |
| hsa-mir-30c-1 | MI0000736 | 3205 | chr1 | 40757284 | 40757372 | + | ✓ |
| hsa-mir-30b | MI0000441 | 2245 | chr8 | 134800520 | 134800607 | − | ✓ |
| hsa-mir-30a | MI0000088 | 15057 | chr6 | 71403551 | 71403621 | − | ✓ |
| hsa-mir-3085 | MI0039500 | — | chr10 | 97875813 | 97875897 | − | — |
| hsa-mir-3074 | MI0014181 | 4 | chr9 | 95086014 | 95086094 | − | — |
| hsa-mir-3065 | MI0014228 | 39 | chr17 | 81125877 | 81125955 | + | ✓ |
| hsa-mir-3064 | MI0017375 | — | chr17 | 64500774 | 64500839 | − | — |
| hsa-mir-3059 | MI0039499 | 8 | chr12 | 86745068 | 86745149 | − | ✓ |
| hsa-mir-302f | MI0006418 | — | chr18 | 30298910 | 30298960 | + | — |
| hsa-mir-302e | MI0006417 | — | chr11 | 7234766 | 7234837 | + | — |
| hsa-mir-302d | MI0000774 | 10 | chr4 | 112648004 | 112648071 | − | — |
| hsa-mir-302c | MI0000773 | — | chr4 | 112648363 | 112648430 | − | — |
| hsa-mir-302b | MI0000772 | — | chr4 | 112648485 | 112648557 | − | — |
| hsa-mir-302a | MI0000738 | 42 | chr4 | 112648183 | 112648251 | − | ✓ |
| hsa-mir-301b | MI0005568 | 140 | chr22 | 21652981 | 21653058 | + | ✓ |
| hsa-mir-301a | MI0000745 | 227 | chr17 | 59151136 | 59151221 | − | ✓ |
| hsa-mir-300 | MI0005525 | — | chr14 | 101041363 | 101041445 | + | — |
| hsa-mir-29c | MI0000735 | 11179 | chr1 | 207801852 | 207801939 | − | ✓ |
| hsa-mir-29b-2 | MI0000107 | 8561 | chr1 | 207802443 | 207802523 | − | ✓ |
| hsa-mir-29b-1 | MI0000105 | 8558 | chr7 | 130877459 | 130877539 | − | ✓ |
| hsa-mir-29a | MI0000087 | 11796 | chr7 | 130876747 | 130876810 | − | ✓ |
| hsa-mir-299 | MI0000744 | 14 | chr14 | 101023794 | 101023856 | + | ✓ |
| hsa-mir-298 | MI0005523 | — | chr20 | 58818226 | 58818313 | − | — |
| hsa-mir-297 | MI0005775 | 3 | chr4 | 110860582 | 110860647 | − | — |
| hsa-mir-296 | MI0000747 | 12 | chr20 | 58817615 | 58817694 | − | ✓ |
| hsa-mir-2909 | MI0013083 | — | chr17 | 37033745 | 37033813 | + | — |
| hsa-mir-2861 | MI0013006 | 9 | chr9 | 127785918 | 127786007 | + | — |
| hsa-mir-28 | MI0000086 | 3607 | chr3 | 188688781 | 188688866 | + | ✓ |
| hsa-mir-27b | MI0000440 | 14864 | chr9 | 95085445 | 95085541 | + | ✓ |
| hsa-mir-27a | MI0000085 | 8588 | chr19 | 13836440 | 13836517 | − | ✓ |
| hsa-mir-26b | MI0000084 | 17403 | chr2 | 218402646 | 218402722 | + | ✓ |
| hsa-mir-26a-2 | MI0000750 | 36460 | chr12 | 57824609 | 57824692 | − | ✓ |
| hsa-mir-26a-1 | MI0000083 | 26870 | chr3 | 37969404 | 37969480 | + | ✓ |
| hsa-mir-2682 | MI0012063 | — | chr1 | 98045242 | 98045351 | − | — |
| hsa-mir-2681 | MI0012062 | — | chr13 | 101967642 | 101967746 | − | — |
| hsa-mir-25 | MI0000082 | 3122 | chr7 | 100093560 | 100093643 | − | ✓ |
| hsa-mir-2467 | MI0017432 | — | chr2 | 239351724 | 239351804 | − | ✓ |
| hsa-mir-24-2 | MI0000081 | 3749 | chr19 | 13836287 | 13836359 | − | ✓ |
| hsa-mir-24-1 | MI0000080 | 3719 | chr9 | 95086021 | 95086088 | + | ✓ |
| hsa-mir-23c | MI0016010 | 5962 | chrX | 20017088 | 20017187 | − | — |
| hsa-mir-23b | MI0000439 | 11091 | chr9 | 95085208 | 95085304 | + | — |
| hsa-mir-23a | MI0000079 | 10137 | chr19 | 13836587 | 13836659 | − | ✓ |
| hsa-mir-2392 | MI0016870 | — | chr14 | 100814491 | 100814574 | + | — |
| hsa-mir-2355 | MI0015873 | 10 | chr2 | 207109987 | 207110073 | − | — |
| hsa-mir-2278 | MI0011285 | 3 | chr9 | 94809962 | 94810057 | − | — |
| hsa-mir-2277 | MI0011284 | — | chr5 | 93620696 | 93620788 | − | ✓ |
| hsa-mir-2276 | MI0011282 | — | chr13 | 24162416 | 24162504 | + | ✓ |
| hsa-mir-224 | MI0000301 | 237 | chrX | 151958578 | 151958658 | − | ✓ |
| hsa-mir-223 | MI0000300 | 1044 | chrX | 66018870 | 66018979 | + | ✓ |
| hsa-mir-222 | MI0000299 | 3517 | chrX | 45747015 | 45747124 | − | ✓ |
| hsa-mir-221 | MI0000298 | 4317 | chrX | 45746157 | 45746266 | − | ✓ |
| hsa-mir-22 | MI0000078 | 3830 | chr17 | 1713903 | 1713987 | − | ✓ |
| hsa-mir-219b | MI0017299 | — | chr9 | 128392621 | 128392708 | + | ✓ |
| hsa-mir-219a-2 | MI0000740 | 141 | chr9 | 128392618 | 128392714 | − | ✓ |
| hsa-mir-219a-1 | MI0000296 | — | chr6 | 33207835 | 33207944 | + | ✓ |
| hsa-mir-218-2 | MI0000295 | 1077 | chr5 | 168768146 | 168768255 | − | ✓ |
| hsa-mir-218-1 | MI0000294 | 1151 | chr4 | 20528275 | 20528384 | + | ✓ |
| hsa-mir-217 | MI0000293 | 120 | chr2 | 55982967 | 55983076 | − | — |
| hsa-mir-216b | MI0005569 | 5 | chr2 | 56000714 | 56000795 | − | — |
| hsa-mir-216a | MI0000292 | 413 | chr2 | 55988950 | 55989059 | − | ✓ |
| hsa-mir-215 | MI0000291 | 686 | chr1 | 220117853 | 220117962 | − | — |
| hsa-mir-214 | MI0000290 | 459 | chr1 | 172138798 | 172138907 | − | ✓ |
| hsa-mir-212 | MI0000288 | 28 | chr17 | 2050271 | 2050380 | − | ✓ |
| hsa-mir-2117 | MI0010636 | — | chr17 | 43444806 | 43444885 | + | — |
| hsa-mir-2116 | MI0010635 | — | chr15 | 59171183 | 59171262 | − | ✓ |
| hsa-mir-2115 | MI0010634 | 1 | chr3 | 48316360 | 48316459 | − | ✓ |
| hsa-mir-2114 | MI0010633 | — | chrX | 150228004 | 150228083 | + | ✓ |
| hsa-mir-2113 | MI0003939 | 8 | chr6 | 98024531 | 98024621 | + | — |
| hsa-mir-2110 | MI0010629 | 11 | chr10 | 114174105 | 114174179 | − | ✓ |
| hsa-mir-211 | MI0000287 | 500 | chr15 | 31065032 | 31065141 | − | ✓ |
| hsa-mir-210 | MI0000286 | 160 | chr11 | 568089 | 568198 | − | ✓ |
| hsa-mir-21 | MI0000077 | 99440 | chr17 | 59841266 | 59841337 | + | ✓ |
| hsa-mir-20b | MI0001519 | 544 | chrX | 134169809 | 134169877 | − | ✓ |
| hsa-mir-20a | MI0000076 | 7477 | chr13 | 91351065 | 91351135 | + | ✓ |
| hsa-mir-208b | MI0005570 | 49 | chr14 | 23417987 | 23418063 | − | ✓ |
| hsa-mir-208a | MI0000251 | 90 | chr14 | 23388596 | 23388666 | − | ✓ |
| hsa-mir-206 | MI0000490 | 375 | chr6 | 52144349 | 52144434 | + | — |
| hsa-mir-2054 | MI0010488 | — | chr4 | 125507259 | 125507307 | + | — |
| hsa-mir-2053 | MI0010487 | — | chr8 | 112643493 | 112643583 | + | — |
| hsa-mir-2052 | MI0010486 | — | chr8 | 74705693 | 74705747 | + | — |
| hsa-mir-205 | MI0000285 | 6213 | chr1 | 209432133 | 209432242 | + | ✓ |
| hsa-mir-204 | MI0000284 | 400 | chr9 | 70809975 | 70810084 | − | ✓ |
| hsa-mir-203b | MI0017343 | — | chr14 | 104117418 | 104117503 | − | — |
| hsa-mir-203a | MI0000283 | 524 | chr14 | 104117405 | 104117514 | + | ✓ |
| hsa-mir-202 | MI0003130 | 190 | chr10 | 133247511 | 133247620 | − | ✓ |
| hsa-mir-200c | MI0000650 | 4878 | chr12 | 6963699 | 6963766 | + | ✓ |
| hsa-mir-200b | MI0000342 | 3832 | chr1 | 1167104 | 1167198 | + | ✓ |
| hsa-mir-200a | MI0000737 | 2312 | chr1 | 1167863 | 1167952 | + | ✓ |
| hsa-mir-19b-2 | MI0000075 | 11771 | chrX | 134169671 | 134169766 | − | ✓ |
| hsa-mir-19b-1 | MI0000074 | 11311 | chr13 | 91351192 | 91351278 | + | ✓ |
| hsa-mir-19a | MI0000073 | 10618 | chr13 | 91350891 | 91350972 | + | ✓ |
| hsa-mir-199b | MI0000282 | 9394 | chr9 | 128244721 | 128244830 | − | ✓ |
| hsa-mir-199a-2 | MI0000281 | 10237 | chr1 | 172144535 | 172144644 | − | ✓ |
| hsa-mir-199a-1 | MI0000242 | 10013 | chr19 | 10817426 | 10817496 | − | ✓ |
| hsa-mir-198 | MI0000240 | 7 | chr3 | 120395668 | 120395729 | − | — |
| hsa-mir-1976 | MI0009986 | 1 | chr1 | 26554542 | 26554593 | + | — |
| hsa-mir-1973 | MI0009983 | 5 | — | | | | |
| hsa-mir-1972-2 | MI0015977 | 94 | chr16 | 70030346 | 70030422 | + | — |
| hsa-mir-1972-1 | MI0009982 | 94 | chr16 | 15010321 | 15010397 | − | — |
| hsa-mir-197 | MI0000239 | 195 | chr1 | 109598893 | 109598967 | + | ✓ |
| hsa-mir-196b | MI0001150 | 1662 | chr7 | 27169480 | 27169563 | − | ✓ |
| hsa-mir-196a-2 | MI0000279 | 1231 | chr12 | 53991738 | 53991847 | + | ✓ |
| hsa-mir-196a-1 | MI0000238 | 1430 | chr17 | 48632490 | 48632559 | − | ✓ |
| hsa-mir-195 | MI0000489 | 2034 | chr17 | 7017615 | 7017701 | − | ✓ |
| hsa-mir-194-2 | MI0000732 | 155 | chr11 | 64891355 | 64891439 | − | ✓ |
| hsa-mir-194-1 | MI0000488 | 167 | chr1 | 220118157 | 220118241 | − | — |
| hsa-mir-193b | MI0003137 | 657 | chr16 | 14303967 | 14304049 | + | ✓ |
| hsa-mir-193a | MI0000487 | 540 | chr17 | 31559996 | 31560083 | + | ✓ |
| hsa-mir-192 | MI0000234 | 781 | chr11 | 64891137 | 64891246 | − | ✓ |
| hsa-mir-1915 | MI0008336 | 3 | chr10 | 21496562 | 21496641 | − | — |
| hsa-mir-1914 | MI0008335 | — | chr20 | 63941465 | 63941544 | − | — |
| hsa-mir-1913 | MI0008334 | 2 | chr6 | 166509354 | 166509433 | − | — |
| hsa-mir-1912 | MI0008333 | 8 | chrX | 114651544 | 114651623 | + | ✓ |
| hsa-mir-1911 | MI0008332 | — | chrX | 114763184 | 114763263 | + | — |
| hsa-mir-1910 | MI0008331 | 5 | chr16 | 85741621 | 85741700 | − | — |
| hsa-mir-191 | MI0000465 | 8868 | chr3 | 49020618 | 49020709 | − | ✓ |
| hsa-mir-190b | MI0005545 | 8 | chr1 | 154193665 | 154193743 | − | — |
| hsa-mir-190a | MI0000486 | 15 | chr15 | 62823957 | 62824041 | + | ✓ |
| hsa-mir-1909 | MI0008330 | 2 | chr19 | 1816159 | 1816238 | − | — |
| hsa-mir-1908 | MI0008329 | 20 | chr11 | 61815161 | 61815240 | − | — |
| hsa-mir-18b | MI0001518 | 2965 | chrX | 134170041 | 134170111 | − | ✓ |
| hsa-mir-18a | MI0000072 | 3057 | chr13 | 91350751 | 91350821 | + | ✓ |
| hsa-mir-188 | MI0000484 | 28 | chrX | 50003503 | 50003588 | + | ✓ |
| hsa-mir-187 | MI0000274 | 111 | chr18 | 35904818 | 35904926 | − | ✓ |
| hsa-mir-186 | MI0000483 | 4207 | chr1 | 71067631 | 71067716 | − | ✓ |
| hsa-mir-185 | MI0000482 | 1871 | chr22 | 20033139 | 20033220 | + | ✓ |
| hsa-mir-1843 | MI0032314 | 7 | chr1 | 175968370 | 175968479 | − | — |
| hsa-mir-184 | MI0000481 | 127 | chr15 | 79209788 | 79209871 | + | ✓ |
| hsa-mir-183 | MI0000273 | 490 | chr7 | 129774905 | 129775014 | − | ✓ |
| hsa-mir-1827 | MI0008195 | 29 | chr12 | 100189884 | 100189949 | + | — |
| hsa-mir-1825 | MI0008193 | — | chr20 | 32237795 | 32237847 | + | — |
| hsa-mir-182 | MI0000272 | 1322 | chr7 | 129770383 | 129770492 | − | ✓ |
| hsa-mir-181d | MI0003139 | 896 | chr19 | 13874875 | 13875011 | + | ✓ |
| hsa-mir-181c | MI0000271 | 180 | chr19 | 13874699 | 13874808 | + | ✓ |
| hsa-mir-181b-2 | MI0000683 | 894 | chr9 | 124693710 | 124693798 | + | ✓ |
| hsa-mir-181b-1 | MI0000270 | 893 | chr1 | 198858873 | 198858982 | − | ✓ |
| hsa-mir-181a-2 | MI0000269 | 3589 | chr9 | 124692442 | 124692551 | + | ✓ |
| hsa-mir-181a-1 | MI0000289 | 3606 | chr1 | 198859044 | 198859153 | − | ✓ |
| hsa-mir-17 | MI0000071 | 9521 | chr13 | 91350605 | 91350688 | + | ✓ |
| hsa-mir-16-2 | MI0000115 | 10438 | chr3 | 160404745 | 160404825 | + | ✓ |
| hsa-mir-16-1 | MI0000070 | 10589 | chr13 | 50048973 | 50049061 | − | ✓ |
| hsa-mir-15b | MI0000438 | 6198 | chr3 | 160404588 | 160404685 | + | ✓ |
| hsa-mir-15a | MI0000069 | 4045 | chr13 | 50049119 | 50049201 | − | ✓ |
| hsa-mir-1587 | MI0016905 | — | chrX | 39837561 | 39837613 | + | — |
| hsa-mir-155 | MI0000681 | 355 | chr21 | 25573980 | 25574044 | + | ✓ |
| hsa-mir-154 | MI0000480 | 2 | chr14 | 101059755 | 101059838 | + | ✓ |
| hsa-mir-1539 | MI0007260 | — | chr18 | 49487373 | 49487422 | + | — |
| hsa-mir-1538 | MI0007259 | — | chr16 | 69565808 | 69565868 | − | — |
| hsa-mir-1537 | MI0007258 | 2 | chr1 | 235853000 | 235853060 | − | — |
| hsa-mir-153-2 | MI0000464 | 75 | chr7 | 157574336 | 157574422 | − | ✓ |
| hsa-mir-153-1 | MI0000463 | 69 | chr2 | 219294111 | 219294200 | − | ✓ |
| hsa-mir-152 | MI0000462 | 2307 | chr17 | 48037161 | 48037247 | − | ✓ |
| hsa-mir-151b | MI0003772 | 2711 | chr14 | 100109419 | 100109514 | − | — |
| hsa-mir-151a | MI0000809 | 4889 | chr8 | 140732564 | 140732653 | − | — |
| hsa-mir-150 | MI0000479 | 699 | chr19 | 49500785 | 49500868 | − | ✓ |
| hsa-mir-149 | MI0000478 | 130 | chr2 | 240456001 | 240456089 | + | ✓ |
| hsa-mir-148b | MI0000811 | 2746 | chr12 | 54337216 | 54337314 | + | ✓ |
| hsa-mir-148a | MI0000253 | 15136 | chr7 | 25949919 | 25949986 | − | ✓ |
| hsa-mir-147b | MI0005544 | 32 | chr15 | 45433050 | 45433129 | + | ✓ |
| hsa-mir-147a | MI0000262 | 44 | chr9 | 120244979 | 120245050 | − | — |
| hsa-mir-1471 | MI0007076 | — | chr2 | 231892242 | 231892298 | − | — |
| hsa-mir-1470 | MI0007075 | — | chr19 | 15449548 | 15449608 | + | — |
| hsa-mir-146b | MI0003129 | 2237 | chr10 | 102436512 | 102436584 | + | ✓ |
| hsa-mir-146a | MI0000477 | 2369 | chr5 | 160485352 | 160485450 | + | ✓ |
| hsa-mir-1469 | MI0007074 | 10 | chr15 | 96333261 | 96333307 | + | — |
| hsa-mir-1468 | MI0003782 | — | chrX | 63786002 | 63786087 | − | — |
| hsa-mir-145 | MI0000461 | 29896 | chr5 | 149430646 | 149430733 | + | ✓ |
| hsa-mir-144 | MI0000460 | 640 | chr17 | 28861533 | 28861618 | − | ✓ |
| hsa-mir-143 | MI0000459 | 104879 | chr5 | 149428918 | 149429023 | + | ✓ |
| hsa-mir-142 | MI0000458 | 3620 | chr17 | 58331232 | 58331318 | − | ✓ |
| hsa-mir-141 | MI0000457 | 1700 | chr12 | 6964097 | 6964191 | + | ✓ |
| hsa-mir-140 | MI0000456 | 5403 | chr16 | 69933081 | 69933180 | + | ✓ |
| hsa-mir-139 | MI0000261 | 195 | chr11 | 72615063 | 72615130 | − | ✓ |
| hsa-mir-138-2 | MI0000455 | 159 | chr16 | 56858518 | 56858601 | + | ✓ |
| hsa-mir-138-1 | MI0000476 | 180 | chr3 | 44114212 | 44114310 | + | ✓ |
| hsa-mir-137 | MI0000454 | 246 | chr1 | 98046070 | 98046171 | − | ✓ |
| hsa-mir-136 | MI0000475 | 77 | chr14 | 100884702 | 100884783 | + | ✓ |
| hsa-mir-135b | MI0000810 | 189 | chr1 | 205448302 | 205448398 | − | ✓ |
| hsa-mir-135a-2 | MI0000453 | 444 | chr12 | 97563812 | 97563911 | + | ✓ |
| hsa-mir-135a-1 | MI0000452 | 590 | chr3 | 52294219 | 52294308 | − | ✓ |
| hsa-mir-1343 | MI0017320 | — | chr11 | 34941837 | 34941920 | + | ✓ |
| hsa-mir-134 | MI0000474 | 118 | chr14 | 101054687 | 101054759 | + | ✓ |
| hsa-mir-133b | MI0000822 | 224 | chr6 | 52148923 | 52149041 | + | ✓ |
| hsa-mir-133a-2 | MI0000451 | 543 | chr20 | 62564912 | 62565013 | + | ✓ |
| hsa-mir-133a-1 | MI0000450 | 543 | chr18 | 21825698 | 21825785 | − | ✓ |
| hsa-mir-1324 | MI0006657 | — | chr3 | 75630763 | 75630858 | + | — |
| hsa-mir-1323 | MI0003786 | 7 | chr19 | 53671968 | 53672040 | + | — |
| hsa-mir-1322 | MI0006653 | — | chr8 | 10825373 | 10825443 | − | — |
| hsa-mir-1321 | MI0006652 | — | chrX | 85835780 | 85835858 | + | — |
| hsa-mir-132 | MI0000449 | 751 | chr17 | 2049908 | 2050008 | − | ✓ |
| hsa-mir-130b | MI0000748 | 869 | chr22 | 21653304 | 21653385 | + | ✓ |
| hsa-mir-130a | MI0000448 | 1813 | chr11 | 57641198 | 57641286 | + | ✓ |
| hsa-mir-1307 | MI0006444 | 249 | chr10 | 103394253 | 103394401 | − | ✓ |
| hsa-mir-1306 | MI0006443 | 6 | chr22 | 20086058 | 20086142 | + | ✓ |
| hsa-mir-1305 | MI0006372 | — | chr4 | 182169293 | 182169378 | + | — |
| hsa-mir-1304 | MI0006371 | 51 | chr11 | 93733674 | 93733764 | − | ✓ |
| hsa-mir-1303 | MI0006370 | 48 | chr5 | 154685776 | 154685861 | + | — |
| hsa-mir-1302-9 | MI0015978 | 2 | chr9 | 30144 | 30281 | + | — |
| hsa-mir-1302-8 | MI0006369 | — | chr9 | 97363554 | 97363681 | − | — |
| hsa-mir-1302-7 | MI0006368 | 29 | chr8 | 141786242 | 141786313 | − | — |
| hsa-mir-1302-6 | MI0006367 | — | chr7 | 18127220 | 18127309 | − | — |
| hsa-mir-1302-5 | MI0006366 | — | chr20 | 50614636 | 50614785 | − | — |
| hsa-mir-1302-4 | MI0006365 | 1 | chr2 | 207269275 | 207269424 | − | — |
| hsa-mir-1302-3 | MI0006364 | 2 | chr2 | 113582959 | 113583096 | − | — |
| hsa-mir-1302-2 | MI0006363 | 2 | chr1 | 30366 | 30503 | + | — |
| hsa-mir-1302-11 | MI0015980 | 2 | chr19 | 71973 | 72110 | + | — |
| hsa-mir-1302-10 | MI0015979 | 2 | chr15 | 101960459 | 101960596 | − | — |
| hsa-mir-1302-1 | MI0006362 | — | chr12 | 112695034 | 112695176 | − | — |
| hsa-mir-1301 | MI0003815 | 50 | chr2 | 25328640 | 25328721 | − | ✓ |
| hsa-mir-1299 | MI0006359 | 6 | chr9 | 40929010 | 40929092 | − | — |
| hsa-mir-1298 | MI0003938 | 2 | chrX | 114715233 | 114715344 | + | ✓ |
| hsa-mir-1297 | MI0006358 | — | chr13 | 54311972 | 54312048 | − | — |
| hsa-mir-1296 | MI0003780 | 27 | chr10 | 63372957 | 63373048 | − | ✓ |
| hsa-mir-1295b | MI0019146 | — | chr1 | 171101739 | 171101798 | + | — |
| hsa-mir-1295a | MI0006357 | 1 | chr1 | 171101728 | 171101806 | − | — |
| hsa-mir-1294 | MI0006356 | 6 | chr5 | 154347106 | 154347247 | + | ✓ |
| hsa-mir-1293 | MI0006355 | 4 | chr12 | 50234142 | 50234212 | − | — |
| hsa-mir-1292 | MI0006433 | — | chr20 | 2652777 | 2652842 | + | — |
| hsa-mir-1291 | MI0006353 | 8 | chr12 | 48654444 | 48654530 | − | — |
| hsa-mir-1290 | MI0006352 | 798 | chr1 | 18897071 | 18897148 | − | — |
| hsa-mir-129-2 | MI0000473 | 68 | chr11 | 43581394 | 43581483 | + | ✓ |
| hsa-mir-129-1 | MI0000252 | 69 | chr7 | 128207872 | 128207943 | + | ✓ |
| hsa-mir-1289-2 | MI0006351 | 41 | chr5 | 133427596 | 133427706 | − | — |
| hsa-mir-1289-1 | MI0006350 | 146 | chr20 | 35453954 | 35454097 | − | — |
| hsa-mir-1288 | MI0006432 | — | chr17 | 16282014 | 16282088 | + | — |
| hsa-mir-1287 | MI0006349 | 7 | chr10 | 98395218 | 98395307 | − | ✓ |
| hsa-mir-1286 | MI0006348 | — | chr22 | 20249134 | 20249211 | − | — |
| hsa-mir-1285-2 | MI0006347 | 17 | chr2 | 70252918 | 70253005 | − | — |
| hsa-mir-1285-1 | MI0006346 | 392 | chr7 | 92204015 | 92204098 | − | ✓ |
| hsa-mir-1284 | MI0006431 | — | chr3 | 71541970 | 71542089 | − | ✓ |
| hsa-mir-1283-2 | MI0006430 | 102 | chr19 | 53758232 | 53758318 | + | — |
| hsa-mir-1283-1 | MI0003832 | 114 | chr19 | 53688481 | 53688567 | + | — |
| hsa-mir-1282 | MI0006429 | 115 | chr15 | 43793659 | 43793759 | − | — |
| hsa-mir-1281 | MI0006428 | — | chr22 | 41092513 | 41092566 | + | — |
| hsa-mir-128-2 | MI0000727 | 690 | chr3 | 35744476 | 35744559 | + | ✓ |
| hsa-mir-128-1 | MI0000447 | 693 | chr2 | 135665397 | 135665478 | + | ✓ |
| hsa-mir-1279 | MI0006426 | — | chr12 | 69273157 | 69273218 | − | — |
| hsa-mir-1278 | MI0006425 | — | chr1 | 193136503 | 193136583 | + | ✓ |
| hsa-mir-1277 | MI0006419 | 41 | chrX | 118386394 | 118386471 | + | ✓ |
| hsa-mir-1276 | MI0006416 | — | chr15 | 85770496 | 85770578 | − | — |
| hsa-mir-1275 | MI0006415 | 241 | chr6 | 33999972 | 34000051 | − | — |
| hsa-mir-1273h | MI0025512 | 23 | chr16 | 24203116 | 24203231 | + | — |
| hsa-mir-1273c | MI0014171 | 12 | chr6 | 154853360 | 154853436 | + | ✓ |
| hsa-mir-1272 | MI0006408 | 67 | chr15 | 64762387 | 64762515 | − | — |
| hsa-mir-1271 | MI0003814 | 36 | chr5 | 176367946 | 176368031 | + | ✓ |
| hsa-mir-1270 | MI0006407 | — | chr19 | 20399272 | 20399354 | − | — |
| hsa-mir-127 | MI0000472 | 395 | chr14 | 100882979 | 100883075 | + | ✓ |
| hsa-mir-1269b | MI0016888 | 77 | chr17 | 12917268 | 12917342 | − | — |
| hsa-mir-1269a | MI0006406 | 82 | chr4 | 66276824 | 66276928 | + | ✓ |
| hsa-mir-1268b | MI0016748 | 53 | chr17 | 80098828 | 80098877 | + | — |
| hsa-mir-1268a | MI0006405 | 95 | chr15 | 22225278 | 22225329 | − | — |
| hsa-mir-1267 | MI0006404 | 3 | chr13 | 107531171 | 107531248 | − | — |
| hsa-mir-1266 | MI0006403 | — | chr15 | 52277117 | 52277200 | − | — |
| hsa-mir-1265 | MI0006401 | — | chr10 | 14436576 | 14436661 | + | — |
| hsa-mir-1264 | MI0003758 | 1 | chr> | 114652655 | 114652723 | + | — |
| hsa-mir-1263 | MI0006398 | — | chr3 | 164171471 | 164171556 | − | — |
| hsa-mir-1262 | MI0006397 | — | chr1 | 68183518 | 68183610 | − | — |
| hsa-mir-1261 | MI0006396 | 64 | chr11 | 90869121 | 90869202 | − | — |
| hsa-mir-1260b | MI0014197 | 913 | chr11 | 96341438 | 96341526 | + | — |
| hsa-mir-1260a | MI0006394 | 898 | chr14 | 77266218 | 77266290 | + | — |
| hsa-mir-126 | MI0000471 | 4481 | chr9 | 136670602 | 136670686 | + | ✓ |
| hsa-mir-125b-2 | MI0000470 | 11409 | chr21 | 16590237 | 16590325 | + | ✓ |
| hsa-mir-125b-1 | MI0000446 | 13830 | chr11 | 122099757 | 122099844 | − | ✓ |
| hsa-mir-125a | MI0000469 | 3993 | chr19 | 51693254 | 51693339 | + | ✓ |
| hsa-mir-1258 | MI0006392 | — | chr2 | 179860836 | 179860908 | − | — |
| hsa-mir-1257 | MI0006391 | 8 | chr20 | 61953546 | 61953662 | − | — |
| hsa-mir-1256 | MI0006390 | — | chr1 | 20988314 | 20988432 | − | ✓ |
| hsa-mir-1255b-2 | MI0006436 | 4 | chr1 | 167998660 | 167998726 | + | — |
| hsa-mir-1255b-1 | MI0006435 | 4 | chr4 | 36426366 | 36426428 | − | — |
| hsa-mir-1255a | MI0006389 | 6 | chr4 | 101330302 | 101330414 | − | ✓ |
| hsa-mir-1253 | MI0006387 | — | chr17 | 2748078 | 2748182 | − | — |
| hsa-mir-1252 | MI0006434 | — | chr12 | 79419257 | 79419321 | + | — |
| hsa-mir-1251 | MI0006386 | — | chr12 | 97491909 | 97491978 | + | — |
| hsa-mir-1250 | MI0006385 | 41 | chr17 | 81133196 | 81133308 | − | — |
| hsa-mir-1249 | MI0006384 | 9 | chr22 | 45200954 | 45201019 | − | ✓ |
| hsa-mir-1248 | MI0006383 | 7 | chr3 | 186786672 | 186786777 | + | — |
| hsa-mir-1247 | MI0006382 | 25 | chr14 | 101560287 | 101560422 | − | ✓ |
| hsa-mir-1246 | MI0006381 | 7472 | chr2 | 176600980 | 176601052 | − | — |
| hsa-mir-1245b | MI0017431 | — | chr2 | 188978093 | 188978161 | − | — |
| hsa-mir-1245a | MI0006380 | — | chr2 | 188978092 | 188978161 | + | — |
| hsa-mir-1244-4 | MI0031511 | — | chr12 | 12111952 | 12112036 | + | — |
| hsa-mir-1244-3 | MI0015975 | — | chr12 | 9239467 | 9239551 | − | — |
| hsa-mir-1244-2 | MI0015974 | — | chr5 | 118974586 | 118974670 | + | — |
| hsa-mir-1244-1 | MI0006379 | — | chr2 | 231713314 | 231713398 | + | — |
| hsa-mir-1243 | MI0006373 | — | chr4 | 113106863 | 113106955 | + | — |
| hsa-mir-124-3 | MI0000445 | 256 | chr20 | 63178500 | 63178586 | + | ✓ |
| hsa-mir-124-2 | MI0000444 | 247 | chr8 | 64379149 | 64379257 | + | ✓ |
| hsa-mir-124-1 | MI0000443 | 254 | chr8 | 9903388 | 9903472 | − | ✓ |
| hsa-mir-1238 | MI0006328 | — | chr19 | 10552122 | 10552204 | + | — |
| hsa-mir-1237 | MI0006327 | — | chr11 | 64368602 | 64368703 | + | — |
| hsa-mir-1236 | MI0006326 | — | chr6 | 31956839 | 31956940 | − | — |
| hsa-mir-1234 | MI0006324 | 1236 | chr8 | 144400086 | 144400165 | − | — |
| hsa-mir-1233-2 | MI0015973 | 2 | chr15 | 34528290 | 34528371 | − | — |
| hsa-mir-1233-1 | MI0006323 | 2 | chr15 | 34382069 | 34382150 | − | — |
| hsa-mir-1231 | MI0006321 | — | chr1 | 201808611 | 201808702 | + | — |
| hsa-mir-122b | MI0017383 | 136 | chr18 | 58451080 | 58451152 | − | ✓ |
| hsa-mir-1229 | MI0006319 | 3 | chr5 | 179798278 | 179798346 | − | — |
| hsa-mir-1228 | MI0006318 | 4 | chr12 | 57194504 | 57194576 | + | — |
| hsa-mir-1227 | MI0006316 | — | chr19 | 2234062 | 2234149 | − | — |
| hsa-mir-1226 | MI0006313 | 5 | chr3 | 47849555 | 47849629 | + | — |
| hsa-mir-1225 | MI0006311 | — | chr16 | 2090195 | 2090284 | − | — |
| hsa-mir-1224 | MI0003764 | 18 | chr3 | 184241405 | 184241489 | + | — |
| hsa-mir-122 | MI0000442 | 1201 | chr18 | 58451074 | 58451158 | + | ✓ |
| hsa-mir-12136 | MI0039740 | 457 | chr1 | 632615 | 632685 | − | — |
| hsa-mir-12135 | MI0039739 | 225 | chr15 | 73299910 | 73299979 | + | — |
| hsa-mir-12133 | MI0039735 | — | chr1 | 94543702 | 94543779 | + | — |
| hsa-mir-12132 | MI0039734 | — | chr1 | 61846075 | 61846176 | + | — |
| hsa-mir-12131 | MI0039733 | — | chr6 | 151102675 | 151102776 | + | — |
| hsa-mir-12130 | MI0039732 | — | chr5 | 116255743 | 116255834 | + | — |
| hsa-mir-12129 | MI0039731 | — | chrX | 153335462 | 153335537 | + | — |
| hsa-mir-12128 | MI0039730 | 55 | chr16 | 85207796 | 85207895 | + | — |
| hsa-mir-12127 | MI0039729 | — | chr3 | 10280351 | 10280452 | + | — |
| hsa-mir-12126 | MI0039728 | 18 | chr9 | 130241387 | 130241488 | + | — |
| hsa-mir-12125 | MI0039727 | — | chr5 | 168165845 | 168165929 | + | — |
| hsa-mir-12124 | MI0039726 | 6 | chr3 | 128846808 | 128846908 | + | — |
| hsa-mir-12123 | MI0039725 | — | chr8 | 79332188 | 79332274 | + | — |
| hsa-mir-12122 | MI0039724 | — | chr20 | 49863859 | 49863953 | + | — |
| hsa-mir-12121 | MI0039723 | 35 | chr14 | 104865314 | 104865411 | + | — |
| hsa-mir-12120 | MI0039722 | — | chrY | 13479177 | 13479266 | + | — |
| hsa-mir-12119 | MI0039721 | — | chr7 | 99409172 | 99409264 | + | — |
| hsa-mir-12118 | MI0039720 | — | chr5 | 173303730 | 173303817 | + | — |
| hsa-mir-12117 | MI0039719 | 12 | chr9 | 33108692 | 33108765 | + | — |
| hsa-mir-12116 | MI0039718 | 59 | chr1 | 179803924 | 179804025 | + | — |
| hsa-mir-12115 | MI0039717 | 100 | chr4 | 22515312 | 22515385 | + | — |
| hsa-mir-12114 | MI0039716 | 13 | chr22 | 50407442 | 50407524 | + | — |
| hsa-mir-12113 | MI0039715 | — | chr4 | 11783178 | 11783248 | + | — |
| hsa-mir-1208 | MI0006341 | — | chr8 | 128150116 | 128150188 | + | — |
| hsa-mir-1207 | MI0006340 | — | chr8 | 128049152 | 128049238 | + | — |
| hsa-mir-1206 | MI0006339 | — | chr8 | 128008898 | 128008956 | + | — |
| hsa-mir-1205 | MI0006338 | — | chr8 | 127960633 | 127960695 | + | — |
| hsa-mir-1204 | MI0006337 | — | chr8 | 127795962 | 127796028 | + | — |
| hsa-mir-1203 | MI0006335 | — | chr17 | 48156427 | 48156511 | − | — |
| hsa-mir-1202 | MI0006334 | 28 | chr6 | 155946797 | 155946879 | + | — |
| hsa-mir-1200 | MI0006332 | — | chr7 | 36919357 | 36919432 | − | — |
| hsa-mir-1199 | MI0020340 | — | chr19 | 14073361 | 14073479 | + | — |
| hsa-mir-1197 | MI0006656 | — | chr14 | 101025564 | 101025651 | + | — |
| hsa-mir-1193 | MI0014205 | 52 | chr14 | 101030052 | 101030129 | + | — |
| hsa-mir-1185-2 | MI0003821 | 2 | chr14 | 101044198 | 101044283 | + | ✓ |
| hsa-mir-1185-1 | MI0003844 | 2 | chr14 | 101042977 | 101043062 | + | ✓ |
| hsa-mir-1184-3 | MI0015972 | 180 | chrX | 155457517 | 155457615 | + | — |
| hsa-mir-1184-2 | MI0015971 | 180 | chrX | 155383100 | 155383198 | − | — |
| hsa-mir-1184-1 | MI0006277 | 180 | chrX | 154887360 | 154887458 | − | — |
| hsa-mir-1183 | MI0006276 | — | chr7 | 21471058 | 21471146 | + | — |
| hsa-mir-1182 | MI0006275 | — | chr1 | 231019828 | 231019924 | − | — |
| hsa-mir-1181 | MI0006274 | 3 | chr19 | 10403458 | 10403538 | − | — |
| hsa-mir-1180 | MI0006273 | 103 | chr17 | 19344506 | 19344574 | − | ✓ |
| hsa-mir-1179 | MI0006272 | — | chr15 | 88608107 | 88608197 | + | — |
| hsa-mir-1178 | MI0006271 | — | chr12 | 119713634 | 119713724 | − | — |
| hsa-mir-11401 | MI0036560 | 8 | chr16 | 87853896 | 87853964 | + | — |
| hsa-mir-11400 | MI0036559 | 12 | chr7 | 142474527 | 142474603 | + | — |
| hsa-mir-11399 | MI0036558 | — | chr1 | 112882050 | 112882129 | + | — |
| hsa-mir-11181 | MI0035972 | — | chr15 | 87946206 | 87946268 | − | — |
| hsa-mir-10b | MI0000267 | 7790 | chr2 | 176150303 | 176150412 | + | — |
| hsa-mir-10a | MI0000266 | 5181 | chr17 | 48579838 | 48579947 | − | ✓ |
| hsa-mir-107 | MI0000114 | 11858 | chr10 | 89592747 | 89592827 | − | ✓ |
| hsa-mir-106b | MI0000734 | 1841 | chr7 | 100093993 | 100094074 | − | ✓ |
| hsa-mir-106a | MI0000113 | 7942 | chrX | 134170198 | 134170278 | − | ✓ |
| hsa-mir-10527 | MI0033674 | 2 | chr12 | 63823663 | 63823727 | + | — |
| hsa-mir-10526 | MI0033673 | — | chr11 | 122152103 | 122152156 | − | — |
| hsa-mir-10525 | MI0033672 | — | chr7 | 74595968 | 74596026 | + | — |
| hsa-mir-10524 | MI0033671 | 12 | chr6 | 78539263 | 78539309 | − | — |
| hsa-mir-10523 | MI0033669 | 36 | chr5 | 172915083 | 172915158 | + | — |
| hsa-mir-10522 | MI0033668 | — | chr5 | 17155930 | 17155992 | − | — |
| hsa-mir-105-2 | MI0000112 | 15 | chrX | 152394412 | 152394492 | − | ✓ |
| hsa-mir-105-1 | MI0000111 | 15 | chrX | 152392219 | 152392299 | − | ✓ |
| hsa-mir-10401 | MI0033425 | 156 | chr21 | 8206563 | 8206618 | + | — |
| hsa-mir-10400 | MI0033424 | 16 | chr8 | 144462485 | 144462539 | − | — |
| hsa-mir-103b-2 | MI0007262 | — | chr20 | 3917502 | 3917563 | − | — |
| hsa-mir-103b-1 | MI0007261 | 233 | chr5 | 168560904 | 168560965 | + | — |
| hsa-mir-103a-2 | MI0000108 | 12432 | chr20 | 3917494 | 3917571 | + | ✓ |
| hsa-mir-103a-1 | MI0000109 | 12432 | chr5 | 168560896 | 168560973 | − | ✓ |
| hsa-mir-10399 | MI0033423 | 19 | chr7 | 139044100 | 139044157 | − | — |
| hsa-mir-10398 | MI0033422 | 8 | chr6 | 41733554 | 41733610 | − | — |
| hsa-mir-10397 | MI0033421 | — | chr5 | 10402364 | 10402431 | + | — |
| hsa-mir-10396b | MI0033426 | — | chr21 | 8436324 | 8436374 | + | ✓ |
| hsa-mir-10396a | MI0033420 | — | chr21 | 8987617 | 8987665 | + | — |
| hsa-mir-10395 | MI0033419 | — | chr19 | 12703601 | 12703664 | − | — |
| hsa-mir-10394 | MI0033418 | — | chr19 | 58393364 | 58393446 | + | — |
| hsa-mir-10393 | MI0033417 | — | chr15 | 44717844 | 44717893 | + | — |
| hsa-mir-10392 | MI0033416 | 8 | chr11 | 64878523 | 64878609 | − | — |
| hsa-mir-10226 | MI0033056 | 169 | chr17 | 48032882 | 48032951 | + | — |
| hsa-mir-101-2 | MI0000739 | 6084 | chr9 | 4850297 | 4850375 | + | ✓ |
| hsa-mir-101-1 | MI0000103 | 6056 | chr1 | 65058434 | 65058508 | − | ✓ |
| hsa-mir-100 | MI0000102 | 10013 | chr11 | 122152229 | 122152308 | − | ✓ |
| hsa-mir-1-2 | MI0000437 | 13316 | chr18 | 21829004 | 21829088 | − | — |
| hsa-mir-1-1 | MI0000651 | 6511 | chr20 | 62554306 | 62554376 | + | ✓ |
| hsa-let-7i | MI0000434 | 17040 | chr12 | 62603686 | 62603769 | + | ✓ |
| hsa-let-7g | MI0000433 | 102832 | chr3 | 52268278 | 52268361 | − | ✓ |
| hsa-let-7f-2 | MI0000068 | 118700 | chrX | 53557192 | 53557274 | − | ✓ |
| hsa-let-7f-1 | MI0000067 | 109279 | chr9 | 94176347 | 94176433 | + | ✓ |
| hsa-let-7e | MI0000066 | 50938 | chr19 | 51692786 | 51692864 | + | ✓ |
| hsa-let-7d | MI0000065 | 4649 | chr9 | 94178834 | 94178920 | + | ✓ |
| hsa-let-7c | MI0000064 | 135622 | chr21 | 16539828 | 16539911 | + | ✓ |
| hsa-let-7b | MI0000063 | 83403 | chr22 | 46113686 | 46113768 | + | ✓ |
| hsa-let-7a-3 | MI0000062 | 142757 | chr22 | 46112749 | 46112822 | + | ✓ |
| hsa-let-7a-2 | MI0000061 | 142652 | chr11 | 122146522 | 122146593 | − | ✓ |
| hsa-let-7a-1 | MI0000060 | 145261 | chr9 | 94175957 | 94176036 | + | ✓ |

### DETAILED DESCRIPTION

The invention discloses the combination of a truncated portion of the full-length DUX4 protein, a promoter that increases translation of the truncated DUX4 protein in muscle cells specifically, and a capsid protein that helps to deliver the genome to the appropriate cells.

There are multiple methods for truncating the DUX4-FL protein to make the protein less pathogenic, wherein a single or possibly combinations of mutations, deletions, and fusions of the genome yield possible therapeutic sequences. The invention utilizes a recombinant AAV virus; recombinant viruses can be used to express therapeutic proteins in a subject as a form of genetic therapy. The therapy seeking to deliver that protein may utilize a recombinant viral genome that has the region of interest that will code the desired protein along with untranslated regions at the 5′ and 3′ ends of the genome. It may also include a promoter that will drive the gene of interest and it may include uninteresting that enhances stability and exportation out of the nucleus. The promoter may be used to identify preferential cellular transcription sites, so whether the gene is translated more often in skin, blood, bone, muscle, nervous, or other tissue types.

For example, and non-exclusively, DUX4-S is the first 159 amino acids of DUX4-FL (SEQ ID NO. 3). This version of the truncated portion of the full-length DUX4-FL sequence may result in reduced pathogenesis of muscle disorders because the full DUX4 amino acid sequence is no longer translated, and thus would not yield harmful effects to the myocytes.

Furthermore, the invention may be delivered to a subject by any pharmaceutically acceptable dosage delivery method, which may be preferentially parenteral or another method that accomplishes successful delivery of the genome to a cell that can transcribe and translate a truncated DUX4 sequence.

### Adeno Associated Virus Vector

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV 12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

Previous approaches to identify AAV sequences correlated with tropism have relied upon the comparison of highly related extant serotypes with distinct characteristics, random domain swaps between unrelated serotypes, or consideration of higher-order structure, to identify motifs that define liver tropism. For example, mapping determinants of AAV tropism have been carried out by comparing highly related serotypes. One such example is the single-amino acid change (E531K) between AAV1 and AAV6 that improves murine liver transduction in AAV1. See Wu et al. (2006) J. Virol., 80(22): 11393-7, incorporated by reference herein. Another example is a reciprocal domain swap between AAV2 and AAV8 that alters tropism, but fails to define any robust specific tissue-targeting motifs. See Raupp et al. (201) J. Virol., 86 (17): 9396-408, incorporated by reference herein. Further, global consideration of structure has only highlighted gross differences between better- or worse-liver-transducers that are more observational than useful in practice. Nam et al (2007) J. Virol., 81(22): 12260-71.

AAVs exhibiting modified tissue tropism that may be used with the present invention are described in U.S. Pat. Nos. 9,695,220, 9,719,070; 10,119,125; 10,526,584; U.S. Patent Application Publication No. 2018-0369414; U.S. Patent Application Publication No. 2020-0123504; U.S. Patent Application Publication No. 2020-0318082; PCT International Patent Application Publication No. WO 2015/054653; PCT International Patent Application Publication No. WO 2016/179496; PCT International Patent Application Publication No. WO 2017/100791; and PCT International Patent Application Publication No. WO 2019/217911, the entirety of the contents of each of which are incorporated by reference herein.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the tropism for nervous cells might be reduced or eliminated and/or the muscle specificity increased such that the nervous specificity appears reduced in comparison, thus enhancing the specificity for muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as International Patent Application Publications WO 2021/050974 and WO) 2021/077000 and PCT International Application No. PCT/US2021/042812, the contents of which are incorporated by reference herein.

Additional AAV vectors are described in International Patent Application Publication WO 2019/2071632, the contents of which are incorporated by reference herein.

Further AAV vectors are described in International Patent Application Publications WO 2020/086881 and WO 2020/235543, the contents of each of which are incorporated by reference herein.

Further AAV vectors are described in International Patent Application Publications WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### Promoter

The invention may contain a muscle specific promoter or another promoter. The promoter may be linked to the nucleic acid sequence so that the transcription preferably occurs within myocytes. Promoter regions enable the host cells to replicate the AAV delivered nucleic acid only in those cell types and tissues or organs in which the desired protein should be created. Here, the muscle specific promoter is included because it is principally desired that the proteins only be translated in myocytes. Specificity of the cell type into which the nucleic acid is delivered and thus the proteins translated is desired because of the adverse effects that may ensue from delivering the nucleic acid and having it translated in cells in which that nucleic acid and thus protein is not needed.

The myocyte specific promoter may be coupled or otherwise associated with a truncated DUX4 sequence. In some embodiments, the promoter may be directly attached, and in others there may be a linker molecule or another indirect coupling method to attach to the truncated DUX4 sequence. In some embodiments, there may be an associated polypeptide or other particle that is coupled to the truncated DUX4 sequence.

In some embodiments, the muscle specific promoter yields increased muscle cell potency, muscle cell specificity, reduced immunogenicity, or any combination thereof. As used herein the terms “muscle-specific”, “muscle cell specificity”, “muscle cell potency,” “myocyte specific” and the like, refer to the increased specificity, selectivity, or potency, of the muscle-specific targeting moieties and compositions incorporating said muscle-specific targeting moieties of the present invention for myocytes relative to non-muscle cells. In some embodiments, the cell specificity, or selectivity, or potency, or a combination thereof of a muscle-specific targeting moiety or composition incorporating a muscle-specific targeting moiety described herein is at least 2 to at least 500 times more specific, selective, and/or potent for/in a muscle cell relative to a non-muscle cell.

In some embodiments, the myocyte-selective promoter utilized is MHCK7. MHCK7 is a 770 base pair length promoter that is small enough to be included in an AAV vector. MHCK7 directs expression in fast and slow skeletal and cardiac muscle, with low expression in the liver, lung, and spleen. It is less active in smooth muscle. The MHCK7 promoter is associated with high levels of expression in skeletal muscles, including the diaphragm, and includes an enhancer to especially drive expression in the heart, whereas expression in off-target tissues is minimal.

In some embodiments, the promoters described herein are inserted into an AAV protein (e.g., an AAV capsid protein) that has reduced specificity (or no detectable, measurable, or clinically relevant interaction) for one or more non-muscle cell types. Exemplary non-muscle cell types include, but are not limited to, liver, kidney, lung, heart, spleen, central or peripheral nervous system cells, bone, immune, stomach, intestine, eye, skin cells and the like. In some embodiments, the non-muscle cells are liver cells.

The term “operably linked” refers to the association of two or more nucleic acid molecules on a single nucleic acid fragment so that the function of one is affected by the other.

Further exemplary tissue specific promoters include U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or TH promoter sequence.

Muscle specific promoters are described in International Patent Application Publications WO 2020/006458 and WO 2021/126880, the contents of each of which are incorporated by reference herein.

Further muscle specific promoters are described in U.S. Pat. Nos. 9,133,482; 10,105,453; 10,301,367; U.S. Patent Publication No. 2020-0360534; PCT International Patent Publication Nos. WO 2020/006458; WO 2021/035120; WO 2021/053124; and WO 2021/077000, the contents of each of which are incorporated by reference herein.

It may be convenient to use an RNA polymerase II or III promoter; these are known to the person skilled in the art and reviewed in e.g. Kornberg 1999. However, transcripts from an RNA II polymerase often have complex transcription terminators and transcripts are polyadenylated; this may hamper with the requirements of the miRNA strand which because both its 5′ and 3′ ends need to be precisely defined in order to achieve the required secondary structure to produce a functional molecule. These drawbacks can however be circumvented. In case an RNA polymerase II or III promoter is used, the polynucleotide encoding the miRNA strand may also encode self-processing ribozymes and may be operably linked to an RNA polymerase II or III promoter; as such the polynucleotide encodes a pre-miRNA strand comprising the miRNA strand and self-processing ribozymes, wherein, when transcribed, the miRNA strand is released by the self-processing ribozymes from the pre-miRNA strand de transcript.

Preferably, in a composition according to the present invention the AAV vector is comprised of an RNA polymerase II promoter or III promoter, and encodes a pre-miRNA strand comprising the miRNA strand and self-processing ribozymes, wherein, when transcribed, the miRNA strand is released by the self-processing ribozymes from the pre-miRNA strand transcript. Conveniently, multiple pre-miRNA strands and multiple self-processing ribozymes may be encoded by a single polynucleotide, operably linked to one or more RNA polymerase II promoters.

RNA polymerase II or III promoters that are inducible and/or tissue-specific have been previously described. RNA polymerase promoters are known in the art and further described in U.S. Pat. No. 11,149,288, the contents of which is incorporated by reference herein.

### Capsid Protein

The capsid protein is the shell or coating of the virus that enables its delivery into the host. Without the protein, the nucleic acids would be destroyed by the host without entering into the host cells and beginning transcription and translation. The capsid protein may be in the natural conformation of a naturally occurring AAV, or it may be modified.

In certain example embodiments, the AAV capsid protein is an engineered AAV capsid protein having reduced or eliminated uptake in a non-muscle cell as compared to a corresponding wild-type AAV capsid polypeptide.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter.

Described herein are various embodiments of engineered viral capsids, such as adeno-associated virus (AAV) capsids, that can be engineered to confer cell-specific tropism, such as muscle specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-specific tropism, reduced immunogenicity, or both to the engineered viral particle. The engineered viral capsids described herein can include one or more engineered viral capsid proteins described herein. The engineered viral capsids described herein can include one or more engineered viral capsid proteins described herein that can contain a muscle-specific targeting moiety containing or composed of an n-mer motif described elsewhere herein.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

The engineered viral capsid can contain 1-60 engineered capsid proteins. In some embodiments, the engineered viral capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered viral capsid can contain 0-59 wild-type viral capsid proteins. In some embodiments, the engineered viral capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type viral capsid proteins.

In some embodiments, the engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV capsid can contain 0-59 wild-type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins.

In some embodiments, the engineered viral capsid protein can have an n-mer amino acid motif, where n can be at least 3 amino acids. In some embodiments, n can be 3, 4, 5, 6, 7, 8, 9, 10, 11, 12, 13, 14, or 15 amino acids. In some embodiments, an engineered AAV capsid can have a 6-mer or 7-mer amino acid motif. In some embodiments, the n-mer amino acid motif can be inserted between two amino acids in the wild-type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parvovirus capsids (see e.g., DiMattia et al. 2012. J. Virol. 86 (12): 6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In some embodiments, one or more n-mer motifs can be inserted between two amino acids in one or more of the 12 variable regions in the wild-type AVV capsid proteins. In some embodiments, the one or more n-mer motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid protein. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer inserted between amino acids 588 and 589 of an AAV9 viral protein. In some embodiments, the engineered capsid can have a 7-mer motif inserted between amino acids 588 and 589 of an AAV9 viral protein. In other embodiments, the motif inserted is a 10-mer motif, with replacement of amino acids 586-88 and an insertion before 589. SEQ ID NO. 2 is a reference AAV9 capsid sequence for at least referencing the insertion sites discussed above. It will be appreciated that n-mers can be inserted in analogous positions in AAV viral proteins of other serotypes. In some embodiments as previously discussed, the n-mer(s) can be inserted between any two contiguous amino acids within the AAV viral protein and in some embodiments the insertion is made in a variable region.

In some embodiments, the first 1, 2, 3, or 4 amino acids of an n-mer motif can replace 1, 2, 3, or 4 amino acids of a polypeptide into which it is inserted and preceding the insertion site. In some embodiments, the amino acids of the n-mer motif that replace 1 or more amino acids of the polypeptide into which the n-mer motif is inserted come before or immediately before an “RGD” in an n-mer motif. For example, in one or more of the 10-mer inserts shown in e.g., Tables 2-3, the first three amino acids shown can replace 1-3 amino acids into a polypeptide to which they may be inserted. Using an AAV as another non-limiting example, one or more of the n-mer motifs can be inserted into e.g., and AAV9 capsid prolylpeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the n-mer motif is inserted are replaced by the n-mer motif.

In some embodiments, the AAV capsids or other viral capsids or compositions can be muscle-specific. In some embodiments, muscle-specificity of the engineered AAV or other viral capsid or other composition is conferred by a muscle specific n-mer motif incorporated in the engineered AAV or other viral capsid or other composition described herein. While not intending to be bound by theory, it is believed that the n-mer motif confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a muscle cell. In some embodiments, the cell surface receptor is AAV receptor (AAVR). In some embodiments, the cell surface receptor is a muscle cell specific AAV receptor. In some embodiments, the cell surface receptor or other molecule is a cell surface receptor or other molecule selectively expressed on the surface of a muscle cell. In some embodiments, the cell surface receptor or molecule is an integrin or dimer thereof. In some embodiments, the cell surface receptor or molecule is an Vb6 integrin heterodimer.

In some embodiments, a muscle specific engineered viral particle or other composition described herein containing the muscle-specific capsid, n-mer motif, or muscle-specific targeting moiety described herein can have an increased uptake, delivery rate, transduction rate, efficiency, amount, or a combination thereof in a muscle cell as compared to other cells types and/or other virus particles (including but not limited to AAVs) and other compositions that do not contain the muscle-specific n-mer motif of the present invention.

First- and second-generation muscle specific AAV capsids were developed using a muscle specific promoter and the resulting capsid libraries were screened in mice and non-human primates as described elsewhere herein and/or in e.g., U.S. Provisional Application Ser. Nos. 62/899,453, 62/916,207, 63/018,454, and 63/242,008. First and second generation myo AAV capsids were further optimized in mice and non-human primates as previously described to generate enhanced myoAAV capsids.

Tables 1 and 2 show the top hits of enhanced muscle specific n-mer motifs and their encoding sequence in rank order within each table. Enhanced MyoAAV (eMyoAAV) capsid variants can transduce mouse muscle more effectively as compared to the first generation MyoAAV after systemic delivery. First and second generation myoAAV capsid variants are dependent on the aVb6 integrin heterodimer for transduction of human primary myotubes.

Tables 3 and 4 show top-ranking capsid variants produced in rounds of directed evolution of capsid variants for skeletal muscle specificity. As shown in the Tables above with respect to those variant n-mer inserts containing P-motifs, the first three amino acids of the variant sequences shown are amino acids that replaced amino acids corresponding to positions 596, 597, and 598 of an AAV9 capsid polypeptide. Thus, the P-motif, for example, was inserted between amino acids at positions 598 and 599 of an AAV9 vector.

### DUX4-FL and Truncated Versions Thereof

DUX4 is the double homeobox protein 4, and is expressed in myocytes. DUX4-FL (SEQ ID NO. 1) is the double homeobox protein 4 gene; DUX4-FL is the wild type of the gene that is pathogenic in patients with FSHD. DUX4-FL causes pathogenesis by overproduction of the DUX4 protein. DUX4-FL is located on the D4Z4 region of the chromosome 4.

Together with a linker and a V5 epitope 17 amino acids long, truncated versions of the DUX4 protein include DUX4-S, which is the first 159 amino acids of DUX4-FL (SEQ ID NO. 3), delMid, which is amino acids 1-159, 343-424, delDC2, which is amino acids 1-374, delDC1/2, which is amino acids 1-342, S+DC1, which is amino acids 1-159, 343-374, S+DC2, which is 1-159, 375-424, S+375-397, which is amino acids 1-159, 375-397, S+398-424, which is amino acids 1-159, 398-424, or del405-424, which is amino acids 1-404. The linker is amino acid sequence LEGTRFE, and the V5 epitope is amino acid sequence GKPIPNPLLGLDSTRTG. These truncations are described in Mitsuhashi et al., (Biology Open (2018 Apr. 26; 7(4), bio033977, doi: 10.1242/bio.033977), which is incorporated by reference herein as if expressed in its entirety

Advantageously, truncated DUN4 genes may cause reduced expression of the DUX4 protein. Truncated DUX4 genes do not result in the same pathology, wherein the DUX4 protein is not over expressed.

In aspects of the invention, truncated forms of DUN4 are expressed by a nucleic acid molecule. “Nucleic acid molecule” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

In aspects of the invention, the truncated form of DUX4 may be a functional variant of the polypeptide. A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are substituted, mutated, inserted, or deleted), such that the polypeptide or encoded polypeptide is capable of performing at least one function of the encoded parent polypeptide with at least 50% efficiency of activity of the parent polypeptide.

In aspects of the invention, the functional variant may have at least 70% homology, at least 80% homology, at least 90% homology, at least 95% homology, or at least 99% homology with the reference/parent polypeptide disclosed herein.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound. In specific aspects, variants are at least 60% as efficient, at least 70% as efficient, at least 80% efficient, at least 90% as efficient, at least 95% as efficient, or at least 99% as efficient as the reference/parent polypeptides disclosed herein.

The invention discloses compositions and methods for treating Facioscapulohumeral Muscular Dystrophy (“FSHD”) that comprise a nucleic acid sequence encoding a modified copy of the double homeobox protein 4 (“DUX4”). Without being bound to a mechanism of action, the present invention benefits from the insight that the DUX4 gene presents in FSHD patients as a full-length form, known as DUX4-FL, and that modifications to the DUX4-FL gene can result in non-pathogenic expression of a truncated DUX4 protein. The invention further benefits from the discovery that adeno-associated virus (“AAV”) vectors may be modified to target muscle tissue and detarget liver tissue in rio, allowing for effective delivery of the gene product to a subject.

Accordingly, aspects of the invention provide an adeno-associated virus (AAV) vector comprising: a nucleic acid sequence encoding a truncated portion of the full-length DUX-4FL sequence operably linked to a muscle specific promoter, and a capsid protein. The full-length sequence of the DUX-4FL protein (SEQ ID NO. 1) and variants of the protein are discussed in Mitsuhashi et al. (2018) Biology Open 7 (4): bio033977, the entirety of the contents of which are incorporated by reference herein in their entirety.

For example, the truncated portion of the full-length DUX-4FL sequence may comprise amino acids 1-159 of the full-length amino acid sequence of DUX-4FL (SEQ ID NO. 3). The truncated portion of the full-length DUX-4FL sequence may comprise amino acids 19-159 of a full-length amino acid sequence of DUX-4FL (SEQ ID NO. 4). The truncated portion of the full-length DUX-4FL sequence may comprise amino acids 1-343 of a full-length amino acid sequence of DUX-4FL (SEQ ID NO.: 5).

Specific portions of the DUX-4FL sequence may be removed. For example, the truncated portion of the full-length DUX-4FL sequence may comprise a full-length amino acid sequence of DUX-4FL (SEQ ID NO. 1) in which amino acids 344-404 and 405-424 have been removed.

In aspects of the invention, the capsid protein may comprise at least one modification that results in reduced liver-tropism of the AAV vector. The capsid protein may comprise at least one modification that results in preferential targeting of the AAV vector to muscle tissue. The capsid protein may comprise at least one modification that results in reduced liver-tropism of the AAV vector, and at least one modification that results in preferential targeting of the AAV vector to muscle tissue.

For example, the capsid protein may comprise at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

The capsid protein may comprise at least one modification that is a replacement of amino acids 586-88 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide. For example, the capsid protein may comprise at least one modification that is a replacement of amino acids 586-88 and an insertion between amino acids 588 and 589 in an AAV9 capsid polypeptide and the insertion may be selected from the sequences in Table 1 or Table 2, hereinbelow.

In aspects of the invention, the muscle specific promoter may be MHCK7. For example, the AAV vector may comprise the truncated DUX-4FL sequence that comprises amino acids 1-159 of a full-length amino acid sequence of DUX-4FL (SEQ ID NO. 1), a capsid protein comprising at least one modification that is a replacement of amino acids 586-88 and an insertion between amino acids 588 and 589 in an AAV9 capsid polypeptide wherein the insertion is selected from the sequences in Tables 1-4, and the muscle specific promoter may be MHCK7.

Aspects of the invention further provide a method of treating a DUX-4 associated disorder in a subject, the method comprising: administering to a subject a composition comprising a nucleic acid sequence encoding a truncated portion of the full-length DUX-4FL sequence or an amino acid sequence expressed by the truncated portion of the full-length DUX-4FL sequence.

The adeno-associated virus (AAV) vector may comprise a nucleic acid sequence encoding the truncated portion of the full-length DUX-4FL sequence operably linked to a muscle specific promoter, and a capsid protein.

For example, the truncated portion of the full-length DUX-4FL sequence may comprise amino acids 1-159 of the full-length amino acid sequence of DUX-4FL (SEQ ID NO. 3). The truncated portion of the full-length DUX-4FL sequence may comprise amino acids 19-159 of a full-length amino acid sequence of DUX-4FL (SEQ ID NO. 4). The truncated portion of the full-length DUX-4FL sequence may comprise amino acids 1-343 of a full-length amino acid sequence of DUX-4FL (SEQ ID NO. 5).

Specific portions of the DUX-4FL sequence may be removed. For example, the truncated portion of the full-length DUX-4FL sequence may comprise a full-length amino acid sequence of DUX-4FL (SEQ ID NO. 1) in which amino acids 344-404 and 405-424 have been removed.

In aspects of the invention, the capsid protein may comprise at least one modification that results in reduced liver-tropism of the AAV vector. The capsid protein may comprise at least one modification that results in preferential targeting of the AAV vector to muscle tissue. The capsid protein may comprise at least one modification that results in reduced liver-tropism of the AAV vector, and at least one modification that results in preferential targeting of the AAV vector to muscle tissue.

For example, the capsid protein may comprise at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

The capsid protein may comprise at least one modification that is a replacement of amino acids 586-88 and an insertion between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide. For example, the capsid protein may comprise at least one modification that is a replacement of amino acids 586-88 and an insertion between amino acids 588 and 589 in an AAV9 capsid polypeptide and the insertion may be selected from the sequences in Tables 1-4, hereinbelow.

In aspects of the invention, the muscle specific promoter may be MHCK7. For example, the AAV vector may comprise the truncated portion of the full-length DUX-4FL sequence that comprises amino acids 1-159 of a full-length amino acid sequence of DUX-4FL (SEQ ID NO.: 3), a capsid protein comprising at least one modification that is a replacement of amino acids 586-88 and an insertion between amino acids 588 and 589 in an AAV9 capsid polypeptide wherein the insertion is selected from the sequences in Tables 1-4, and the muscle specific promoter may be MHCK7.

In aspects of the method, the DUX4 associated disorder may be facioscapulohumeral muscular dystrophy (FSHD). In aspects of the method, the composition may be administered to the subject intramuscularly.

In aspects of the method, transcription of the nucleic acid results in expression of a truncated DUX4 protein. Expression of the truncated DUX4 protein may inhibit activity and/or expression of the full-length DUX4 protein. The truncated DUX4 protein may inhibit Trim36 and Wfdc3 expression.

Further embodiments of the invention comprise an adeno-associated virus (AAV) vector comprising a promoter sequence, a miRNA strand that binds to DUX4-FL and a capsid protein comprising at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In aspects of the invention, the promoter sequence may be a promoter sequence transcribed by RNA polymerase II or III, U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or a TH promoter sequence. The miRNA strand may be selected from the group of miDUX4, miRN92, miRNA-17, miRNA-18a, miRNA-19a, miRNA-20a, miRNA-19b-1, mi-RNA-26a, miRNA-126, miRNA-335, let-7a and let-7b, miRNA-34, miR-34a, miRNA-10b, miRNA-208, miRNA-499, miRNA-195, miRNA-29a, miRNA-29b, or miRNA-29c.

The miRNA strand may also be selected from the group of SEQ ID NOs. 1250-1305. The miRNA strand may also comprise 5-6 thymidines at the 5′ end.

In an embodiment of the invention, the capsid protein comprises at least one modification that results in reduced liver-tropism of the AAV vector, or at least one modification that results in preferential targeting of the AAV vector to muscle tissue, or both. The capsid protein may be selected from the sequences in Tables 1-4.

In an embodiment of the invention, the vector further comprises a nuclear export sequence enabling nuclear spreading.

### Micro RNA

MiRNA-based therapies, including miRNA inhibition and miRNA replacement, may be used to treat many diseases such as hepatitis C viral infection, muscular dystrophies, neurodegenerative diseases, peripheral neuropathies, chronic heart failure and post-myocardial infarction remodeling and cancers. In addition, miRNA directed regulation of gene expression may improve traditional gene therapy approaches in which the vector payload is a protein coding gene. Systemically delivered AAV vectors preferentially transduce the liver, resulting in high-level transgene expression in that organ if a liver-active promoter is used. As described in detail herein, the insertion of liver-specific miR-122 binding sites reduce transgene expression in the liver when a liver-specific promoter is used.

The nucleic acid molecule of the disclosure comprises a mature guide strand of a miRNA to knockdown expression of the DUX4 gene. This system may be used with any tissue specific promoter or gene expression control element and any miRNA sequences. For example, in order to promote expression of miRNA sequence in skeletal muscle and to detarget expression of the miRNA in liver and heart tissue, the nucleic acid molecule comprises the mature guide stand of the miRNA in which the binding sites for liver specific miR-122 and/or the binding site for heart specific miR-208 are inserted within the loop of the mature guide strand or at the 5′ or 3′ end of the mature guide strand of the miRNA.

There are two miR-208 sequences in the human and mouse genome (miR-208a and miR-208b). To avoid a run of 5 U's (pol III promoter termination sequence), in the above exemplary sequences, a single base in the binding site was mutated to a “c” (lower-case bolded “c”). This change was included because it creates a perfect binding site for mir-208b, but will have a mismatch with mir-208a. If detargeting expression of a miRNA in skeletal muscle is desired, binding sites for miR-1, miR-206 or miR-133 are inserted within the loop of the mature guide strand of the miRNA or at the 5′ or 3′ end of the mature guide strand of the miRNA.

There are two miR-208 sequences in the human and mouse genome (miR-208a and miR-208b). To avoid a run of 5 U's (pol III promoter termination sequence), in the following exemplary sequences, a single base in the binding site was mutated to a “c” (lower-case bolded “c”). This change was included because it creates a perfect binding site for mir-208b, but will have a mismatch with mir-208a.

The nucleic acid molecules of the disclosure may comprise the sequence of the mature guide strand of any miRNA transcript sequence desired to have tissue-specific expression. For example, in one embodiment, skeletal expression of DUX4 miRNA is contemplated. Exemplary DUX4 miRNA sequences are provided in International Patent Application No. PCT/US2012/047999 (WO 2013/016352) and US patent publication no. US 2012-20225034 incorporated by reference herein in their entirety.

Two examples of miDUX4 are miDUX4-1; and miDUX4-2. Exemplary nucleotide sequences comprising the DUX4 miRNA and the binding site for either miR-122 or miR-208 are provided in Table 5.

Any human miRNA may be expressed using the nucleic acid molecules of the disclosure, including those set out in the miRBase: the microRNA database websites (miRBase.org), which are incorporated by reference herein in its entirety. Examples include but not limited to the following: miR-122, miR-124, miR-142, miR-155, miR-21, miR-17-92, miR-17, miR-18a, miR-19a, miR-20a, miR-19b-1, miR-26a, miR-126, miR-335, let-7 family: let-7a and let-7b, miR-34 (miR-34a), miR-10b, miR-208, miR-499, miR-195, miR-29a, miR-29b, and miR-29c. Additional exemplary miRNA are set out below in Table 6 above. Any of these miRNA may be used with different detargeting sequences, depending of the desired tissue specificity and desired detargeting.

The AAV vector of claim 35, wherein the miRNA strand is selected from the group of in table 5.

The AAV vector of claim 35, wherein the miRNA strand further comprises 5-6 thymidines at the 5′ end. The AAV vector of claim 35, wherein the miRNA strand is the binding site for miRNA-122 or miRNA 208.

Embodiments of the invention further provide methods of decreasing gene expression in a cell. The method may include providing the AAV vector to a subject, the vector comprised as noted within this section.

Further microRNA sequences are described in U.S. Patent Publication Nos. 2020-0248179, 2019-0300903, 2019-0136235, 2019-0024083, 2017-0029849, and 2014-0322169, the contents of each of which are incorporated by reference herein.

### Pharmaceutical Composition

Some embodiments of the invention may include any acceptable form of providing the AAV vector to a subject. For example, the AAV vector may be provided to the subject in the form of a composition or formulation comprising the AAV vector. The expression vector of this invention can be formulated and administered to treat a variety of disease states by any means that produces contact of the active ingredient with the agent's site of action in the body of the subject. The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more muscle-specific targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1 ×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10², 1×10³, 1×10⁴, 1×10⁵, 1 ×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### DUX4 and Facioscapulohumeral Muscular Dystrophy

Muscular dystrophies (MDs) are a group of genetic diseases. The group is characterized by progressive weakness and degeneration of the skeletal muscles that control movement or breathing. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Facioscapulohumeral muscular dystrophy (FSHD) is a complex autosomal dominant disorder characterized by progressive and asymmetric weakness of facial, shoulder and limb muscles. Symptoms typically arise in adulthood with most patients showing clinical features before age thirty. About five percent of patients develop symptoms as infants or juveniles and these are generally more severely affected. Clinical presentation can vary from mild (some limited muscle weakness) to severe (wheelchair dependence). Historically, FSHD was classified as the third most common MD, affecting one in 20,000 individuals worldwide. However, recent data indicate FSHD is the most common MD in Europe, suggesting its worldwide incidence could be as high as 1 in 8,333. Typical FSHD cases (FSHD1A, heretofore referred to as FSHD) are linked to heterozygous chromosomal deletions that decrease the copy number of 3.3 kilobase (kb) D4Z4 repeats on human chromosome 4q35. Simplistically, normal individuals have 11-100 tandemly-repeated D4Z4 copies on both 4q35 alleles, while patients with FSHD have one normal and one contracted allele containing 1-10 repeats. In addition, FSHD-associated D4Z4 contractions must occur on specific disease-permissive chromosome 4q35 backgrounds (called 4qA). Importantly, no genes are completely lost or structurally mutated as a result of FSHD-associated deletions. Instead, genetic changes associated with FSHD give rise to expression of the toxic DUX4 gene, which is damaging to muscle. FSHD2 (also known as FSHD1B) is phenotypically identical to FSHD1, is associated with DUX4 expression, and requires the 4qA chromosomal background. FSHD2 is not associated with D4Z4 repeat contraction, but is instead caused by mutation in the SMCHD1 gene, which is a chromatin regulator normally involved in repressing the DUX4 locus at 4qA. Mutated SMCHD1 proteins fail to participate in adding heterochromatin to the 4qA DUX4 allele, thereby allowing DUX4 gene expression.

In the leading FSHD pathogenesis model, D4Z4 contractions are proposed to cause epigenetic changes that permit expression of the DUX4 gene. As a result, the aberrant over-expression of otherwise silent or near-silent DUN4 gene, and the genes it regulates, may ultimately cause FSHD. This model is consistent with data showing normal 4q35 D4Z4 repeats have heterochromatin characteristics, while FSHD-linked D424 repeats contain marks more indicative of actively transcribed euchromatin. These transcription-permissive epigenetic changes, coupled with the observation that complete monosomic D4Z4 deletions (i.e., zero repeats) do not cause FSHD, support the hypothesis that D4Z4 repeats harbor potentially myopathic open reading frames (ORFs), which are abnormally expressed in FSHD muscles. This notion was initially considered in 1994, when a D4Z4-localized ORF, called DUX4, was first identified. However, the locus had some characteristics of an unexpressed pseudogene and DUX4 was therefore summarily dismissed as an FSHD candidate. For many years thereafter, the search for FSHD-related genes was mainly focused outside the D4Z4 repeats, and although some intriguing candidates emerged from these studies, no single gene had been conclusively linked to FSHD development. This slow progress led to the re-emergence of DUX4 as an FSHD candidate in 2007.

The role of DUX4 in FSHD pathogenesis can be explained as follows. First, D4Z4 repeats contain identical DUX4 coding regions, and D4Z4 repeats also harbor smaller sense and antisense transcripts, including some resembling microRNAs. Over-expressed DUX4 transcripts and a .about.50 kDa full-length DUX4 protein are found in biopsies and cell lines from FSHD patients. These data are consistent with a transcriptional de-repression model of FSHD pathogenesis. In addition, unlike pseudogenes, D4Z4 repeats and DUX4 likely have functional importance, since tandemly-arrayed D4Z4 repeats are conserved in at least eleven different placental mammalian species (non-placental animals lack D4Z4 repeats), with the greatest sequence conservation occurring within the DUX4 ORF. Second, over-expressed DUX4 is toxic to tissue culture cells and embryonic progenitors of developing lower organisms in vivo. This toxicity occurs at least partly through a pro-apoptotic mechanism, indicated by Caspase-3 activation in DUX4 transfected cells, and presence of TUNEL-positive nuclei in developmentally arrested Xenopus embryos injected with DUX4 mRNA at the two-cell stage. These findings are consistent with studies showing some pro-apoptotic proteins, including Caspase-3, are present in FSHD patient muscles. In addition to stimulating apoptosis, DUX4 may negatively regulate myogenesis. Human DUX4 inhibits differentiation of mouse C2C12 myoblasts in vitro, potentially by interfering with PAX3 and/or PAX7, and causes developmental arrest and reduced staining of some muscle markers when delivered to progenitor cells of zebrafish or Xenopus embryos. Finally, aberrant DUX4 function is directly associated with potentially important molecular changes seen in FSHD patient muscles. Specifically, full-length human DUX4 encodes an approximately 50 kDa double homeodomain transcription factor, and DUX4 targets can be found at elevated levels in FSHD patient muscles. These data support that DUX4 catalyzes numerous downstream molecular changes that are incompatible with maintaining normal muscle integrity.

### Application of Nuclear Spreading to Facioscapulohumeral Muscular Dystrophy (FSHD)

A DUY4 Dominant Negative can compete with full-length DUX4 to prevent DUX4-mediated transcription. One such construct is S+375-397 DUX4, which contains IIOX1 and IIOX2 DNA binding domains, which but only part of the C-terminal domain (FIG. 8A). This construct was identified by Mitsuhashi et al. as a potential inhibitory construct which can bind the DUX4 promoter without being toxic to cells.

Muscle cells include cells including multiple nuclei, thus a DUX4 dominant negative may require efficient spreading to multiple myonuclei. Thus, if delivered by AAV, not all myonuclei will generate DUX4 dominant negative RNA, and if the DUX4 dominant negative has nuclear localization activity, then activity of the dominant negative will be limited to only a few nuclei. Addition of nuclear export sequences may enable the DUN4 dominant negative to spread across the myofiber.

In an experiment, C2C12 mouse myoblast stable cell lines were created and used: reporter cell line containing Zscan4 promoter driving tdTomato (Zscan4 is a promoter activated by DUX4); cell line that contains tet-inducible DUX4-Halo (Halo is a fluorescent protein that can be visualized upon addition of a small molecule dye); cell line that stably expresses S+375-397 DUX4 dominant negative fused to GFP; and cell line that stably expresses S+375-397 DUX4 dominant negative fused to GFP and an ALYREF nuclear export signal. Cell lines were plated at varying ratios onto a gelatin substrate that facilitates formation of myotubes. The cells were then differentiated into myotubes; each myotube contains a mixture of nuclei from all the different cell lines. Once differentiated (7 days post serum withdrawal), DUX4-Halo is induced with doxycycline-containing media (500 nanograms per milliliter (ng/ml)).

Myotubes were imaged 72 hours following doxycycline induction. S+375-397 dominant negative prevents binding of DUN4-Halo to the Zscan promoter, limiting expression of tdTomato, with the ALYREF construct performing more efficiently.

The ALYREF dominant negative fusion prevents tdTomato expression more effectively than the dominant negative without the ALYREF sequence providing enhanced nuclear-cytoplasmic trafficking which is beneficial to limit DUX4 transcriptional activation.

Nuclear spreading is further described in the patent application PCT/US2021/033680, the entirety of the contents of which is incorporated by reference herein in its entirety.

### EXAMPLES

Adeno-associated viral vectors comprising a nucleic acid sequence encoding Dux4 and adeno-associated viral vectors comprising a nucleic acid sequence encoding truncated forms of Dux4 were administered to mice. Expression of Dux4 regulated genes were analyzed.

FIG. 1 shows the expression rates of protein coding gene Wfdc3 in response to full and truncated forms of Dux4. Wfdc3 is the Whey Acidic Four-Disulfide Core Domain Protein 3; it functions as a protease inhibitor and contains eight cysteines forming four disulfide bonds at the core of the protein. Expression of the gene may be associated with FSHD. Experimental mice were intramuscularly administered an inert control, the full-length DUX4-FL sequence, the truncated DUX4-S sequence, a 1:1 co-administered mixture of DUX4-FL and DUX4-S, and a 1:10 1 co-administered mixture of DUX4-FL and DUX4-S. Wfdc3 was found to be upregulated in those mice that received only the DUX4-FL full sequence, as well as upregulated to a lesser degree in those mice that received a 1:1 co-administered mixture of DUX4-FL and DUX4-S. Wfdc3 was not upregulated in mice that received the inert control, DUX4-S alone, or the 1:10 1 mixture of DUX4-FL and DUX4-S.

FIG. 2 shows the expression rates of protein coding gene Trim36 in response to varying forms of the DUX4 sequence. Trim36 is the tripartite motif containing 36 gene; it includes three zinc-binding domains, a ring, B-box type 1 and B-box type 2, and a coiled-coil region. Expression of the gene is related to the innate immune system pathway, and the gene is involved in chromosome segregation and regulation of the cell cycle. Experimental mice were intramuscularly administered an inert control, the full-length DUX4-FL sequence, the truncated DUX4-S sequence, a 1:1 co-administered mixture of DUX4-FL and DUX4-S, and a 1:10 1 co-administered mixture of DUX4-FL and DUX4-S. Trim36 was found to be upregulated in those mice that received only the DUX4-FL full sequence, as well as upregulated to a lesser degree in those mice that received a 1:1 co-administered mixture of DUX4-FL and DUX4-S. Trim36 expression matched the inert control in mice receiving DUX4S alone. Trim36 expression was lower than the vehicle in mice that received the 1:10 1 mixture of DUX4-FL and DUX4-S.

### CONCLUSION

Expression of truncated forms of DUX4 inhibit the expression of DUX4 controlled genes, even in the presence of full-length DUX4. Accordingly, DUX4 associated conditions, such as FSHD, may be treated by expression of truncated forms of DUX4.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Select Sequences
SEQ ID NO. 1: Full-length DUX4-FL amino acid
sequence.
MALPTPSDSTLPAEARGRGRRRRLVWTPSQSEALRACFERNPYPGIAT
RERLAQAIGIPEPRVQIWFQNERSRQLRQHRRESRPWPGRRGPPEGRR
KRTAVTGSQTALLLRAFEKDRFPGIAAREELARETGLPESRIQIWFQN
RRARHPGQGGRAPAQAGGLCSAAPGGGHPAPSWVAFAHTGAWGTGLPA
PHVPCAPGALPQGAFVSQAARAAPALQPSQAAPAEGISQPAPARGDFA
YAAPAPPDGALSHPQAPRWPPHPQKSREDRDPQRDGLPGPCAVAQPGP
AQAGPQGQGVLAPPTSQGSPWWGWGRGPQVAGAAWEPQAGAAPPPQPA
PPDASASARQGQMQGIPAPSQALQEPAPWSALPCGLLLDELLASPEFL
QQAQPLLETEAPGELEASEEAASLEAPLSEEEYRALLEEL
SEQ ID NO. 2: AAV9 Capsid Reference Sequence
MAADGYLPDWLEDNLSEGIREWWALKPGAPQPKANQQHQDNARGLVLP
GYKYLGPGNGLDKGEPVNAADAAALEHDKAYDQQLKAGDNPYLKYNHA
DAEFQERLKEDTSFGGNLGRAVFQAKKRLLEPLGLVEEAAKTAPGKKR
PVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNR
FHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSL
DRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHK
EGEDRFFPLSGSLIFGKQGTGRDNVDADKVMITNEEEIKTTNPVATES
YGQVATNIIQSAQAQAQTGWVQNQGILPGMVWQDRDVYLQGPIWAKIP
IITDGNFIIPSPLMGGFGMKHPPPQILIKNTPVPADPPTAFNKDKLNS
FITQYSTGQVSVEIEWELQKENSKRWNPEIQYTSNYYKSNNVEFAVNT
EGVYSEPRPIGTRYLTRNL
SEQ ID NO. 3: DUX4-FL Truncated
MALPTPSDSTLPAEARGRGRRRRLVWTPSQSEALRACFERNPYPGIAT
RERLAQAIGIPEPRVQIWFQNERSRQLRQHRRESRPWPGRRGPPEGRR
KRTAVTGSQTALLLRAFEKDRFPGIAAREELARETGLPESRIQIWFQN
RRARHPGQGGRAPAQ
SEQ ID NO. 4: DUX4-FL Truncated
GRRRRLVWTPSQSEALRACFERNPYPGIATRERLAQAIGIPEPRVQIW
FQNERSRQLRQHRRESRPWPGRRGPPEGRRKRTAVTGSQTALLLRAFE
KDRFPGIAAREELARETGLPESRIQIWFQNRRARHPGQGGRAPAQ
SEQ ID NO. 5: DUX4-FL Truncated
MALPTPSDSTLPAEARGRGRRRRLVWTPSQSEALRACFERNPYPGIAT
RERLAQAIGIPEPRVQIWFQNERSRQLRQHRRESRPWPGRRGPPEGRR
KRTAVTGSQTALLLRAFEKDRFPGIAAREELARETGLPESRIQIWFQN
RRARHPGQGGRAPAQAGGLCSAAPGGGHPAPSWVAFAHTGAWGTGLPA
PHVPCAPGALPQGAFVSQAARAAPALQPSQAAPAEGISQPAPARGDFA
YAAPAPPDGALSHPQAPRWPPHPQKSREDRDPQRDGLPGPCAVAQPGP
AQAGPQGQGVLAPPTSQGSPWWGWGRGPQVAGAAWEPQAGAAPPPQPA
PPDASAS

1. An adeno-associated virus (AAV) vector comprising:
a promoter sequence,
a miRNA strand that binds to DUX4-FL, and
a capsid protein comprising at least one modification that is an insertion between any two contiguous amino acids between amino acids 262-269, 327-332, 382-386, 452-460, 488-505, 527-539, 545-558, 581-593, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

2. The AAV vector of claim 1, wherein the promoter sequence is a promoter sequence transcribed by RNA polymerase II or III, U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or TH promoter sequence.

3. The AAV vector of claim 1, wherein the miRNA strand is selected from the group of miDUX4, miRN92, miRNA-17, miRNA-18a, miRNA-19a, miRNA-20a, miRNA-19b-1, mi-RNA-26a, miRNA-126, miRNA-335, let-7a and let-7b, miRNA-34, miR-34a, miRNA-10b, miRNA-208, miRNA-499, miRNA-195, miRNA-29a, miRNA-29b, or miRNA-29c.

4. The AAV vector of claim 1, wherein the miRNA strand is selected from the group of SEQ ID NOs. 1250-1305.

5. The AAV vector of claim 1, wherein the miRNA strand further comprises 5-6 thymidines at the 5′ end.

6. The AAV vector of claim 1, wherein the capsid protein comprises at least one modification that results in reduced liver-tropism of the AAV vector.

7. The AAV vector of claim 1, wherein the capsid protein comprises at least one modification that results in preferential targeting of the AAV vector to muscle tissue.

8. The AAV vector of claim 1, wherein the capsid protein may be selected from the sequences in Tables 1-4.

9. The AAV vector of claim 1, wherein the vector further comprises a nuclear export sequence enabling nuclear spreading.

10. A method of inhibiting expression of a gene in a cell, the method comprising administering to a subject an adeno-associated virus (AAV) vector comprising a promoter sequence, a miRNA strand that binds to DUX4-FL, and a capsid protein comprising at least one modification that is an insertion between any two contiguous amino acids between amino acids 262-269, 327-332, 382-386, 452-460, 488-505, 527-539, 545-558, 581-593, 704-714, or any combination thereof in an AAV9) capsid polypeptide or in an analogous position in an AAV1, AAV2, AAV3, AAV4, AAV5, AAV6, AAV7, AAV8, AAV rh.74, AAV rh.10 capsid polypeptide.

11. The method of claim 8, wherein the promoter sequence is a promoter sequence transcribed by RNA polymerase II or III, U6 promoter sequence, MHCK7 promoter sequence, CK6 promoter sequence, tMCK promoter sequence, CK5 promoter sequence, MCK promoter sequence, HAS promoter sequence, MPZ promoter sequence, desmin promoter sequence, APOA2 promoter sequence, hAAT promoter sequence, INS promoter sequence, IRS2 promoter sequence, MYH6 promoter sequence, MYL2 promoter sequence, TNNI3 promoter sequence, SYN1 promoter sequence, GFAP promoter sequence, NES promoter sequence, MBP promoter sequence, or TH promoter sequence.

12. The method of claim 8, wherein the miRNA strand is selected from the group of miDUX4, miRN92, miRNA-17, miRNA-18a, miRNA-19a, miRNA-20a, miRNA-19b-1, mi-RNA-26a, miRNA-126, miRNA-335, let-7a and let-7b, miRNA-34, miR-34a, miRNA-10b, miRNA-208, miRNA-499, miRNA-195, miRNA-29a, miRNA-29b, or miRNA-29c.

13. The method of claim 8, wherein the miRNA strand is selected from the group of SEQ ID NOs 1250-1305.

14. The method of claim 8, wherein the miRNA strand further comprises 5-6 thymidines at the 5′ end.

15. The method of claim 8, wherein the capsid protein comprises at least one modification that results in reduced liver-tropism of the AAV vector.

16. The method of claim 8, wherein the capsid protein comprises at least one modification that results in preferential targeting of the AAV vector to muscle tissue.

17. The method of claim 8, wherein the capsid protein may be selected from the sequences in Tables 1-4.

18. The method of claim 8, wherein the vector further comprises a nuclear export sequence enabling nuclear spreading.


SEQUENCE LISTING (1305 sequences)

SEQ ID NO: 1 - protein, 424 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPQKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 2 - protein, 736 aa, Homo sapiens
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 159 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQ

SEQ ID NO: 4 - protein, 141 aa, Homo sapiens
GRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQ

SEQ ID NO: 5 - protein, 343 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPQKSREDRDPQRDGLPGPCAVAQPGPAQAGPQGQGVLAPPTSQGSPWWGWGRGPQVAGAAWEPQAGAAPPPQPAPPDASAS

SEQ ID NO: 6 - protein, 10 aa, Homo sapiens
NISRGDQGYQ

SEQ ID NO: 7 - protein, 10 aa, Homo sapiens
APARGDQGSQ

SEQ ID NO: 8 - protein, 10 aa, Homo sapiens
AVSRGDRMEF

SEQ ID NO: 9 - protein, 10 aa, Homo sapiens
SPSRGDQGRT

SEQ ID NO: 10 - protein, 10 aa, Homo sapiens
QMGRGDMGIK

SEQ ID NO: 11 - protein, 10 aa, Homo sapiens
EYRRGDKADI

SEQ ID NO: 12 - protein, 10 aa, Homo sapiens
ESRRGDKEPL

SEQ ID NO: 13 - protein, 10 aa, Homo sapiens
AHMRGDLGGT

SEQ ID NO: 14 - protein, 10 aa, Homo sapiens
VWQRGDKMDM

SEQ ID NO: 15 - protein, 10 aa, Homo sapiens
SIGRGDTGHM

SEQ ID NO: 16 - protein, 10 aa, Homo sapiens
NIARGDAGQY

SEQ ID NO: 17 - protein, 10 aa, Homo sapiens
SVSRGDQGLH

SEQ ID NO: 18 - protein, 10 aa, Homo sapiens
QIGRGDITHG

SEQ ID NO: 19 - protein, 10 aa, Homo sapiens
EVRRGDLHGT

SEQ ID NO: 20 - protein, 10 aa, Homo sapiens
SVSRGDVHTM

SEQ ID NO: 21 - protein, 10 aa, Homo sapiens
MVTRGDLGTR

SEQ ID NO: 22 - protein, 10 aa, Homo sapiens
NGGRGDTTHF

SEQ ID NO: 23 - protein, 10 aa, Homo sapiens
TMGRGDMNSL

SEQ ID NO: 24 - protein, 10 aa, Homo sapiens
AISRGDQGLS

SEQ ID NO: 25 - protein, 10 aa, Homo sapiens
NDARGDKSTY

SEQ ID NO: 26 - protein, 10 aa, Homo sapiens
VGLRGDLTGS

SEQ ID NO: 27 - protein, 10 aa, Homo sapiens
EMRRGDLGTS

SEQ ID NO: 28 - protein, 10 aa, Homo sapiens
AMSRGDMGMA

SEQ ID NO: 29 - protein, 10 aa, Homo sapiens
NVARGDQVNY

SEQ ID NO: 30 - protein, 10 aa, Homo sapiens
ASVRGDLSGS

SEQ ID NO: 31 - protein, 10 aa, Homo sapiens
SAARGDTERL

SEQ ID NO: 32 - protein, 10 aa, Homo sapiens
VPGRGDLNTM

SEQ ID NO: 33 - protein, 10 aa, Homo sapiens
TSVRGDHGTL

SEQ ID NO: 34 - protein, 10 aa, Homo sapiens
TPSRGDLGQT

SEQ ID NO: 35 - protein, 10 aa, Homo sapiens
AGHRGDTGVI

SEQ ID NO: 36 - protein, 10 aa, Homo sapiens
APARGDQGSM

SEQ ID NO: 37 - protein, 10 aa, Homo sapiens
MSLRGDLNGS

SEQ ID NO: 38 - protein, 10 aa, Homo sapiens
EAKRGDVHSI

SEQ ID NO: 39 - protein, 10 aa, Homo sapiens
SAQRGDVQAV

SEQ ID NO: 40 - protein, 10 aa, Homo sapiens
STARGDQGDR

SEQ ID NO: 41 - protein, 10 aa, Homo sapiens
QISRGDLGIN

SEQ ID NO: 42 - protein, 10 aa, Homo sapiens
TFTRGDMTMN

SEQ ID NO: 43 - protein, 10 aa, Homo sapiens
QEGRGDLNMR

SEQ ID NO: 44 - protein, 10 aa, Homo sapiens
AHARGDTSSL

SEQ ID NO: 45 - protein, 10 aa, Homo sapiens
TNGRGDAGTL

SEQ ID NO: 46 - protein, 10 aa, Homo sapiens
AFGRGDQGQL

SEQ ID NO: 47 - protein, 10 aa, Homo sapiens
ADGRGDRSSL

SEQ ID NO: 48 - protein, 10 aa, Homo sapiens
DGRRGDGHSL

SEQ ID NO: 49 - protein, 10 aa, Homo sapiens
EKLRGDLHST

SEQ ID NO: 50 - protein, 10 aa, Homo sapiens
EARRGDASAM

SEQ ID NO: 51 - protein, 10 aa, Homo sapiens
MGTRGDKMDF

SEQ ID NO: 52 - protein, 10 aa, Homo sapiens
STSRGDRESY

SEQ ID NO: 53 - protein, 10 aa, Homo sapiens
CQPRGDTTRC

SEQ ID NO: 54 - protein, 10 aa, Homo sapiens
MHTRGDKMDF

SEQ ID NO: 55 - protein, 10 aa, Homo sapiens
YSARGDTSGL

SEQ ID NO: 56 - protein, 10 aa, Homo sapiens
STMRGDHEKL

SEQ ID NO: 57 - protein, 10 aa, Homo sapiens
TQMRGDGVTL

SEQ ID NO: 58 - protein, 10 aa, Homo sapiens
TTRRGDMGDN

SEQ ID NO: 59 - protein, 10 aa, Homo sapiens
ANGRGDRLEL

SEQ ID NO: 60 - protein, 10 aa, Homo sapiens
ENQRGDLSGR

SEQ ID NO: 61 - protein, 10 aa, Homo sapiens
STGRGDLGQA

SEQ ID NO: 62 - protein, 10 aa, Homo sapiens
SSARGDYSEV

SEQ ID NO: 63 - protein, 10 aa, Homo sapiens
STARGDAATY

SEQ ID NO: 64 - protein, 10 aa, Homo sapiens
VNTRGDTQKL

SEQ ID NO: 65 - protein, 10 aa, Homo sapiens
AMSRGDHASL

SEQ ID NO: 66 - protein, 10 aa, Homo sapiens
QAARGDVNKL

SEQ ID NO: 67 - protein, 10 aa, Homo sapiens
RGTRGDTVEL

SEQ ID NO: 68 - protein, 10 aa, Homo sapiens
ASTRGDYAGV

SEQ ID NO: 69 - protein, 10 aa, Homo sapiens
DDRRGDKLPL

SEQ ID NO: 70 - protein, 10 aa, Homo sapiens
SGSRGDLNAV

SEQ ID NO: 71 - protein, 10 aa, Homo sapiens
EYRRGDQQIQ

SEQ ID NO: 72 - protein, 10 aa, Homo sapiens
TYVRGDRAEV

SEQ ID NO: 73 - protein, 10 aa, Homo sapiens
QSLRGDLNGS

SEQ ID NO: 74 - protein, 10 aa, Homo sapiens
EGRRGDTTSL

SEQ ID NO: 75 - protein, 10 aa, Homo sapiens
AENRGDRSSL

SEQ ID NO: 76 - protein, 10 aa, Homo sapiens
VAGRGDRSEL

SEQ ID NO: 77 - protein, 10 aa, Homo sapiens
DPARGDIGAR

SEQ ID NO: 78 - protein, 10 aa, Homo sapiens
VIGRGDKALQ

SEQ ID NO: 79 - protein, 10 aa, Homo sapiens
SFERGDKNSL

SEQ ID NO: 80 - protein, 10 aa, Homo sapiens
QIARGDIASV

SEQ ID NO: 81 - protein, 10 aa, Homo sapiens
TISRGDLGGA

SEQ ID NO: 82 - protein, 10 aa, Homo sapiens
AMIRGDMTHS

SEQ ID NO: 83 - protein, 10 aa, Homo sapiens
TMRRGDLNDS

SEQ ID NO: 84 - protein, 10 aa, Homo sapiens
AYSRGDLGNH

SEQ ID NO: 85 - protein, 10 aa, Homo sapiens
EERRGDTHRL

SEQ ID NO: 86 - protein, 10 aa, Homo sapiens
SQLRGDAGTI

SEQ ID NO: 87 - protein, 10 aa, Homo sapiens
AMVRGDAHQL

SEQ ID NO: 88 - protein, 10 aa, Homo sapiens
TTSRGDLNSV

SEQ ID NO: 89 - protein, 10 aa, Homo sapiens
WEQRGDMIGK

SEQ ID NO: 90 - protein, 10 aa, Homo sapiens
YTTRGDLQSN

SEQ ID NO: 91 - protein, 10 aa, Homo sapiens
MSQRGDLSHQ

SEQ ID NO: 92 - protein, 10 aa, Homo sapiens
ASTRGDTTHM

SEQ ID NO: 93 - protein, 10 aa, Homo sapiens
SDTRGDTHRL

SEQ ID NO: 94 - protein, 10 aa, Homo sapiens
TLGRGDQYTS

SEQ ID NO: 95 - protein, 10 aa, Homo sapiens
SYARGDVREV

SEQ ID NO: 96 - protein, 10 aa, Homo sapiens
ETRRGDLSQN

SEQ ID NO: 97 - protein, 10 aa, Homo sapiens
TIGRGDLMDK

SEQ ID NO: 98 - protein, 10 aa, Homo sapiens
SYTRGDLSQN

SEQ ID NO: 99 - protein, 10 aa, Homo sapiens
SKLRGDASEI

SEQ ID NO: 100 - protein, 10 aa, Homo sapiens
SMARGDKAEI

SEQ ID NO: 101 - protein, 10 aa, Homo sapiens
GAVRGDHSSL

SEQ ID NO: 102 - protein, 10 aa, Homo sapiens
METRGDMSNR

SEQ ID NO: 103 - protein, 10 aa, Homo sapiens
AYTRGDKDAL

SEQ ID NO: 104 - protein, 10 aa, Homo sapiens
ENRRGDITQS

SEQ ID NO: 105 - protein, 10 aa, Homo sapiens
MLLRGDKNDF

SEQ ID NO: 106 - protein, 10 aa, Homo sapiens
KVQRGDASDW

SEQ ID NO: 107 - protein, 10 aa, Homo sapiens
AFTRGDMQTA

SEQ ID NO: 108 - protein, 10 aa, Homo sapiens
QPKRGDVQAQ

SEQ ID NO: 109 - protein, 10 aa, Homo sapiens
ASLRGDMGVS

SEQ ID NO: 110 - protein, 10 aa, Homo sapiens
AYSRGDKESF

SEQ ID NO: 111 - protein, 10 aa, Homo sapiens
RFQRGDLTDA

SEQ ID NO: 112 - protein, 10 aa, Homo sapiens
SDARGDTMKL

SEQ ID NO: 113 - protein, 10 aa, Homo sapiens
SGARGDTVSL

SEQ ID NO: 114 - protein, 10 aa, Homo sapiens
DEKRGDQKHL

SEQ ID NO: 115 - protein, 10 aa, Homo sapiens
NGVRGDVQNF

SEQ ID NO: 116 - protein, 10 aa, Homo sapiens
MTLRGDLGGS

SEQ ID NO: 117 - protein, 10 aa, Homo sapiens
GQERGDLGTR

SEQ ID NO: 118 - protein, 10 aa, Homo sapiens
RGARGDLVDA

SEQ ID NO: 119 - protein, 10 aa, Homo sapiens
QYSRGDHTDL

SEQ ID NO: 120 - protein, 10 aa, Homo sapiens
MNTRGDVHAM

SEQ ID NO: 121 - protein, 10 aa, Homo sapiens
TVRRGDLATE

SEQ ID NO: 122 - protein, 10 aa, Homo sapiens
SVGRGDKADI

SEQ ID NO: 123 - protein, 10 aa, Homo sapiens
SHNRGDTGTM

SEQ ID NO: 124 - protein, 10 aa, Homo sapiens
TNTRGDKESV

SEQ ID NO: 125 - protein, 10 aa, Homo sapiens
SIGRGDQYTI

SEQ ID NO: 126 - protein, 10 aa, Homo sapiens
QTSRGDAGSW

SEQ ID NO: 127 - protein, 10 aa, Homo sapiens
AISRGDVQSL

SEQ ID NO: 128 - protein, 10 aa, Homo sapiens
NAQRGDHGQL

SEQ ID NO: 129 - protein, 10 aa, Homo sapiens
LNSRGDQASV

SEQ ID NO: 130 - protein, 10 aa, Homo sapiens
SGNRGDIGTF

SEQ ID NO: 131 - protein, 10 aa, Homo sapiens
NVGRGDQATM

SEQ ID NO: 132 - protein, 10 aa, Homo sapiens
SYSRGDTGRL

SEQ ID NO: 133 - protein, 10 aa, Homo sapiens
SHLRGDQALA

SEQ ID NO: 134 - protein, 10 aa, Homo sapiens
YFARGDMTMN

SEQ ID NO: 135 - protein, 10 aa, Homo sapiens
QSQRGDLNPM

SEQ ID NO: 136 - protein, 10 aa, Homo sapiens
EQRRGDKTEL

SEQ ID NO: 137 - protein, 10 aa, Homo sapiens
VSARGDAGQL

SEQ ID NO: 138 - protein, 10 aa, Homo sapiens
EASRGDKGTH

SEQ ID NO: 139 - protein, 10 aa, Homo sapiens
LSTRGDMGMQ

SEQ ID NO: 140 - protein, 10 aa, Homo sapiens
ANMRGDQLHT

SEQ ID NO: 141 - protein, 10 aa, Homo sapiens
ENTRGDLTHA

SEQ ID NO: 142 - protein, 10 aa, Homo sapiens
IERRGDSAGL

SEQ ID NO: 143 - protein, 10 aa, Homo sapiens
SAMRGDGVSL

SEQ ID NO: 144 - protein, 10 aa, Homo sapiens
TVSRGDQGLS

SEQ ID NO: 145 - protein, 10 aa, Homo sapiens
SIVRGDLNST

SEQ ID NO: 146 - protein, 10 aa, Homo sapiens
DVARGDKTNF

SEQ ID NO: 147 - protein, 10 aa, Homo sapiens
WSQRGDLSGT

SEQ ID NO: 148 - protein, 10 aa, Homo sapiens
LNQRGDVSNM

SEQ ID NO: 149 - protein, 10 aa, Homo sapiens
AALRGDLSGS

SEQ ID NO: 150 - protein, 10 aa, Homo sapiens
RVARGDITDI

SEQ ID NO: 151 - protein, 10 aa, Homo sapiens
STNRGDLNQV

SEQ ID NO: 152 - protein, 10 aa, Homo sapiens
DSRRGDSVSL

SEQ ID NO: 153 - protein, 10 aa, Homo sapiens
SVGRGDQSQM

SEQ ID NO: 154 - protein, 10 aa, Homo sapiens
SNTRGDTNSL

SEQ ID NO: 155 - protein, 10 aa, Homo sapiens
DNRRGDGTTM

SEQ ID NO: 156 - protein, 10 aa, Homo sapiens
TPSRGDQGRL

SEQ ID NO: 157 - protein, 10 aa, Homo sapiens
NYSRGDSMTL

SEQ ID NO: 158 - protein, 10 aa, Homo sapiens
MQARGDAGTL

SEQ ID NO: 159 - protein, 10 aa, Homo sapiens
TQSRGDLSGA

SEQ ID NO: 160 - protein, 10 aa, Homo sapiens
FAQRGDLTGV

SEQ ID NO: 161 - protein, 10 aa, Homo sapiens
APVRGDLIGT

SEQ ID NO: 162 - protein, 10 aa, Homo sapiens
EPKRGDLSNT

SEQ ID NO: 163 - protein, 10 aa, Homo sapiens
SVGRGDTYPL

SEQ ID NO: 164 - protein, 10 aa, Homo sapiens
QTARGDMTGH

SEQ ID NO: 165 - protein, 10 aa, Homo sapiens
AGARGDLENR

SEQ ID NO: 166 - protein, 10 aa, Homo sapiens
MSTRGDLNNV

SEQ ID NO: 167 - protein, 10 aa, Homo sapiens
EKQRGDLNSM

SEQ ID NO: 168 - protein, 10 aa, Homo sapiens
IKTRGDLGHE

SEQ ID NO: 169 - protein, 10 aa, Homo sapiens
IGRRGDMELS

SEQ ID NO: 170 - protein, 10 aa, Homo sapiens
TTGRGDLKEV

SEQ ID NO: 171 - protein, 10 aa, Homo sapiens
TDKRGDQNTV

SEQ ID NO: 172 - protein, 10 aa, Homo sapiens
SNVRGDREAV

SEQ ID NO: 173 - protein, 10 aa, Homo sapiens
YANRGDLSHQ

SEQ ID NO: 174 - protein, 10 aa, Homo sapiens
SMMRGDAGQL

SEQ ID NO: 175 - protein, 10 aa, Homo sapiens
EARRGDSQGL

SEQ ID NO: 176 - protein, 10 aa, Homo sapiens
MPTRGDQTKF

SEQ ID NO: 177 - protein, 10 aa, Homo sapiens
AEGRGDQYTR

SEQ ID NO: 178 - protein, 10 aa, Homo sapiens
GPGRGDQTTL

SEQ ID NO: 179 - protein, 10 aa, Homo sapiens
ATGRGDLGQA

SEQ ID NO: 180 - protein, 10 aa, Homo sapiens
AVARGDQGLI

SEQ ID NO: 181 - DNA, 30 bp, Homo sapiens
  source 1..30: mol_type = other DNA
gctgagggtagaggagaccagtatactcgt

SEQ ID NO: 182 - DNA, 30 bp, Homo sapiens
  source 1..30: mol_type = other DNA
gggccgggtagaggagaccagactacgttg

SEQ ID NO: 183 - DNA, 30 bp, Homo sapiens
  source 1..30: mol_type = other DNA
gcggaaggcagaggagaccaatacacaagg

SEQ ID NO: 184 - DNA, 30 bp, Homo sapiens
  source 1..30: mol_type = other DNA
gcgactggtagaggagacctgggtcaggct

SEQ ID NO: 185 - DNA, 30 bp, Homo sapiens
  source 1..30: mol_type = other DNA
gcggtggcgagaggagaccagggtcttatt

SEQ ID NO: 186 - DNA, 30 bp, Homo sapiens
  source 1..30: mol_type = other DNA
aacatctccagaggagaccaaggttaccaa

SEQ ID NO: 187 - DNA, 30 bp, Homo sapiens
  source 1..30: mol_type = other DNA
gcgccggctagaggagaccaggggagtcag

SEQ ID NO: 188 - DNA, 30 bp, Homo sapiens
  source 1..30: mol_type = other DNA
gccgttagcagaggagaccggatggaattc

SEQ ID NO: 189 - DNA, 30 bp, Homo sapiens
  source 1..30: mol_type = other DNA
cagatgggtagaggagacatggggattaag

SEQ ID NO: 190 - DNA, 30 bp, Homo sapiens
  source 1..30: mol_type = other DNA
gagtatcggagaggagacaaggcggatatt

SEQ ID NO: 191 - DNA, 30 bp, Homo sapiens
  source 1..30: mol_type = other DNA
gagagtcggagaggagacaaggagccgctg

SEQ ID NO: 192 - DNA, 30 bp, Homo sapiens
  source 1..30: mol_type = other DNA
gcacacatgagaggagacctaggcggcacg

SEQ ID NO: 193 - DNA, 30 bp, Homo sapiens
  source 1..30: mol_type = other DNA
gtgtggcaaagaggagacaaaatggacatg

SEQ ID NO: 194 - DNA, 30 bp, Homo sapiens
  source 1..30: mol_type = other DNA
tctattgggagaggagacacgggtcatatg

SEQ ID NO: 195 - DNA, 30 bp, Homo sapiens
  source 1..30: mol_type = other DNA
aatattgctagaggagacgctggtcagtat

SEQ ID NO: 196 - DNA, 30 bp, Homo sapiens
  source 1..30: mol_type = other DNA
gcagtagcaagaggagaccaaggcttaatc

SEQ ID NO: 197 - DNA, 30 bp, Homo sapiens
  source 1..30: mol_type = other DNA
tcggtctcgagaggagaccaaggattgcac

SEQ ID NO: 198 - DNA, 30 bp, Homo sapiens
  source 1..30: mol_type = other DNA
gaatacaggagaggagacaaagcagacatc

SEQ ID NO: 199 - DNA, 30 bp, Homo sapiens
  source 1..30: mol_type = other DNA
gcgcatatgagaggagacttgggggggact

SEQ ID NO: 200 - DNA, 30 bp, Homo sapiens
  source 1..30: mol_type = other DNA
cagattggtagaggagacattactcatggg

SEQ ID NO: 201 - DNA, 30 bp, Homo sapiens
  source 1..30: mol_type = other DNA
gaagtcagaagaggagacttgcacgggaca

SEQ ID NO: 202 - DNA, 30 bp, Homo sapiens
  source 1..30: mol_type = other DNA
agtgtctcaagaggagacgtgcacacgatg

SEQ ID NO: 203 - DNA, 30 bp, Homo sapiens
  source 1..30: mol_type = other DNA
atggtgactagaggagaccttggtacgcgg

SEQ ID NO: 204 - DNA, 30 bp, Homo sapiens
  source 1..30: mol_type = other DNA
aacggcgggagaggagacacgacgcacttc

SEQ ID NO: 205 - DNA, 30 bp, Homo sapiens
  source 1..30: mol_type = other DNA
acgatggggagaggagacatgaattcgctt

SEQ ID NO: 206 - DNA, 30 bp, Homo sapiens
  source 1..30: mol_type = other DNA
gcgattagtagaggagaccagggtctttct

SEQ ID NO: 207 - DNA, 30 bp, Homo sapiens
  source 1..30: mol_type = other DNA
aacgacgcaagaggagacaaatccacatac

SEQ ID NO: 208 - DNA, 30 bp, Homo sapiens
  source 1..30: mol_type = other DNA
gtggggctgagaggagacttgacgggttcg

SEQ ID NO: 209 - DNA, 30 bp, Homo sapiens
  source 1..30: mol_type = other DNA
gagatgaggagaggagacctggggacgagt

SEQ ID NO: 210 - DNA, 30 bp, Homo sapiens
  source 1..30: mol_type = other DNA
gcgatgagtagaggagacatggggatggcg

SEQ ID NO: 211 - DNA, 30 bp, Homo sapiens
  source 1..30: mol_type = other DNA
gcaacgggcagaggagacttaggccaagca

SEQ ID NO: 212 - DNA, 30 bp, Homo sapiens
  source 1..30: mol_type = other DNA
aacgtagcaagaggagaccaagttaactac

SEQ ID NO: 213 - DNA, 30 bp, Homo sapiens
  source 1..30: mol_type = other DNA
agcatcggaagaggagacaccggccacatg

SEQ ID NO: 214 - DNA, 30 bp, Homo sapiens
  source 1..30: mol_type = other DNA
gcatccgtaagaggagacctatcaggctca

SEQ ID NO: 215 - DNA, 30 bp, Homo sapiens
  source 1..30: mol_type = other DNA
agcgccgcgagaggagacactgaacggcta

SEQ ID NO: 216 - DNA, 30 bp, Homo sapiens
  source 1..30: mol_type = other DNA
gccatgtcaagaggagacatgggtatggct

SEQ ID NO: 217 - DNA, 30 bp, Homo sapiens
  source 1..30: mol_type = other DNA
agtccatcgagaggagaccaaggacgcact

SEQ ID NO: 218 - DNA, 30 bp, Homo sapiens
  source 1..30: mol_type = other DNA
gtgcctgggagaggagaccttaatactatg

SEQ ID NO: 219 - DNA, 30 bp, Homo sapiens
  source 1..30: mol_type = other DNA
ggtcctgggagaggagaccaaacgaccctt

SEQ ID NO: 220 - DNA, 30 bp, Homo sapiens
  source 1..30: mol_type = other DNA
acatcggtcagaggagaccacggaacattg

SEQ ID NO: 221 - DNA, 30 bp, Homo sapiens
  source 1..30: mol_type = other DNA
acgccttcgagaggagaccttgggcagact

SEQ ID NO: 222 - DNA, 30 bp, Homo sapiens
  source 1..30: mol_type = other DNA
gctggacacagaggagacacaggagtcatc

SEQ ID NO: 223 - DNA, 30 bp, Homo sapiens
  source 1..30: mol_type = other DNA
gcgccggcgagaggagaccagggtagtatg

SEQ ID NO: 224 - DNA, 30 bp, Homo sapiens
  source 1..30: mol_type = other DNA
atgtcgttgagaggagacttgaatgggtcg

SEQ ID NO: 225 - DNA, 30 bp, Homo sapiens
  source 1..30: mol_type = other DNA
gaggctaagagaggagacgtgcattctatt

SEQ ID NO: 226 - DNA, 30 bp, Homo sapiens
  source 1..30: mol_type = other DNA
agtgctcagagaggagacgtgcaggcggtg

SEQ ID NO: 227 - DNA, 30 bp, Homo sapiens
  source 1..30: mol_type = other DNA
agtacggcgagaggagaccagggggatagg

SEQ ID NO: 228 - DNA, 30 bp, Homo sapiens
  source 1..30: mol_type = other DNA
cagattagtagaggagacctggggattaat

SEQ ID NO: 229 - DNA, 30 bp, Homo sapiens
  source 1..30: mol_type = other DNA
acgttcacaagaggagacatgacgatgaac

SEQ ID NO: 230 - DNA, 30 bp, Homo sapiens
  source 1..30: mol_type = other DNA
caagaaggaagaggagacctgaacatgagg

SEQ ID NO: 231 - DNA, 30 bp, Homo sapiens
  source 1..30: mol_type = other DNA
gcacacgcaagaggagacaccagctccctg

SEQ ID NO: 232 - DNA, 30 bp, Homo sapiens
  source 1..30: mol_type = other DNA
acgaatgggagaggagacgctgggactctg

SEQ ID NO: 233 - DNA, 30 bp, Homo sapiens
  source 1..30: mol_type = other DNA
gctttcggcagaggagaccaagggcaacta

SEQ ID NO: 234 - DNA, 30 bp, Homo sapiens
  source 1..30: mol_type = other DNA
gctgatgggagaggagaccgttcgtctctg

SEQ ID NO: 235 - DNA, 30 bp, Homo sapiens
  source 1..30: mol_type = other DNA
gacggacgcagaggagacggacacagcctt

SEQ ID NO: 236 - DNA, 30 bp, Homo sapiens
  source 1..30: mol_type = other DNA
gagaagttgagaggagaccttcattcgact

SEQ ID NO: 237 - DNA, 30 bp, Homo sapiens
  source 1..30: mol_type = other DNA
gaggcgcgtagaggagacgcttcggcgatg

SEQ ID NO: 238 - DNA, 30 bp, Homo sapiens
  source 1..30: mol_type = other DNA
atgggaacgagaggagacaaaatggacttc

SEQ ID NO: 239 - DNA, 30 bp, Homo sapiens
  source 1..30: mol_type = other DNA
tcgacttcgagaggagaccgggagtcgtat

SEQ ID NO: 240 - DNA, 30 bp, Homo sapiens
  source 1..30: mol_type = other DNA
tgtcagccgagaggagacacgactcggtgt

SEQ ID NO: 241 - DNA, 30 bp, Homo sapiens
  source 1..30: mol_type = other DNA
atgcacacgagaggagacaaaatggacttc

SEQ ID NO: 242 - DNA, 30 bp, Homo sapiens
  source 1..30: mol_type = other DNA
tacagtgccagaggagacacaagtggcctg

SEQ ID NO: 243 - DNA, 30 bp, Homo sapiens
  source 1..30: mol_type = other DNA
tcgacgatgagaggagaccatgagaagttg

SEQ ID NO: 244 - DNA, 30 bp, Homo sapiens
  source 1..30: mol_type = other DNA
acccaaatgagaggagacggggtcacacta

SEQ ID NO: 245 - DNA, 30 bp, Homo sapiens
  source 1..30: mol_type = other DNA
actactcgtagaggagacatgggggataat

SEQ ID NO: 246 - DNA, 30 bp, Homo sapiens
  source 1..30: mol_type = other DNA
gcgaatggtagaggagacaggctggagttg

SEQ ID NO: 247 - DNA, 30 bp, Homo sapiens
  source 1..30: mol_type = other DNA
gctgtgtctagaggagacaggatggagttt

SEQ ID NO: 248 - DNA, 30 bp, Homo sapiens
  source 1..30: mol_type = other DNA
gagaatcagagaggagacttgtcgggtcgg

SEQ ID NO: 249 - DNA, 30 bp, Homo sapiens
  source 1..30: mol_type = other DNA
tcgactggtagaggagacctggggcaggct

SEQ ID NO: 250 - DNA, 30 bp, Homo sapiens
  source 1..30: mol_type = other DNA
atgcatactagaggagacaagatggatttt

SEQ ID NO: 251 - DNA, 30 bp, Homo sapiens
  source 1..30: mol_type = other DNA
agtagtgcgagaggagactatagtgaggtg

SEQ ID NO: 252 - DNA, 30 bp, Homo sapiens
  source 1..30: mol_type = other DNA
gcgtttgggagaggagaccagggtcagctt

SEQ ID NO: 253 - DNA, 30 bp, Homo sapiens
  source 1..30: mol_type = other DNA
agtacggcgagaggagacgcggcgacttat

SEQ ID NO: 254 - DNA, 30 bp, Homo sapiens
  source 1..30: mol_type = other DNA
gtcaacacaagaggagacactcaaaaactt

SEQ ID NO: 255 - DNA, 30 bp, Homo sapiens
  source 1..30: mol_type = other DNA
gcgatgagtagaggagaccatgcgtcgctt

SEQ ID NO: 256 - DNA, 30 bp, Homo sapiens
  source 1..30: mol_type = other DNA
caggctgctagaggagacgttaataagctg

SEQ ID NO: 257 - DNA, 30 bp, Homo sapiens
  source 1..30: mol_type = other DNA
aggggtacgagaggagacacggttgagttg

SEQ ID NO: 258 - DNA, 30 bp, Homo sapiens
  source 1..30: mol_type = other DNA
accacccggagaggagacatgggagacaac

SEQ ID NO: 259 - DNA, 30 bp, Homo sapiens
  source 1..30: mol_type = other DNA
gctagtacgagaggagactatgcgggtgtt

SEQ ID NO: 260 - DNA, 30 bp, Homo sapiens
  source 1..30: mol_type = other DNA
gacgacaggagaggagacaaactgcccctt

SEQ ID NO: 261 - DNA, 30 bp, Homo sapiens
  source 1..30: mol_type = other DNA
tcggggtcgagaggagacctgaatgcggtg

SEQ ID NO: 262 - DNA, 30 bp, Homo sapiens
  source 1..30: mol_type = other DNA
gagtataggagaggagaccagcagattcag

SEQ ID NO: 263 - DNA, 30 bp, Homo sapiens
  source 1..30: mol_type = other DNA
acgtacgtcagaggagacagagcagaagtg

SEQ ID NO: 264 - DNA, 30 bp, Homo sapiens
  source 1..30: mol_type = other DNA
caatcactaagaggagacttgaacggatcc

SEQ ID NO: 265 - DNA, 30 bp, Homo sapiens
  source 1..30: mol_type = other DNA
gagggtcggagaggagacacgacgtcgttg

SEQ ID NO: 266 - DNA, 30 bp, Homo sapiens
  source 1..30: mol_type = other DNA
gcggagaatagaggagaccggtcgagtttg

SEQ ID NO: 267 - DNA, 30 bp, Homo sapiens
  source 1..30: mol_type = other DNA
gtggctgggagaggagaccgtagtgagctg

SEQ ID NO: 268 - DNA, 30 bp, Homo sapiens
  source 1..30: mol_type = other DNA
gatccggcgagaggagacattggtgcgcgt

SEQ ID NO: 269 - DNA, 30 bp, Homo sapiens
  source 1..30: mol_type = other DNA
gaaatgcgcagaggagacttgggaacttca

SEQ ID NO: 270 - DNA, 30 bp, Homo sapiens
  source 1..30: mol_type = other DNA
gtttggcagagaggagacaagatggatatg

SEQ ID NO: 271 - DNA, 30 bp, Homo sapiens
  source 1..30: mol_type = other DNA
gtgattgggagaggagacaaggcgcttcag

SEQ ID NO: 272 - DNA, 30 bp, Homo sapiens
  source 1..30: mol_type = other DNA
agcttcgaaagaggagacaaaaactctctt

SEQ ID NO: 273 - DNA, 30 bp, Homo sapiens
  source 1..30: mol_type = other DNA
cagattgctagaggagacattgctagtgtg

SEQ ID NO: 274 - DNA, 30 bp, Homo sapiens
  source 1..30: mol_type = other DNA
acgattagtagaggagacctgggtggggct

SEQ ID NO: 275 - DNA, 30 bp, Homo sapiens
  source 1..30: mol_type = other DNA
gcgatgattagaggagacatgacgcatagt

SEQ ID NO: 276 - DNA, 30 bp, Homo sapiens
  source 1..30: mol_type = other DNA
acgagtgtgagaggagaccatgggacgctg

SEQ ID NO: 277 - DNA, 30 bp, Homo sapiens
  source 1..30: mol_type = other DNA
acgatgcggagaggagacctgaatgatagt

SEQ ID NO: 278 - DNA, 30 bp, Homo sapiens
  source 1..30: mol_type = other DNA
gcctactccagaggagacctaggcaaccac

SEQ ID NO: 279 - DNA, 30 bp, Homo sapiens
  source 1..30: mol_type = other DNA
gaggagcggagaggagacactcatcggctg

SEQ ID NO: 280 - DNA, 30 bp, Homo sapiens
  source 1..30: mol_type = other DNA
tcacaactaagaggagacgcggggacaatc

SEQ ID NO: 281 - DNA, 30 bp, Homo sapiens
  source 1..30: mol_type = other DNA
gcgatggtgagaggagacgcgcatcagctg

SEQ ID NO: 282 - DNA, 30 bp, Homo sapiens
  source 1..30: mol_type = other DNA
acgactagtagaggagacttgaattcggtt

SEQ ID NO: 283 - DNA, 30 bp, Homo sapiens
  source 1..30: mol_type = other DNA
tgggagcagagaggagacatgattgggaag

SEQ ID NO: 284 - DNA, 30 bp, Homo sapiens
  source 1..30: mol_type = other DNA
tacaccaccagaggagacctccaatcaaac

SEQ ID NO: 285 - DNA, 30 bp, Homo sapiens
  source 1..30: mol_type = other DNA
atgtcgcagagaggagacctttcgcatcag

SEQ ID NO: 286 - DNA, 30 bp, Homo sapiens
  source 1..30: mol_type = other DNA
gcgagtactagaggagacacgacgcatatg

SEQ ID NO: 287 - DNA, 30 bp, Homo sapiens
  source 1..30: mol_type = other DNA
tcagacaccagaggagacacccacagactg

SEQ ID NO: 288 - DNA, 30 bp, Homo sapiens
  source 1..30: mol_type = other DNA
acgctgggtagaggagaccagtatacgagt

SEQ ID NO: 289 - DNA, 30 bp, Homo sapiens
  source 1..30: mol_type = other DNA
tcgtatgcgagaggagacgttagggaggtt

SEQ ID NO: 290 - DNA, 30 bp, Homo sapiens
  source 1..30: mol_type = other DNA
gagactcgtagaggagacctgagtcagaat

SEQ ID NO: 291 - DNA, 30 bp, Homo sapiens
  source 1..30: mol_type = other DNA
acaatcgggagaggagacctgatggacaaa

SEQ ID NO: 292 - DNA, 30 bp, Homo sapiens
  source 1..30: mol_type = other DNA
agttacaccagaggagaccttagccaaaac

SEQ ID NO: 293 - DNA, 30 bp, Homo sapiens
  source 1..30: mol_type = other DNA
tcgaagctgagaggagacgctagtgagatt

SEQ ID NO: 294 - DNA, 30 bp, Homo sapiens
  source 1..30: mol_type = other DNA
tcgatggcgagaggagacaaggcggagatt

SEQ ID NO: 295 - DNA, 30 bp, Homo sapiens
  source 1..30: mol_type = other DNA
ggagccgtcagaggagaccactcatcgtta

SEQ ID NO: 296 - DNA, 30 bp, Homo sapiens
  source 1..30: mol_type = other DNA
atggagacgagaggagacatgagtaatcgt

SEQ ID NO: 297 - DNA, 30 bp, Homo sapiens
  source 1..30: mol_type = other DNA
gcgtacacaagaggagacaaagacgcacta

SEQ ID NO: 298 - DNA, 30 bp, Homo sapiens
  source 1..30: mol_type = other DNA
gagaatcggagaggagacattacgcagtcg

SEQ ID NO: 299 - DNA, 30 bp, Homo sapiens
  source 1..30: mol_type = other DNA
tccacgggcagaggagaccttggacaagca

SEQ ID NO: 300 - DNA, 30 bp, Homo sapiens
  source 1..30: mol_type = other DNA
ggtgctgttagaggagaccatagtagtctt

SEQ ID NO: 301 - DNA, 30 bp, Homo sapiens
  source 1..30: mol_type = other DNA
tctcagcttagaggagacgctggtacgatt

SEQ ID NO: 302 - DNA, 30 bp, Homo sapiens
  source 1..30: mol_type = other DNA
atgctcctaagaggagacaaaaacgacttc

SEQ ID NO: 303 - DNA, 30 bp, Homo sapiens
  source 1..30: mol_type = other DNA
aaggtgcagagaggagacgcgagtgattgg

SEQ ID NO: 304 - DNA, 30 bp, Homo sapiens
  source 1..30: mol_type = other DNA
gcgtttacgagaggagacatgcagactgct

SEQ ID NO: 305 - DNA, 30 bp, Homo sapiens
  source 1..30: mol_type = other DNA
cagcctaagagaggagacgttcaggctcag

SEQ ID NO: 306 - DNA, 30 bp, Homo sapiens
  source 1..30: mol_type = other DNA
gctagtctgagaggagacatgggggtgagt

SEQ ID NO: 307 - DNA, 30 bp, Homo sapiens
  source 1..30: mol_type = other DNA
gcctactccagaggagacaaagaatcgttc

SEQ ID NO: 308 - DNA, 30 bp, Homo sapiens
  source 1..30: mol_type = other DNA
cggttccaaagaggagacttgaccgacgct

SEQ ID NO: 309 - DNA, 30 bp, Homo sapiens
  source 1..30: mol_type = other DNA
tctgatgctagaggagacacgatgaagctg

SEQ ID NO: 310 - DNA, 30 bp, Homo sapiens
  source 1..30: mol_type = other DNA
agcggggccagaggagacacagtaagtctc

SEQ ID NO: 311 - DNA, 30 bp, Homo sapiens
  source 1..30: mol_type = other DNA
gatgagaagagaggagaccagaagcatctt

SEQ ID NO: 312 - DNA, 30 bp, Homo sapiens
  source 1..30: mol_type = other DNA
aacggagtaagaggagacgtccaaaacttc

SEQ ID NO: 313 - DNA, 30 bp, Homo sapiens
  source 1..30: mol_type = other DNA
atgacgttgagaggagacctgggtgggtcg

SEQ ID NO: 314 - DNA, 30 bp, Homo sapiens
  source 1..30: mol_type = other DNA
gaggctaggagaggagacgctagtgcgatg

SEQ ID NO: 315 - DNA, 30 bp, Homo sapiens
  source 1..30: mol_type = other DNA
gggcaggagagaggagacctggggactcgg

SEQ ID NO: 316 - DNA, 30 bp, Homo sapiens
  source 1..30: mol_type = other DNA
agaggcgccagaggagacctagtcgacgct

SEQ ID NO: 317 - DNA, 30 bp, Homo sapiens
  source 1..30: mol_type = other DNA
caatactccagaggagaccacaccgacctt

SEQ ID NO: 318 - DNA, 30 bp, Homo sapiens
  source 1..30: mol_type = other DNA
atgaacaccagaggagacgtgcacgctatg

SEQ ID NO: 319 - DNA, 30 bp, Homo sapiens
  source 1..30: mol_type = other DNA
acggtacggagaggagacctagcaaccgaa

SEQ ID NO: 320 - DNA, 30 bp, Homo sapiens
  source 1..30: mol_type = other DNA
aatattgcgagaggagacgctggtcagtat

SEQ ID NO: 321 - DNA, 30 bp, Homo sapiens
  source 1..30: mol_type = other DNA
tctgttgggagaggagacaaggctgatatt

SEQ ID NO: 322 - DNA, 30 bp, Homo sapiens
  source 1..30: mol_type = other DNA
tcacacaacagaggagacaccggaaccatg

SEQ ID NO: 323 - DNA, 30 bp, Homo sapiens
  source 1..30: mol_type = other DNA
acgaacaccagaggagacaaagaatcagta

SEQ ID NO: 324 - DNA, 30 bp, Homo sapiens
  source 1..30: mol_type = other DNA
tcgattggtagaggagaccagtatacgatt

SEQ ID NO: 325 - DNA, 30 bp, Homo sapiens
  source 1..30: mol_type = other DNA
cagacgagtagaggagacgcggggtcttgg

SEQ ID NO: 326 - DNA, 30 bp, Homo sapiens
  source 1..30: mol_type = other DNA
gctgaaaacagaggagaccgaagcagccta

SEQ ID NO: 327 - DNA, 30 bp, Homo sapiens
  source 1..30: mol_type = other DNA
gcgatttcgagaggagacgttcagtcgttg

SEQ ID NO: 328 - DNA, 30 bp, Homo sapiens
  source 1..30: mol_type = other DNA
aacgcacaaagaggagaccacgggcaactg

SEQ ID NO: 329 - DNA, 30 bp, Homo sapiens
  source 1..30: mol_type = other DNA
ctcaactcgagaggagaccaagcctccgtc

SEQ ID NO: 330 - DNA, 30 bp, Homo sapiens
  source 1..30: mol_type = other DNA
tcgggaaacagaggagacataggtacattc

SEQ ID NO: 331 - DNA, 30 bp, Homo sapiens
  source 1..30: mol_type = other DNA
aacgtgggaagaggagaccaagccacaatg

SEQ ID NO: 332 - DNA, 30 bp, Homo sapiens
  source 1..30: mol_type = other DNA
tcgtattctagaggagacactggtcggctt

SEQ ID NO: 333 - DNA, 30 bp, Homo sapiens
  source 1..30: mol_type = other DNA
agccacttaagaggagaccaagcgttggcc

SEQ ID NO: 334 - DNA, 30 bp, Homo sapiens
  source 1..30: mol_type = other DNA
tacttcgccagaggagacatgactatgaac

SEQ ID NO: 335 - DNA, 30 bp, Homo sapiens
  source 1..30: mol_type = other DNA
cagtcgcagagaggagaccttaatcctatg

SEQ ID NO: 336 - DNA, 30 bp, Homo sapiens
  source 1..30: mol_type = other DNA
gagcagaggagaggagacaagacggagctg

SEQ ID NO: 337 - DNA, 30 bp, Homo sapiens
  source 1..30: mol_type = other DNA
gtgtctgcgagaggagacgcgggtcagttg

SEQ ID NO: 338 - DNA, 30 bp, Homo sapiens
  source 1..30: mol_type = other DNA
tctccgtctagaggagaccaggggcggacg

SEQ ID NO: 339 - DNA, 30 bp, Homo sapiens
  source 1..30: mol_type = other DNA
gaggctagtagaggagacaagggtacgcat

SEQ ID NO: 340 - DNA, 30 bp, Homo sapiens
  source 1..30: mol_type = other DNA
ctgtctactagaggagacatgggtatgcag

SEQ ID NO: 341 - DNA, 30 bp, Homo sapiens
  source 1..30: mol_type = other DNA
gcgaatatgagaggagaccagctgcatact

SEQ ID NO: 342 - DNA, 30 bp, Homo sapiens
  source 1..30: mol_type = other DNA
gagaatacgagaggagaccttactcatgcg

SEQ ID NO: 343 - DNA, 30 bp, Homo sapiens
  source 1..30: mol_type = other DNA
attgagcgtagaggagactcggcggggctg

SEQ ID NO: 344 - DNA, 30 bp, Homo sapiens
  source 1..30: mol_type = other DNA
agtgcgatgagaggagacggagtgagcttg

SEQ ID NO: 345 - DNA, 30 bp, Homo sapiens
  source 1..30: mol_type = other DNA
acggtttcgagaggagaccaggggttgtct

SEQ ID NO: 346 - DNA, 30 bp, Homo sapiens
  source 1..30: mol_type = other DNA
tcgatcgtcagaggagacctgaactccacg

SEQ ID NO: 347 - DNA, 30 bp, Homo sapiens
  source 1..30: mol_type = other DNA
gacgtagcgagaggagacaaaacaaacttc

SEQ ID NO: 348 - DNA, 30 bp, Homo sapiens
  source 1..30: mol_type = other DNA
tggagccaaagaggagacctgagtggaacc

SEQ ID NO: 349 - DNA, 30 bp, Homo sapiens
  source 1..30: mol_type = other DNA
ctgaaccaaagaggagacgtttccaacatg

SEQ ID NO: 350 - DNA, 30 bp, Homo sapiens
  source 1..30: mol_type = other DNA
gccgccctaagaggagacctatccggctca

SEQ ID NO: 351 - DNA, 30 bp, Homo sapiens
  source 1..30: mol_type = other DNA
agggttgcgagaggagacattacggatatt

SEQ ID NO: 352 - DNA, 30 bp, Homo sapiens
  source 1..30: mol_type = other DNA
tcgacgaacagaggagacctcaaccaagtt

SEQ ID NO: 353 - DNA, 30 bp, Homo sapiens
  source 1..30: mol_type = other DNA
gattcgcggagaggagactctgtgagtctt

SEQ ID NO: 354 - DNA, 30 bp, Homo sapiens
  source 1..30: mol_type = other DNA
agtgttgggagaggagaccagtctcagatg

SEQ ID NO: 355 - DNA, 30 bp, Homo sapiens
  source 1..30: mol_type = other DNA
tccaacacgagaggagacacaaactcccta

SEQ ID NO: 356 - DNA, 30 bp, Homo sapiens
  source 1..30: mol_type = other DNA
gataatcggagaggagacggtacgactatg

SEQ ID NO: 357 - DNA, 30 bp, Homo sapiens
  source 1..30: mol_type = other DNA
acgccttcgagaggagaccaaggaagacta

SEQ ID NO: 358 - DNA, 30 bp, Homo sapiens
  source 1..30: mol_type = other DNA
aactacagcagaggagactcaatgacgctt

SEQ ID NO: 359 - DNA, 30 bp, Homo sapiens
  source 1..30: mol_type = other DNA
atgcaggctagaggagacgctgggactctg

SEQ ID NO: 360 - DNA, 30 bp, Homo sapiens
  source 1..30: mol_type = other DNA
acgcagtcgagaggagacttgtctggtgcg

SEQ ID NO: 361 - DNA, 30 bp, Homo sapiens
  source 1..30: mol_type = other DNA
tttgcgcagagaggagacttgactggggtt

SEQ ID NO: 362 - DNA, 30 bp, Homo sapiens
  source 1..30: mol_type = other DNA
gcgccggtgagaggagacctgattggtacg

SEQ ID NO: 363 - DNA, 30 bp, Homo sapiens
  source 1..30: mol_type = other DNA
tcgaatactagaggagacacgaatagtttg

SEQ ID NO: 364 - DNA, 30 bp, Homo sapiens
  source 1..30: mol_type = other DNA
gagccgaagagaggagacttgagtaatacg

SEQ ID NO: 365 - DNA, 30 bp, Homo sapiens
  source 1..30: mol_type = other DNA
tcggttgggagaggagacacttatcctctg

SEQ ID NO: 366 - DNA, 30 bp, Homo sapiens
  source 1..30: mol_type = other DNA
caaacagccagaggagacatgactggccac

SEQ ID NO: 367 - DNA, 30 bp, Homo sapiens
  source 1..30: mol_type = other DNA
gctggtgcgagaggagacttggagaatcgg

SEQ ID NO: 368 - DNA, 30 bp, Homo sapiens
  source 1..30: mol_type = other DNA
atgagcactagaggagacctaaacaacgtc

SEQ ID NO: 369 - DNA, 30 bp, Homo sapiens
  source 1..30: mol_type = other DNA
gaaaaacaaagaggagacctcaacagcatg

SEQ ID NO: 370 - DNA, 30 bp, Homo sapiens
  source 1..30: mol_type = other DNA
attaagacgagaggagaccttggtcatgag

SEQ ID NO: 371 - DNA, 30 bp, Homo sapiens
  source 1..30: mol_type = other DNA
attgggaggagaggagacatggagttgtct

SEQ ID NO: 372 - DNA, 30 bp, Homo sapiens
  source 1..30: mol_type = other DNA
actacgggtagaggagacttgaaggaggtt

SEQ ID NO: 373 - DNA, 30 bp, Homo sapiens
  source 1..30: mol_type = other DNA
acggataagagaggagaccagaatactgtg

SEQ ID NO: 374 - DNA, 30 bp, Homo sapiens
  source 1..30: mol_type = other DNA
tcgaatgttagaggagaccgggaggcggtt

SEQ ID NO: 375 - DNA, 30 bp, Homo sapiens
  source 1..30: mol_type = other DNA
tcagtcggcagaggagaccaaagtcaaatg

SEQ ID NO: 376 - DNA, 30 bp, Homo sapiens
  source 1..30: mol_type = other DNA
tacgccaacagaggagacctgagtcaccaa

SEQ ID NO: 377 - DNA, 30 bp, Homo sapiens
  source 1..30: mol_type = other DNA
caaatatcgagaggagacctaggtatcaac

SEQ ID NO: 378 - DNA, 30 bp, Homo sapiens
  source 1..30: mol_type = other DNA
tcgatgatgagaggagacgctgggcagttg

SEQ ID NO: 379 - DNA, 30 bp, Homo sapiens
  source 1..30: mol_type = other DNA
gaggcgcggagaggagacagtcaggggctg

SEQ ID NO: 380 - DNA, 30 bp, Homo sapiens
  source 1..30: mol_type = other DNA
atgcccacaagaggagaccaaaccaaattc

SEQ ID NO: 381 - protein, 10 aa, Homo sapiens
SNSRGDYNSL

SEQ ID NO: 382 - protein, 10 aa, Homo sapiens
QERRGDYTSM

SEQ ID NO: 383 - protein, 10 aa, Homo sapiens
ASTRGDHGVL

SEQ ID NO: 384 - protein, 10 aa, Homo sapiens
ENRRGDFNNT

SEQ ID NO: 385 - protein, 10 aa, Homo sapiens
AHMRGDLGGT

SEQ ID NO: 386 - protein, 10 aa, Homo sapiens
NGGRGDTTHF

SEQ ID NO: 387 - protein, 10 aa, Homo sapiens
SAARGDTERL

SEQ ID NO: 388 - protein, 10 aa, Homo sapiens
TSVRGDHGTL

SEQ ID NO: 389 - protein, 10 aa, Homo sapiens
EKLRGDLHST

SEQ ID NO: 390 - protein, 10 aa, Homo sapiens
ENQRGDLSGR

SEQ ID NO: 391 - protein, 10 aa, Homo sapiens
AMIRGDMTHS

SEQ ID NO: 392 - protein, 10 aa, Homo sapiens
EERRGDTHRL

SEQ ID NO: 393 - protein, 10 aa, Homo sapiens
ENRRGDITQS

SEQ ID NO: 394 - protein, 10 aa, Homo sapiens
NAQRGDHGQL

SEQ ID NO: 395 - protein, 10 aa, Homo sapiens
SHLRGDQALA

SEQ ID NO: 396 - protein, 10 aa, Homo sapiens
SNVRGDREAV

SEQ ID NO: 397 - protein, 10 aa, Homo sapiens
SSARGDRDYL

SEQ ID NO: 398 - protein, 10 aa, Homo sapiens
STVRGDYTSV

SEQ ID NO: 399 - protein, 10 aa, Homo sapiens
SYQRGDQHNM

SEQ ID NO: 400 - protein, 10 aa, Homo sapiens
NDTRGDRSYM

SEQ ID NO: 401 - protein, 10 aa, Homo sapiens
SGSRGDLSGH

SEQ ID NO: 402 - protein, 10 aa, Homo sapiens
DKPRGDRQLL

SEQ ID NO: 403 - protein, 10 aa, Homo sapiens
SNNRGDYTTM

SEQ ID NO: 404 - protein, 10 aa, Homo sapiens
QGGRGDLSTL

SEQ ID NO: 405 - protein, 10 aa, Homo sapiens
SKDRGDYQTL

SEQ ID NO: 406 - protein, 10 aa, Homo sapiens
ATARGDQMKL

SEQ ID NO: 407 - protein, 10 aa, Homo sapiens
QHMRGDLTGA

SEQ ID NO: 408 - protein, 10 aa, Homo sapiens
DGTRGDLGRY

SEQ ID NO: 409 - protein, 10 aa, Homo sapiens
DSRRGDYANL

SEQ ID NO: 410 - protein, 10 aa, Homo sapiens
SAMRGDYTST

SEQ ID NO: 411 - protein, 10 aa, Homo sapiens
ENRRGDAQLL

SEQ ID NO: 412 - protein, 10 aa, Homo sapiens
TTLRGDHSQL

SEQ ID NO: 413 - protein, 10 aa, Homo sapiens
DAGRGDMHRM

SEQ ID NO: 414 - protein, 10 aa, Homo sapiens
QHTRGDLSST

SEQ ID NO: 415 - protein, 10 aa, Homo sapiens
QVYRGDRESV

SEQ ID NO: 416 - protein, 10 aa, Homo sapiens
SSQRGDLAGT

SEQ ID NO: 417 - protein, 10 aa, Homo sapiens
TDVRGDRGTF

SEQ ID NO: 418 - protein, 10 aa, Homo sapiens
SSVRGDREVT

SEQ ID NO: 419 - protein, 10 aa, Homo sapiens
ENRRGDLTNA

SEQ ID NO: 420 - protein, 10 aa, Homo sapiens
SYARGDVHSI

SEQ ID NO: 421 - protein, 10 aa, Homo sapiens
DSSRGDLNLR

SEQ ID NO: 422 - protein, 10 aa, Homo sapiens
SNARGDYSNM

SEQ ID NO: 423 - protein, 10 aa, Homo sapiens
SSVRGDHSVL

SEQ ID NO: 424 - protein, 10 aa, Homo sapiens
SAFRGDLHAT

SEQ ID NO: 425 - protein, 10 aa, Homo sapiens
SDRRGDQLLY

SEQ ID NO: 426 - protein, 10 aa, Homo sapiens
SGVRGDRLAV

SEQ ID NO: 427 - protein, 10 aa, Homo sapiens
ASLRGDLSST

SEQ ID NO: 428 - protein, 10 aa, Homo sapiens
TNGRGDLHGM

SEQ ID NO: 429 - protein, 10 aa, Homo sapiens
TNTRGDHGML

SEQ ID NO: 430 - protein, 10 aa, Homo sapiens
EDRRGDLLRT

SEQ ID NO: 431 - protein, 10 aa, Homo sapiens
SSLRGDLLHS

SEQ ID NO: 432 - protein, 10 aa, Homo sapiens
DGRRGDRESI

SEQ ID NO: 433 - protein, 10 aa, Homo sapiens
TGTRGDTMTW

SEQ ID NO: 434 - protein, 10 aa, Homo sapiens
SATRGDHNVL

SEQ ID NO: 435 - protein, 10 aa, Homo sapiens
TDRRGDSGTL

SEQ ID NO: 436 - protein, 10 aa, Homo sapiens
TSARGDLIHT

SEQ ID NO: 437 - protein, 10 aa, Homo sapiens
AVERGDRLML

SEQ ID NO: 438 - protein, 10 aa, Homo sapiens
DRSRGDTHVL

SEQ ID NO: 439 - protein, 10 aa, Homo sapiens
ESTRGDLRVV

SEQ ID NO: 440 - protein, 10 aa, Homo sapiens
TTIRGDYREL

SEQ ID NO: 441 - protein, 10 aa, Homo sapiens
TFIRGDLAGA

SEQ ID NO: 442 - protein, 10 aa, Homo sapiens
MTSRGDLTTY

SEQ ID NO: 443 - protein, 10 aa, Homo sapiens
ADRRGDQLYS

SEQ ID NO: 444 - protein, 10 aa, Homo sapiens
GYVRGDLGQH

SEQ ID NO: 445 - protein, 10 aa, Homo sapiens
DASRGDRTSL

SEQ ID NO: 446 - protein, 10 aa, Homo sapiens
TEVRGDRSQV

SEQ ID NO: 447 - protein, 10 aa, Homo sapiens
SEVRGDRLSI

SEQ ID NO: 448 - protein, 10 aa, Homo sapiens
SDRRGDLSGA

SEQ ID NO: 449 - protein, 10 aa, Homo sapiens
SVTRGDRVVI

SEQ ID NO: 450 - protein, 10 aa, Homo sapiens
SEGRGDRMAL

SEQ ID NO: 451 - protein, 10 aa, Homo sapiens
SSTRGDHLSL

SEQ ID NO: 452 - protein, 10 aa, Homo sapiens
SSTRGDQIYV

SEQ ID NO: 453 - protein, 10 aa, Homo sapiens
SAQRDRMGSP

SEQ ID NO: 454 - protein, 10 aa, Homo sapiens
TGTRGDIATF

SEQ ID NO: 455 - protein, 10 aa, Homo sapiens
SMQRGDLLST

SEQ ID NO: 456 - protein, 10 aa, Homo sapiens
EDVRGDRSKL

SEQ ID NO: 457 - protein, 10 aa, Homo sapiens
TSVRGDQSQY

SEQ ID NO: 458 - protein, 10 aa, Homo sapiens
ADRRGDTGLF

SEQ ID NO: 459 - protein, 10 aa, Homo sapiens
NTYRGDRDSL

SEQ ID NO: 460 - protein, 10 aa, Homo sapiens
SRGRGDLNDL

SEQ ID NO: 461 - protein, 10 aa, Homo sapiens
MDRRGDASIL

SEQ ID NO: 462 - protein, 10 aa, Homo sapiens
SIWRGDRTEV

SEQ ID NO: 463 - protein, 10 aa, Homo sapiens
NSQRGDYSGM

SEQ ID NO: 464 - protein, 10 aa, Homo sapiens
SSVRGDIGGI

SEQ ID NO: 465 - protein, 10 aa, Homo sapiens
EIRRGDLLAQ

SEQ ID NO: 466 - protein, 10 aa, Homo sapiens
SNIRGDHAVM

SEQ ID NO: 467 - protein, 10 aa, Homo sapiens
LDTRGDRSQV

SEQ ID NO: 468 - protein, 10 aa, Homo sapiens
ANTRGDHQNF

SEQ ID NO: 469 - protein, 10 aa, Homo sapiens
TSVRGDLSMN

SEQ ID NO: 470 - protein, 10 aa, Homo sapiens
TTTRGDYLNT

SEQ ID NO: 471 - protein, 10 aa, Homo sapiens
AERRGDIKEY

SEQ ID NO: 472 - protein, 10 aa, Homo sapiens
SHARGDLGST

SEQ ID NO: 473 - protein, 10 aa, Homo sapiens
TSQRGDYVSL

SEQ ID NO: 474 - protein, 10 aa, Homo sapiens
VNVRGDRGEV

SEQ ID NO: 475 - protein, 10 aa, Homo sapiens
SYRRGDHDQL

SEQ ID NO: 476 - protein, 10 aa, Homo sapiens
SGTRGDRVDL

SEQ ID NO: 477 - protein, 10 aa, Homo sapiens
SHFRGDLHTS

SEQ ID NO: 478 - protein, 10 aa, Homo sapiens
SDRRGDLSVP

SEQ ID NO: 479 - protein, 10 aa, Homo sapiens
SGTRGDTLIL

SEQ ID NO: 480 - protein, 10 aa, Homo sapiens
ENTRGDLHGK

SEQ ID NO: 481 - protein, 10 aa, Homo sapiens
DRARGDTHVL

SEQ ID NO: 482 - protein, 10 aa, Homo sapiens
ASGRGDIAGL

SEQ ID NO: 483 - protein, 10 aa, Homo sapiens
EGRRGDLFQA

SEQ ID NO: 484 - protein, 10 aa, Homo sapiens
RSERGDRLEI

SEQ ID NO: 485 - protein, 10 aa, Homo sapiens
AAYRGDAHVL

SEQ ID NO: 486 - protein, 10 aa, Homo sapiens
NDSRGDQHRL

SEQ ID NO: 487 - protein, 10 aa, Homo sapiens
SSARGDHSQL

SEQ ID NO: 488 - protein, 10 aa, Homo sapiens
TNERGDRLSI

SEQ ID NO: 489 - protein, 10 aa, Homo sapiens
SNSRGDYTSV

SEQ ID NO: 490 - protein, 10 aa, Homo sapiens
NVTRGDHTVM

SEQ ID NO: 491 - protein, 10 aa, Homo sapiens
TMVRGDVKGL

SEQ ID NO: 492 - protein, 10 aa, Homo sapiens
VQLRGDLAST

SEQ ID NO: 493 - protein, 10 aa, Homo sapiens
MNRRGDYSEQ

SEQ ID NO: 494 - protein, 10 aa, Homo sapiens
ANTRGDLSPV

SEQ ID NO: 495 - protein, 10 aa, Homo sapiens
TGMRGDAGSL

SEQ ID NO: 496 - protein, 10 aa, Homo sapiens
LDRRGDFSSA

SEQ ID NO: 497 - protein, 10 aa, Homo sapiens
MYHRGDMTSV

SEQ ID NO: 498 - protein, 10 aa, Homo sapiens
GSMRGDLHST

SEQ ID NO: 499 - protein, 10 aa, Homo sapiens
SSSRGDFSSV

SEQ ID NO: 500 - protein, 10 aa, Homo sapiens
TGTRGDLHTY

SEQ ID NO: 501 - protein, 10 aa, Homo sapiens
YTQRGDLATV

SEQ ID NO: 502 - protein, 10 aa, Homo sapiens
TDVRGDRMYV

SEQ ID NO: 503 - protein, 10 aa, Homo sapiens
SNSRGDLSVN

SEQ ID NO: 504 - protein, 10 aa, Homo sapiens
QSGRGDQMTL

SEQ ID NO: 505 - protein, 10 aa, Homo sapiens
MNTRGDLQQS

SEQ ID NO: 506 - protein, 10 aa, Homo sapiens
AGSRGDLQTV

SEQ ID NO: 507 - protein, 10 aa, Homo sapiens
SNMRGDQTYV

SEQ ID NO: 508 - protein, 10 aa, Homo sapiens
TPSRGDMSNV

SEQ ID NO: 509 - protein, 10 aa, Homo sapiens
NSTRGDLHMA

SEQ ID NO: 510 - protein, 10 aa, Homo sapiens
ANTRGDLSVS

SEQ ID NO: 511 - protein, 10 aa, Homo sapiens
TDRRGDTGLL

SEQ ID NO: 512 - protein, 10 aa, Homo sapiens
DGVRGDRATW

SEQ ID NO: 513 - protein, 10 aa, Homo sapiens
EPSRGDRMVL

SEQ ID NO: 514 - protein, 10 aa, Homo sapiens
SGVRGDRIII

SEQ ID NO: 515 - protein, 10 aa, Homo sapiens
TSMRGDYTHV

SEQ ID NO: 516 - protein, 10 aa, Homo sapiens
ASSRGDLTVI

SEQ ID NO: 517 - protein, 10 aa, Homo sapiens
ETRRGDLSLQ

SEQ ID NO: 518 - protein, 10 aa, Homo sapiens
NNARGDQSMH

SEQ ID NO: 519 - protein, 10 aa, Homo sapiens
TDRRGDSSAL

SEQ ID NO: 520 - protein, 10 aa, Homo sapiens
SSQRGDLTGV

SEQ ID NO: 521 - protein, 10 aa, Homo sapiens
QTARGDVMVH

SEQ ID NO: 522 - protein, 10 aa, Homo sapiens
VSQRGDLNAV

SEQ ID NO: 523 - protein, 10 aa, Homo sapiens
TMTRGDLAAN

SEQ ID NO: 524 - protein, 10 aa, Homo sapiens
NASRGDHSSL

SEQ ID NO: 525 - protein, 10 aa, Homo sapiens
SNTRGDMGLT

SEQ ID NO: 526 - protein, 10 aa, Homo sapiens
MYSRGDTHSL

SEQ ID NO: 527 - protein, 10 aa, Homo sapiens
ADQRGDRAPL

SEQ ID NO: 528 - protein, 10 aa, Homo sapiens
SQNRGDLANT

SEQ ID NO: 529 - protein, 10 aa, Homo sapiens
SNRRGDTEMQ

SEQ ID NO: 530 - protein, 10 aa, Homo sapiens
SGSRGDVSAL

SEQ ID NO: 531 - protein, 10 aa, Homo sapiens
ESTRGDRGTL

SEQ ID NO: 532 - protein, 10 aa, Homo sapiens
SAVRGDAALH

SEQ ID NO: 533 - protein, 10 aa, Homo sapiens
LSSRGDVNRL

SEQ ID NO: 534 - protein, 10 aa, Homo sapiens
EQRRGDIQTI

SEQ ID NO: 535 - protein, 10 aa, Homo sapiens
SPVRGDHGAL

SEQ ID NO: 536 - protein, 10 aa, Homo sapiens
LSRRGDYQEL

SEQ ID NO: 537 - protein, 10 aa, Homo sapiens
DSRRGDASYH

SEQ ID NO: 538 - protein, 10 aa, Homo sapiens
NHQRGDLSSS

SEQ ID NO: 539 - protein, 10 aa, Homo sapiens
SWNRGDISGL

SEQ ID NO: 540 - protein, 10 aa, Homo sapiens
TGTRGDLGTM

SEQ ID NO: 541 - protein, 10 aa, Homo sapiens
QQLRGDTHTL

SEQ ID NO: 542 - protein, 10 aa, Homo sapiens
AGLRGDRDSL

SEQ ID NO: 543 - protein, 10 aa, Homo sapiens
QVYRGDRDQL

SEQ ID NO: 544 - protein, 10 aa, Homo sapiens
AGVRGDRVTI

SEQ ID NO: 545 - protein, 10 aa, Homo sapiens
NSARGDLLHS

SEQ ID NO: 546 - protein, 10 aa, Homo sapiens
NHSRGDLTGV

SEQ ID NO: 547 - protein, 10 aa, Homo sapiens
NAYRGDTSAF

SEQ ID NO: 548 - protein, 10 aa, Homo sapiens
SANRGDIMHT

SEQ ID NO: 549 - protein, 10 aa, Homo sapiens
QSARGDLVSY

SEQ ID NO: 550 - protein, 10 aa, Homo sapiens
SFVRGDVRTL

SEQ ID NO: 551 - protein, 10 aa, Homo sapiens
SDMRGDRSVY

SEQ ID NO: 552 - protein, 10 aa, Homo sapiens
SGIRGDRYPI

SEQ ID NO: 553 - protein, 10 aa, Homo sapiens
SNVRGDMAHS

SEQ ID NO: 554 - protein, 10 aa, Homo sapiens
ASSRGDLQQT

SEQ ID NO: 555 - protein, 10 aa, Homo sapiens
NSVRGDLNQT

SEQ ID NO: 556 - protein, 10 aa, Homo sapiens
GPGRGDQTTL

SEQ ID NO: 557 - DNA, 30 bp, Homo sapiens
  source 1..30: mol_type = other DNA
tcaaactccagaggagactacaactccttg

SEQ ID NO: 558 - DNA, 30 bp, Homo sapiens
  source 1..30: mol_type = other DNA
tcgtcggctagaggagaccgtgattatctg

SEQ ID NO: 559 - DNA, 30 bp, Homo sapiens
  source 1..30: mol_type = other DNA
agcacggtcagaggagactacacgtccgtg

SEQ ID NO: 560 - DNA, 30 bp, Homo sapiens
  source 1..30: mol_type = other DNA
caggagcggagaggagactatacgagtatg

SEQ ID NO: 561 - DNA, 30 bp, Homo sapiens; identical to SEQ ID NO: 204
  source 1..30: mol_type = other DNA

SEQ ID NO: 562 - DNA, 30 bp, Homo sapiens
  source 1..30: mol_type = other DNA
gcgagtactagaggagaccatggtgtgttg

SEQ ID NO: 563 - DNA, 30 bp, Homo sapiens
  source 1..30: mol_type = other DNA
tcgtaccaaagaggagaccaacacaacatg

SEQ ID NO: 564 - DNA, 30 bp, Homo sapiens
  source 1..30: mol_type = other DNA
aacgacacaagaggagaccgatcttacatg

SEQ ID NO: 565 - DNA, 30 bp, Homo sapiens
  source 1..30: mol_type = other DNA
gaaaacaggagaggagacttcaacaacact

SEQ ID NO: 566 - DNA, 30 bp, Homo sapiens
  source 1..30: mol_type = other DNA
tccggctcgagaggagacttgtcaggacac

SEQ ID NO: 567 - DNA, 30 bp, Homo sapiens; identical to SEQ ID NO: 298
  source 1..30: mol_type = other DNA

SEQ ID NO: 568 - DNA, 30 bp, Homo sapiens
  source 1..30: mol_type = other DNA
gataagccgagaggagaccggcagttgttg

SEQ ID NO: 569 - DNA, 30 bp, Homo sapiens; identical to SEQ ID NO: 279
  source 1..30: mol_type = other DNA

SEQ ID NO: 570 - DNA, 30 bp, Homo sapiens; identical to SEQ ID NO: 275
  source 1..30: mol_type = other DNA

SEQ ID NO: 571 - DNA, 30 bp, Homo sapiens
  source 1..30: mol_type = other DNA
tcaaacaacagaggagactacacgacaatg

SEQ ID NO: 572 - DNA, 30 bp, Homo sapiens
  source 1..30: mol_type = other DNA
cagggtgggagaggagacttgtcgacgttg

SEQ ID NO: 573 - DNA, 30 bp, Homo sapiens
  source 1..30: mol_type = other DNA
tcgaaggatagaggagactatcagactctt

SEQ ID NO: 574 - DNA, 30 bp, Homo sapiens
  source 1..30: mol_type = other DNA
gctacggcgagaggagaccagatgaagctg

SEQ ID NO: 575 - DNA, 30 bp, Homo sapiens
  source 1..30: mol_type = other DNA
cagcatatgagaggagacttgactggtgcg

SEQ ID NO: 576 - DNA, 30 bp, Homo sapiens
  source 1..30: mol_type = other DNA
gacggcacgagaggagaccttggaaggtac

SEQ ID NO: 577 - DNA, 30 bp, Homo sapiens
  source 1..30: mol_type = other DNA
gactctcggagaggagactacgcaaacctc

SEQ ID NO: 578 - DNA, 30 bp, Homo sapiens
  source 1..30: mol_type = other DNA
tcggcgatgagaggagactatactagtact

SEQ ID NO: 579 - DNA, 30 bp, Homo sapiens
  source 1..30: mol_type = other DNA
gagaatcggagaggagacgcgcagctgctg

SEQ ID NO: 580 - DNA, 30 bp, Homo sapiens
  source 1..30: mol_type = other DNA
acgacgctgagaggagaccatagtcagctt

SEQ ID NO: 581 - DNA, 30 bp, Homo sapiens
  source 1..30: mol_type = other DNA
gacgcagggagaggagacatgcaccgtatg

SEQ ID NO: 582 - DNA, 30 bp, Homo sapiens
  source 1..30: mol_type = other DNA
cagcatacgagaggagacttgagttctact

SEQ ID NO: 583 - DNA, 30 bp, Homo sapiens
  source 1..30: mol_type = other DNA
caagtttacagaggagacagggaatccgtg

SEQ ID NO: 584 - DNA, 30 bp, Homo sapiens
  source 1..30: mol_type = other DNA
tcgagtcagagaggagacttggctggtact

SEQ ID NO: 585 - DNA, 30 bp, Homo sapiens
  source 1..30: mol_type = other DNA
acggatgttagaggagacagggggacgttt

SEQ ID NO: 586 - DNA, 30 bp, Homo sapiens
  source 1..30: mol_type = other DNA
tcgtctgtgagaggagaccgggaggtgacg

SEQ ID NO: 587 - DNA, 30 bp, Homo sapiens
  source 1..30: mol_type = other DNA
gagaataggagaggagacctgactaatgcg

SEQ ID NO: 588 - DNA, 30 bp, Homo sapiens
  source 1..30: mol_type = other DNA
tcgtatgcgagaggagacgttcattctatt

SEQ ID NO: 589 - DNA, 30 bp, Homo sapiens
  source 1..30: mol_type = other DNA
gacagctcaagaggagacctcaacctccgg

SEQ ID NO: 590 - DNA, 30 bp, Homo sapiens
  source 1..30: mol_type = other DNA
tcgaacgcgagaggagactactcaaacatg

SEQ ID NO: 591 - DNA, 30 bp, Homo sapiens
  source 1..30: mol_type = other DNA
tcgtctgtgagaggagaccattcggtgctt

SEQ ID NO: 592 - DNA, 30 bp, Homo sapiens
  source 1..30: mol_type = other DNA
tcggcttttagaggagacctgcatgctact

SEQ ID NO: 593 - DNA, 30 bp, Homo sapiens
  source 1..30: mol_type = other DNA
tccgccatgagaggagactacacaagcacg

SEQ ID NO: 594 - DNA, 30 bp, Homo sapiens; identical to SEQ ID NO: 328
  source 1..30: mol_type = other DNA

SEQ ID NO: 595 - DNA, 30 bp, Homo sapiens
  source 1..30: mol_type = other DNA
tcggatcggagaggagaccagctgttgtat

SEQ ID NO: 596 - DNA, 30 bp, Homo sapiens
  source 1..30: mol_type = other DNA
agcggcgtaagaggagaccgcctagccgta

SEQ ID NO: 597 - DNA, 30 bp, Homo sapiens
  source 1..30: mol_type = other DNA
gccagcctaagaggagacctcagctcaacg

SEQ ID NO: 598 - DNA, 30 bp, Homo sapiens
  source 1..30: mol_type = other DNA
acgaacggaagaggagaccttcacggaatg

SEQ ID NO: 599 - DNA, 30 bp, Homo sapiens
  source 1..30: mol_type = other DNA
acgaatacgagaggagaccatgggatgctg

SEQ ID NO: 600 - DNA, 30 bp, Homo sapiens
  source 1..30: mol_type = other DNA
agttccgccagaggagaccgggactacctc

SEQ ID NO: 601 - DNA, 30 bp, Homo sapiens
  source 1..30: mol_type = other DNA
gaggatcggagaggagaccttttgaggact

SEQ ID NO: 602 - DNA, 30 bp, Homo sapiens
  source 1..30: mol_type = other DNA
tcgtcgctgagaggagacctgttgcattct

SEQ ID NO: 603 - DNA, 30 bp, Homo sapiens
  source 1..30: mol_type = other DNA
gaaaaccgtagaggagacctcacgaacgct

SEQ ID NO: 604 - DNA, 30 bp, Homo sapiens
  source 1..30: mol_type = other DNA
gacggcaggagaggagaccgggaatcgatc

SEQ ID NO: 605 - DNA, 30 bp, Homo sapiens
  source 1..30: mol_type = other DNA
actggaaccagaggagacacaatgacatgg

SEQ ID NO: 606 - DNA, 30 bp, Homo sapiens
  source 1..30: mol_type = other DNA
tcgactgttagaggagactatacttctgtt

SEQ ID NO: 607 - DNA, 30 bp, Homo sapiens
  source 1..30: mol_type = other DNA
tcggcaacgagaggagaccacaacgtactg

SEQ ID NO: 608 - DNA, 30 bp, Homo sapiens
  source 1..30: mol_type = other DNA
acggatcggagaggagacagtggtactctg

SEQ ID NO: 609 - DNA, 30 bp, Homo sapiens
  source 1..30: mol_type = other DNA
acgagtgctagaggagacctgattcatacg

SEQ ID NO: 610 - DNA, 30 bp, Homo sapiens
  source 1..30: mol_type = other DNA
agtggggtgagaggagaccggctggcggtg

SEQ ID NO: 611 - DNA, 30 bp, Homo sapiens
  source 1..30: mol_type = other DNA
gcggttgagagaggagaccgtcttatgctg

SEQ ID NO: 612 - DNA, 30 bp, Homo sapiens
  source 1..30: mol_type = other DNA
gacaggtcgagaggagacacgcacgtactc

SEQ ID NO: 613 - DNA, 30 bp, Homo sapiens
  source 1..30: mol_type = other DNA
gagtcgacgagaggagaccttcgtgttgtg

SEQ ID NO: 614 - DNA, 30 bp, Homo sapiens
  source 1..30: mol_type = other DNA
acgacgattagaggagactatcgtgagttg

SEQ ID NO: 615 - DNA, 30 bp, Homo sapiens
  source 1..30: mol_type = other DNA
acgttcatcagaggagacctggcaggtgcg

SEQ ID NO: 616 - DNA, 30 bp, Homo sapiens
  source 1..30: mol_type = other DNA
atgacgagcagaggagacttgacaacatac

SEQ ID NO: 617 - DNA, 30 bp, Homo sapiens
  source 1..30: mol_type = other DNA
gcggatcgtagaggagaccagctttattcg

SEQ ID NO: 618 - DNA, 30 bp, Homo sapiens; identical to SEQ ID NO: 192
  source 1..30: mol_type = other DNA

SEQ ID NO: 619 - DNA, 30 bp, Homo sapiens
  source 1..30: mol_type = other DNA
gggtacgtcagaggagacctggggcaacac

SEQ ID NO: 620 - DNA, 30 bp, Homo sapiens
  source 1..30: mol_type = other DNA
gatgcgagtagaggagacaggactagtttg

SEQ ID NO: 621 - DNA, 30 bp, Homo sapiens
  source 1..30: mol_type = other DNA
acggaggtgagaggagacaggtcgcaggtt

SEQ ID NO: 622 - DNA, 30 bp, Homo sapiens
  source 1..30: mol_type = other DNA
tcgcatctgagaggagaccaggctcttgcg

SEQ ID NO: 623 - DNA, 30 bp, Homo sapiens
  source 1..30: mol_type = other DNA
tcggaggtgagaggagaccggttgagtatt

SEQ ID NO: 624 - DNA, 30 bp, Homo sapiens; identical to SEQ ID NO: 215
  source 1..30: mol_type = other DNA

SEQ ID NO: 625 - DNA, 30 bp, Homo sapiens
  source 1..30: mol_type = other DNA
tcagaccgtagaggagacctgtcgggcgct

SEQ ID NO: 626 - DNA, 30 bp, Homo sapiens
  source 1..30: mol_type = other DNA
agcgtcacaagaggagacagagtagtcatc

SEQ ID NO: 627 - DNA, 30 bp, Homo sapiens
  source 1..30: mol_type = other DNA
tcggaggggagaggagacaggatggctctt

SEQ ID NO: 628 - DNA, 30 bp, Homo sapiens
  source 1..30: mol_type = other DNA
tcgtctactagaggagaccatctttcgctg

SEQ ID NO: 629 - DNA, 30 bp, Homo sapiens
  source 1..30: mol_type = other DNA
gctatgatcagaggagacatgacccactcc

SEQ ID NO: 630 - DNA, 30 bp, Homo sapiens
  source 1..30: mol_type = other DNA
tcgtcgacgagaggagaccagatttatgtg

SEQ ID NO: 631 - DNA, 30 bp, Homo sapiens
  source 1..30: mol_type = other DNA
gaggagcggagaggagacactcatcggttg

SEQ ID NO: 632 - DNA, 30 bp, Homo sapiens
  source 1..30: mol_type = other DNA
tcgaattcgagaggagactataatagtctt

SEQ ID NO: 633 - DNA, 30 bp, Homo sapiens
  source 1..30: mol_type = other DNA
tccgcacaaagggatcgtatgggttctccg

SEQ ID NO: 634 - DNA, 30 bp, Homo sapiens
  source 1..30: mol_type = other DNA
acgggtacgagaggagacattgcgactttt

SEQ ID NO: 635 - DNA, 30 bp, Homo sapiens
  source 1..30: mol_type = other DNA
tcgatgcagagaggagacctgctttctact

SEQ ID NO: 636 - DNA, 30 bp, Homo sapiens
  source 1..30: mol_type = other DNA
gaggatgtgagaggagacaggagtaagctt

SEQ ID NO: 637 - DNA, 30 bp, Homo sapiens; identical to SEQ ID NO: 199
  source 1..30: mol_type = other DNA

SEQ ID NO: 638 - DNA, 30 bp, Homo sapiens
  source 1..30: mol_type = other DNA
acgagtgtgagaggagaccagagtcagtat

SEQ ID NO: 639 - DNA, 30 bp, Homo sapiens
  source 1..30: mol_type = other DNA
gcggatcggagaggagacacggggttgttt

SEQ ID NO: 640 - DNA, 30 bp, Homo sapiens
  source 1..30: mol_type = other DNA
aacacttacagaggagaccgagacagcctg

SEQ ID NO: 641 - DNA, 30 bp, Homo sapiens
  source 1..30: mol_type = other DNA
acggatgttagaggagaccgggggacgttt

SEQ ID NO: 642 - DNA, 30 bp, Homo sapiens
  source 1..30: mol_type = other DNA
tctcgtgggagaggagacctgaatgatttg

SEQ ID NO: 643 - DNA, 30 bp, Homo sapiens
  source 1..30: mol_type = other DNA
agcaacgtaagaggagacagggaagccgtc

SEQ ID NO: 644 - DNA, 30 bp, Homo sapiens
  source 1..30: mol_type = other DNA
atggacaggagaggagacgcctccatactg

SEQ ID NO: 645 - DNA, 30 bp, Homo sapiens
  source 1..30: mol_type = other DNA
tcgatatggagaggagaccgtacagaagtc

SEQ ID NO: 646 - DNA, 30 bp, Homo sapiens
  source 1..30: mol_type = other DNA
aactcacaaagaggagactacagtggaatg

SEQ ID NO: 647 - DNA, 30 bp, Homo sapiens
  source 1..30: mol_type = other DNA
tctagcgtaagaggagacatcggggggatc

SEQ ID NO: 648 - DNA, 30 bp, Homo sapiens; identical to SEQ ID NO: 248
  source 1..30: mol_type = other DNA

SEQ ID NO: 649 - DNA, 30 bp, Homo sapiens
  source 1..30: mol_type = other DNA
gagattcggagaggagaccttctggctcag

SEQ ID NO: 650 - DNA, 30 bp, Homo sapiens
  source 1..30: mol_type = other DNA
tcgaacatcagaggagaccacgcagtcatg

SEQ ID NO: 651 - DNA, 30 bp, Homo sapiens
  source 1..30: mol_type = other DNA
ctggatacgagaggagaccggagtcaggtt

SEQ ID NO: 652 - DNA, 30 bp, Homo sapiens
  source 1..30: mol_type = other DNA
gcgaatacgagaggagaccatcagaatttt

SEQ ID NO: 653 - DNA, 30 bp, Homo sapiens
  source 1..30: mol_type = other DNA
caggtgtatagaggagaccgtgagtctgtt

SEQ ID NO: 654 - DNA, 30 bp, Homo sapiens
  source 1..30: mol_type = other DNA
acgtcggtgagaggagacctttcgatgaat

SEQ ID NO: 655 - DNA, 30 bp, Homo sapiens
  source 1..30: mol_type = other DNA
gcctcaacgagaggagaccacggagttctc

SEQ ID NO: 656 - DNA, 30 bp, Homo sapiens
  source 1..30: mol_type = other DNA
acgacgacgagaggagactatcttaatacg

SEQ ID NO: 657 - DNA, 30 bp, Homo sapiens
  source 1..30: mol_type = other DNA
gcggagaggagaggagacattaaggagtat

SEQ ID NO: 658 - DNA, 30 bp, Homo sapiens
  source 1..30: mol_type = other DNA
tcgcatgctagaggagacttgggttcgact

SEQ ID NO: 659 - DNA, 30 bp, Homo sapiens
  source 1..30: mol_type = other DNA
acgtctcagagaggagactatgtttctctt

SEQ ID NO: 660 - DNA, 30 bp, Homo sapiens; identical to SEQ ID NO: 182
  source 1..30: mol_type = other DNA

SEQ ID NO: 661 - DNA, 30 bp, Homo sapiens
  source 1..30: mol_type = other DNA
gtgaatgtgagaggagaccgtggtgaggtg

SEQ ID NO: 662 - DNA, 30 bp, Homo sapiens
  source 1..30: mol_type = other DNA
tcgtatcgtagaggagaccatgatcagctt

SEQ ID NO: 663 - DNA, 30 bp, Homo sapiens
  source 1..30: mol_type = other DNA
agcggcacaagaggagacagggtagacctc

SEQ ID NO: 664 - DNA, 30 bp, Homo sapiens
  source 1..30: mol_type = other DNA
tcccacttcagaggagacttgcacacgtcc

SEQ ID NO: 665 - DNA, 30 bp, Homo sapiens
  source 1..30: mol_type = other DNA
tcggatcgtagaggagacctttcggtgcct

SEQ ID NO: 666 - DNA, 30 bp, Homo sapiens
  source 1..30: mol_type = other DNA
agcggcaccagaggagacacccttatactc

SEQ ID NO: 667 - DNA, 30 bp, Homo sapiens
  source 1..30: mol_type = other DNA
gaaaacaccagaggagacctccacggcaaa

SEQ ID NO: 668 - DNA, 30 bp, Homo sapiens
  source 1..30: mol_type = other DNA
gatcgggcgagaggagacacgcatgtgctg

SEQ ID NO: 669 - DNA, 30 bp, Homo sapiens
  source 1..30: mol_type = other DNA
gcgtctggtagaggagacattgcggggctt

SEQ ID NO: 670 - DNA, 30 bp, Homo sapiens
  source 1..30: mol_type = other DNA
gagggtcgtagaggagacctgtttcaggcg

SEQ ID NO: 671 - DNA, 30 bp, Homo sapiens
  source 1..30: mol_type = other DNA
agatccgaaagaggagacaggctagaaatc

SEQ ID NO: 672 - DNA, 30 bp, Homo sapiens
  source 1..30: mol_type = other DNA
gcggcctacagaggagacgcacacgtgctc

SEQ ID NO: 673 - DNA, 30 bp, Homo sapiens
  source 1..30: mol_type = other DNA
aacgactcgagaggagaccaacaccgattg

SEQ ID NO: 674 - DNA, 30 bp, Homo sapiens
  source 1..30: mol_type = other DNA
tcatcggcaagaggagaccactctcaactg

SEQ ID NO: 675 - DNA, 30 bp, Homo sapiens
  source 1..30: mol_type = other DNA
acgaatgagagaggagaccgtctttcgatt

SEQ ID NO: 676 - DNA, 30 bp, Homo sapiens
  source 1..30: mol_type = other DNA
agcaactccagaggagactacaccagtgtc

SEQ ID NO: 677 - DNA, 30 bp, Homo sapiens
  source 1..30: mol_type = other DNA
aacgtcacaagaggagaccacaccgttatg

SEQ ID NO: 678 - DNA, 30 bp, Homo sapiens
  source 1..30: mol_type = other DNA
acgatggtgagaggagacgttaaggggctt

SEQ ID NO: 679 - DNA, 30 bp, Homo sapiens
  source 1..30: mol_type = other DNA
accgggacaagaggagacatagccacgttc

SEQ ID NO: 680 - DNA, 30 bp, Homo sapiens
  source 1..30: mol_type = other DNA
gtgcagctgagaggagacttggcttctact

SEQ ID NO: 681 - DNA, 30 bp, Homo sapiens
  source 1..30: mol_type = other DNA
atgaaccggagaggagactactcggaacaa

SEQ ID NO: 682 - DNA, 30 bp, Homo sapiens
  source 1..30: mol_type = other DNA
gcgaatactagaggagacctgagtccggtt

SEQ ID NO: 683 - DNA, 30 bp, Homo sapiens
  source 1..30: mol_type = other DNA
acgggtatgagaggagacgctgggagtctt

SEQ ID NO: 684 - DNA, 30 bp, Homo sapiens
  source 1..30: mol_type = other DNA
ctagaccgcagaggagacttcagctccgcg

SEQ ID NO: 685 - DNA, 30 bp, Homo sapiens
  source 1..30: mol_type = other DNA
atgtaccacagaggagacatgacttcggtg

SEQ ID NO: 686 - DNA, 30 bp, Homo sapiens
  source 1..30: mol_type = other DNA
gggtcgatgagaggagacttgcattcgacg

SEQ ID NO: 687 - DNA, 30 bp, Homo sapiens
  source 1..30: mol_type = other DNA
tcgtcgtccagaggagacttcagctctgtt

SEQ ID NO: 688 - DNA, 30 bp, Homo sapiens
  source 1..30: mol_type = other DNA
acgggtacgagaggagacttgcatacttat

SEQ ID NO: 689 - DNA, 30 bp, Homo sapiens
  source 1..30: mol_type = other DNA
tacacccaaagaggagaccttgccaccgtc

SEQ ID NO: 690 - DNA, 30 bp, Homo sapiens
  source 1..30: mol_type = other DNA
acggatgttagaggagaccggatgtatgtg

SEQ ID NO: 691 - DNA, 30 bp, Homo sapiens; identical to SEQ ID NO: 220
  source 1..30: mol_type = other DNA

SEQ ID NO: 692 - DNA, 30 bp, Homo sapiens
  source 1..30: mol_type = other DNA
tcgaactccagaggagacctatcggtaaac

SEQ ID NO: 693 - DNA, 30 bp, Homo sapiens
  source 1..30: mol_type = other DNA
cagtctgggagaggagaccagatgacgttg

SEQ ID NO: 694 - DNA, 30 bp, Homo sapiens
  source 1..30: mol_type = other DNA
atgaacaccagaggagacttgcaacaatcg

SEQ ID NO: 695 - DNA, 30 bp, Homo sapiens
  source 1..30: mol_type = other DNA
gcgggatcaagaggagacctacaaacggtg

SEQ ID NO: 696 - DNA, 30 bp, Homo sapiens
  source 1..30: mol_type = other DNA
tcgaacatgagaggagaccaaacttacgtc

SEQ ID NO: 697 - DNA, 30 bp, Homo sapiens
  source 1..30: mol_type = other DNA
acgccaagtagaggagacatgtccaacgtc

SEQ ID NO: 698 - DNA, 30 bp, Homo sapiens
  source 1..30: mol_type = other DNA
aacagcacaagaggagacctacacatggca

SEQ ID NO: 699 - DNA, 30 bp, Homo sapiens
  source 1..30: mol_type = other DNA
gctaacaccagaggagacctctcagtcagt

SEQ ID NO: 700 - DNA, 30 bp, Homo sapiens
  source 1..30: mol_type = other DNA
accgaccgaagaggagacaccggcttactg

SEQ ID NO: 701 - DNA, 30 bp, Homo sapiens
  source 1..30: mol_type = other DNA
gacggcgtaagaggagaccgtgcaacatgg

SEQ ID NO: 702 - DNA, 30 bp, Homo sapiens
  source 1..30: mol_type = other DNA
gagaatcgtagaggagactttaataatacg

SEQ ID NO: 703 - DNA, 30 bp, Homo sapiens
  source 1..30: mol_type = other DNA
gagcctagtagaggagacaggatggtgttg

SEQ ID NO: 704 - DNA, 30 bp, Homo sapiens
  source 1..30: mol_type = other DNA
agcggcgttagaggagaccggatcataatc

SEQ ID NO: 705 - DNA, 30 bp, Homo sapiens
  source 1..30: mol_type = other DNA
acgtcgatgagaggagactatacgcatgtt

SEQ ID NO: 706 - DNA, 30 bp, Homo sapiens
  source 1..30: mol_type = other DNA
gcgtcgagtagaggagacttgactgttatt

SEQ ID NO: 707 - DNA, 30 bp, Homo sapiens
  source 1..30: mol_type = other DNA
gagacgcgtagaggagacctgtcgcttcag

SEQ ID NO: 708 - DNA, 30 bp, Homo sapiens
  source 1..30: mol_type = other DNA
aacaacgcaagaggagaccaatcaatgcac

SEQ ID NO: 709 - DNA, 30 bp, Homo sapiens
  source 1..30: mol_type = other DNA
acggataggagaggagactctagtgcgctt

SEQ ID NO: 710 - DNA, 30 bp, Homo sapiens
  source 1..30: mol_type = other DNA
tcaagccaaagaggagacttgacaggtgtg

SEQ ID NO: 711 - DNA, 30 bp, Homo sapiens
  source 1..30: mol_type = other DNA
cagacggctagaggagacgttatggttcat

SEQ ID NO: 712 - DNA, 30 bp, Homo sapiens
  source 1..30: mol_type = other DNA
gtgtcgcagagaggagacctgaatgctgtt

SEQ ID NO: 713 - DNA, 30 bp, Homo sapiens
  source 1..30: mol_type = other DNA
acgatgaccagaggagacctcgctgctaac

SEQ ID NO: 714 - DNA, 30 bp, Homo sapiens
  source 1..30: mol_type = other DNA
aatgcttcgagaggagaccattcgtcgttg

SEQ ID NO: 715 - DNA, 30 bp, Homo sapiens
  source 1..30: mol_type = other DNA
tcaaacaccagaggagacatgggcctcacg

SEQ ID NO: 716 - DNA, 30 bp, Homo sapiens
  source 1..30: mol_type = other DNA
atgtattcgagaggagacactcatagtctg

SEQ ID NO: 717 - DNA, 30 bp, Homo sapiens
  source 1..30: mol_type = other DNA
gcggatcagagaggagacagggctccgctt

SEQ ID NO: 718 - DNA, 30 bp, Homo sapiens
  source 1..30: mol_type = other DNA
caagaaaggagaggagactacacctctatg

SEQ ID NO: 719 - DNA, 30 bp, Homo sapiens
  source 1..30: mol_type = other DNA
tcccaaaacagaggagacctagccaacacg

SEQ ID NO: 720 - DNA, 30 bp, Homo sapiens; identical to SEQ ID NO: 236
  source 1..30: mol_type = other DNA

SEQ ID NO: 721 - DNA, 30 bp, Homo sapiens
  source 1..30: mol_type = other DNA
caaggcggaagaggagacctgagtacactg

SEQ ID NO: 722 - DNA, 30 bp, Homo sapiens
  source 1..30: mol_type = other DNA
tcgaataggagaggagacactgagatgcag

SEQ ID NO: 723 - DNA, 30 bp, Homo sapiens
  source 1..30: mol_type = other DNA
tcgggtagtagaggagacgtgagtgctttg

SEQ ID NO: 724 - DNA, 30 bp, Homo sapiens
  source 1..30: mol_type = other DNA
gagtcgacgagaggagaccgtggtacgctg

SEQ ID NO: 725 - DNA, 30 bp, Homo sapiens
  source 1..30: mol_type = other DNA
tcggcggtgagaggagacgcggcgcttcat

SEQ ID NO: 726 - DNA, 30 bp, Homo sapiens
  source 1..30: mol_type = other DNA
ctgagcagcagaggagacgttaaccgcctt

SEQ ID NO: 727 - DNA, 30 bp, Homo sapiens
  source 1..30: mol_type = other DNA
gagcagaggagaggagacattcagactatt

SEQ ID NO: 728 - DNA, 30 bp, Homo sapiens
  source 1..30: mol_type = other DNA
gctgcgtatagaggagacgcgcatgttctt

SEQ ID NO: 729 - DNA, 30 bp, Homo sapiens
  source 1..30: mol_type = other DNA
tcgccggtgagaggagaccatggggctttg

SEQ ID NO: 730 - DNA, 30 bp, Homo sapiens
  source 1..30: mol_type = other DNA
gattcgaggagaggagactatgcgaatctg

SEQ ID NO: 731 - DNA, 30 bp, Homo sapiens
  source 1..30: mol_type = other DNA
ttgagtcggagaggagactatcaggagttg

SEQ ID NO: 732 - DNA, 30 bp, Homo sapiens
  source 1..30: mol_type = other DNA
tcctacgcaagaggagacgtccactccatc

SEQ ID NO: 733 - DNA, 30 bp, Homo sapiens
  source 1..30: mol_type = other DNA
gactcacgcagaggagacgcgtcgtaccac

SEQ ID NO: 734 - DNA, 30 bp, Homo sapiens
  source 1..30: mol_type = other DNA
aaccaccaaagaggagacctgagctcgagt

SEQ ID NO: 735 - DNA, 30 bp, Homo sapiens
  source 1..30: mol_type = other DNA
tcctcaacgagaggagaccacctgtctttg

SEQ ID NO: 736 - DNA, 30 bp, Homo sapiens
  source 1..30: mol_type = other DNA
tcgtggaacagaggagacatatctggcctt

SEQ ID NO: 737 - DNA, 30 bp, Homo sapiens
  source 1..30: mol_type = other DNA
acgggtactagaggagacctggggacgatg

SEQ ID NO: 738 - DNA, 30 bp, Homo sapiens
  source 1..30: mol_type = other DNA
cagcagcttagaggagacacgcatactctt

SEQ ID NO: 739 - DNA, 30 bp, Homo sapiens
  source 1..30: mol_type = other DNA
gcggggttgagaggagaccgtgattcgctt

SEQ ID NO: 740 - DNA, 30 bp, Homo sapiens
  source 1..30: mol_type = other DNA
caggtttatagaggagaccgggatcagttg

SEQ ID NO: 741 - DNA, 30 bp, Homo sapiens
  source 1..30: mol_type = other DNA
gcaggagtaagaggagacagagtgaccatc

SEQ ID NO: 742 - DNA, 30 bp, Homo sapiens
  source 1..30: mol_type = other DNA
aactcagccagaggagaccttctgcactcc

SEQ ID NO: 743 - DNA, 30 bp, Homo sapiens
  source 1..30: mol_type = other DNA
aaccacagtagaggagacctgacaggcgtt

SEQ ID NO: 744 - DNA, 30 bp, Homo sapiens
  source 1..30: mol_type = other DNA
aacgcttacagaggagacacatccgcgttc

SEQ ID NO: 745 - DNA, 30 bp, Homo sapiens
  source 1..30: mol_type = other DNA
tcggctaacagaggagacataatgcacacg

SEQ ID NO: 746 - DNA, 30 bp, Homo sapiens
  source 1..30: mol_type = other DNA
caatccgccagaggagacctcgtcagttac

SEQ ID NO: 747 - DNA, 30 bp, Homo sapiens
  source 1..30: mol_type = other DNA
gatagtaggagaggagacgctagttatcat

SEQ ID NO: 748 - DNA, 30 bp, Homo sapiens
  source 1..30: mol_type = other DNA
tcgtttgtgagaggagacgttcgtacgctg

SEQ ID NO: 749 - DNA, 30 bp, Homo sapiens
  source 1..30: mol_type = other DNA
agcgacatgagaggagaccgatctgtgtac

SEQ ID NO: 750 - DNA, 30 bp, Homo sapiens
  source 1..30: mol_type = other DNA
tccggaatcagaggagaccgctacccaata

SEQ ID NO: 751 - DNA, 30 bp, Homo sapiens
  source 1..30: mol_type = other DNA
tccaacgtcagaggagacatggctcacagc

SEQ ID NO: 752 - DNA, 30 bp, Homo sapiens
  source 1..30: mol_type = other DNA
actgaggttagaggagaccgttcgcaggtt

SEQ ID NO: 753 - DNA, 30 bp, Homo sapiens
  source 1..30: mol_type = other DNA
gcgagcagcagaggagacctgcaacaaacg

SEQ ID NO: 754 - DNA, 30 bp, Homo sapiens
  source 1..30: mol_type = other DNA
cagtctgctagaggagaccttgtgtcttat

SEQ ID NO: 755 - DNA, 30 bp, Homo sapiens
  source 1..30: mol_type = other DNA
gacgcatctagaggagaccgaacatccctc

SEQ ID NO: 756 - DNA, 30 bp, Homo sapiens
  source 1..30: mol_type = other DNA
aacagtgtcagaggagacctcaaccaaact

SEQ ID NO: 757 - protein, 10 aa, Homo sapiens
TTGRGDQARL

SEQ ID NO: 758 - protein, 10 aa, Homo sapiens
TTLRGDHSQL

SEQ ID NO: 759 - protein, 10 aa, Homo sapiens
TNLRGDYQLT

SEQ ID NO: 760 - protein, 10 aa, Homo sapiens
STSRGDYVTH

SEQ ID NO: 761 - protein, 10 aa, Homo sapiens
MPARGDLVQT

SEQ ID NO: 762 - protein, 10 aa, Homo sapiens
SGVRGDRLAV

SEQ ID NO: 763 - protein, 10 aa, Homo sapiens
SNERGDQHRL

SEQ ID NO: 764 - protein, 10 aa, Homo sapiens
STLRGDYAVS

SEQ ID NO: 765 - protein, 10 aa, Homo sapiens
SNARGDIMTT

SEQ ID NO: 766 - protein, 10 aa, Homo sapiens
EASRGDLQMR

SEQ ID NO: 767 - protein, 10 aa, Homo sapiens
TYARGDLSGH

SEQ ID NO: 768 - protein, 10 aa, Homo sapiens
DSSRGDRSSV

SEQ ID NO: 769 - protein, 10 aa, Homo sapiens
SQSRGDLVPL

SEQ ID NO: 770 - protein, 10 aa, Homo sapiens
SATRGDYSIL

SEQ ID NO: 771 - protein, 10 aa, Homo sapiens
QATRGDLQHT

SEQ ID NO: 772 - protein, 10 aa, Homo sapiens
HDDRGDRRML

SEQ ID NO: 773 - protein, 10 aa, Homo sapiens
SSHRGDLLAT

SEQ ID NO: 774 - protein, 10 aa, Homo sapiens
SYQRGDQHNM

SEQ ID NO: 775 - protein, 10 aa, Homo sapiens
SHQRGDYNTL

SEQ ID NO: 776 - protein, 10 aa, Homo sapiens
MDQRGDMSVV

SEQ ID NO: 777 - protein, 10 aa, Homo sapiens
QIARGDLHVP

SEQ ID NO: 778 - protein, 10 aa, Homo sapiens
TYTRGDQHMM

SEQ ID NO: 779 - protein, 10 aa, Homo sapiens
DASRGDRTSL

SEQ ID NO: 780 - protein, 10 aa, Homo sapiens
SDRRGDLHPQ

SEQ ID NO: 781 - protein, 10 aa, Homo sapiens
SATRGDHSTL

SEQ ID NO: 782 - protein, 10 aa, Homo sapiens
AYQRGDFTSV

SEQ ID NO: 783 - protein, 10 aa, Homo sapiens
TGNRGDLQSV

SEQ ID NO: 784 - protein, 10 aa, Homo sapiens
AWRRGDLENA

SEQ ID NO: 785 - protein, 10 aa, Homo sapiens
NAARGDHGLL

SEQ ID NO: 786 - protein, 10 aa, Homo sapiens
SYSRGDHQAL

SEQ ID NO: 787 - protein, 10 aa, Homo sapiens
DGVRGDRTVV

SEQ ID NO: 788 - protein, 10 aa, Homo sapiens
SSERGDKMST

SEQ ID NO: 789 - protein, 10 aa, Homo sapiens
TSMRGDYTHV

SEQ ID NO: 790 - protein, 10 aa, Homo sapiens
SGVRGDRIII

SEQ ID NO: 791 - protein, 10 aa, Homo sapiens
ENRRGDAQLL

SEQ ID NO: 792 - protein, 10 aa, Homo sapiens
TVYRGDREQH

SEQ ID NO: 793 - protein, 10 aa, Homo sapiens
LDRRGDLLNS

SEQ ID NO: 794 - protein, 10 aa, Homo sapiens
ESVRGDLRST

SEQ ID NO: 795 - protein, 10 aa, Homo sapiens
DKYRGDQRGL

SEQ ID NO: 796 - protein, 10 aa, Homo sapiens
TDRRGDAHIL

SEQ ID NO: 797 - protein, 10 aa, Homo sapiens
SQTRGDTSLM

SEQ ID NO: 798 - protein, 10 aa, Homo sapiens
DATRGDTRML

SEQ ID NO: 799 - protein, 10 aa, Homo sapiens
SMQRGDLTHA

SEQ ID NO: 800 - protein, 10 aa, Homo sapiens
SGTRGDTLIL

SEQ ID NO: 801 - protein, 10 aa, Homo sapiens
ANTRGDHQNF

SEQ ID NO: 802 - protein, 10 aa, Homo sapiens
TSQRGDHTQL

SEQ ID NO: 803 - protein, 10 aa, Homo sapiens
SHWRGDTSSL

SEQ ID NO: 804 - protein, 10 aa, Homo sapiens
ISGRGDRQEL

SEQ ID NO: 805 - protein, 10 aa, Homo sapiens
SVLRGDIGTI

SEQ ID NO: 806 - protein, 10 aa, Homo sapiens
QDSRGDLSGK

SEQ ID NO: 807 - protein, 10 aa, Homo sapiens
KPIRGDGAEL

SEQ ID NO: 808 - protein, 10 aa, Homo sapiens
TYMRGDLQHT

SEQ ID NO: 809 - protein, 10 aa, Homo sapiens
TELRGDLSST

SEQ ID NO: 810 - protein, 10 aa, Homo sapiens
SAMRGDHAVL

SEQ ID NO: 811 - protein, 10 aa, Homo sapiens
SWVRGDASTL

SEQ ID NO: 812 - protein, 10 aa, Homo sapiens
QYARGDPHAL

SEQ ID NO: 813 - protein, 10 aa, Homo sapiens
STNRGDIVVH

SEQ ID NO: 814 - protein, 10 aa, Homo sapiens
VNIRGDLHQT

SEQ ID NO: 815 - protein, 10 aa, Homo sapiens
REYRGDSSIL

SEQ ID NO: 816 - protein, 10 aa, Homo sapiens
STLRGDGSMM

SEQ ID NO: 817 - protein, 10 aa, Homo sapiens
SSQRGDITAH

SEQ ID NO: 818 - protein, 10 aa, Homo sapiens
TEFRGDRSTV

SEQ ID NO: 819 - protein, 10 aa, Homo sapiens
TSTRGDYVTV

SEQ ID NO: 820 - protein, 10 aa, Homo sapiens
NHLRGDVSAI

SEQ ID NO: 821 - protein, 10 aa, Homo sapiens
LERRGDLVST

SEQ ID NO: 822 - protein, 10 aa, Homo sapiens
NEYRGDHSHP

SEQ ID NO: 823 - protein, 10 aa, Homo sapiens
NWQRGDTSTL

SEQ ID NO: 824 - protein, 10 aa, Homo sapiens
TDSRGDRTVL

SEQ ID NO: 825 - protein, 10 aa, Homo sapiens
YAVRGDQHTI

SEQ ID NO: 826 - protein, 10 aa, Homo sapiens
FGVRGDTRDL

SEQ ID NO: 827 - protein, 10 aa, Homo sapiens
SSARGDTGML

SEQ ID NO: 828 - protein, 10 aa, Homo sapiens
SAMRGDQMGQ

SEQ ID NO: 829 - protein, 10 aa, Homo sapiens
SHTRGDLVTT

SEQ ID NO: 830 - protein, 10 aa, Homo sapiens
VTTRGDTAIL

SEQ ID NO: 831 - protein, 10 aa, Homo sapiens
EPTRGDRHSI

SEQ ID NO: 832 - protein, 10 aa, Homo sapiens
GSQRGDRLVL

SEQ ID NO: 833 - protein, 10 aa, Homo sapiens
QHYRGDLSTT

SEQ ID NO: 834 - protein, 10 aa, Homo sapiens
METRGDRQQL

SEQ ID NO: 835 - protein, 10 aa, Homo sapiens
EALRGDKVVL

SEQ ID NO: 836 - protein, 10 aa, Homo sapiens
NAVRGDQSYV

SEQ ID NO: 837 - protein, 10 aa, Homo sapiens
SPLRGDNRAL

SEQ ID NO: 838 - protein, 10 aa, Homo sapiens
ENRRGDTHAL

SEQ ID NO: 839 - protein, 10 aa, Homo sapiens
NSQRGDLTGT

SEQ ID NO: 840 - protein, 9 aa, Homo sapiens
QYAPSQGTV

SEQ ID NO: 841 - protein, 10 aa, Homo sapiens
DNSRGDHSRL

SEQ ID NO: 842 - protein, 10 aa, Homo sapiens
NTHRGDYASI

SEQ ID NO: 843 - protein, 10 aa, Homo sapiens
HTTRGDQQPL

SEQ ID NO: 844 - protein, 10 aa, Homo sapiens
EWRRGDQTAL

SEQ ID NO: 845 - protein, 10 aa, Homo sapiens
TAVRGDQTTV

SEQ ID NO: 846 - protein, 10 aa, Homo sapiens
EYLRGDRISQ

SEQ ID NO: 847 - protein, 10 aa, Homo sapiens
SRYRGDQETI

SEQ ID NO: 848 - protein, 10 aa, Homo sapiens
ASLRGDTVTL

SEQ ID NO: 849 - protein, 10 aa, Homo sapiens
VGVRGDSHVL

SEQ ID NO: 850 - protein, 10 aa, Homo sapiens
TNSRGDHTSL

SEQ ID NO: 851 - protein, 10 aa, Homo sapiens
VDQRGDQRAL

SEQ ID NO: 852 - protein, 10 aa, Homo sapiens
ASVRGDRLDV

SEQ ID NO: 853 - protein, 10 aa, Homo sapiens
TSYRGDLSSN

SEQ ID NO: 854 - protein, 10 aa, Homo sapiens
TSDRGDSSNR

SEQ ID NO: 855 - protein, 10 aa, Homo sapiens
SYMRGDLDMR

SEQ ID NO: 856 - protein, 10 aa, Homo sapiens
TDVRGDRSMI

SEQ ID NO: 857 - protein, 10 aa, Homo sapiens
SYVRGDSSTV

SEQ ID NO: 858 - protein, 10 aa, Homo sapiens
QVYRGDRESV

SEQ ID NO: 859 - protein, 10 aa, Homo sapiens
TDRRGDAVSF

SEQ ID NO: 860 - protein, 10 aa, Homo sapiens
TNSRGDLHTT

SEQ ID NO: 861 - protein, 10 aa, Homo sapiens
EAIRGDFKTL

SEQ ID NO: 862 - protein, 10 aa, Homo sapiens
TGIRGDTAGL

SEQ ID NO: 863 - protein, 10 aa, Homo sapiens
TSSRGDVQSL

SEQ ID NO: 864 - protein, 10 aa, Homo sapiens
SSRRGDTNEL

SEQ ID NO: 865 - protein, 10 aa, Homo sapiens
SNARGDHTPL

SEQ ID NO: 866 - protein, 10 aa, Homo sapiens
SELRGDRSPY

SEQ ID NO: 867 - protein, 10 aa, Homo sapiens
VHARGDTSIM

SEQ ID NO: 868 - protein, 10 aa, Homo sapiens
SALRGDHGTL

SEQ ID NO: 869 - protein, 10 aa, Homo sapiens
QTGRGDIAGM

SEQ ID NO: 870 - protein, 10 aa, Homo sapiens
TVARGDQIRL

SEQ ID NO: 871 - protein, 10 aa, Homo sapiens
TNSRGDSVAL

SEQ ID NO: 872 - protein, 10 aa, Homo sapiens
QNARGDSAVI

SEQ ID NO: 873 - protein, 10 aa, Homo sapiens
HYLRGDTTTL

SEQ ID NO: 874 - protein, 10 aa, Homo sapiens
ANARGDLQNT

SEQ ID NO: 875 - protein, 10 aa, Homo sapiens
QNIRGDIRTV

SEQ ID NO: 876 - protein, 10 aa, Homo sapiens
AALRGDHGQM

SEQ ID NO: 877 - protein, 10 aa, Homo sapiens
TTDRGDLSAR

SEQ ID NO: 878 - protein, 10 aa, Homo sapiens
LNVRGDLVHT

SEQ ID NO: 879 - protein, 10 aa, Homo sapiens
YSTRGDHTVL

SEQ ID NO: 880 - protein, 10 aa, Homo sapiens
TNARGDLTVS

SEQ ID NO: 881 - protein, 10 aa, Homo sapiens
TNARGDYTAL

SEQ ID NO: 882 - protein, 10 aa, Homo sapiens
AEGRGDTRGS

SEQ ID NO: 883 - protein, 10 aa, Homo sapiens
TNIRGDQSSV

SEQ ID NO: 884 - protein, 10 aa, Homo sapiens
VDRRGDTMTM

SEQ ID NO: 885 - protein, 10 aa, Homo sapiens
QIGRGDVTPL

SEQ ID NO: 886 - protein, 10 aa, Homo sapiens
MQARGDLLGV

SEQ ID NO: 887 - protein, 10 aa, Homo sapiens
NTQRGDYASV

SEQ ID NO: 888 - protein, 10 aa, Homo sapiens
SDTRGDRITQ

SEQ ID NO: 889 - protein, 10 aa, Homo sapiens
SAIRGDYRDV

SEQ ID NO: 890 - protein, 10 aa, Homo sapiens
VGVRGDVHPL

SEQ ID NO: 891 - protein, 10 aa, Homo sapiens
TVLRGDAERL

SEQ ID NO: 892 - protein, 10 aa, Homo sapiens
ASTRGDLSGQ

SEQ ID NO: 893 - protein, 10 aa, Homo sapiens
DSRRGDGQNL

SEQ ID NO: 894 - protein, 10 aa, Homo sapiens
IDKRGDIQGL

SEQ ID NO: 895 - protein, 10 aa, Homo sapiens
TVTRGDLLVN

SEQ ID NO: 896 - protein, 10 aa, Homo sapiens
DSTRGDRLGL

SEQ ID NO: 897 - protein, 10 aa, Homo sapiens
AANRGDYASL

SEQ ID NO: 898 - protein, 10 aa, Homo sapiens
RDYRGDTSSL

SEQ ID NO: 899 - protein, 10 aa, Homo sapiens
SNIRGDASLH

SEQ ID NO: 900 - protein, 10 aa, Homo sapiens
NLQRGDRTDF

SEQ ID NO: 901 - protein, 10 aa, Homo sapiens
NASRGDHSSM

SEQ ID NO: 902 - protein, 10 aa, Homo sapiens
TSMRGDYTHV

SEQ ID NO: 903 - protein, 10 aa, Homo sapiens
TSRRGDLNDV

SEQ ID NO: 904 - protein, 10 aa, Homo sapiens
QSHRGDVQSL

SEQ ID NO: 905 - protein, 10 aa, Homo sapiens
STTRGDYQSV

SEQ ID NO: 906 - protein, 10 aa, Homo sapiens
YSTRGDHAML

SEQ ID NO: 907 - protein, 10 aa, Homo sapiens
TSARGDLIHT

SEQ ID NO: 908 - protein, 10 aa, Homo sapiens
ATARGDRESF

SEQ ID NO: 909 - protein, 10 aa, Homo sapiens
ASTRGDYSTM

SEQ ID NO: 910 - protein, 10 aa, Homo sapiens
SNNRGDVQRL

SEQ ID NO: 911 - protein, 10 aa, Homo sapiens
ERLRGDQHII

SEQ ID NO: 912 - protein, 10 aa, Homo sapiens
SSIRGDHATL

SEQ ID NO: 913 - protein, 10 aa, Homo sapiens
NPTRGDIATV

SEQ ID NO: 914 - protein, 10 aa, Homo sapiens
MSTRGDLSVN

SEQ ID NO: 915 - protein, 10 aa, Homo sapiens
MDRRGDITYA

SEQ ID NO: 916 - protein, 10 aa, Homo sapiens
NATRGDLMSA

SEQ ID NO: 917 - protein, 10 aa, Homo sapiens
SDHRGDRTYQ

SEQ ID NO: 918 - protein, 10 aa, Homo sapiens
NWVRGDLSTA

SEQ ID NO: 919 - protein, 10 aa, Homo sapiens
SSHRGDLSAT

SEQ ID NO: 920 - protein, 10 aa, Homo sapiens
NATRGDYREI

SEQ ID NO: 921 - protein, 10 aa, Homo sapiens
DTLRGDLQTA

SEQ ID NO: 922 - protein, 10 aa, Homo sapiens
IAVRGDLTPV

SEQ ID NO: 923 - protein, 10 aa, Homo sapiens
TLLRGDLAHS

SEQ ID NO: 924 - protein, 10 aa, Homo sapiens
MNTRGDHTVL

SEQ ID NO: 925 - protein, 10 aa, Homo sapiens
TGHRGDIAVY

SEQ ID NO: 926 - protein, 10 aa, Homo sapiens
TSNRGDQMSN

SEQ ID NO: 927 - protein, 10 aa, Homo sapiens
SATRGDHTAI

SEQ ID NO: 928 - protein, 10 aa, Homo sapiens
SSVRGDREHA

SEQ ID NO: 929 - protein, 10 aa, Homo sapiens
TNSRGDLVVH

SEQ ID NO: 930 - protein, 10 aa, Homo sapiens
ETRRGDLSSQ

SEQ ID NO: 931 - protein, 10 aa, Homo sapiens
TYARGDITLT

SEQ ID NO: 932 - protein, 10 aa, Homo sapiens
SASRGDTLHL

SEQ ID NO: 933 - protein, 10 aa, Homo sapiens
DNQRGDLSSR

SEQ ID NO: 934 - protein, 10 aa, Homo sapiens
TQSRGDLRDA

SEQ ID NO: 935 - protein, 10 aa, Homo sapiens
ENRRGDLTNA

SEQ ID NO: 936 - protein, 10 aa, Homo sapiens
TSIRGDISSV

SEQ ID NO: 937 - protein, 10 aa, Homo sapiens
ISARGDTMTL

SEQ ID NO: 938 - protein, 10 aa, Homo sapiens
SRVRGDQDHW

SEQ ID NO: 939 - protein, 10 aa, Homo sapiens
SGNRGDIASY

SEQ ID NO: 940 - protein, 10 aa, Homo sapiens
NEQRGDRYNH

SEQ ID NO: 941 - protein, 10 aa, Homo sapiens
EALRGDVRAL

SEQ ID NO: 942 - protein, 10 aa, Homo sapiens
VDRRGDYTQM

SEQ ID NO: 943 - protein, 10 aa, Homo sapiens
NNVRGDYTSV

SEQ ID NO: 944 - protein, 10 aa, Homo sapiens
QTYRGDASVL

SEQ ID NO: 945 - protein, 10 aa, Homo sapiens
SNYRGDHGSL

SEQ ID NO: 946 - protein, 10 aa, Homo sapiens
TSSRGDLREV

SEQ ID NO: 947 - protein, 10 aa, Homo sapiens
QYTRGDVSNL

SEQ ID NO: 948 - protein, 10 aa, Homo sapiens
SDLRGDVRER

SEQ ID NO: 949 - protein, 10 aa, Homo sapiens
QNARGDTTAL

SEQ ID NO: 950 - protein, 10 aa, Homo sapiens
NSLRGDRDTM

SEQ ID NO: 951 - protein, 10 aa, Homo sapiens
SNSRGDQAQV

SEQ ID NO: 952 - protein, 10 aa, Homo sapiens
EATRGDRSTL

SEQ ID NO: 953 - protein, 10 aa, Homo sapiens
VPQRGDITNL

SEQ ID NO: 954 - protein, 10 aa, Homo sapiens
DKIRGDLRSI

SEQ ID NO: 955 - protein, 10 aa, Homo sapiens
SAARGDYSIV

SEQ ID NO: 956 - protein, 10 aa, Homo sapiens
VGLRGDLIGQ

SEQ ID NO: 957 - protein, 10 aa, Homo sapiens
QAGRGDLTVI

SEQ ID NO: 958 - protein, 10 aa, Homo sapiens
SKTRGDFQEA

SEQ ID NO: 959 - protein, 10 aa, Homo sapiens
GTARGDYSML

SEQ ID NO: 960 - protein, 10 aa, Homo sapiens
ISTRGDLVTI

SEQ ID NO: 961 - protein, 10 aa, Homo sapiens
TGTRGDREYM

SEQ ID NO: 962 - protein, 10 aa, Homo sapiens
MTTRGDLSIT

SEQ ID NO: 963 - protein, 10 aa, Homo sapiens
STSRGDMTSV

SEQ ID NO: 964 - protein, 10 aa, Homo sapiens
GPARGDFTSS

SEQ ID NO: 965 - protein, 10 aa, Homo sapiens
VDRRGDLGHV

SEQ ID NO: 966 - protein, 10 aa, Homo sapiens
NSGRGDMSQL

SEQ ID NO: 967 - protein, 10 aa, Homo sapiens
SQGRGDLERY

SEQ ID NO: 968 - protein, 10 aa, Homo sapiens
VFGRGDQYTL

SEQ ID NO: 969 - protein, 10 aa, Homo sapiens
IVGRGDLHLQ

SEQ ID NO: 970 - protein, 10 aa, Homo sapiens
SYSRGDTSRL

SEQ ID NO: 971 - protein, 10 aa, Homo sapiens
MSSRGDRATI

SEQ ID NO: 972 - protein, 10 aa, Homo sapiens
MSSRGDLTRV

SEQ ID NO: 973 - protein, 10 aa, Homo sapiens
QHTRGDFGNL

SEQ ID NO: 974 - protein, 10 aa, Homo sapiens
ETRRGDLSSQ

SEQ ID NO: 975 - protein, 10 aa, Homo sapiens
TGTRGDLRAI

SEQ ID NO: 976 - protein, 10 aa, Homo sapiens
QMARGDLSTA

SEQ ID NO: 977 - protein, 10 aa, Homo sapiens
LVERGDRIMN

SEQ ID NO: 978 - protein, 10 aa, Homo sapiens
GLVRGDLNAR

SEQ ID NO: 979 - protein, 10 aa, Homo sapiens
NNPRGDLSSV

SEQ ID NO: 980 - protein, 10 aa, Homo sapiens
HEKRGDLSNS

SEQ ID NO: 981 - protein, 10 aa, Homo sapiens
SQARGDLHQT

SEQ ID NO: 982 - protein, 10 aa, Homo sapiens
FTGRGDLRTA

SEQ ID NO: 983 - protein, 10 aa, Homo sapiens
QSIRGDTGHL

SEQ ID NO: 984 - protein, 10 aa, Homo sapiens
SAGRGDMSVL

SEQ ID NO: 985 - protein, 10 aa, Homo sapiens
SERRGDEHKW

SEQ ID NO: 986 - protein, 10 aa, Homo sapiens
TTGRGDLVSL

SEQ ID NO: 987 - protein, 10 aa, Homo sapiens
LGLRGDQIGQ

SEQ ID NO: 988 - protein, 10 aa, Homo sapiens
VALRGDLTHQ

SEQ ID NO: 989 - protein, 10 aa, Homo sapiens
MIGRGDQAPL

SEQ ID NO: 990 - protein, 10 aa, Homo sapiens
FQARGDHSTL

SEQ ID NO: 991 - protein, 10 aa, Homo sapiens
NSVRGDHSSL

SEQ ID NO: 992 - protein, 10 aa, Homo sapiens
WPNRGDLQST

SEQ ID NO: 993 - protein, 10 aa, Homo sapiens
SASRGDYQTL

SEQ ID NO: 994 - protein, 10 aa, Homo sapiens
TMQRGDLHTI

SEQ ID NO: 995 - protein, 10 aa, Homo sapiens
TALRGDLAGT

SEQ ID NO: 996 - protein, 10 aa, Homo sapiens
VNARGDFSQT

SEQ ID NO: 997 - protein, 10 aa, Homo sapiens
GWIRGDRTSM

SEQ ID NO: 998 - protein, 10 aa, Homo sapiens
TFSRGDHTVL

SEQ ID NO: 999 - protein, 10 aa, Homo sapiens
SGARGDLVGV

SEQ ID NO: 1000 - protein, 10 aa, Homo sapiens
NNVRGDYTSV

SEQ ID NO: 1001 - protein, 10 aa, Homo sapiens
QTARGDHVVL

SEQ ID NO: 1002 - protein, 10 aa, Homo sapiens
TLIRGDLTRS

SEQ ID NO: 1003 - protein, 10 aa, Homo sapiens
VTTRGDRLEL

SEQ ID NO: 1004 - protein, 10 aa, Homo sapiens
EASRGDLQMR

SEQ ID NO: 1005 - protein, 10 aa, Homo sapiens
TYARGDLSGH

SEQ ID NO: 1006 - protein, 10 aa, Homo sapiens
DSSRGDRSSV

SEQ ID NO: 1007 - protein, 10 aa, Homo sapiens
SQSRGDLVPL

SEQ ID NO: 1008 - protein, 10 aa, Homo sapiens
MPARGDLVQT

SEQ ID NO: 1009 - protein, 10 aa, Homo sapiens
SGVRGDRLAV

SEQ ID NO: 1010 - protein, 10 aa, Homo sapiens
SNERGDQHRL

SEQ ID NO: 1011 - protein, 10 aa, Homo sapiens
STLRGDYAVS

SEQ ID NO: 1012 - protein, 10 aa, Homo sapiens
SNARGDIMTT

SEQ ID NO: 1013 - protein, 10 aa, Homo sapiens
TTGRGDQARL

SEQ ID NO: 1014 - protein, 10 aa, Homo sapiens
TTLRGDHSQL

SEQ ID NO: 1015 - protein, 10 aa, Homo sapiens
TNLRGDYQLT

SEQ ID NO: 1016 - protein, 10 aa, Homo sapiens
STSRGDYVTH

SEQ ID NO: 1017 - protein, 10 aa, Homo sapiens
SATRGDYSIL

SEQ ID NO: 1018 - protein, 10 aa, Homo sapiens
QATRGDLQHT

SEQ ID NO: 1019 - protein, 10 aa, Homo sapiens
HDDRGDRRML

SEQ ID NO: 1020 - protein, 10 aa, Homo sapiens
SSHRGDLLAT

SEQ ID NO: 1021 - protein, 10 aa, Homo sapiens
SYQRGDQHNM

SEQ ID NO: 1022 - protein, 10 aa, Homo sapiens
SHQRGDYNTL

SEQ ID NO: 1023 - protein, 10 aa, Homo sapiens
MDQRGDMSVV

SEQ ID NO: 1024 - protein, 10 aa, Homo sapiens
QIARGDLHVP

SEQ ID NO: 1025 - protein, 10 aa, Homo sapiens
TYTRGDQHMM

SEQ ID NO: 1026 - protein, 10 aa, Homo sapiens
DASRGDRTSL

SEQ ID NO: 1027 - protein, 10 aa, Homo sapiens
SDRRGDLHPQ

SEQ ID NO: 1028 - protein, 10 aa, Homo sapiens
SATRGDHSTL

SEQ ID NO: 1029 - protein, 10 aa, Homo sapiens
AYQRGDFTSV

SEQ ID NO: 1030 - protein, 10 aa, Homo sapiens
TGNRGDLQSV

SEQ ID NO: 1031 - protein, 10 aa, Homo sapiens
AWRRGDLENA

SEQ ID NO: 1032 - protein, 10 aa, Homo sapiens
NAARGDHGLL

SEQ ID NO: 1033 - protein, 10 aa, Homo sapiens
SYSRGDHQAL

SEQ ID NO: 1034 - protein, 10 aa, Homo sapiens
DGVRGDRTVV

SEQ ID NO: 1035 - protein, 10 aa, Homo sapiens
SSERGDKMST

SEQ ID NO: 1036 - protein, 10 aa, Homo sapiens
TSMRGDYTHV

SEQ ID NO: 1037 - protein, 10 aa, Homo sapiens
SGVRGDRIII

SEQ ID NO: 1038 - protein, 10 aa, Homo sapiens
ENRRGDAQLL

SEQ ID NO: 1039 - protein, 10 aa, Homo sapiens
TVYRGDREQH

SEQ ID NO: 1040 - protein, 10 aa, Homo sapiens
LDRRGDLLNS

SEQ ID NO: 1041 - protein, 10 aa, Homo sapiens
ESVRGDLRST

SEQ ID NO: 1042 - protein, 10 aa, Homo sapiens
DKYRGDQRGL

SEQ ID NO: 1043 - protein, 10 aa, Homo sapiens
TDRRGDAHIL

SEQ ID NO: 1044 - protein, 10 aa, Homo sapiens
SQTRGDTSLM

SEQ ID NO: 1045 - protein, 10 aa, Homo sapiens
DATRGDTRML

SEQ ID NO: 1046 - protein, 10 aa, Homo sapiens
SMQRGDLTHA

SEQ ID NO: 1047 - protein, 10 aa, Homo sapiens
SGTRGDTLIL

SEQ ID NO: 1048 - protein, 10 aa, Homo sapiens
ANTRGDHQNF

SEQ ID NO: 1049 - protein, 10 aa, Homo sapiens
TSQRGDHTQL

SEQ ID NO: 1050 - protein, 10 aa, Homo sapiens
SHWRGDTSSL

SEQ ID NO: 1051 - protein, 10 aa, Homo sapiens
ISGRGDRQEL

SEQ ID NO: 1052 - protein, 10 aa, Homo sapiens
SVLRGDIGTI

SEQ ID NO: 1053 - protein, 10 aa, Homo sapiens
QDSRGDLSGK

SEQ ID NO: 1054 - protein, 10 aa, Homo sapiens
KPIRGDGAEL

SEQ ID NO: 1055 - protein, 10 aa, Homo sapiens
TYMRGDLQHT

SEQ ID NO: 1056 - protein, 10 aa, Homo sapiens
TELRGDLSST

SEQ ID NO: 1057 - protein, 10 aa, Homo sapiens
SAMRGDHAVL

SEQ ID NO: 1058 - protein, 10 aa, Homo sapiens
SWVRGDASTL

SEQ ID NO: 1059 - protein, 10 aa, Homo sapiens
QYARGDPHAL

SEQ ID NO: 1060 - protein, 10 aa, Homo sapiens
STNRGDIVVH

SEQ ID NO: 1061 - protein, 10 aa, Homo sapiens
VNIRGDLHQT

SEQ ID NO: 1062 - protein, 10 aa, Homo sapiens
REYRGDSSIL

SEQ ID NO: 1063 - protein, 10 aa, Homo sapiens
STLRGDGSMM

SEQ ID NO: 1064 - protein, 10 aa, Homo sapiens
SSQRGDITAH

SEQ ID NO: 1065 - protein, 10 aa, Homo sapiens
TEFRGDRSTV

SEQ ID NO: 1066 - protein, 10 aa, Homo sapiens
TSTRGDYVTV

SEQ ID NO: 1067 - protein, 10 aa, Homo sapiens
NHLRGDVSAI

SEQ ID NO: 1068 - protein, 10 aa, Homo sapiens
LERRGDLVST

SEQ ID NO: 1069 - protein, 10 aa, Homo sapiens
NEYRGDHSHP

SEQ ID NO: 1070 - protein, 10 aa, Homo sapiens
NWQRGDTSTL

SEQ ID NO: 1071 - protein, 10 aa, Homo sapiens
TDSRGDRTVL

SEQ ID NO: 1072 - protein, 10 aa, Homo sapiens
YAVRGDQHTI

SEQ ID NO: 1073 - protein, 10 aa, Homo sapiens
FGVRGDTRDL

SEQ ID NO: 1074 - protein, 10 aa, Homo sapiens
SSARGDTGML

SEQ ID NO: 1075 - protein, 10 aa, Homo sapiens
SAMRGDQMGQ

SEQ ID NO: 1076 - protein, 10 aa, Homo sapiens
SHTRGDLVTT

SEQ ID NO: 1077 - protein, 10 aa, Homo sapiens
VTTRGDTAIL

SEQ ID NO: 1078 - protein, 10 aa, Homo sapiens
EPTRGDRHSI

SEQ ID NO: 1079 - protein, 10 aa, Homo sapiens
GSQRGDRLVL

SEQ ID NO: 1080 - protein, 10 aa, Homo sapiens
QHYRGDLSTT

SEQ ID NO: 1081 - protein, 10 aa, Homo sapiens
METRGDRQQL

SEQ ID NO: 1082 - protein, 10 aa, Homo sapiens
EALRGDKVVL

SEQ ID NO: 1083 - protein, 10 aa, Homo sapiens
NAVRGDQSYV

SEQ ID NO: 1084 - protein, 10 aa, Homo sapiens
SPLRGDNRAL

SEQ ID NO: 1085 - protein, 10 aa, Homo sapiens
ENRRGDTHAL

SEQ ID NO: 1086 - protein, 10 aa, Homo sapiens
NSQRGDLTGT

SEQ ID NO: 1087 - protein, 10 aa, Homo sapiens
DNSRGDHSRL

SEQ ID NO: 1088 - protein, 10 aa, Homo sapiens
NTHRGDYASI

SEQ ID NO: 1089 - protein, 10 aa, Homo sapiens
HTTRGDQQPL

SEQ ID NO: 1090 - protein, 10 aa, Homo sapiens
EWRRGDQTAL

SEQ ID NO: 1091 - protein, 10 aa, Homo sapiens
TAVRGDQTTV

SEQ ID NO: 1092 - protein, 10 aa, Homo sapiens
EYLRGDRISQ

SEQ ID NO: 1093 - protein, 10 aa, Homo sapiens
SRYRGDQETI

SEQ ID NO: 1094 - protein, 10 aa, Homo sapiens
ASLRGDTVTL

SEQ ID NO: 1095 - protein, 10 aa, Homo sapiens
VGVRGDSHVL

SEQ ID NO: 1096 - protein, 10 aa, Homo sapiens
TNSRGDHTSL

SEQ ID NO: 1097 - protein, 10 aa, Homo sapiens
VDQRGDQRAL

SEQ ID NO: 1098 - protein, 10 aa, Homo sapiens
ASVRGDRLDV

SEQ ID NO: 1099 - protein, 10 aa, Homo sapiens
TSYRGDLSSN

SEQ ID NO: 1100 - protein, 10 aa, Homo sapiens
TSDRGDSSNR

SEQ ID NO: 1101 - protein, 10 aa, Homo sapiens
SYMRGDLDMR

SEQ ID NO: 1102 - protein, 10 aa, Homo sapiens
TDVRGDRSMI

SEQ ID NO: 1103 - protein, 10 aa, Homo sapiens
SYVRGDSSTV

SEQ ID NO: 1104 - protein, 10 aa, Homo sapiens
QVYRGDRESV

SEQ ID NO: 1105 - protein, 10 aa, Homo sapiens
TDRRGDAVSF

SEQ ID NO: 1106 - protein, 10 aa, Homo sapiens
TNSRGDLHTT

SEQ ID NO: 1107 - protein, 10 aa, Homo sapiens
EAIRGDFKTL

SEQ ID NO: 1108 - protein, 10 aa, Homo sapiens
TGIRGDTAGL

SEQ ID NO: 1109 - protein, 10 aa, Homo sapiens
TSSRGDVQSL

SEQ ID NO: 1110 - protein, 10 aa, Homo sapiens
SSRRGDTNEL

SEQ ID NO: 1111 - protein, 10 aa, Homo sapiens
SNARGDHTPL

SEQ ID NO: 1112 - protein, 10 aa, Homo sapiens
SELRGDRSPY

SEQ ID NO: 1113 - protein, 10 aa, Homo sapiens
VHARGDTSIM

SEQ ID NO: 1114 - protein, 10 aa, Homo sapiens
SALRGDHGTL

SEQ ID NO: 1115 - protein, 10 aa, Homo sapiens
QTGRGDIAGM

SEQ ID NO: 1116 - protein, 10 aa, Homo sapiens
TVARGDQIRL

SEQ ID NO: 1117 - protein, 10 aa, Homo sapiens
TNSRGDSVAL

SEQ ID NO: 1118 - protein, 10 aa, Homo sapiens
QNARGDSAVI

SEQ ID NO: 1119 - protein, 10 aa, Homo sapiens
HYLRGDTTTL

SEQ ID NO: 1120 - protein, 10 aa, Homo sapiens
ANARGDLQNT

SEQ ID NO: 1121 - protein, 10 aa, Homo sapiens
QNIRGDIRTV

SEQ ID NO: 1122 - protein, 10 aa, Homo sapiens
AALRGDHGQM

SEQ ID NO: 1123 - protein, 10 aa, Homo sapiens
TTDRGDLSAR

SEQ ID NO: 1124 - protein, 10 aa, Homo sapiens
LNVRGDLVHT

SEQ ID NO: 1125 - protein, 10 aa, Homo sapiens
YSTRGDHTVL

SEQ ID NO: 1126 - protein, 10 aa, Homo sapiens
TNARGDLTVS

SEQ ID NO: 1127 - protein, 10 aa, Homo sapiens
TNARGDYTAL

SEQ ID NO: 1128 - protein, 10 aa, Homo sapiens
AEGRGDTRGS

SEQ ID NO: 1129 - protein, 10 aa, Homo sapiens
TNIRGDQSSV

SEQ ID NO: 1130 - protein, 10 aa, Homo sapiens
VDRRGDTMTM

SEQ ID NO: 1131 - protein, 10 aa, Homo sapiens
QIGRGDVTPL

SEQ ID NO: 1132 - protein, 10 aa, Homo sapiens
MQARGDLLGV

SEQ ID NO: 1133 - protein, 10 aa, Homo sapiens
NTQRGDYASV

SEQ ID NO: 1134 - protein, 10 aa, Homo sapiens
SDTRGDRITQ

SEQ ID NO: 1135 - protein, 10 aa, Homo sapiens
SAIRGDYRDV

SEQ ID NO: 1136 - protein, 10 aa, Homo sapiens
VGVRGDVHPL

SEQ ID NO: 1137 - protein, 10 aa, Homo sapiens
TVLRGDAERL

SEQ ID NO: 1138 - protein, 10 aa, Homo sapiens
ASTRGDLSGQ

SEQ ID NO: 1139 - protein, 10 aa, Homo sapiens
DSRRGDGQNL

SEQ ID NO: 1140 - protein, 10 aa, Homo sapiens
IDKRGDIQGL

SEQ ID NO: 1141 - protein, 10 aa, Homo sapiens
TVTRGDLLVN

SEQ ID NO: 1142 - protein, 10 aa, Homo sapiens
DSTRGDRLGL

SEQ ID NO: 1143 - protein, 10 aa, Homo sapiens
AANRGDYASL

SEQ ID NO: 1144 - protein, 10 aa, Homo sapiens
RDYRGDTSSL

SEQ ID NO: 1145 - protein, 10 aa, Homo sapiens
SNIRGDASLH

SEQ ID NO: 1146 - protein, 10 aa, Homo sapiens
NLQRGDRTDF

SEQ ID NO: 1147 - protein, 10 aa, Homo sapiens
NASRGDHSSM

SEQ ID NO: 1148 - protein, 10 aa, Homo sapiens
TSMRGDYTHV

SEQ ID NO: 1149 - protein, 10 aa, Homo sapiens
TSRRGDLNDV

SEQ ID NO: 1150 - protein, 10 aa, Homo sapiens
QSHRGDVQSL

SEQ ID NO: 1151 - protein, 10 aa, Homo sapiens
STTRGDYQSV

SEQ ID NO: 1152 - protein, 10 aa, Homo sapiens
YSTRGDHAML

SEQ ID NO: 1153 - protein, 10 aa, Homo sapiens
TSARGDLIHT

SEQ ID NO: 1154 - protein, 10 aa, Homo sapiens
ATARGDRESF

SEQ ID NO: 1155 - protein, 10 aa, Homo sapiens
ASTRGDYSTM

SEQ ID NO: 1156 - protein, 10 aa, Homo sapiens
SNNRGDVQRL

SEQ ID NO: 1157 - protein, 10 aa, Homo sapiens
ERLRGDQHII

SEQ ID NO: 1158 - protein, 10 aa, Homo sapiens
SSIRGDHATL

SEQ ID NO: 1159 - protein, 10 aa, Homo sapiens
NPTRGDIATV

SEQ ID NO: 1160 - protein, 10 aa, Homo sapiens
MSTRGDLSVN

SEQ ID NO: 1161 - protein, 10 aa, Homo sapiens
MDRRGDITYA

SEQ ID NO: 1162 - protein, 10 aa, Homo sapiens
NATRGDLMSA

SEQ ID NO: 1163 - protein, 10 aa, Homo sapiens
SDHRGDRTYQ

SEQ ID NO: 1164 - protein, 10 aa, Homo sapiens
NWVRGDLSTA

SEQ ID NO: 1165 - protein, 10 aa, Homo sapiens
SSHRGDLSAT

SEQ ID NO: 1166 - protein, 10 aa, Homo sapiens
NATRGDYREI

SEQ ID NO: 1167 - protein, 10 aa, Homo sapiens
DTLRGDLQTA

SEQ ID NO: 1168 - protein, 10 aa, Homo sapiens
IAVRGDLTPV

SEQ ID NO: 1169 - protein, 10 aa, Homo sapiens
TLLRGDLAHS

SEQ ID NO: 1170 - protein, 10 aa, Homo sapiens
MNTRGDHTVL

SEQ ID NO: 1171 - protein, 10 aa, Homo sapiens
TGHRGDIAVY

SEQ ID NO: 1172 - protein, 10 aa, Homo sapiens
TSNRGDQMSN

SEQ ID NO: 1173 - protein, 10 aa, Homo sapiens
SATRGDHTAI

SEQ ID NO: 1174 - protein, 10 aa, Homo sapiens
SSVRGDREHA

SEQ ID NO: 1175 - protein, 10 aa, Homo sapiens
TNSRGDLVVH

SEQ ID NO: 1176 - protein, 10 aa, Homo sapiens
ETRRGDLSSQ

SEQ ID NO: 1177 - protein, 10 aa, Homo sapiens
TYARGDITLT

SEQ ID NO: 1178 - protein, 10 aa, Homo sapiens
SASRGDTLHL

SEQ ID NO: 1179 - protein, 10 aa, Homo sapiens
DNQRGDLSSR

SEQ ID NO: 1180 - protein, 10 aa, Homo sapiens
TQSRGDLRDA

SEQ ID NO: 1181 - protein, 10 aa, Homo sapiens
ENRRGDLTNA

SEQ ID NO: 1182 - protein, 10 aa, Homo sapiens
TSIRGDISSV

SEQ ID NO: 1183 - protein, 10 aa, Homo sapiens
ISARGDTMTL

SEQ ID NO: 1184 - protein, 10 aa, Homo sapiens
SRVRGDQDHW

SEQ ID NO: 1185 - protein, 10 aa, Homo sapiens
SGNRGDIASY

SEQ ID NO: 1186 - protein, 10 aa, Homo sapiens
NEQRGDRYNH

SEQ ID NO: 1187 - protein, 10 aa, Homo sapiens
EALRGDVRAL

SEQ ID NO: 1188 - protein, 10 aa, Homo sapiens
VDRRGDYTQM

SEQ ID NO: 1189 - protein, 10 aa, Homo sapiens
NNVRGDYTSV

SEQ ID NO: 1190 - protein, 10 aa, Homo sapiens
QTYRGDASVL

SEQ ID NO: 1191 - protein, 10 aa, Homo sapiens
SNYRGDHGSL

SEQ ID NO: 1192 - protein, 10 aa, Homo sapiens
TSSRGDLREV

SEQ ID NO: 1193 - protein, 10 aa, Homo sapiens
QYTRGDVSNL

SEQ ID NO: 1194 - protein, 10 aa, Homo sapiens
SDLRGDVRER

SEQ ID NO: 1195 - protein, 10 aa, Homo sapiens
QNARGDTTAL

SEQ ID NO: 1196 - protein, 10 aa, Homo sapiens
NSLRGDRDTM

SEQ ID NO: 1197 - protein, 10 aa, Homo sapiens
SNSRGDQAQV

SEQ ID NO: 1198 - protein, 10 aa, Homo sapiens
EATRGDRSTL

SEQ ID NO: 1199 - protein, 10 aa, Homo sapiens
VPQRGDITNL

SEQ ID NO: 1200 - protein, 10 aa, Homo sapiens
DKIRGDLRSI

SEQ ID NO: 1201 - protein, 10 aa, Homo sapiens
SAARGDYSIV

SEQ ID NO: 1202 - protein, 10 aa, Homo sapiens
VGLRGDLIGQ

SEQ ID NO: 1203 - protein, 10 aa, Homo sapiens
QAGRGDLTVI

SEQ ID NO: 1204 - protein, 10 aa, Homo sapiens
SKTRGDFQEA

SEQ ID NO: 1205 - protein, 10 aa, Homo sapiens
GTARGDYSML

SEQ ID NO: 1206 - protein, 10 aa, Homo sapiens
ISTRGDLVTI

SEQ ID NO: 1207 - protein, 10 aa, Homo sapiens
TGTRGDREYM

SEQ ID NO: 1208 - protein, 10 aa, Homo sapiens
MTTRGDLSIT

SEQ ID NO: 1209 - protein, 10 aa, Homo sapiens
STSRGDMTSV

SEQ ID NO: 1210 - protein, 10 aa, Homo sapiens
GPARGDFTSS

SEQ ID NO: 1211 - protein, 10 aa, Homo sapiens
VDRRGDLGHV

SEQ ID NO: 1212 - protein, 10 aa, Homo sapiens
NSGRGDMSQL

SEQ ID NO: 1213 - protein, 10 aa, Homo sapiens
SQGRGDLERY

SEQ ID NO: 1214 - protein, 10 aa, Homo sapiens
VFGRGDQYTL

SEQ ID NO: 1215 - protein, 10 aa, Homo sapiens
IVGRGDLHLQ

SEQ ID NO: 1216 - protein, 10 aa, Homo sapiens
SYSRGDTSRL

SEQ ID NO: 1217 - protein, 10 aa, Homo sapiens
MSSRGDRATI

SEQ ID NO: 1218 - protein, 10 aa, Homo sapiens
MSSRGDLTRV

SEQ ID NO: 1219 - protein, 10 aa, Homo sapiens
QHTRGDFGNL

SEQ ID NO: 1220 - protein, 10 aa, Homo sapiens
ETRRGDLSSQ

SEQ ID NO: 1221 - protein, 10 aa, Homo sapiens
TGTRGDLRAI

SEQ ID NO: 1222 - protein, 10 aa, Homo sapiens
QMARGDLSTA

SEQ ID NO: 1223 - protein, 10 aa, Homo sapiens
LVERGDRIMN

SEQ ID NO: 1224 - protein, 10 aa, Homo sapiens
GLVRGDLNAR

SEQ ID NO: 1225 - protein, 10 aa, Homo sapiens
NNPRGDLSSV

SEQ ID NO: 1226 - protein, 10 aa, Homo sapiens
HEKRGDLSNS

SEQ ID NO: 1227 - protein, 10 aa, Homo sapiens
SQARGDLHQT

SEQ ID NO: 1228 - protein, 10 aa, Homo sapiens
FTGRGDLRTA

SEQ ID NO: 1229 - protein, 10 aa, Homo sapiens
QSIRGDTGHL

SEQ ID NO: 1230 - protein, 10 aa, Homo sapiens
SAGRGDMSVL

SEQ ID NO: 1231 - protein, 10 aa, Homo sapiens
SERRGDEHKW

SEQ ID NO: 1232 - protein, 10 aa, Homo sapiens
TTGRGDLVSL

SEQ ID NO: 1233 - protein, 10 aa, Homo sapiens
LGLRGDQIGQ

SEQ ID NO: 1234 - protein, 10 aa, Homo sapiens
VALRGDLTHQ

SEQ ID NO: 1235 - protein, 10 aa, Homo sapiens
MIGRGDQAPL

SEQ ID NO: 1236 - protein, 10 aa, Homo sapiens
FQARGDHSTL

SEQ ID NO: 1237 - protein, 10 aa, Homo sapiens
NSVRGDHSSL

SEQ ID NO: 1238 - protein, 10 aa, Homo sapiens
WPNRGDLQST

SEQ ID NO: 1239 - protein, 10 aa, Homo sapiens
SASRGDYQTL

SEQ ID NO: 1240 - protein, 10 aa, Homo sapiens
TMQRGDLHTI

SEQ ID NO: 1241 - protein, 10 aa, Homo sapiens
TALRGDLAGT

SEQ ID NO: 1242 - protein, 10 aa, Homo sapiens
VNARGDFSQT

SEQ ID NO: 1243 - protein, 10 aa, Homo sapiens
GWIRGDRTSM

SEQ ID NO: 1244 - protein, 10 aa, Homo sapiens
TFSRGDHTVL

SEQ ID NO: 1245 - protein, 10 aa, Homo sapiens
SGARGDLVGV

SEQ ID NO: 1246 - protein, 10 aa, Homo sapiens
NNVRGDYTSV

SEQ ID NO: 1247 - protein, 10 aa, Homo sapiens
QTARGDHVVL

SEQ ID NO: 1248 - protein, 10 aa, Homo sapiens
TLIRGDLTRS

SEQ ID NO: 1249 - protein, 10 aa, Homo sapiens
VTTRGDRLEL

SEQ ID NO: 1250 - DNA, 91 bp, synthetic construct
  misc_feature 1..91: note = Synthetic Polynucleotide
  misc_feature 1..91: note = mi405
  source 1..91: mol_type = other DNA
ctcgagtgagcgatccaggattcagatctggtttctatttagtgtgataatggtgtttaaaccagatctgaatcctggactgcctactagt

SEQ ID NO: 1251 - DNA, 88 bp, synthetic construct
  misc_feature 1..88: note = Synthetic Polynucleotide
  misc_feature 1..88: note = mi1155
  source 1..88: mol_type = other DNA
ctcgagtgagcgacaggcgcaacctctcctagaatatttagtgtgataatggtgtttctaggagaggttgcgcctgctgcctactagt

SEQ ID NO: 1252 - DNA, 114 bp, synthetic construct
  misc_feature 1..114: note = Synthetic Polynucleotide
  misc_feature 1..114: note = mi405
  source 1..114: mol_type = other DNA
ctcgagtatttagtgtgataatggtgtttctcgggtgagcgatccaggattcagatctggtttctgaaagccacagatgggaaaccagatctgaatcctggactgcctactagt

SEQ ID NO: 1253 - DNA, 115 bp, synthetic construct
  misc_feature 1..115: note = Synthetic Polynucleotide
  misc_feature 1..115: note = mi1155
  source 1..115: mol_type = other DNA
ctcgagtatttagtgtgataatggtgtttctcgggtgagcgacaggcgcaacctctcctagaactgtaaagccacagatgggttctaggagaggttgcgcctgctgcctactagt

SEQ ID NO: 1254 - DNA, 114 bp, synthetic construct
  misc_feature 1..114: note = Synthetic Polynucleotide
  misc_feature 1..114: note = mi405
  source 1..114: mol_type = other DNA
ctcgagtgagcgatccaggattcagatctggtttctgaaagccacagatgggaaaccagatctgaatcctggactgcctactagatatttagtgtgataatggtgtttactagt

SEQ ID NO: 1255 - DNA, 115 bp, synthetic construct
  misc_feature 1..115: note = Synthetic Polynucleotide
  misc_feature 1..115: note = mi1155
  source 1..115: mol_type = other DNA
ctcgagtgagcgacaggcgcaacctctcctagaactgtaaagccacagatgggttctaggagaggttgcgcctgctgcctactagatatttagtgtgataatggtgtttactagt

SEQ ID NO: 1256 - DNA, 93 bp, synthetic construct
  misc_feature 1..93: note = Synthetic Polynucleotide
  misc_feature 1..93: note = mi405
  source 1..93: mol_type = other DNA
ctcgagtgagcgatccaggattcagatctggtttctacgagccttttgctcgtcttatggaaaccagatctgaatcctggactgcctactagt

SEQ ID NO: 1257 - DNA, 93 bp, synthetic construct
  misc_feature 1..93: note = Synthetic Polynucleotide
  misc_feature 1..93: note = mi1155
  source 1..93: mol_type = other DNA
ctcgagtgagcgacaggcgcaacctctcctagaactacgagccttttgctcgtcttatggttctaggagaggttgcgcctgctgcctactagt

SEQ ID NO: 1258 - DNA, 114 bp, synthetic construct
  misc_feature 1..114: note = Synthetic Polynucleotide
  misc_feature 1..114: note = mi405
  source 1..114: mol_type = other DNA
ctcgagacgagccttttgctcgtcttatctcgggtgagcgatccaggattcagatctggtttctgtaaagccacagatgggaaaccagatctgaatcctggactgcctactagt

SEQ ID NO: 1259 - DNA, 114 bp, synthetic construct
  misc_feature 1..114: note = Synthetic Polynucleotide
  misc_feature 1..114: note = mi1155
  source 1..114: mol_type = other DNA
ctcgagacgagctttttgctcgtcttatctcgggtgagcgacaggcgcaacctctcctagaactgtaaagccacagatgggttctaggagaggttgcgcctgctgcctactagt

SEQ ID NO: 1260 - DNA, 114 bp, synthetic construct
  misc_feature 1..114: note = Synthetic Polynucleotide
  misc_feature 1..114: note = mi405
  source 1..114: mol_type = other DNA
ctcgagtgagcgatccaggattcagatctggtttctgtaaagccacagatgggaaaccagatctgaatcctggactgcctactagaacgagccttttgctcgtcttatactagt

SEQ ID NO: 1261 - DNA, 114 bp, synthetic construct
  misc_feature 1..114: note = Synthetic Polynucleotide
  misc_feature 1..114: note = mi1155
  source 1..114: mol_type = other DNA
ctcgagtgagcgacaggcgcaacctctcctagaactgtaaagccacagatgggttctaggagaggttgcgcctgctgcctactagaacgagccttttgctcgtcttatactagt

SEQ ID NO: 1262 - DNA, 143 bp, synthetic construct
  misc_feature 1..143: note = Synthetic Polynucleotide
  misc_feature 1..143: note = mi405
  source 1..143: mol_type = other DNA
ctcgagtatttagtgtgataatggtgtttctcgggtgagcgatccaggattcagatctggtttctgtaaagccacagatgggaaaccagatctgaatcctggactgcctactagaacgagccttttgctcgtcttatactagt

SEQ ID NO: 1263 - DNA, 143 bp, synthetic construct
  misc_feature 1..143: note = Synthetic Polynucleotide
  misc_feature 1..143: note = mi1155
  source 1..143: mol_type = other DNA
ctcgagtatttagtgtgataatggtgtttctcgggtgagcgacaggcgcaacctctcctagaactgtaaagccacagatgggttctaggagaggttgcgcctgctgcctactagaacgagccttttgctcgtcttatactagt

SEQ ID NO: 1264 - DNA, 137 bp, synthetic construct
  misc_feature 1..137: note = Synthetic Polynucleotide
  misc_feature 1..137: note = mi405
  source 1..137: mol_type = other DNA
ctcgagtatttagtgtgataatggtgtttacgagccttttgctcgtcttatctcgggtgagcgatccaggattcagatctggtttctgtaaagccacagatgggaaaccagatctgaatcctggactgcctactagt

SEQ ID NO: 1265 - DNA, 137 bp, synthetic construct
  misc_feature 1..137: note = Synthetic Polynucleotide
  misc_feature 1..137: note = mi1155
  source 1..137: mol_type = other DNA
ctcgagtatttagtgtgataatggtgtttacgagccttttgctcgtcttatctcgggtgagcgacaggcgcaacctctcctagaactgtaaagccacagatgggttctaggagaggttgcgcctgctgcctactagt

SEQ ID NO: 1266 - DNA, 137 bp, synthetic construct
  misc_feature 1..137: note = Synthetic Polynucleotide
  misc_feature 1..137: note = mi405
  source 1..137: mol_type = other DNA
ctcgagtgagcgatccaggattcagatctggtttctgtaaagccacagatgggaaaccagatctgaatcctggactgcctactagatatttagtgtgataatggtgtttacgagccttttgctcgtcttatactagt

SEQ ID NO: 1267 - DNA, 137 bp, synthetic construct
  misc_feature 1..137: note = Synthetic Polynucleotide
  misc_feature 1..137: note = mi1155
  source 1..137: mol_type = other DNA
ctcgagtgagcgacaggcgcaacctctcctagaactgtaaagccacagatgggttctaggagaggttgcgcctgctgcctactagatatttagtgtgataatggtgtttacgagccttttgctcgtcttatactagt

SEQ ID NO: 1268 - DNA, 122 bp, synthetic construct
  misc_feature 1..122: note = Synthetic Polynucleotide
  misc_feature 1..122: note = mi405
  source 1..122: mol_type = other DNA
ctcgagacgagccttttgctcgtcttatctcgggtgagcgatccaggattcagatctggtttcttatttagtgtgataatggtgtttggaaaccagatctgaatcctggactgcctactagt

SEQ ID NO: 1269 - DNA, 122 bp, synthetic construct
  misc_feature 1..122: note = Synthetic Polynucleotide
  misc_feature 1..122: note = mi1155
  source 1..122: mol_type = other DNA
ctcgagacgagccttttgctcgtcttatctcgggtgagcgacaggcgcaacctctcctagaacttatttagtgtgataatggtgtttggttctaggagaggttgcgcctgctgcctactagt

SEQ ID NO: 1270 - DNA, 122 bp, synthetic construct
  misc_feature 1..122: note = Synthetic Polynucleotide
  misc_feature 1..122: note = mi405
  source 1..122: mol_type = other DNA
ctcgagtgagcgatccaggattcagatctggtttcttatttagtgtgataatggtgtttggaaaccagatctgaatcctggactgcctactagaacgagccttttgctcgtcttatactagt

SEQ ID NO: 1271 - DNA, 121 bp, synthetic construct
  misc_feature 1..121: note = Synthetic Polynucleotide
  misc_feature 1..121: note = mi1155
  source 1..121: mol_type = other DNA
tcgagtgagcgacaggcgcaacctctcctagaacttatttagtgtgataatggtgtttggttctaggagaggttgcgcctgctgcctactagaacgagccttttgctcgtcttatactagt

SEQ ID NO: 1272 - DNA, 122 bp, synthetic construct
  misc_feature 1..122: note = Synthetic Polynucleotide
  misc_feature 1..122: note = mi405
  source 1..122: mol_type = other DNA
ctcgagtatttagtgtgataatggtgtttctcgggtgagcgatccaggattcagatctggtttctacgagccttttgctcgtcttatggaaaccagatctgaatcctggactgcctactagt

SEQ ID NO: 1273 - DNA, 122 bp, synthetic construct
  misc_feature 1..122: note = Synthetic Polynucleotide
  misc_feature 1..122: note = mi1155
  source 1..122: mol_type = other DNA
ctcgagtatttagtgtgataatggtgtttctcgggtgagcgacaggcgcaacctctcctagaactacgagccttttgctcgtcttatggttctaggagaggttgcgcctgctgcctactagt

SEQ ID NO: 1274 - DNA, 122 bp, synthetic construct
  misc_feature 1..122: note = Synthetic Polynucleotide
  misc_feature 1..122: note = mi405
  source 1..122: mol_type = other DNA
ctcgagtgagcgatccaggattcagatctggtttctacgagccttttgctcgtcttatggaaaccagatctgaatcctggactgcctactagatatttagtgtgataatggtgtttactagt

SEQ ID NO: 1275 - DNA, 122 bp, synthetic construct
  misc_feature 1..122: note = Synthetic Polynucleotide
  misc_feature 1..122: note = mi1155
  source 1..122: mol_type = other DNA
ctcgagtgagcgacaggcgcaacctctcctagaactacgagccttttgctcgtcttatggttctaggagaggttgcgcctgctgcctactagatatttagtgtgataatggtgtttactagt

SEQ ID NO: 1276 - DNA, 115 bp, synthetic construct
  misc_feature 1..115: note = Synthetic Polynucleotide
  misc_feature 1..115: note = mi405
  source 1..115: mol_type = other DNA
ctcgagtgagcgatccaggattcagatctggtttcttttagtgtgataatggtgtttgacgagctttttgctcgtcttatggaaaccagatctgaatcctggactgcctactagt

SEQ ID NO: 1277 - DNA, 115 bp, synthetic construct
  misc_feature 1..115: note = Synthetic Polynucleotide
  misc_feature 1..115: note = mi1155
  source 1..115: mol_type = other DNA
ctcgagtgagcgacaggcgcaacctctcctagaacttttagtgtgataatggtgtttgacgagctttttgctcgtcttatggttctaggagaggttgcgcctgctgcctactagt

SEQ ID NO: 1278 - RNA, 91 nt, synthetic construct
  misc_feature 1..91: note = Synthetic Polynucleotide
  misc_feature 1..91: note = mi405
  source 1..91: mol_type = other RNA
ctcgagtgagcgatccaggattcagatctggtttctatttagtgtgataatggtgtttaaaccagatctgaatcctggactgcctactagt

SEQ ID NO: 1279 - RNA, 88 nt, synthetic construct
  misc_feature 1..88: note = Synthetic Polynucleotide
  misc_feature 1..88: note = mi1155
  source 1..88: mol_type = other RNA
ctcgagtgagcgacaggcgcaacctctcctagaatatttagtgtgataatggtgtttctaggagaggttgcgcctgctgcctactagt

SEQ ID NO: 1280 - RNA, 114 nt, synthetic construct
  misc_feature 1..114: note = Synthetic Polynucleotide
  misc_feature 1..114: note = mi405
  source 1..114: mol_type = other RNA
ctcgagtatttagtgtgataatggtgtttctcgggtgagcgatccaggattcagatctggtttctgaaagccacagatgggaaaccagatctgaatcctggactgcctactagt

SEQ ID NO: 1281 - RNA, 115 nt, synthetic construct
  misc_feature 1..115: note = Synthetic Polynucleotide
  misc_feature 1..115: note = mi1155
  source 1..115: mol_type = other RNA
ctcgagtatttagtgtgataatggtgtttctcgggtgagcgacaggcgcaacctctcctagaactgtaaagccacagatgggttctaggagaggttgcgcctgctgcctactagt

SEQ ID NO: 1282 - RNA, 114 nt, synthetic construct
  misc_feature 1..114: note = Synthetic Polynucleotide
  misc_feature 1..114: note = mi405
  source 1..114: mol_type = other RNA
ctcgagtgagcgatccaggattcagatctggtttctgaaagccacagatgggaaaccagatctgaatcctggactgcctactagatatttagtgtgataatggtgtttactagt

SEQ ID NO: 1283 - RNA, 115 nt, synthetic construct
  misc_feature 1..115: note = Synthetic Polynucleotide
  misc_feature 1..115: note = mi1155
  source 1..115: mol_type = other RNA
ctcgagtgagcgacaggcgcaacctctcctagaactgtaaagccacagatgggttctaggagaggttgcgcctgctgcctactagatatttagtgtgataatggtgtttactagt

SEQ ID NO: 1284 - RNA, 93 nt, synthetic construct
  misc_feature 1..93: note = Synthetic Polynucleotide
  misc_feature 1..93: note = mi405
  source 1..93: mol_type = other RNA
ctcgagtgagcgatccaggattcagatctggtttctacgagccttttgctcgtcttatggaaaccagatctgaatcctggactgcctactagt

SEQ ID NO: 1285 - RNA, 93 nt, synthetic construct
  misc_feature 1..93: note = Synthetic Polynucleotide
  misc_feature 1..93: note = mi1155
  source 1..93: mol_type = other RNA
ctcgagtgagcgacaggcgcaacctctcctagaactacgagccttttgctcgtcttatggttctaggagaggttgcgcctgctgcctactagt

SEQ ID NO: 1286 - RNA, 114 nt, synthetic construct
  misc_feature 1..114: note = Synthetic Polynucleotide
  misc_feature 1..114: note = mi405
  source 1..114: mol_type = other RNA
ctcgagacgagccttttgctcgtcttatctcgggtgagcgatccaggattcagatctggtttctgtaaagccacagatgggaaaccagatctgaatcctggactgcctactagt

SEQ ID NO: 1287 - RNA, 114 nt, synthetic construct
  misc_feature 1..114: note = Synthetic Polynucleotide
  misc_feature 1..114: note = mi1155
  source 1..114: mol_type = other RNA
ctcgagacgagctttttgctcgtcttatctcgggtgagcgacaggcgcaacctctcctagaactgtaaagccacagatgggttctaggagaggttgcgcctgctgcctactagt

SEQ ID NO: 1288 - RNA, 114 nt, synthetic construct
  misc_feature 1..114: note = Synthetic Polynucleotide
  misc_feature 1..114: note = mi405
  source 1..114: mol_type = other RNA
ctcgagtgagcgatccaggattcagatctggtttctgtaaagccacagatgggaaaccagatctgaatcctggactgcctactagaacgagccttttgctcgtcttatactagt

SEQ ID NO: 1289 - RNA, 114 nt, synthetic construct
  misc_feature 1..114: note = Synthetic Polynucleotide
  misc_feature 1..114: note = mi1155
  source 1..114: mol_type = other RNA
ctcgagtgagcgacaggcgcaacctctcctagaactgtaaagccacagatgggttctaggagaggttgcgcctgctgcctactagaacgagccttttgctcgtcttatactagt

SEQ ID NO: 1290 - RNA, 143 nt, synthetic construct
  misc_feature 1..143: note = Synthetic Polynucleotide
  misc_feature 1..143: note = mi405
  source 1..143: mol_type = other RNA
ctcgagtatttagtgtgataatggtgtttctcgggtgagcgatccaggattcagatctggtttctgtaaagccacagatgggaaaccagatctgaatcctggactgcctactagaacgagccttttgctcgtcttatactagt

SEQ ID NO: 1291 - RNA, 143 nt, synthetic construct
  misc_feature 1..143: note = Synthetic Polynucleotide
  misc_feature 1..143: note = mi1155
  source 1..143: mol_type = other RNA
ctcgagtatttagtgtgataatggtgtttctcgggtgagcgacaggcgcaacctctcctagaactgtaaagccacagatgggttctaggagaggttgcgcctgctgcctactagaacgagccttttgctcgtcttatactagt

SEQ ID NO: 1292 - RNA, 137 nt, synthetic construct
  misc_feature 1..137: note = Synthetic Polynucleotide
  misc_feature 1..137: note = mi405
  source 1..137: mol_type = other RNA
ctcgagtatttagtgtgataatggtgtttacgagccttttgctcgtcttatctcgggtgagcgatccaggattcagatctggtttctgtaaagccacagatgggaaaccagatctgaatcctggactgcctactagt

SEQ ID NO: 1293 - RNA, 137 nt, synthetic construct
  misc_feature 1..137: note = Synthetic Polynucleotide
  misc_feature 1..137: note = mi1155
  source 1..137: mol_type = other RNA
ctcgagtatttagtgtgataatggtgtttacgagccttttgctcgtcttatctcgggtgagcgacaggcgcaacctctcctagaactgtaaagccacagatgggttctaggagaggttgcgcctgctgcctactagt

SEQ ID NO: 1294 - RNA, 137 nt, synthetic construct
  misc_feature 1..137: note = Synthetic Polynucleotide
  misc_feature 1..137: note = mi405
  source 1..137: mol_type = other RNA
ctcgagtgagcgatccaggattcagatctggtttctgtaaagccacagatgggaaaccagatctgaatcctggactgcctactagatatttagtgtgataatggtgtttacgagccttttgctcgtcttatactagt

SEQ ID NO: 1295 - RNA, 137 nt, synthetic construct
  misc_feature 1..137: note = Synthetic Polynucleotide
  misc_feature 1..137: note = mi1155
  source 1..137: mol_type = other RNA
ctcgagtgagcgacaggcgcaacctctcctagaactgtaaagccacagatgggttctaggagaggttgcgcctgctgcctactagatatttagtgtgataatggtgtttacgagccttttgctcgtcttatactagt

SEQ ID NO: 1296 - RNA, 122 nt, synthetic construct
  misc_feature 1..122: note = Synthetic Polynucleotide
  misc_feature 1..122: note = mi405
  source 1..122: mol_type = other RNA
ctcgagacgagccttttgctcgtcttatctcgggtgagcgatccaggattcagatctggtttcttatttagtgtgataatggtgtttggaaaccagatctgaatcctggactgcctactagt

SEQ ID NO: 1297 - RNA, 122 nt, synthetic construct
  misc_feature 1..122: note = Synthetic Polynucleotide
  misc_feature 1..122: note = mi1155
  source 1..122: mol_type = other RNA
ctcgagacgagccttttgctcgtcttatctcgggtgagcgacaggcgcaacctctcctagaacttatttagtgtgataatggtgtttggttctaggagaggttgcgcctgctgcctactagt

SEQ ID NO: 1298 - RNA, 122 nt, synthetic construct
  misc_feature 1..122: note = Synthetic Polynucleotide
  misc_feature 1..122: note = mi405
  source 1..122: mol_type = other RNA
ctcgagtgagcgatccaggattcagatctggtttcttatttagtgtgataatggtgtttggaaaccagatctgaatcctggactgcctactagaacgagccttttgctcgtcttatactagt

SEQ ID NO: 1299 - RNA, 122 nt, synthetic construct
  misc_feature 1..122: note = Synthetic Polynucleotide
  misc_feature 1..122: note = mi1155
  source 1..122: mol_type = other RNA
ctcgagtgagcgacaggcgcaacctctcctagaacttatttagtgtgataatggtgtttggttctaggagaggttgcgcctgctgcctactagaacgagccttttgctcgtcttatactagt

SEQ ID NO: 1300 - RNA, 122 nt, synthetic construct
  misc_feature 1..122: note = Synthetic Polynucleotide
  misc_feature 1..122: note = mi405
  source 1..122: mol_type = other RNA
ctcgagtatttagtgtgataatggtgtttctcgggtgagcgatccaggattcagatctggtttctacgagccttttgctcgtcttatggaaaccagatctgaatcctggactgcctactagt

SEQ ID NO: 1301 - RNA, 122 nt, synthetic construct
  misc_feature 1..122: note = Synthetic Polynucleotide
  misc_feature 1..122: note = mi1155
  source 1..122: mol_type = other RNA
ctcgagtatttagtgtgataatggtgtttctcgggtgagcgacaggcgcaacctctcctagaactacgagccttttgctcgtcttatggttctaggagaggttgcgcctgctgcctactagt

SEQ ID NO: 1302 - RNA, 122 nt, synthetic construct
  misc_feature 1..122: note = Synthetic Polynucleotide
  misc_feature 1..122: note = mi405
  source 1..122: mol_type = other RNA
ctcgagtgagcgatccaggattcagatctggtttctacgagccttttgctcgtcttatggaaaccagatctgaatcctggactgcctactagatatttagtgtgataatggtgtttactagt

SEQ ID NO: 1303 - RNA, 122 nt, synthetic construct
  misc_feature 1..122: note = Synthetic Polynucleotide
  misc_feature 1..122: note = mi1155
  source 1..122: mol_type = other RNA
ctcgagtgagcgacaggcgcaacctctcctagaactacgagccttttgctcgtcttatggttctaggagaggttgcgcctgctgcctactagatatttagtgtgataatggtgtttactagt

SEQ ID NO: 1304 - RNA, 115 nt, synthetic construct
  misc_feature 1..115: note = Synthetic Polynucleotide
  misc_feature 1..115: note = mi405
  source 1..115: mol_type = other RNA
ctcgagtgagcgatccaggattcagatctggtttcttttagtgtgataatggtgtttgacgagctttttgctcgtcttatggaaaccagatctgaatcctggactgcctactagt

SEQ ID NO: 1305 - RNA, 115 nt, synthetic construct
  misc_feature 1..115: note = Synthetic Polynucleotide
  misc_feature 1..115: note = mi1155
  source 1..115: mol_type = other RNA
ctcgagtgagcgacaggcgcaacctctcctagaacttttagtgtgataatggtgtttgacgagctttttgctcgtcttatggttctaggagaggttgcgcctgctgcctactagt